(19) United States
(12) Patent Application Publication
(10) Pub. No.: US 2024/0158375 A1
(43) Pub. Date: May 16, 2024

(54) COMPOUNDS AND METHODS FOR THE TREATMENT OF OCULAR DISORDERS

(71) Applicant: Azura Ophthalmics Ltd., Tel Aviv (IL)

(72) Inventors: Ian HOLMES, Melbourne, Victoria (AU); Yair ALSTER, Tel Aviv (IL); Hila BARASH, Shoham (IL); Charles BOSWORTH, Las Vegas, NV (US); Omer RAFAELI, Udim (IL); Robert M. BURK, Laguna Beach, CA (US); Marc GLEESON, Longueville (AU); Mark Richard STEWART, Cambridge (GB); Soultana KATSINA, London (GB)

(21) Appl. No.: 18/033,053

(22) PCT Filed: Oct. 20, 2021
(86) PCT No.: PCT/IB2021/000723

§ 371 Date: Apr. 20, 2023

Related U.S. Application Data
(60) Provisional application No. 63/094,791, filed on Oct. 21, 2020.

(51) Int. Cl. C07D 405/12; C07C 321/14; C07D 211/62; C07D 339/04; C07D 401/12; C07D 401/14; C07D 409/12; C07D 417/12

(52) U.S. Cl. CPC C07D 405/12; C07C 321/14; C07D 211/62; C07D 339/04; C07D 401/12; C07D 401/14; C07D 409/12; C07D 417/12

(57) ABSTRACT

Described herein are compositions and methods for the treatment or prevention of ocular surface disorders including meibomian gland dysfunction, blepharitis, dry eye disease and other inflammatory and/or infectious diseases of the anterior surface of the eye(s). Said compositions and methods comprise keratolytic conjugates which demonstrate keratolytic activity, and anti-inflammatory or other desirable activities. Topical administration of said compositions to the eyelid margin or surrounding areas provides therapeutic benefit to patients suffering from ocular surface disorders.

### CROSS-REFERENCE TO RELATED APPLICATIONS

This application claims the benefit of U.S. Provisional Application No. 63/094,791, filed Oct. 21, 2020, the content of which is incorporated by reference herein in its entirety.

### BACKGROUND OF THE DISCLOSURE

Restasis (0.05% cyclosporine A, Allergan) was approved by the Food and Drug Administration (FDA) to increase tear production in patients whose tear production is presumed to be suppressed due to ocular inflammation associated with keratoconjunctivitis sicca. Xiidra® (lifitegrast ophthalmic solution) 5% is indicated for the treatment of signs and symptoms of dry eye disease (DED).

### SUMMARY OF THE DISCLOSURE

Provided in certain embodiments herein are compounds, pharmaceutical (e.g., ophthalmic) compositions, and methods of treatment. In specific embodiments, methods of treatment provided herein include the treatment of ocular and/or periocular indications or abnormalities. In some embodiments, the ocular and/or periocular indications or abnormalities treated by or with a composition or compound provided herein are indications or abnormalities that have multifactorial etiologies and/or interactions. In certain embodiments provided herein are compounds (and compositions comprising such compounds) that have multifunctional efficacies, such as when administered in or around the eye (e.g., to the ocular surface, the eyelid, such as the eyelid margin or the inner surface of the eyelid, or the like).

In some embodiments, provided herein is a method of treating inflammation or hyperkeratosis (e.g., of the eye or skin).

In certain embodiments, methods provided herein involve the method of treating meibomian gland dysfunction (MGD).

Currently there are no approved pharmacological agents useful for the treatment of MGD. The recognition that terminal duct obstruction from hyperkeratinization of the ductal epithelium on meibomian glands is a core mechanism behind meibomian gland dysfunction (MGD) is consistent with clinical experience demonstrating that effective treatments for MGD require resolution of ductal obstruction and evacuation of glandular contents (Nichols et al, 2011; Lane et al, 2012; Blackie et al, 2015). Warm compresses and thermal/mechanical devises (e.g., LipiFlow) are used in an attempt to raise the internal temperature of the meibomian glands over the normal melting point for meibum (i.e., 32° C. to 40° C.) in an attempt to resolve terminal duct obstruction (Lane ne et al, 2012). Unfortunately, warm compresses are unable to achieve this benefit for severely obstructed glands which can having a melting point>40° C. Current technology for removing keratinized obstruction of the meibomian gland also includes physical removal methods (e.g., debridement and gland probing), which are quite painful to patients.

Subsequent to a period of MGD, various stages of inflammatory or bacterial disease at the ocular surface are frequently observed because meibomian gland obstruction can cause a cascade of events that include further deterioration of the glands (Knop, IOVS, 2011) from stasis of the meibum in the secretory glands, mechanical pressure and stress from glandular obstruction, and increased bacterial growth that is associated with the downstream release of bacterial lipases, toxic mediators, and/or inflammatory mediators. All these factors reduce the quality and/or quantity of meibum the glands can release which in turn can cause chronic mechanical traumatization of the conjunctival, corneal and eyelid tissues which will lead to further tissue damage and the release of inflammatory mediators. Thus, many patients suffering from MGD also have inflammatory disease affecting their conjunctiva, cornea, larcrimal gland, lids or goblet cells causing comorbid conditions such as dry eye syndrome or blepharitis for which there is an unmet medical need.

For example, literature has used the terms posterior blepharitis and MGD as if they were synonymous, but these terms are not interchangeable. Posterior blepharitis describes inflammatory conditions of the posterior lid margin, of which MGD can be one possible cause. In its earliest stages, MGD may not be associated with clinical signs characteristic of posterior blepharitis. At this stage, affected individuals may be symptomatic, but alternatively, they may be asymptomatic and the condition regarded as subclinical. As MGD progresses, symptoms develop and lid margin signs, such as changes in meibum expressibility and quality and lid margin redness, may become more visible. At this point, an MGD-related posterior blepharitis is said to be present.

In certain embodiments, provided herein are methods of treating ocular (or dermatological) disorders associated with keratosis (e.g., lid keratosis, surface ocular keratosis, and/or gland blockage—such as in MGD), microbial infiltration/infection (e.g., bacterial infiltration/infection), and/or inflammation (such as inflammation associated keratosis or not associated with keratosis). In certain instances, disorders of the skin and/or eye (and/or surround tissue/skin) are difficult to differentially diagnose and/or have multiple etiologies. For example, in some instances, it can be difficult to distinguish between ocular disorders that involve (1) inflammation only, (2) inflammation associated with keratolytic activity, (3) inflammation associated with both keratolytic activity (e.g., inducing keratosis) and microbial infiltration, (4) keratolytic activity, but not inflammation and/or microbial infiltration, or various other combinations. In some instances, compounds and compositions provided herein can be used in such ocular and/or dermatological indications without the need for differential diagnosis (which can be difficult, e.g., because of similar symptom scores, etc.). Further, many ocular and/or dermatological disorders involve multiple etiologies, such inflammation, microbial infiltration, keratolytic activity, or various combinations thereof. As a result, therapeutic agents, such as those described herein, that target multiple etiologies are beneficial in providing therapeutic efficacy, such as by targeting both an underlying condition (e.g., keratolytic activity and/or microbial infiltration) and a symptom, such as inflammation or dry eye.

As such, provided herein are compounds, compositions, methods, and formulations for the treatment of ocular (e.g., periocular) or dermatological disorders, such as those having abnormalities having multifactorial etiologies. In specific embodiments, ocular disorders include, by way of non-limiting example, surface disorders, such as MGD, dry eye and associated inflammatory and bacterial disease.

In some embodiments provided herein is a compound, or a pharmaceutically acceptable salt or solvate (e.g., or a stereoisomer) thereof, having the structure of Formula (I):

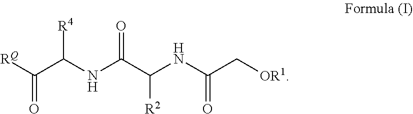

In some embodiments, R¹ is aryl, cycloalkyl, heterocyclyl, or heteroaryl, wherein the aryl, cycloalkyl, heterocyclyl, or heteroaryl is optionally substituted. In some embodiments, R² is substituted or unsubstituted alkyl. In some embodiments, R⁴ is -L^{a}-R^{4x}, wherein L^{a} is a bond, alkylene, or heteroalkylene, and R^{4X} is absent, substituted or unsubstituted cycloalkyl, substituted or unsubstituted heterocycloalkyl, substituted or unsubstituted aryl, or substituted or unsubstituted heteroaryl. In some embodiments, R^{Q} is -L′-D. In some embodiments, D is a keratolytic agent (e.g., or a radical thereof). In some embodiments, L′ is a (e.g., hydrolyzable) linker.

In some embodiments, L′ comprises one or more linker group, each linker group being independently selected from the group consisting of a bond, oxo, —O—, —S—, halo, optionally substituted alkyl(alkylenyl), optionally substituted heteroalkyl(heteroalkylenyl), disulfide, ester, and amide. In some embodiments, L′ comprises one or more linker group, each linker group is independently optionally substituted alkyl(alkylene) or optionally substituted heteroalkyl (heteroalkylene). In some embodiments, L′ comprises one or more linker group, each linker group being independently selected from the group consisting of a bond, —O—, —S—, optionally substituted alkyl (alkylenyl), and optionally substituted heteroalkyl (heteroalkylenyl). In some embodiments, L′ comprises one or more linker group, each linker group is independently optionally substituted alkyl (alkylene) or optionally substituted heteroalkyl (heteroalkylene).

In some embodiments, L′ is alkyl (alkylene) substituted with oxo and one or more of alkyl and heteroalkyl. In some embodiments, the alkyl or heteroalkyl is substituted with one or more halo, alkyl, or haloalkyl. In some embodiments, the alkyl or heteroalkyl is substituted with one or more alkyl or haloalkyl. In some embodiments, L′ comprises one or more linker group, each linker group being independently selected from a bond, —O—, —S—, halo, (C═O), —(C═O)alkyl-, —(C═O)heteroalkyl-, —(C═O)O—, —(C═O)Oalkyl-, —(C═O)Oheteroalkyl-, —(C═O)S—, —(C═O)Salkyl-, —(C═O)Sheteroalkyl-, alkylene, or heteroalkylene, where each alkyl, heteroalkyl, alkylene, or heteroalkyl is independently optionally substituted. In some embodiments, L′ comprises one or more linker group, each linker group being independently selected from a bond, —O—, —S—, —(C═O)alkyl-, —(C═O)heteroalkyl-, —(C═O)Oalkyl-, —(C═O)Oheteroalkyl-, —(C═O)Salkyl-, —(C═O)Sheteroalkyl-, alkylene, or heteroalkylene, where each alkyl, heteroalkyl, alkylene, or heteroalkyl is independently optionally substituted. In some embodiments, L′ comprises one or more linker group, each linker group being independently selected from —O—, (C═O), —(C═O)alkyl-, —(C═O)heteroalkyl-, —(C═O)O—, —(C═O)Oalkyl-, —(C═O)Oheteroalkyl-, —(C═O)OalkylO—, —(C═O)OheteroalkylO—, —(C═O)S—, —(C═O)S alkyl-, —(C═O)Sheteroalkyl-, alkylene, and heteroalkylene. In some embodiments, L′ comprises —O—, —(C═O)alkyl-, —(C═O)O—, —(C═O)Oalkyl-, and/or —(C═O)OalkylO—. In some embodiments, L′ comprises one or more linker group, each linker group being independently selected from —O—, —(C═O)alkyl-, —(C═O)heteroalkyl-, —(C═O)Oalkyl-, —(C═O)Oheteroalkyl-, —(C═O)OalkylO—, —(C═O)OheteroalkylO—, —(C═O)Salkyl-, —(C═O)Sheteroalkyl-, alkylene, and heteroalkylene. In some embodiments, L′ comprises —O—, —(C═O)alkyl-, —(C═O)Oalkyl-, and/or —(C═O)OalkylO—.

In some embodiments, the linker comprises the structure of Formula (A):

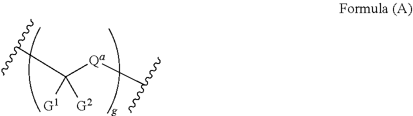

wherein:
   Q^{a} is a bond, —O—, —S—, or optionally substituted amino;
   each G¹ and G² is independently hydrogen, halo, alkyl, heteroalkyl, or cycloalkyl, wherein the alkyl or cycloalkyl is optionally substituted; and
   g is 1-20.

In some embodiments, the compound comprises more than one linker of Formula (A). In some embodiments, Q^{a} is a bond or —O—. In some embodiments, Q^{a} is —O— and each G¹ and G² is independently hydrogen, alkyl, or cycloalkyl, wherein the alkyl or cycloalkyl are optionally substituted. In some embodiments, Q^{a} is a bond or —O— and each G¹ is hydrogen and each G² is independently alkyl or haloalkyl. In some embodiments, Q^{a} is a bond or —O— and each G¹ is hydrogen and each G² is methyl. In some embodiments, Q^{a} is a bond or —O— and each G¹ and G² is hydrogen. In some embodiments, Q^{a} is —O—, each G¹ is hydrogen, and each G² is methyl. In some embodiments, Q^{a} is —O— and each G¹ and G² is hydrogen.

In some embodiments, g is 1-20. In some embodiments, g is 1-10. In some embodiments, g is 1-5. In some embodiments, g is 2. In some embodiments, g is 1.

In some embodiments, g is 1 or 2, Q^{a} is a bond and each G¹ is hydrogen, and each G² is methyl. In some embodiments, g is 1 or 2, Q^{a} is a bond, and each G¹ and G² is hydrogen. In some embodiments, g is 1 or 2, Q^{a} is —O—, each G¹ is hydrogen, and each G² is methyl. In some embodiments, g is 1 or 2, Q^{a} is —O—, and each G¹ and G² is hydrogen.

In some embodiments, the linker comprises one or more bond, —O—, methylene,

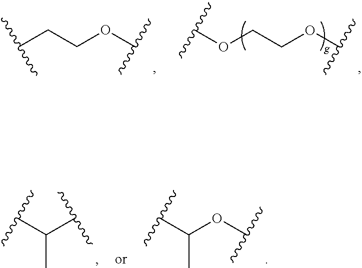

In some embodiments, g is 1-20. In some embodiments, g is 1-10. In some embodiments, g is 1-8. In some embodiments, g is 1, 2, 3, 4, 5, 6, 7, or 8.

In some embodiments, the linker comprises one or more of:

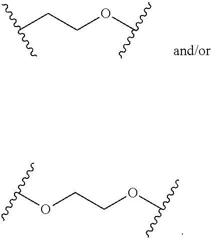 [SMILES: *CCO* ; *OCCO*]

In some embodiments, the linker comprises a bond, methylene,

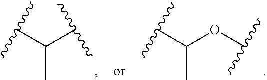

In some embodiments, the linker comprises:

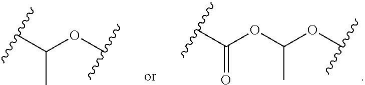

In some embodiments, the linker comprises:

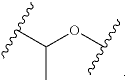 [SMILES: *OC(*)C]

In some embodiments, the linker is:

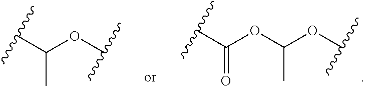

In some embodiments, any linker or L provided herein is attached to the rest of a molecule provided herein to form a ketal. In some embodiments, any linker or L provided herein is attached to the rest of a molecule provided herein to form an ester.

In some embodiments, D comprises a radical of one or more keratolytic group (e.g., each radical of the one or more keratolytic group being independently selected from the group consisting of a radical of glycolic acid (GA), a radical of thioglycolic acid (TGA), a radical of lactic acid (Lac), a radical of thiolactic acid (TLac), a radical of lipoic acid (Lip), a radical of lipoic acid sulfoxide (Lipox), a radical of dihydrolipoic acid (diHLip), a radical of N-acetyl cysteine (NAC), a radical of cysteine (Cys), a radical of glutathione (GSH), a radical of captopril (Cap), and a radical of bucillamine (Buc)).

In some embodiments, D comprises a radical of one or more keratolytic group, each radical of the one or more keratolytic group being independently selected from the group consisting of a radical of glycolic acid (GA), a radical of thioglycolic acid (TGA), a radical of lactic acid (Lac), a radical of thiolactic acid (TLac), a radical of lipoic acid (Lip), a radical of lipoic acid sulfoxide (Lipox), a radical of dihydrolipoic acid (diHLip), a radical of N-acetyl cysteine (NAC), a radical of cysteine (Cys), a radical of glutathione (GSH), a radical of captopril (Cap), and a radical of bucillamine (Buc).

In some embodiments, D comprises a thiol radical of one or more keratolytic group, each thiol radical of the one or more keratolytic group being independently selected from the group consisting of a thiol radical of thioglycolic acid (TGA), a thiol radical of thiolactic acid (TLac), a thiol radical of dihydrolipoic acid (diHLip), a thiol radical of N-acetyl cysteine (NAC), a thiol radical of cysteine (Cys), a thiol radical of glutathione (GSH), a thiol radical of captopril (Cap), and a thiol radical of bucillamine (Buc).

In some embodiments, the (e.g., thiol) radical of the keratolytic agent comprises a (e.g., thiol) radical of one or more keratolytic group, each (e.g., thiol) radical of the one or more keratolytic group being independently selected from the group consisting of [Lac-Lac]·, [Lac-NAC]·, [Cys-Cys]·, [diHLip-NAC-NAC]·, [diHLip-NAC]·, [diHLip-Cap-Cap]·, [diHLip-Cap]·, [diHLip-Cys-Cys]·, [diHLip-Cys]·, [diHLip-Lipox-Lipox]·, and [diHLip-Lipox]·.

In some embodiments, D is (e.g., linear or branched) unsubstituted or substituted alkoxy, (e.g., linear or branched) unsubstituted or substituted alkyl, (e.g., linear or branched) unsubstituted or substituted heteroalkyl, or unsubstituted or substituted heterocycloalkyl. In some embodiments, D is unsubstituted alkoxy. In some embodiments, D is unsubstituted alkyl.

In some embodiments, D is substituted alkyl, being substituted with one or more substituent, each substituent being independently selected from the group consisting of —OH, optionally substituted C₁-C₆ alkoxy (e.g., being optionally substituted with one or more substituent, each substituent being independently selected from the group consisting of oxo, —OH, C₁-C₄ alkoxy), —SH, optionally substituted (e.g., saturated) heterocycloalkyl (e.g., dithiolanyl or dithiolanyl oxide), optionally substituted C₁-C₄ heteroalkyl (e.g., —NH(C═O)C₁-C₄ alkyl), substituted or unsubstituted (e.g., unsaturated) cycloalkyl (e.g., being substituted with one or more C₁-C₄ alkyl), and amino.

In some embodiments, the substituted heterocycloalkyl is saturated (e.g., dithiolanyl, dithiolanyl sulfone, or dithiolanyl oxide).

In some embodiments, D is substituted alkyl, being substituted with one or more substituent, each substituent being independently selected from the group consisting of —OH, optionally substituted C₁-C₆ alkoxy (e.g., being optionally substituted with one or more substituent, each substituent being independently selected from the group consisting of oxo, —OH, C₁-C₄ alkoxy), —SH, optionally substituted (saturated) heterocycloalkyl (e.g., dithiolanyl or dithiolanyl oxide), optionally substituted C₁-C₄ heteroalkyl (e.g., —NH(C═O)C₁-C₄ alkyl), and amino.

In some embodiments, D is substituted (e.g., linear or branched) alkoxy, being substituted with one or more substituent, each substituent being independently selected from the group consisting of oxo, C₁-C₄ alkyl, C₁-C₄ alkoxy, optionally substituted heterocycloalkyl (e.g., 1,3-dioxanyl being optionally substituted with C₁-C₄ alkyl), —OH (e.g., substituted branched alkoxy being substituted with one or more —OH), substituted unsaturated cycloalkyl (e.g., being substituted with one or more C₁-C₄ alkyl), and optionally substituted C₁-C₄ heteroalkyl (e.g., substituted branched alkoxy being substituted with one or more —O(C═O)C₁-C₄ alkyl, wherein the C₁-C₄ alkyl is further optionally substituted (e.g., with —OH)).

In some embodiments, D is unsubstituted or substituted (e.g., N-substituted) heterocycloalkyl, being substituted with optionally substituted alkyl (e.g., being optionally substituted with one or more substituent, each substituent being independently selected from the group consisting of oxo, C₁-C₆ alkyl, and —SH).

In some embodiments, D comprises:

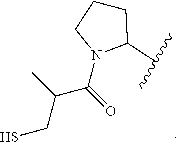 [SMILES: *C1CCCN1C(=O)C(C)CS]

In some embodiments, D comprises:

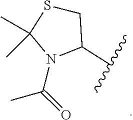 [SMILES: *C1CSC(C)(C)N1C(C)=O]

In some embodiments, D comprises:

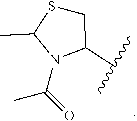 [SMILES: *C1CSC(C)N1C(C)=O]

In some embodiments, D comprises:

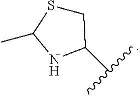 [SMILES: *C1CSC(C)N1]

In some embodiments, D is substituted or unsubstituted (e.g., linear or branched) heteroalkyl comprising one or more —C—O—C— (e.g., within the (e.g., linear or branched) heteroalkyl chain).

In some embodiments, D is substituted or unsubstituted (e.g., linear or branched) heteroalkyl comprising one or more ester, one or more carbonate, one or more amide, and/or one or more disulfide (e.g., within the (e.g., linear or branched) heteroalkyl chain).

In some embodiments, D is substituted or unsubstituted (e.g., linear or branched) heteroalkyl comprising one carbonate (e.g., within the (e.g., linear or branched) heteroalkyl chain).

In some embodiments, D is substituted or unsubstituted (e.g., linear or branched) heteroalkyl comprising one or two ester (e.g., within the (e.g., linear or branched) heteroalkyl chain).

In some embodiments, D is substituted or unsubstituted (e.g., linear or branched) heteroalkyl comprising one ester (e.g., within the (e.g., linear or branched) heteroalkyl chain).

In some embodiments, D is substituted or unsubstituted (e.g., linear or branched) heteroalkyl comprising one ester and one carbonate (e.g., within the (e.g., linear or branched) heteroalkyl chain).

In some embodiments, D is substituted or unsubstituted (e.g., linear or branched) heteroalkyl comprising one or two ester and one amide (e.g., within the (e.g., linear or branched) heteroalkyl chain).

In some embodiments, D is substituted or unsubstituted (e.g., linear or branched) heteroalkyl comprising one ester and one amide (e.g., within the (e.g., linear or branched) heteroalkyl chain).

In some embodiments, D is substituted or unsubstituted (e.g., linear or branched) heteroalkyl comprising one or two amide (e.g., within the (e.g., linear or branched) heteroalkyl chain).

In some embodiments, D is substituted or unsubstituted (e.g., linear or branched) heteroalkyl comprising one or two disulfide (e.g., within the (e.g., linear or branched) heteroalkyl chain).

In some embodiments, D is substituted or unsubstituted (e.g., linear or branched) heteroalkyl comprising one or two disulfide and one ester (e.g., within the (e.g., linear or branched) heteroalkyl chain).

In some embodiments, D is (e.g., linear or branched) unsubstituted or substituted heteroalkyl, being substituted with one or more substituent, each substituent being independently selected from the group consisting of thioalkyl (e.g., —CH₂SH), amino, —COOH, —SH, C₁-C₄ alkyl, acetamide, and optionally substituted heterocylcoalkyl (e.g., N-attached heterocycloalkyl substituted with COOH)

In some embodiments, D is substituted branched heteroalkyl.

In some embodiments, D comprises:

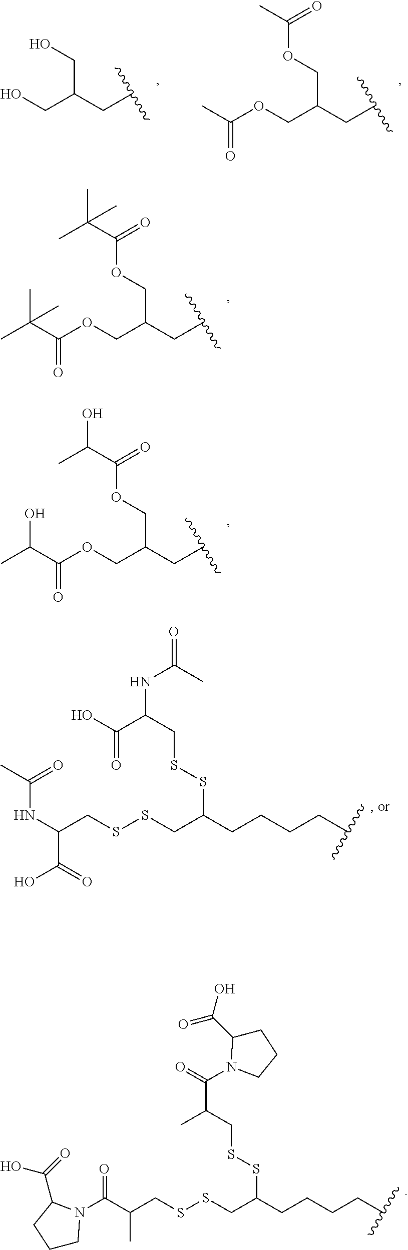 [SMILES: *CC(CO)CO ; *CC(COC(C)=O)COC(C)=O ; *CC(COC(=O)C(C)(C)C)COC(=O)C(C)(C)C ; *CC(COC(=O)C(C)O)COC(=O)C(C)O ; *CCCCC(CSSCC(NC(C)=O)C(=O)O)SSCC(NC(C)=O)C(=O)O ; *CCCCC(CSSCC(C)C(=O)N1CCCC1C(=O)O)SSCC(C)C(=O)N1CCCC1C(=O)O]

In some embodiments, D comprises: HOCH₂—, HOCH(CH₃)—, HO(CH₂CH₂O)₄CH₂—, HO(CH₂CH₂O)₄CH₂CH₂—, HOCH₂ (C═O)O—, HOCH(CH₃)(C═O)O—, HO(CH₂CH₂O)₄CH₂ (C═O)O—, HO(CH₂CH₂O)₄CH₂CH₂ (C═O)O—, CH₃O(C═O)O—, CH₃CH₂O(C═O)O—, (CH₃)₂CO(C—O)O—, (CH₃)₃CO(C—O)O—, CH₃ (C═O)O—, CH₃CH₂ (C═O)O—, (CH₃)₂C(C═O)O—, (CH₃)₃C(C═O)O—, HOCH₂ (C═O)O—, HO(CH₃) CH(C═O)O—, HO(CH₃) CH(C═O)O(CH₃) CH(C═O)O—, CH₃ (C═O)O(CH₃) CH(C═O)O—, CH₃O(C═O)O(CH₃) CH(C═O)O—, CH₃O(C═O)(CH₃) CHO(C═O)O—, CH₃CH₂O(C═O)(CH₃) CHO(C═O)O—, HOCH₂ (HOCH₂)CHCH₂O(C═O)O—, CH₃ (C═O)OCH₂ (CH₃(C═O)OCH₂)CHCH₂O(C═O)O—, (CH₃)₃C(C═O)OCH₂ ((CH₃)₃C(C═O)OCH₂)CHCH₂O(C═O)O—, HO(CH₃) CH(C═O)OCH₂ (HO(CH₃) CH(C═O)OCH₂)CHCH₂O(C═O)O—, HSCH₂ (C═O)O—, HS(CH₃) CH(C═O)O—, HSCH₂ (NH₂)CH(C═O)O—, HSCH₂ (CH₃(C═O)NH)CH(C═O)O—, HOOC(NH₂)CHCH₂CH₂ (C═O)NH(HSCH₂)CH(C═O)NHCH₂ (C═O)O—, O(C═O)CH(NH₂)CH₂CH₂ (C═O)NHCH(CH₂SH)(C═O)NHCH₂COOH, HS(CH₃)₂C(C═O)NH(SHCH₂)CH(C═O)O—, HOOC(NH₂)CHCH₂S SCH₂CH(NH₂)(C═O)O—, HSCH₂ (CH₃(C═O)NH)CH(C═O)OCH(CH₃)(C═O)O—,

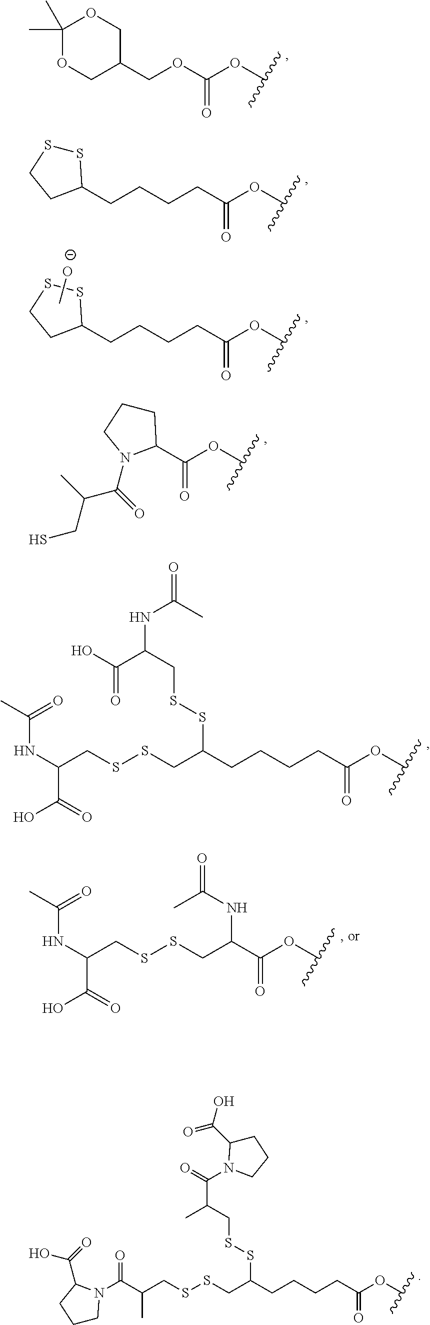

In some embodiments, D comprises:

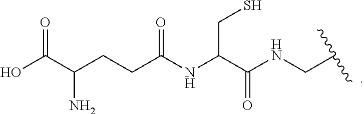 [SMILES: *CNC(=O)C(CS)NC(=O)CCC(N)C(=O)O]

In some embodiments, D comprises:

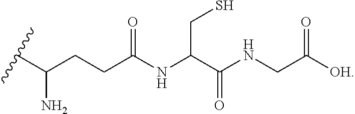 [SMILES: *C(N)CCC(=O)NC(CS)C(=O)NCC(=O)O]

In some embodiments, D comprises:

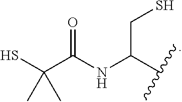 [SMILES: *C(CS)NC(=O)C(C)(C)S]

In some embodiments, D comprises:

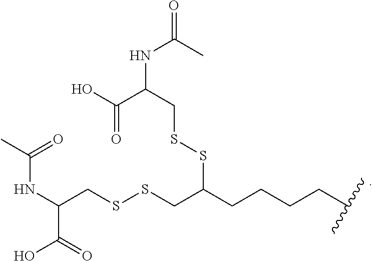 [SMILES: *CCCCC(CSSCC(NC(C)=O)C(=O)O)SSCC(NC(C)=O)C(=O)O]

In some embodiments, D comprises:

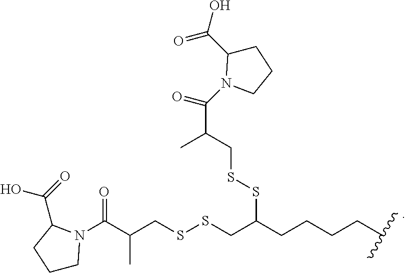 [SMILES: *CCCCC(CSSCC(C)C(=O)N1CCCC1C(=O)O)SSCC(C)C(=O)N1CCCC1C(=O)O]

In some embodiments, D comprises:

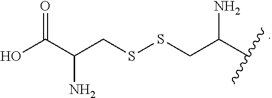 [SMILES: *C(N)CSSCC(N)C(=O)O]

In some embodiments, D comprises:

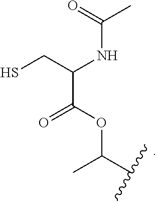 [SMILES: *C(C)OC(=O)C(CS)NC(C)=O]

In some embodiments, D comprises:

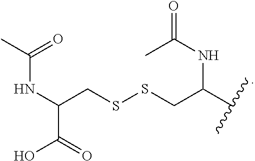 [SMILES: *C(CSSCC(NC(C)=O)C(=O)O)NC(C)=O]

In some embodiments, D comprises:

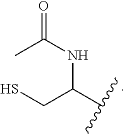 [SMILES: *C(CS)NC(C)=O]

In some embodiments, D-L′ is:

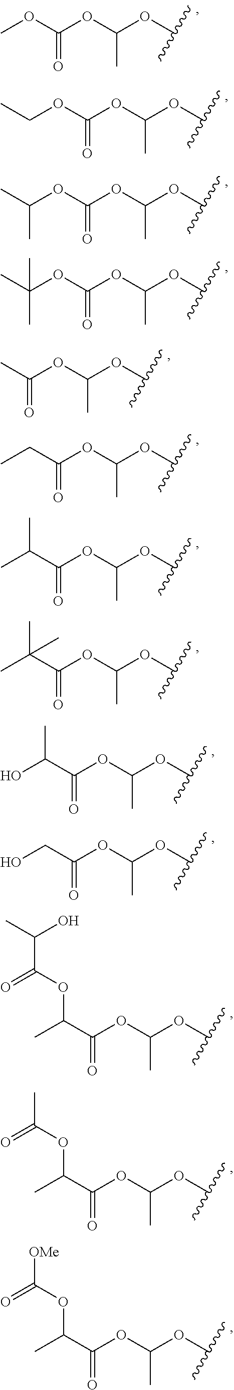

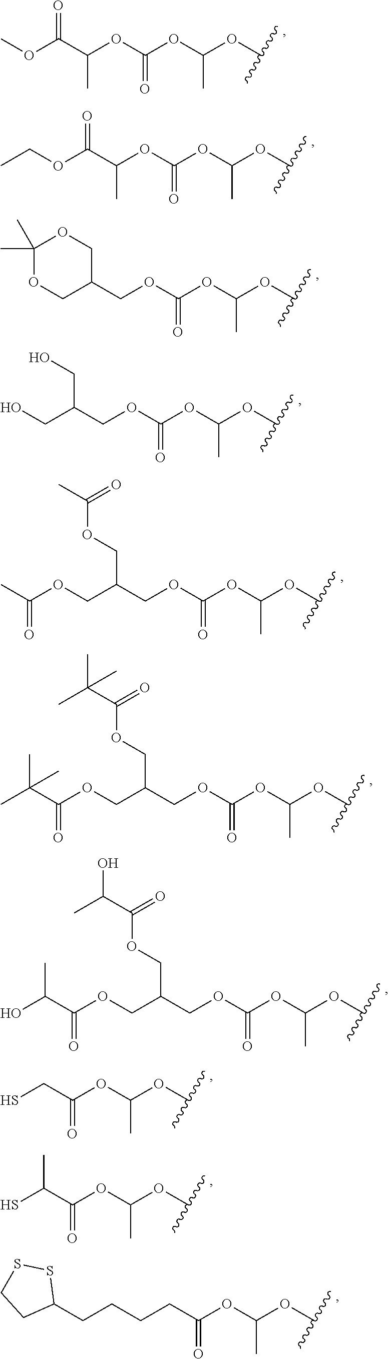

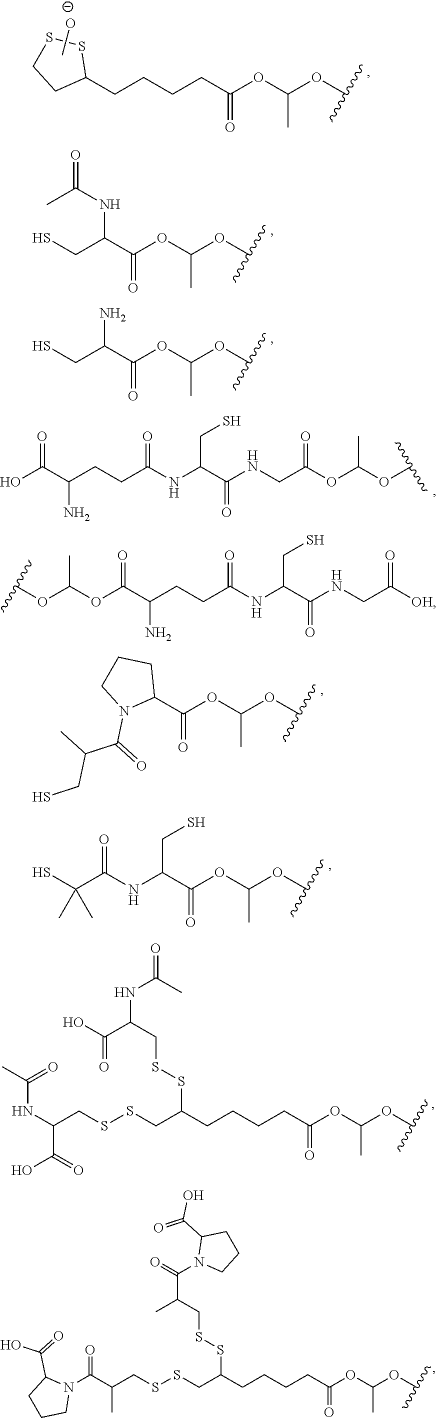

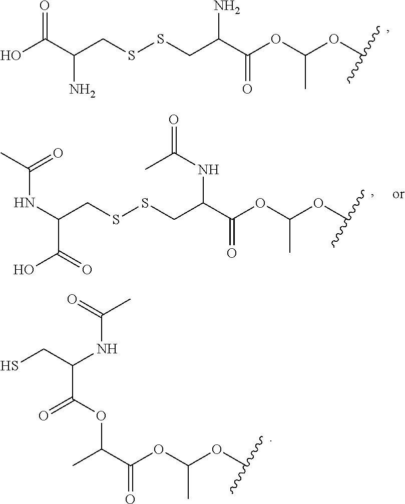

In some embodiments, D is a “keratolytic agent” radical that, upon release, hydrolysis, or other mechanism metabolizes or otherwise produces (e.g., when administered to an individual or patient, such as in or around the eye, such as the eyelid margin) an active keratolytic agent (e.g., a carboxylic acid and/or a thiol). In some instances, upon release (e.g., by hydrolysis or other mechanism), D produces a plurality of active keratolytic agents. In some instances, the active keratolytic agent comprises one or more of —SH, —OH, COOH (or COO—), or disulfide. In some embodiments, the active keratolytic agent is a carboxylic acid. In some embodiments, the active keratolytic agent is selected from the group consisting of acetic acid, glycolic acid, lactic acid, lipoic acid, pivalic acid, isobutryic acid, butyric acid, propionic acid, formic acid, and carbonic acid. In some embodiments, the active keratolytic agent is a thiol. In some embodiments, the active keratolytic agent is a carboxylic acid.

In some embodiments, one or more group of the keratolytic agent (e.g., thiol, hydroxy, carboxylic acid, amide, or amine) is protected or masked (e.g., with optionally substituted C₁-C₆ alkyl (e.g., being optionally substituted with oxo)). In some embodiments, one or more thiol of the keratolytic agent is protected or masked with acetyl. In some embodiments, one or more amine of the keratolytic agent is protected or masked with acetyl. In some embodiments, one or more carboxylic acid of the keratolytic agent is protected or masked with methyl, ethyl, propyl, isopropyl, or t-butyl. In some embodiments, one or more carboxylic acid of the keratolytic agent is protected or masked with ethyl.

In some embodiments, L^{a} is attached to D by a bond.

In some embodiments, any L or linker provided herein comprises one or more substituted or unsubstituted alkoxy (e.g., polyethylene glycol (PEG)).

In some embodiments, any L or linker provided herein comprises a compound having a structure of Formula (B):

—(CH₂CH₂O)_{B}X—.

In some embodiments, X is a bond or (C═O). In some embodiments, X is a bond. In some embodiments, X is (C═O).

In some embodiments, b is an integer from 1-20. In some embodiments, b is an integer from 1-10. In some embodiments, b is an integer from 1-5. In some embodiments, b is 1, 2, 3, 4, 5, 6, 7, 8, 9, or 10. In some embodiments, b is 4. In some embodiments, b is 8.

In some embodiments, any L or linker provided herein is attached to the compound having a structure of Formula (B). In some embodiments, the linker is —O(C═O)(OCR⁸R⁹)_{z}—. In some embodiments, the linker is —O(C═O)OCH(CH₃)—. In some embodiments, the linker is —CH(CH₃) O(C═O)O— and attached to the compound having a structure of Formula (B).

In some embodiments, the linker is —CH(CH₃) O(C═O)O— and attached to —(CH₂CH₂O)₄(C═O)—. In some embodiments, the linker is —CH(CH₃) O(C═O)O— and attached to —(CH₂CH₂O)₄—. In some embodiments, the linker is —CH(CH₃) O(C═O)O— and attached to —(CH₂CH₂O)₈(C═O)—. In some embodiments, the linker is —CH(CH₃) O(C═O)O— and attached to —(CH₂CH₂O)₈—.

In In some embodiments, the compound having the structure of Formula (B) is attached to a keratolytic agent provided herein (e.g., as described elsewhere herein). In some embodiments, the compound having the structure of Formula (B) is attached to and includes at least a portion of a keratolytic agent provided herein (e.g., as described elsewhere herein).

In some embodiments, the compound having the structure of Formula (B) is attached to any R or R′ provided herein (e.g., as described elsewhere herein).

In certain instances, provided herein is a combination of an anti-inflammatory and/or anti-microbial moiety (e.g., having a structure of any formula provided herein, minus the R′) with a keratolytic moieity (e.g., being represented by and/or having a structure of D). In certain embodiments, such moieties are radicals connected by a linker that is a bond, with the keratolytic moiety being hydrolyzable to produce both (1) an anti-inflammatory and (2) one or more active keratolytic agent. In some embodiments, such moieties are radicals connected by a hydrolyzable linker, with the hydrolyzable linker being hydrolyzable, such that both (1) an anti-inflammatory and (2) one or more active keratolytic agent are released (e.g., in vivo, such as after therapeutic (e.g., topical) delivery to the eye and/or skin).

In some embodiments, a compound provided herein comprises a first radical (e.g., a first radical of Formula I (or any other formula provided herein)) that is dimerized with a second radical (e.g., a second radical of Formula I (or any other formula provided herein)). In some embodiments, each radical of Formula I (or any other formula provided herein) is dimerized through an —SH group thereof (e.g., forming an S—S linkage).

In some embodiments, provided herein is a compound, having the structure of Formula (I′):

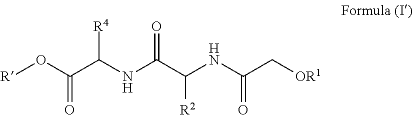

or a pharmaceutically acceptable salt or solvate (e.g., or a stereoisomer) thereof,
wherein:
   R¹ is aryl, cycloalkyl, heterocyclyl, or heteroaryl, wherein the aryl, cycloalkyl, heterocyclyl, or heteroaryl is optionally substituted;
   R² is substituted or unsubstituted alkyl;
   R⁴ is L^{a}_R^{4x}, wherein L^{a} is a bond, alkylene, or heteroalkylene, and R^{4X} is absent, substituted or unsubstituted cycloalkyl, substituted or unsubstituted heterocycloalkyl, substituted or unsubstituted aryl, or substituted or unsubstituted heteroaryl; and
R′ is alkyl substituted with at least one oxo or heteroalkyl substituted with at least one oxo, the alkyl or heteroalkyl being further optionally substituted.

In some embodiments, R¹ is substituted or unsubstituted aryl, substituted or unsubstituted heteroaryl, substituted or unsubstituted cycloalkyl, or substituted or unsubstituted heterocyclyl. In some embodiments, R¹ is substituted aryl. In some embodiments, le is phenyl substituted with alkyl. In some embodiments, R¹ is phenyl substituted with methyl.

In some embodiments, R¹ is:

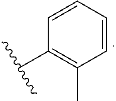 [SMILES: *c1ccccc1C]

In some embodiments, R₂ is unsubstituted alkyl. In some embodiments, R₂ is C₁-C₆ alkyl. In some embodiments, R₂ is isobutyl.

In some embodiments, L^{a} is alkylene or heteroalkylene. In some embodiments, L^{a} is alkylene. In some embodiments, R^{4X} is substituted or unsubstituted aryl or substituted or unsubstituted heteroaryl. In some embodiments, R^{4x} is substituted or unsubstituted aryl. In some embodiments, R⁴ is optionally substituted alkyl-aryl. In some embodiments, L^{a} is alkyl and R^{4x} is substituted or unsubstituted aryl. In some embodiments, L^{a} is alkyl and R^{4x} is substituted or unsubstituted aryl. In some embodiments, L^{a} is alkyl and R^{4x} is substituted aryl (e.g., being substituted with substituted heteroalkyl, being substituted with optionally substituted (e.g., N-attached) heterocycloalkyl. In some embodiments, L^{a} is alkyl and R^{4x} is substituted aryl, the aryl being substituted with heteroalkyl further substituted with N-attached heterocycloalkyl further substituted with formamidyl.

In some embodiments, R¹ is substituted aryl, R² is C₁-C⁶ alkyl, L^{a} is alkyl, and R^{4x} is substituted aryl.

In some embodiments, R⁴ is:

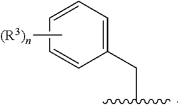

In some embodiments, R³ is substituted heteroalkyl, substituted or unsubstituted cycloalkyl, or substituted or unsubstituted heterocycloalkyl. In some embodiments, R³ is substituted heteroalkyl. In some embodiments, R³ is substituted heteroalkyl, being substituted with optionally substituted (e.g., N-attached) heterocycloalkyl. In some embodiments, R³ is substituted heteroalkyl, being substituted with N-attached heterocycloalkyl substituted with formamidyl.

In some embodiments, R⁴ is:

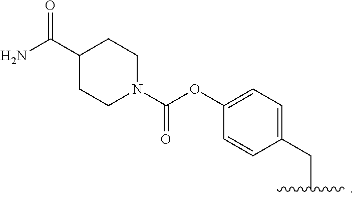 [SMILES: *Cc1ccc(OC(=O)N2CCC(C(N)=O)CC2)cc1]

In some embodiments, le is substituted aryl, R₂ is C₁-C₆ alkyl, and R³ is substituted heteroalkyl, being substituted with N-attached heterocycloalkyl substituted with formamidyl.

In some embodiments, R′ is substituted alkyl, the alkyl being substituted with substituted alkoxy. In some embodiments, R′ is substituted alkyl, the alkyl being substituted with substituted alkoxy, the alkoxy being substituted with one or more substituent, each substituent being independently selected from the group consisting of oxo, hydroxy, optionally substituted C₁-C₆ alkoxy, thiol, optionally substituted heterocycloalkyl, acetamidyl, substituted or unsubstituted (e.g., unsaturated) cycloalkyl, and amino.

In some embodiments, R′ is substituted alkyl, the alkyl being substituted with substituted alkoxy, the alkoxy being substituted with C₁-C₆ alkoxy further substituted with one or more substituent, each substituent being independently selected from the group consisting of oxo, hydroxy, optionally substituted C₁-C₄ alkoxy (e.g., being optionally substituted with hydroxyl), optionally substituted heterocycloalkyl, and hydroxyalkyl. In some embodiments, R′ is substituted alkyl, the alkyl being substituted with substituted alkoxy, the alkoxy being substituted with C₁-C₆ alkoxy further substituted with C₁-C₄ alkoxy optionally substituted with oxo, hydroxy, C₁-C₄ alkoxy, and/or C₁-C₄ alkyl. In some embodiments, R′ is substituted alkyl, the alkyl being substituted with substituted alkoxy, the alkoxy being substituted with 1, 3-dioxane substituted with one or more methyl. In some embodiments, R′ is substituted alkyl, the alkyl being substituted with substituted alkoxy, the alkoxy being substituted with C₁-C₆ alkoxy further substituted with C₁-C₆ alkyl-acyl optionally substituted with hydroxyl. In some embodiments, R′ is substituted alkyl, the alkyl being substituted with substituted alkoxy, the alkoxy being substituted with substituted unsaturated cycloalkyl (e.g., being substituted with one or more C₁-C₄ alkyl).

In some embodiments, R′ is substituted alkyl, the alkyl being substituted with substituted alkoxy, the alkoxy being substituted with heterocycloalkyl (e.g., dithiolanyl or dithiolanyl oxide).

In some embodiments, R′ is substituted alkyl, the alkyl being substituted with substituted alkoxy, the alkoxy being substituted with dithiolanyl or dithiolanyl oxide.

In some embodiments, R′ is substituted alkyl, the alkyl being substituted with substituted alkoxy, the substituted alkoxy being substituted with oxo and one or more other substituent, each other substituent being independently selected from the group consisting of optionally substituted C₁-C₆ alkoxy (e.g., being substituted with one or more substituent, each substituent being independently selected from the group consisting of oxo, hydroxy, optionally substituted C₁-C₄ alkoxy (e.g., being optionally substituted with oxo, hydroxy, C₁-C₄ alkoxy, and/or C₁-C₄ alkyl), optionally substituted heterocycloalkyl (e.g., 1, 3-dioxane substituted with one or more methyl), hydroxyalkyl, optionally substituted C₁-C₆ alkyl-acyl (e.g., the acyl being optionally substituted with hydroxy)), hydroxy, thiol, heterocycloalkyl (e.g., dithiolanyl or dithiolanyl oxide), acetamide, substituted unsaturated cycloalkyl (e.g., being substituted with one or more C₁-C₄ alkyl), and amino. In some embodiments, the C₁-C₆ alkoxy is substituted with one or more substituent, each substituent being independently selected from the group consisting of oxo, hydroxy, optionally substituted C₁-C₄ alkoxy (e.g., being optionally substituted with oxo, hydroxy, C₁-C₄ alkoxy, and/or C₁-C₄ alkyl), optionally substituted heterocycloalkyl (e.g., 1, 3-dioxane substituted with one or more methyl), hydroxyalkyl, optionally substituted C₁-C₆ alkyl-acyl (e.g., the acyl being optionally substituted with hydroxy), and substituted unsaturated cycloalkyl (e.g., being substituted with one or more C₁-C₄ alkyl). In some embodiments, the heterocycloalkyl is dithiolanyl or dithiolanyl oxide.

In some embodiments, R′ is substituted or unsubstituted (e.g., linear or branched) heteroalkyl comprising one or more —C—O—C— (e.g., within the (e.g., linear or branched) heteroalkyl chain).

In some embodiments, R′ is substituted or unsubstituted (e.g., linear or branched) heteroalkyl comprising one or more ester, one or more carbonate, one or more amide, and/or one or more disulfide (e.g., within the (e.g., linear or branched) heteroalkyl chain).

In some embodiments, R′ is substituted or unsubstituted (e.g., linear or branched) heteroalkyl comprising one carbonate (e.g., within the (e.g., linear or branched) heteroalkyl chain).

In some embodiments, R′ is substituted or unsubstituted (e.g., linear or branched) heteroalkyl comprising one or two ester (e.g., within the (e.g., linear or branched) heteroalkyl chain).

In some embodiments, R′ is substituted or unsubstituted (e.g., linear or branched) heteroalkyl comprising one ester (e.g., within the (e.g., linear or branched) heteroalkyl chain).

In some embodiments, R′ is substituted or unsubstituted (e.g., linear or branched) heteroalkyl comprising one ester and one carbonate (e.g., within the (e.g., linear or branched) heteroalkyl chain).

In some embodiments, R′ is substituted or unsubstituted (e.g., linear or branched) heteroalkyl comprising one or two ester and one amide (e.g., within the (e.g., linear or branched) heteroalkyl chain).

In some embodiments, R′ is substituted or unsubstituted (e.g., linear or branched) heteroalkyl comprising one ester and one amide (e.g., within the (e.g., linear or branched) heteroalkyl chain).

In some embodiments, R′ is substituted or unsubstituted (e.g., linear or branched) heteroalkyl comprising one or two amide (e.g., within the (e.g., linear or branched) heteroalkyl chain).

In some embodiments, R′ is substituted or unsubstituted (e.g., linear or branched) heteroalkyl comprising one or two disulfide (e.g., within the (e.g., linear or branched) heteroalkyl chain).

In some embodiments, R′ is substituted or unsubstituted (e.g., linear or branched) heteroalkyl comprising one or two disulfide and one ester (e.g., within the (e.g., linear or branched) heteroalkyl chain).

In some embodiments, R′ is substituted (e.g., linear or branched) heteroalkyl, the (e.g., linear or branched) heteroalkyl being substituted with one or more (heteroalkyl) substituent, each (heteroalkyl) substituent being independently selected from the group consisting of optionally substituted C₁-C₆ alkyl, hydroxy, heterocycloalkyl, thiol, thioalkyl, amino, and carboxylic acid.

In some embodiments, R′ is substituted (e.g., linear or branched) heteroalkyl, the (e.g., linear or branched) heteroalkyl being substituted with substituted C₁-C₆ alkyl, the C₁-C₆ alkyl being substituted with heteroalkyl being further optionally substituted with one or more substituent, each substituent being independently selected from the group consisting of hydroxy, carboxylic acid, optionally substituted N-substituted pyrrolidinyl (e.g., optionally substituted with carboxylic acid)).

In some embodiments, R′ is substituted (e.g., linear or branched) heteroalkyl, the (e.g., linear or branched) heteroalkyl being substituted with heterocycloalkyl. In some embodiments, R′ is substituted (e.g., linear or branched) heteroalkyl, the (e.g., linear or branched) heteroalkyl being substituted with 1,2-dithiolane, 1,2-dithiolane oxide, optionally substituted dioxane (e.g., optionally substituted with one or more C₁-C₆ alkyl), (e.g., N-substituted) pyrrolidine (e.g., substituted with alkyl (e.g., further substituted with oxo, thiol, and C₁-C₃ alkyl)), or substituted (e.g., N-attached) pyrrolidine (e.g., substituted with carboxylic acid).

In some embodiments, R′ is substituted branched heteroalkyl.

In some embodiments, R′ is:

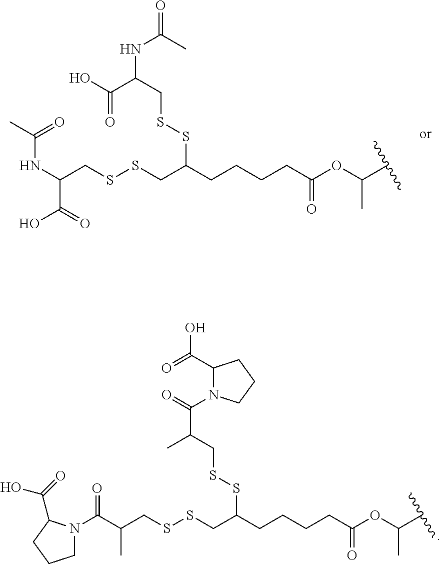 [SMILES: *C(C)OC(=O)CCCCC(CSSCC(NC(C)=O)C(=O)O)SSCC(NC(C)=O)C(=O)O ; *C(C)OC(=O)CCCCC(CSSCC(C)C(=O)N1CCCC1C(=O)O)SSCC(C)C(=O)N1CCCC1C(=O)O]

In some embodiments, R′ is:

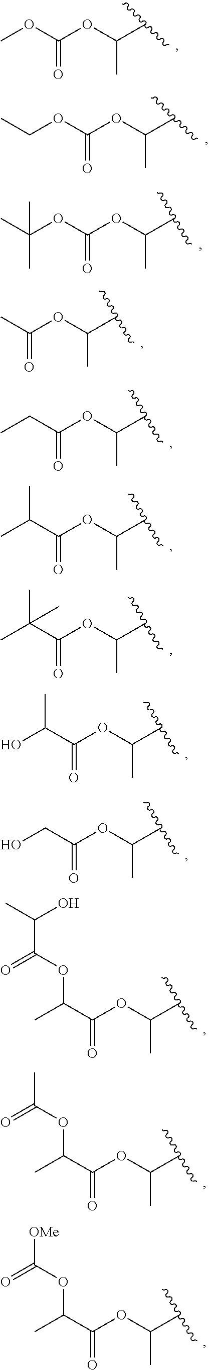

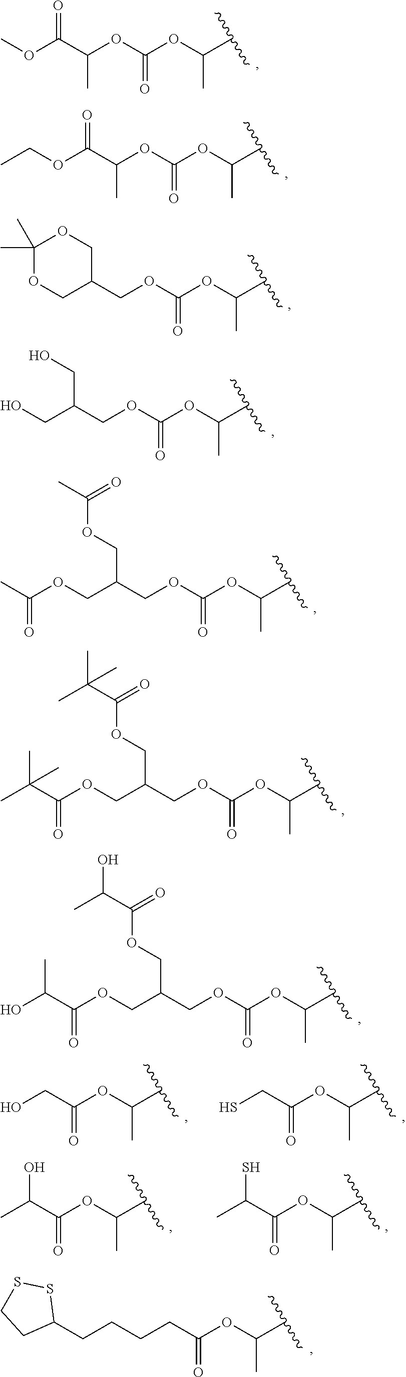

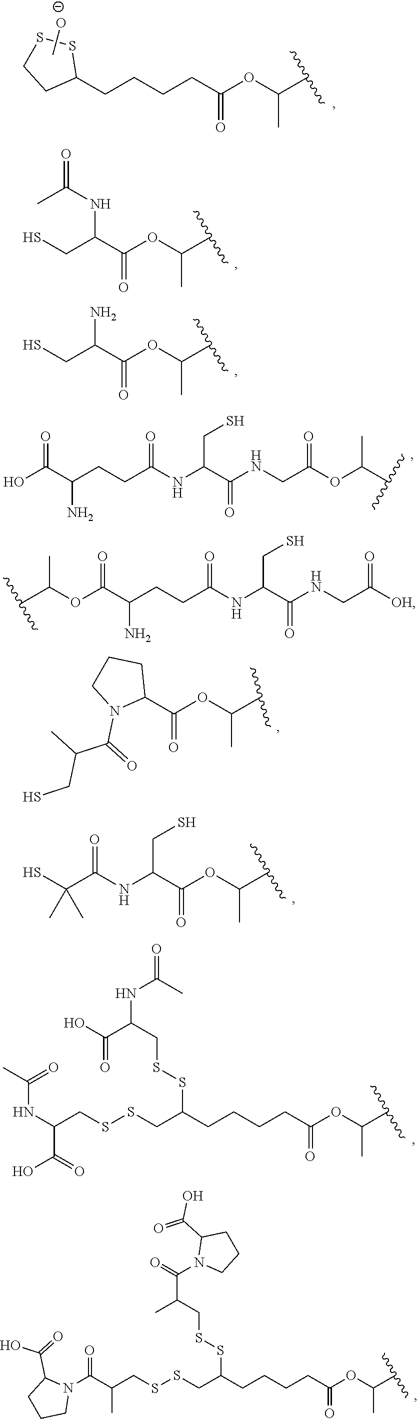

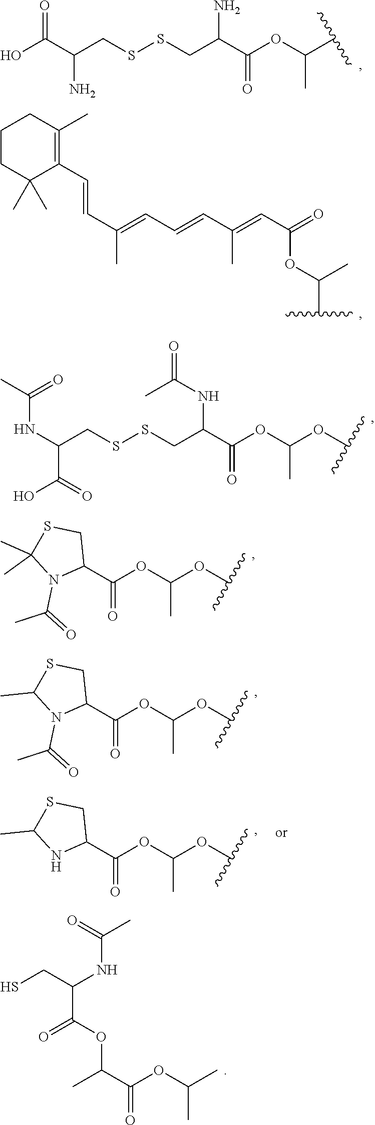

In some embodiments, R′ is:

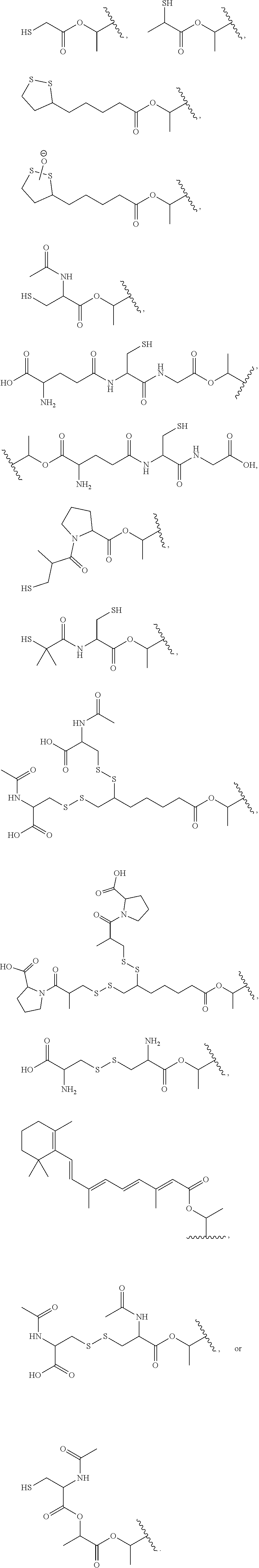

In some embodiments, provided herein is a compound, or a pharmaceutically acceptable salt or solvate (e.g., or a stereoisomer) thereof, having the structure of Formula (Ia):

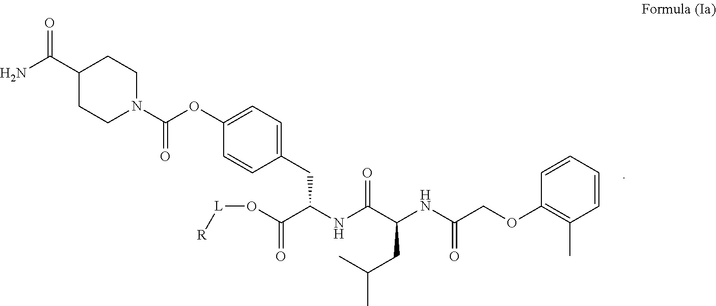

In some embodiments, L is bond, —O(C═O)(OCR⁸R⁹)_{z}—, or —(C═O)(OCR⁸R⁹)_{z}—. In some embodiments, L is bond, —(C═O)O(CR⁸R⁹)_{z}—, or —(C═O)O(CR⁸R⁹)_{z}—. In some embodiments, each R⁸ and R⁹ is independently H, halogen, C₁-C₃-alkyl, C₁-C₃-haloalkyl, C₁-C₃-alkoxy, C₃-C₅-cycloalkyl, or R⁸ and R⁹ are taken together with the atoms to which they are attached to form a C₃-C₅-cycloalkyl. In some embodiments, z is 1-6. In some embodiments, R is substituted or unsubstituted (e.g., straight or branched) alkyl, substituted or unsubstituted (e.g., straight or branched) heteroalkyl, or substituted or unsubstituted heterocycloalkyl (e.g., (N—) substituted with alkyl (e.g., further substituted with oxo and thiol)).

In some embodiments, L is bond. In some embodiments, L is —(C═O)(OCR⁸R⁹)_{z}—. In some embodiments, L is —O(C═O)(OCR⁸R⁹)_{z}—. In some embodiments z is 1-3. In some embodiments, z is 1. In some embodiments, each R⁸ and R⁹ is independently H or C₁-C₃-alkyl. In some embodiments, each R⁸ is H and each R⁹ is C₁-C₃-alkyl. In some embodiments, each R⁸ is H and each R⁹ is CH₃. In some embodiments, each R⁸ and R⁹ is H. In some embodiments, z is 1, R⁸ is H, and R⁹ is H or CH₃.

In some embodiments, L is —(C═O)OCH(CH₃)—.

In some embodiments, L is —O(C═O)OCH(CH₃)—.

In some embodiments, R is substituted (e.g., straight or branched) alkyl, the (e.g., straight or branched) alkyl being substituted with one or more (alkyl) substituent, each (alkyl) substituent being independently selected from the group consisting of hydroxy, optionally substituted alkoxy, oxo, optionally substituted alkyl, optionally substituted heterocycloalkyl, hydroxyalkyl, thiol, acetamide, substituted unsaturated cycloalkyl (e.g., being substituted with one or more C₁-C₄ alkyl), and amino. In some embodiments, R is substituted (e.g., straight or branched) alkyl, the (e.g., straight or branched) alkyl being substituted with substituted alkoxy, being substituted with oxo and hydroxy or oxo and C₁-C₃ alkoxy. In some embodiments, R is substituted (e.g., straight or branched) alkyl, the (e.g., straight or branched) alkyl being substituted with substituted alkyl, being substituted with alkoxy further optionally substituted with oxo, C₁-C₄ alkyl, and/or hydroxy. In some embodiments, R is substituted (e.g., straight or branched) alkyl, the (e.g., straight or branched) alkyl being substituted with substituted heterocycloalkyl, being substituted with dioxane (e.g., 1,3 dioxanyl optionally substituted with methyl), dithiolanyl, or dithiolanyl oxide. In some embodiments, R is substituted (e.g., straight or branched) alkyl, the (e.g., straight or branched) alkyl being substituted with substituted unsaturated cycloalkyl (e.g., being substituted with one or more C₁-C₄ alkyl).

In some embodiments, L is —(C═O)OCH(CH₃)— and R is:

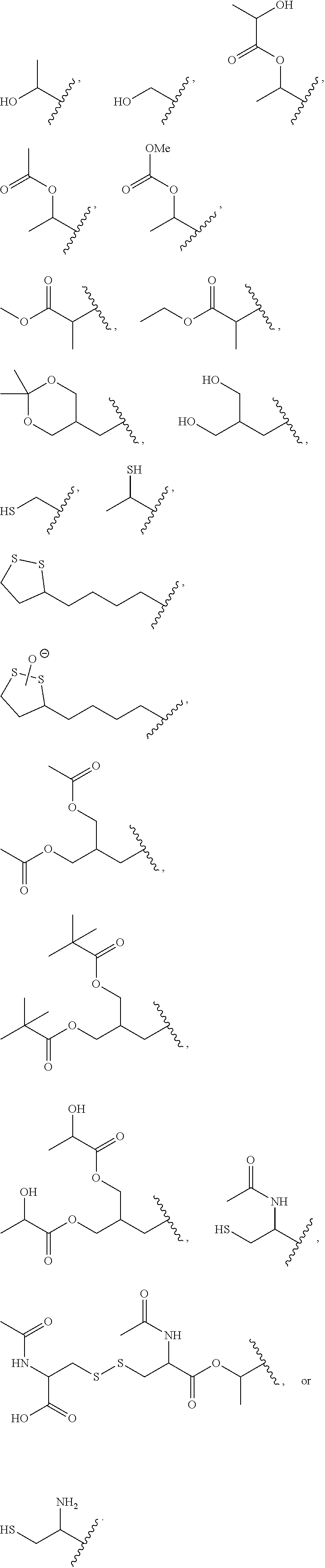

In some embodiments, R is substituted (e.g., linear or branched) heteroalkyl, the (e.g., linear or branched) heteroalkyl being substituted with one or more (heteroalkyl) substituent, each (heteroalkyl) substituent being independently selected from the group consisting of hydroxy, C₁-C₆ alkoxy, thioalkyl, amino, carboxylic acid, C₁-C₆ alkyl, acetamide, thiol, oxo, and optionally substituted (e.g., N-attached) heterocycloalkyl. In some embodiments, R is substituted (e.g., linear or branched) heteroalkyl, the (e.g., linear or branched) heteroalkyl being substituted with substituted (e.g., N-attached) heterocycloalkyl, being substituted with carboxylic acid.

In some embodiments, R is substituted (e.g., linear or branched) heteroalkyl, the (e.g., linear or branched) heteroalkyl being substituted with one or more (heteroalkyl) substituent, each (heteroalkyl) substituent being independently selected from the group consisting of thioalkyl, amino, carboxylic acid, C₁-C₆ alkyl, acetamide, thiol, oxo, and optionally substituted (e.g., N-attached) heterocycloalkyl (e.g., optionally substituted with carboxylic acid). In some embodiments, R is substituted linear heteroalkyl, the linear heteroalkyl being substituted with thioalkyl, amino, and carboxylic acid. In some embodiments, R is substituted linear heteroalkyl, the linear heteroalkyl being substituted with thioalkyl, thiol, and C₁-C₄ alkyl. In some embodiments, R is substituted branched heteroalkyl, the branched heteroalkyl being substituted with one or more carboxylic acid. In some embodiments, R is substituted branched heteroalkyl, the branched heteroalkyl being substituted with one or more C₁-C₄ alkyl, one or more oxo, and one or more N-attached pyrrolidine substituted with carboxylic acid. In some embodiments, R is substituted linear heteroalkyl, the linear heteroalkyl being substituted with amino and carboxylic acid. In some embodiments, R is substituted linear heteroalkyl, the linear heteroalkyl being substituted with thioalkyl. In some embodiments, R is substituted linear heteroalkyl, the linear heteroalkyl being substituted with acetamide and carboxylic acid.

In some embodiments, L is —(C═O)OCH(CH₃)— and R is:

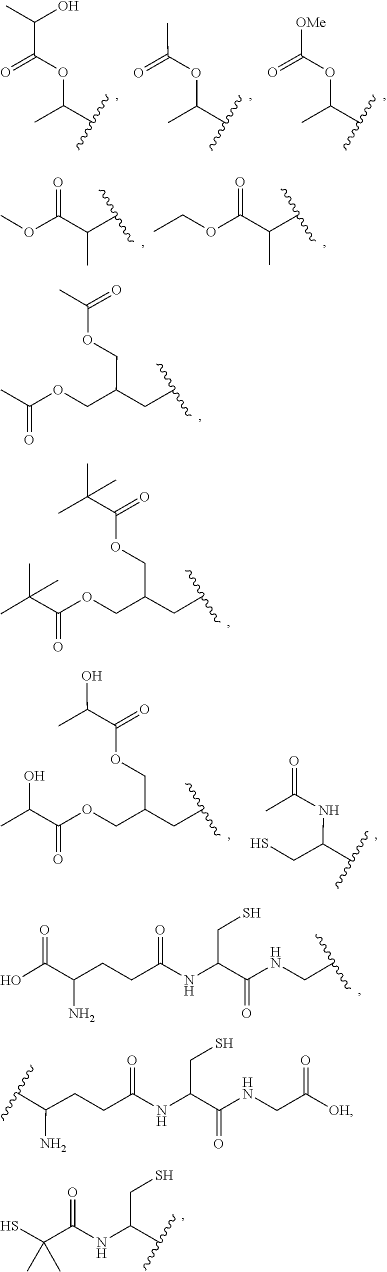

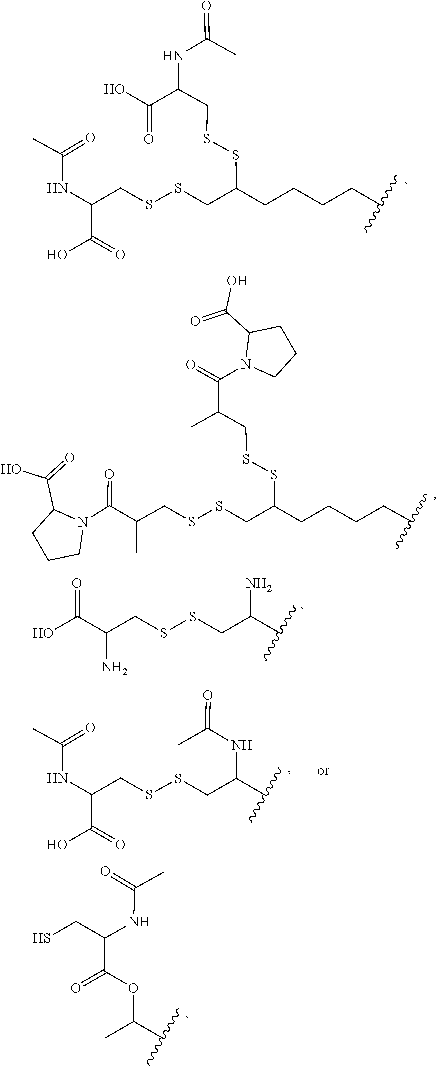

In some embodiments, R is substituted heterocycloalkyl. In some embodiments, R is N-substituted heterocycloalkyl, being substituted with alkyl further substituted with oxo and thiol.

In some embodiments, L is —(C═O)OCH(CH₃)— and R is:

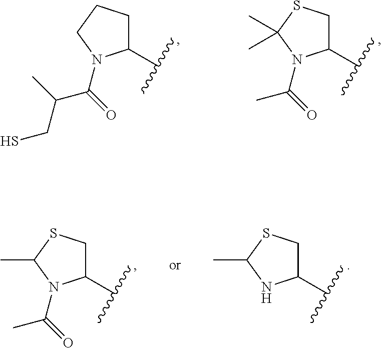

In some embodiments, R is unsubstituted alkyl (e.g., methyl, ethyl, isopropyl, or t-butyl).

In some embodiments, L is —O(C═O)OCH(CH₃)—, and R is unsubstituted alkyl (e.g., methyl, ethyl, isopropyl, or t-butyl).

In some embodiments, the compound is other than a compound having the structure:

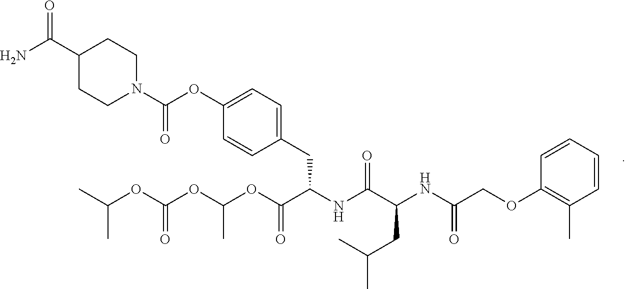 [SMILES: Cc1ccccc1OCC(=O)N[C@@H](CC(C)C)C(=O)N[C@@H](Cc1ccc(OC(=O)N2CCC(C(N)=O)CC2)cc1)C(=O)OC(C)OC(=O)OC(C)C]

In some embodiments, provided herein is a compound, or a pharmaceutically acceptable salt or solvate (e.g., or a stereoisomer) thereof, having the structure of Formula (Ib):

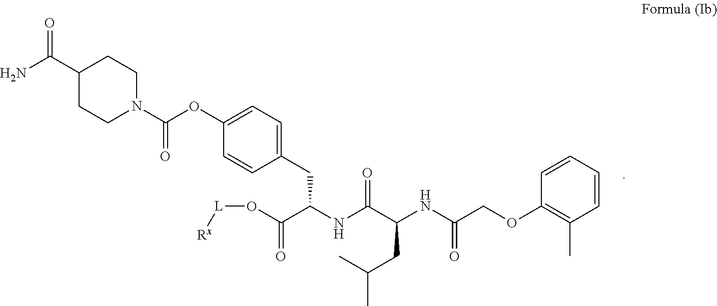

In some embodiments, L is bond, —O(C═O)(OCR⁸R⁹)_{z}—, or —(C═O)(OCR⁸R⁹)_{z}—. In some embodiments, each R⁸ and R⁹ is independently H, halogen, C₁-C₃-alkyl, C₁-C₃-haloalkyl, C₁-C₃-alkoxy, C₃-C₅-cycloalkyl, or R⁸ and R⁹ are taken together with the atoms to which they are attached to form a C₃-C₅-cycloalkyl. In some embodiments, z is 1-6.

In some embodiments, R^{x} is:

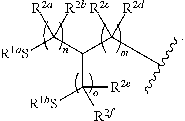

In some embodiments, R^{1a} and R^{1b} are each independently —H or —SR^{1c}. In some embodiments, each R^{1c} is independently substituted or unsubstituted (e.g., straight or branched) alkyl or substituted or unsubstituted (e.g., straight or branched) heteroalkyl. In some embodiments, each R^{2a}, R^{2b}, R^{2c}, R^{2d}, R^{2e}, and R^{2f} is independently H, halogen, C₁-C₃-alkyl, C₁-C₃-haloalkyl, C₁-C₃-alkoxy, C₃-C₅-cycloalkyl, or two of R^{2a} and R^{2b}, R^{2c}, and R^{2d}, or R^{2e}, and R^{2f} are taken together with the atoms to which they are attached to form a C₃-C₅-cycloalkyl. In some embodiments, m is an integer from 1-10. In some embodiments, n and o are each independently an integer from 0-3.

In some embodiments, L, R⁸, R⁹, and z are each described elsewhere herein.

In some embodiments, n and o are each independently 0 or 1. In some embodiments, n is 0 or 1. In some embodiments, n is 1. In some embodiments, o is 0 or 1. In some embodiments, o is 0. In some embodiments, n is 0 and n is 1.

In some embodiments, m is 3-5. In some embodiments, m is 4. In some embodiments, n is 0 and m is 4. In some embodiments, n is 1 and m is 4. In some embodiments, n is 0, n is 1, and m is 4.

In some embodiments, each R^{2a}, R^{2b}, R^{2c}, R^{2d}, R^{2e}, and R^{2f} is independently H, halogen, C₁-C₃alkyl, or C₁-C₃haloalkyl. In some embodiments, each R^{2a}, R^{2b}, R^{2c}, R^{2d}, R^{2e}, and R^{2f} is independently H, halogen, C₁-C₃alkyl, or C₁-C₃haloalkyl, at R^{4a}st one of R^{2a}, R^{2b}, R^{2c}, R^{2d}, R^{2e}, and R^{2f} being halogen, C₁-C₃alkyl, or C₁-C₃haloalkyl. In some embodiments, each R^{2a}, R^{2b}, R^{2c}, R^{2d}, R^{2e}, and R^{2f} is H.

In some embodiments, R^{x} is:

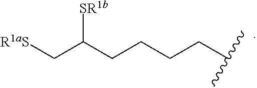

In some embodiments, R^{1a} and R^{1b} are each independently —H or —SR^{1c}. In some embodiments, each R^{1c} is independently substituted or unsubstituted (e.g., straight or branched) alkyl (e.g., substituted with one or more (alkyl) substituent, each (alkyl) substituent being independently selected from the group consisting of carboxylic acid, —SH, thioalkyl, acetamide, amino, oxo, optionally substituted heterocycloalkyl (e.g., N-attached pyrrolidinyl substituted with —COOH)) or substituted or unsubstituted (e.g., straight or branched) heteroalkyl (e.g., substituted with one or more (heteroalkyl) substituent, each (heteroalkyl) substituent being independently selected from the group consisting of carboxylic acid, amino, thioalkyl, thiol, acetamide, and C₁-C₃ alkyl).

In some embodiments, R^{1a} is —H or —SR^{1c} and R^{1b} is —SR^{1c}, or R^{1a} is —SR^{1c} and R^{1b} is —H or —SR^{1c}. In some embodiments, R^{1a} is —H or SR^{1c} and R^{1b} is —SR^{1c}. In some embodiments, R^{1a} is —H and R^{1b} is —SR^{1c}. In some embodiments, R^{1a} is —SR^{1c} and R^{1b} is —H or SR^{1c}. In some embodiments, R^{1a} is —SR^{1c} and R^{1b} is —SR^{1c}. In some embodiments, R^{1a} and R^{1b} are each —SR^{1c}.

In some embodiments, R^{1a} and R^{1b} each independently comprise a radical of one or more keratolytic group (e.g., each radical of the one or more keratolytic group being independently selected from the group consisting of a radical of glycolic acid (GA), a radical of thioglycolic acid (TGA), a radical of lactic acid (Lac), a radical of thiolactic acid (TLac), a radical of lipoic acid (Lip), a radical of lipoic acid sulfoxide (Lipox), a radical of dihydrolipoic acid (diHLip), a radical of N-acetyl cysteine (NAC), a radical of cysteine (Cys), a radical of glutathione (GSH), a radical of captopril (Cap), and a radical of bucillamine (Buc)).

In some embodiments, R^{1a} and R^{1b} are each independently a radical of one or more keratolytic group, each radical of the one or more keratolytic group being independently selected from the group consisting of a radical of glycolic acid (GA), a radical of thioglycolic acid (TGA), a radical of lactic acid (Lac), a radical of thiolactic acid (TLac), a radical of lipoic acid (Lip), a radical of lipoic acid sulfoxide (Lipox), a radical of dihydrolipoic acid (diHLip), a radical of N-acetyl cysteine (NAC), a radical of cysteine (Cys), a radical of glutathione (GSH), a radical of captopril (Cap), and a radical of bucillamine (Buc).

In some embodiments, R^{1a} and R^{1b} each independently comprise a (thiol) radical of one or more keratolytic group, each (thiol) radical of the one or more keratolytic group being independently selected from the group consisting of a (thiol) radical of thioglycolic acid (TGA), a (thiol) radical of thiolactic acid (TLac), a (thiol) radical of dihydrolipoic acid (diHLip), a (thiol) radical of N-acetyl cysteine (NAC), a (thiol) radical of cysteine (Cys), a (thiol) radical of glutathione (GSH), a (thiol) radical of captopril (Cap), and a (thiol) radical of bucillamine (Buc).

In some embodiments, R^{1a} and R^{1b} are each independently a thiol radical of one or more keratolytic group, each thiol radical of the one or more keratolytic group being independently selected from the group consisting of a thiol radical of thioglycolic acid (TGA), a thiol radical of thiolactic acid (TLac), a thiol radical of dihydrolipoic acid (diHLip), a thiol radical of N-acetyl cysteine (NAC), a thiol radical of cysteine (Cys), a thiol radical of glutathione (GSH), a thiol radical of captopril (Cap), and a thiol radical of bucillamine (Buc).

In some embodiments, the (e.g., thiol) radical of the keratolytic agent comprises a (e.g., thiol) radical of one or more keratolytic group, each (e.g., thiol) radical of the one or more keratolytic group being independently selected from the group consisting of [Lac-Lac]·, [Lac-NAC]·, [Cys-Cys]·, [diHLip-NAC-NAC]·, [diHLip-NAC]·, [diHLip-Cap-Cap]·, [diHLip-Cap]·, [diHLip-Cys-Cys]·, [diHLip-Cys]·, [diHLip-Lipox-Lipox]·, and [diHLip-Lipox]·.

In some embodiments, the thiol radical of the keratolytic group is the point of attachment of R^{1a} and/or R^{1b} to the rest of the molecule. In some embodiments, (the thiol radical of) R^{1a} and/or R^{1b} each independently attach to the rest of the molecule to form a disulfide bond.

In some embodiments, R^{1a} and R^{1b} are each independently —H or:

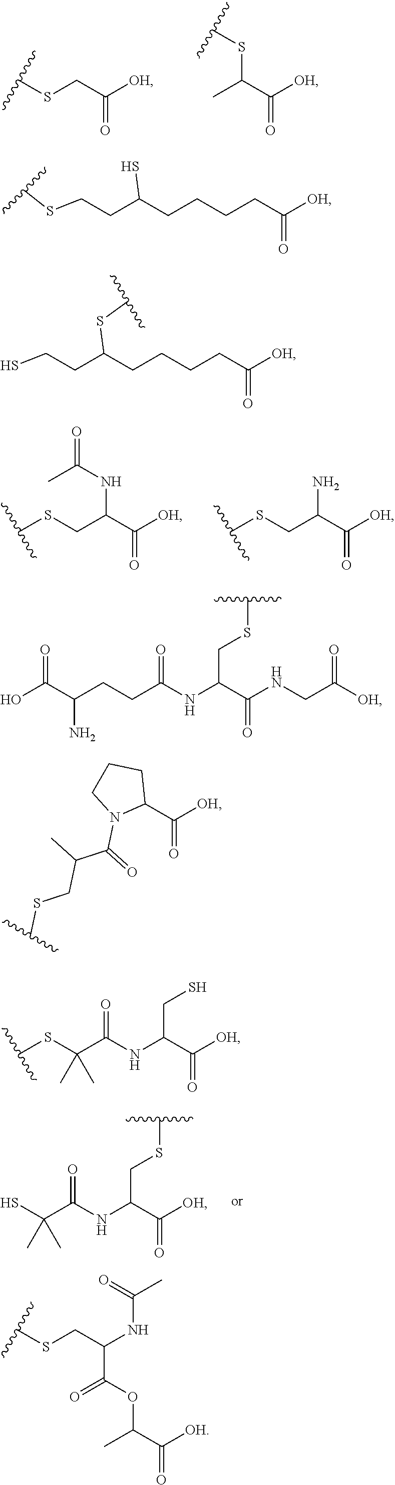

In some embodiments, R^{1a} and R^{1b} are the same. In some embodiments, R^{1a} and R^{1b} are each —SR^{1c} and the same. In some embodiments, R^{1a} and R^{1b} are different. In some embodiments, R^{1a} and R^{1b} are each SR^{1c} and different.

In some embodiments, L is —(C═O)OCH(CH₃)— and R^{x} is:

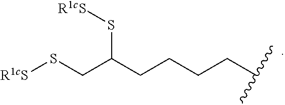

In some embodiments, each R^{1c} is independently substituted or unsubstituted (e.g., straight or branched) alkyl or substituted or unsubstituted (e.g., straight or branched) heteroalkyl. In some embodiments, each R^{1c} is independently substituted (e.g., straight or branched) alkyl or substituted (e.g., straight or branched) heteroalkyl. In some embodiments, each R^{1c} is independently substituted (e.g., straight or branched) alkyl. In some embodiments, each R^{1c} is (the same) substituted (e.g., straight or branched) alkyl. In some embodiments, each R^{1c} is (a different) substituted (e.g., straight or branched) alkyl.

In some embodiments, each R^{1c} is independently substituted (e.g., straight or branched) heteroalkyl. In some embodiments, each R^{1c} is (the same) substituted (e.g., straight or branched) heteroalkyl. In some embodiments, each R^{1c} is (a different) substituted (e.g., straight or branched) heteroalkyl.

In some embodiments, one of R^{1c} is substituted (e.g., straight or branched) alkyl and the other is substituted (e.g., straight or branched) heteroalkyl.

In some embodiments, each R^{1c} is the same. In some embodiments, each R^{1c} is different.

In some embodiments, each R^{1c} is independently substituted (e.g., straight or branched) alkyl, the substituted alkyl being substituted with one or more (alkyl) substituent, each (alkyl) substituent being independently selected from the group consisting of carboxylic acid, —SH, thioalkyl (e.g., —CH₂SH), acetamide (e.g., —NH(C═O)CH₃), amino, oxo, and optionally substituted heterocycloalkyl (e.g., N-attached pyrrolidinyl substituted with —COOH).

In some embodiments, the optionally substituted heterocycloalkyl is:

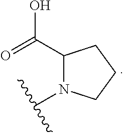 [SMILES: *N1CCCC1C(=O)O]

In some embodiments, each R^{1c} is independently substituted (e.g., straight or branched) heteroalkyl, the substituted heteroalkyl being substituted with one or more (heteroalkyl) substituent, each (heteroalkyl) substituent being independently selected from the group consisting of carboxylic acid, amino, thioalkyl (e.g., —CH₂SH), thiol, acetamide (e.g., —NH(C═O)CH₃), and C₁-C₃ alkyl.

In some embodiments, R^{1c} is:

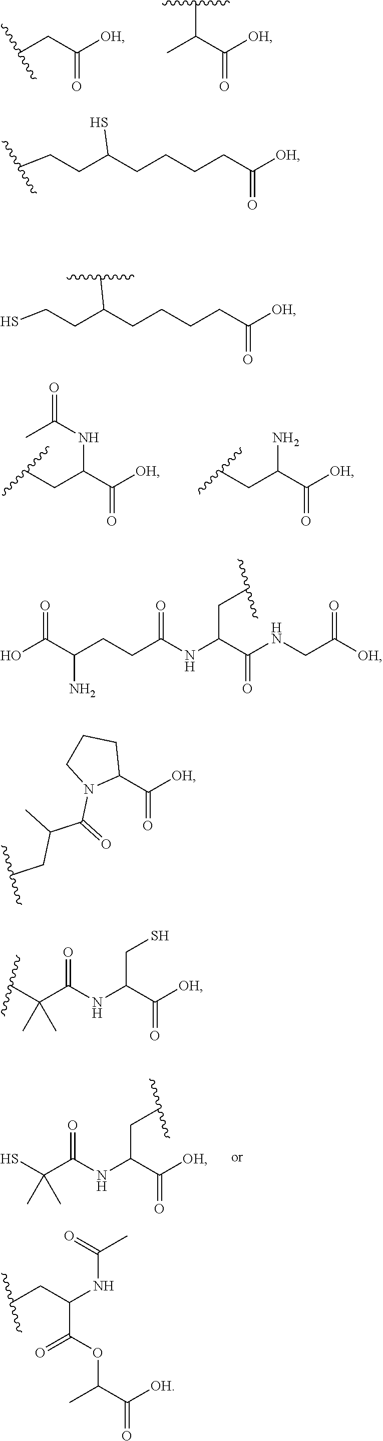

In some embodiments, R^{1a}, R^{1b}, and each R^{1c} each independently comprise one or more substituent that is a carboxylic acid or an ester. In some embodiments, R^{1a}, R^{1b}, and each R^{1c} each each independently comprise one or more substituent that is a carboxylic acid (e.g., —(C═O)OH). In some embodiments, R^{1a} comprises one or more substituent that is a carboxylic acid (e.g., —(C═O)OH). In some embodiments, R^{1b} comprises one or more substituent that is a carboxylic acid (e.g., —(C═O)OH). In some embodiments, each R^{1c} independently comprises one or more substituent that is a carboxylic acid (e.g., —(C═O)OH). In some embodiments, R^{1a}, R^{1b}, and each R^{1c} each independently comprise one or more substituent that is an ester (e.g., —(C═O)O—C₁-C₄alkyl). In some embodiments, R^{1a} comprises one or more substituent that is an ester (e.g., —(C═O)O—C₁-C₄alkyl). In some embodiments, R^{1b} comprises one or more substituent that is an ester (e.g., —(C═O)O—C₁-C₄alkyl). In some embodiments, each R^{1c} independently comprises one or more substituent that is an ester (e.g., —(C═O)O—C₁-C₄alkyl).

In some embodiments, the —(C═O)OH of R^{1a}, R^{1b}, and/or R^{1c} is optionally esterified (e.g., —(C═O)OH or —(C═O)O—C₁-C₄alkyl). In some embodiments, the C₁-C₄alkyl is methyl, ethyl, propyl, isopropyl, butyl, or t-butyl.

In some embodiments, provided herein is a compound, or a pharmaceutically acceptable salt or solvate (e.g., or a stereoisomer) thereof, having the structure of Formula (Ic):

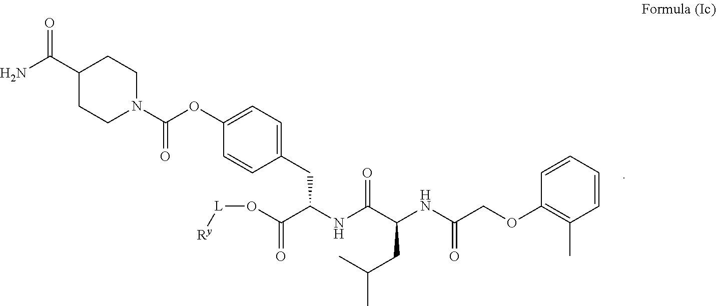

In some embodiments, L is bond, —O(C═O)(OCR⁸R⁹)_{z}—, or —(C═O)(OCR⁸R⁹)_{z}—. In some embodiments, each R⁸ and R⁹ is independently H, halogen, C₁-C₃-alkyl, C₁-C₃-haloalkyl, C₁-C₃-alkoxy, C₃-C₅-cycloalkyl, or R⁸ and R⁹ are taken together with the atoms to which they are attached to form a C₃-C₅-cycloalkyl. In some embodiments, z is 1-6.

In some embodiments, L, R⁸, R⁹, and z are each described elsewhere herein.

In some embodiments, R^{y} is:

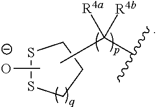

In some embodiments, each R^{4a} and R^{4b} is independently H, halogen, or substituted or unsubstituted alkyl. In some embodiments, p is an integer from 1-10. In some embodiments, q is an integer from 1-3.

In some embodiments, q is 1 or 2. In some embodiments, q is 1. In some embodiments, p is an integer from 3-5. In some embodiments, p is 4. In some embodiments, q is 1 and p is 4.

In some embodiments, each R^{4a} and R^{4b} is independently H or substituted or unsubstituted alkyl. In some embodiments, each R^{4a} and R^{4b} is independently H, halogen, C₁-C₃alkyl, or C₁-C₃haloalkyl. In some embodiments, each R^{4a} and R_{4b} is H.

In some embodiments, q is 1, p is an integer from 3-5, and each R^{4a} and R^{4b} is independently H, halogen, C₁-C₃alkyl, or C₁-C₃haloalkyl. In some embodiments, q is 1, p is 4, and each R^{4a} and R^{4b} is H.

In some embodiments, L is —(C═O)OCH(CH₃)— and R^{y} is:

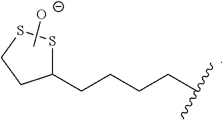

In some embodiments, provided herein is a compound having the structure of Formula (Id):

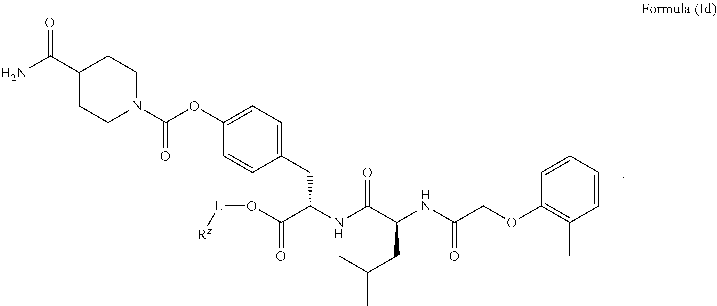

In some embodiments, L is bond, —O(C═O)(OCR⁸R⁹)_{z}—, or —(C═O)(OCR⁸R⁹)_{z}. In some embodiments, each R⁸ and R⁹ is independently H, halogen, C₁-C₃-alkyl, C₁-C₃-haloalkyl, C₁-C₃-alkoxy, C₃-C₅-cycloalkyl, or R⁸ and R⁹ are taken together with the atoms to which they are attached to form a C₃-C₅-cycloalkyl. In some embodiments, z is 1-6.

In some embodiments, L, R⁸, R⁹, and z are each described elsewhere herein.

In some embodiments, R^{z} is:

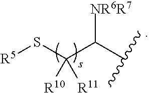

In some embodiments, R⁵ is —SR^{1c}. In some embodiments, R^{1c} is substituted or unsubstituted (e.g., straight or branched) alkyl (e.g., substituted with one or more (alkyl) substituent, each (alkyl) substituent being independently selected from the group consisting of carboxylic acid, —SH, thioalkyl, acetamide, amino, oxo, optionally substituted heterocycloalkyl (e.g., N-attached pyrrolidinyl substituted with —COOH)) or substituted or unsubstituted (e.g., straight or branched) heteroalkyl (e.g., substituted with one or more (heteroalkyl) substituent, each (heteroalkyl) substituent being independently selected from the group consisting of carboxylic acid, amino, thioalkyl, thiol, acetamide, and C₁-C₃ alkyl). In some embodiments, R⁶ and R⁷ are each independently H, substituted or unsubstituted alkyl, or substituted or unsubstituted heteroalkyl. In some embodiments, each R¹⁰ and R¹¹ is independently H, halogen, C₁-C₃-alkyl, C₁-C₃-haloalkyl, C₁-C₃-alkoxy, C₃-C₅-cycloalkyl, or two or more of R¹⁰ and R¹¹ are taken together with the atoms to which they are attached to form a C₃-C₅-cycloalkyl. In some embodiments, s is an integer from 1-10.

In some embodiments, R⁶ and R⁷ are each independently H or substituted or unsubstituted alkyl (e.g., C₁-C₃ alkyl optionally substituted with oxo). In some embodiments, R⁶ and R⁷ are each independently H or C₁-C₃ alkyl optionally substituted with oxo. In some embodiments, R⁶ and R⁷ are each independently H or —(C═O)CH₃. In some embodiments, R⁶ is H and R⁷ is H or —(C═O)CH₃. In some embodiments, R⁶ is H and R⁷ is —(C═O)CH₃. In some embodiments, R⁶ and R⁷ are H.

In some embodiments, each R¹⁰ and R¹¹ is independently H, halogen, C₁-C₃alkyl, or C₁-C₃haloalkyl. In some embodiments, each R¹⁰ and R¹¹ is H.

In some embodiments, s is 1-3. In some embodiments, s is 1. In some embodiments, s is 1 and R¹⁰ and R¹¹ are H.

In some embodiments, R⁵ comprises a radical of one or more keratolytic group (e.g., each radical of the one or more keratolytic group being independently selected from the group consisting of a radical of glycolic acid (GA), a radical of thioglycolic acid (TGA), a radical of lactic acid (Lac), a radical of thiolactic acid (TLac), a radical of lipoic acid (Lip), a radical of lipoic acid sulfoxide (Lipox), a radical of dihydrolipoic acid (diHLip), a radical of N-acetyl cysteine (NAC), a radical of cysteine (Cys), a radical of glutathione (GSH), a radical of captopril (Cap), and a radical of bucillamine (Buc)).

In some embodiments, R⁵ is a radical of one or more keratolytic group, each radical of the one or more keratolytic group being independently selected from the group consisting of a radical of glycolic acid (GA), a radical of thioglycolic acid (TGA), a radical of lactic acid (Lac), a radical of thiolactic acid (TLac), a radical of lipoic acid (Lip), a radical of lipoic acid sulfoxide (Lipox), a radical of dihydrolipoic acid (diHLip), a radical of N-acetyl cysteine (NAC), a radical of cysteine (Cys), a radical of glutathione (GSH), a radical of captopril (Cap), and a radical of bucillamine (Buc).

In some embodiments, R⁵ comprises a (thiol) radical of one or more keratolytic group, each (thiol) radical of the one or more keratolytic group being independently selected from the group consisting of a (thiol) radical of thioglycolic acid (TGA), a (thiol) radical of thiolactic acid (TLac), a (thiol) radical of dihydrolipoic acid (diHLip), a (thiol) radical of N-acetyl cysteine (NAC), a (thiol) radical of cysteine (Cys), a (thiol) radical of glutathione (GSH), a (thiol) radical of captopril (Cap), and a (thiol) radical of bucillamine (Buc).

In some embodiments R⁵ is a thiol radical of one or more keratolytic group, each thiol radical of the one or more keratolytic group being independently selected from the group consisting of a thiol radical of thioglycolic acid (TGA), a thiol radical of thiolactic acid (TLac), a thiol radical of dihydrolipoic acid (diHLip), a thiol radical of N-acetyl cysteine (NAC), a thiol radical of cysteine (Cys), a thiol radical of glutathione (GSH), a thiol radical of captopril (Cap), and a thiol radical of bucillamine (Buc).

In some embodiments, the (e.g., thiol) radical of the keratolytic agent comprises a (e.g., thiol) radical of one or more keratolytic group, each (e.g., thiol) radical of the one or more keratolytic group being independently selected from the group consisting of [Lac-Lac]·, [Lac-NAC]·, [Cys-Cys]·, [diHLip-NAC-NAC]·, [diHLip-NAC]·, [diHLip-Cap-Cap]·, [diHLip-Cap]·, [diHLip-Cys-Cys]·, [diHLip-Cys]·, [diHLip-Lipox-Lipox]·, and [diHLip-Lipox]·.

In some embodiments, the thiol radical of the keratolytic group is the point of attachment of R⁵ to the rest of the molecule. In some embodiments, R⁵ attaches to the rest of the molecule to form a disulfide bond.

In some embodiments, R⁵ is:

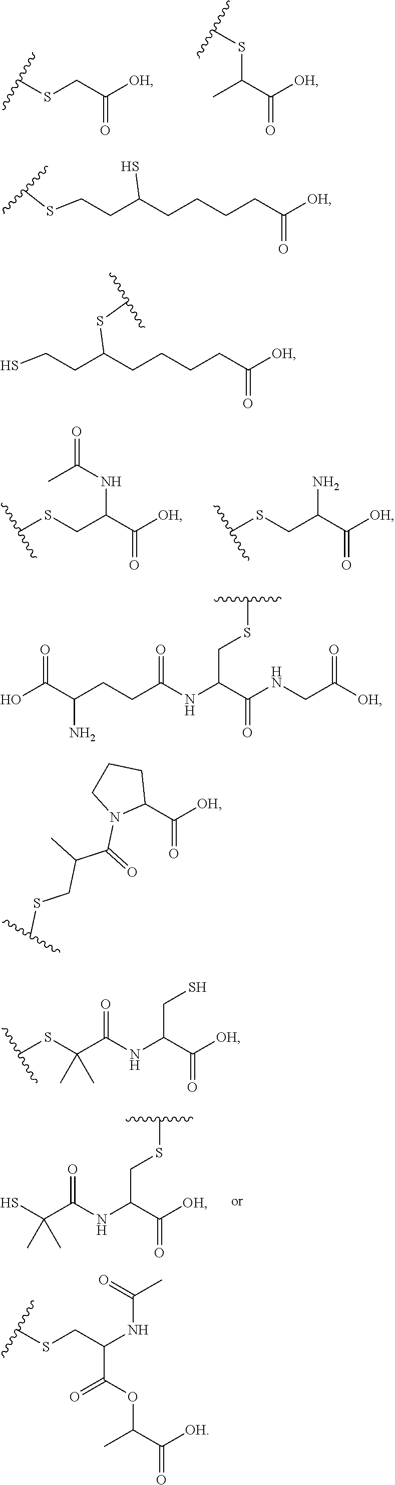

In some embodiments, R⁵ comprises one or more substituent that is a carboxylic acid or an ester. In some embodiments, R⁵ comprises one or more substituent that is a carboxylic acid (e.g., —(C═O)OH). In some embodiments, R⁵ comprises one or more substituent that is an ester (e.g., —(C═O)O—C₁-C₄alkyl).

In some embodiments, the —(C═O)OH of R⁵ is optionally esterified (e.g., —(C═O)OH or —(C═O)O—C₁-C₄alkyl). In some embodiments, the C₁-C₄alkyl is methyl, ethyl, propyl, isopropyl, butyl, or t-butyl.

In some embodiments, R^{z} is:

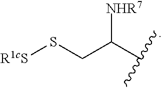

In some embodiments, R⁷ is H or —(C═O)CH₃. In some embodiments, R⁷ is H. In some embodiments, R⁷ is —(C═O)CH₃.

In some embodiments, R^{1c} is described elsewhere herein.

In some embodiments, provided herein is a compound, or a pharmaceutically acceptable salt or solvate (e.g., or a stereoisomer) thereof, having the structure of Formula (II):

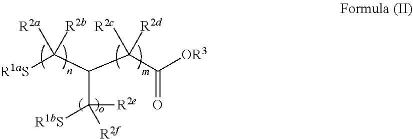

In some embodiments, R^{1a} and R^{1b} are each independently —H or SR^{1c}, either or both of R^{1a} and R^{1b} being —SR^{1c}. In some embodiments, each R^{1c} is independently substituted or unsubstituted (e.g., straight or branched) alkyl (e.g., substituted with one or more (alkyl) substituent, each (alkyl) substituent being independently selected from the group consisting of carboxylic acid, —SH, thioalkyl, acetamide, amino, oxo, optionally substituted heterocycloalkyl (e.g., N-attached pyrrolidinyl substituted with —COOH)) or substituted or unsubstituted (e.g., straight or branched) heteroalkyl (e.g., substituted with one or more heterolalkyl substituent, each (heteroalkyl) substituent being independently selected from the group consisting of carboxylic acid, amino, thioalkyl, thiol, acetamide, and C₁-C₃ alkyl). In some embodiments, each R^{2a}, R^{2b}, R^{2c}, R^{2d}, R^{2e}, and R^{2f} is independently H, halogen, C₁-C₃-alkyl, C₁-C₃-alkoxy, C₃-C₅-cycloalkyl, or two of R^{2a} and R^{2b}, R^{2c} and R^{2d}, or R^{2e} and R^{2f} are taken together with the atoms to which they are attached to form a C₃-C₅-cycloalkyl. In some embodiments, R³ is H or substituted or unsubstituted alkyl. In some embodiments, m is an integer from 1-10. In some embodiments, n and o are each independently an integer from 0-3.

In some embodiments, n, m, o, R^{2a}, R^{2b}, R^{2c}, R^{2d}, R^{2e}, and R^{2f} are each described elsewhere herein.

In some embodiments, the compound, or a pharmaceutically acceptable salt or solvate (e.g., or a stereoisomer) thereof, has the structure of Formula (IIc):

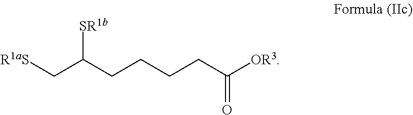

In some embodiments, R^{1a} and R^{1b} are each independently —H or SR^{1c}. In some embodiments, each R^{1c} is independently substituted or unsubstituted (e.g., straight or branched) alkyl (e.g., substituted with one or more (alkyl) substituent, each (alkyl) substituent being independently selected from the group consisting of carboxylic acid, —SH, thioalkyl, acetamide, amino, oxo, optionally substituted heterocycloalkyl (e.g., N-attached pyrrolidinyl substituted with —COOH)) or substituted or unsubstituted (e.g., straight or branched) heteroalkyl (e.g., substituted with one or more (heteroalkyl) substituent, each (heteroalkyl) substituent being independently selected from the group consisting of carboxylic acid, amino, thioalkyl, thiol, acetamide, and C₁-C₃ alkyl). In some embodiments, R³ is H or substituted or unsubstituted alkyl.

In some embodiments, R^{1a} and R^{1b} are each described elsewhere herein.

In some embodiments, the compound, or a pharmaceutically acceptable salt or solvate (e.g., or a stereoisomer) thereof, has the structure of Formula (IId):

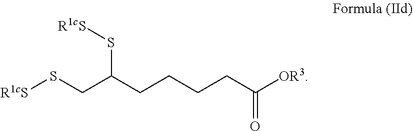

In some embodiments, each R^{1c} is independently substituted or unsubstituted (e.g., straight or branched) alkyl (e.g., substituted with one or more (alkyl) substituent, each (alkyl) substituent being independently selected from the group consisting of carboxylic acid, —SH, thioalkyl, acetamide, amino, oxo, optionally substituted heterocycloalkyl (e.g., N-attached pyrrolidinyl substituted with —COOH)) or substituted or unsubstituted (e.g., straight or branched) heteroalkyl (e.g., substituted with one or more (heteroalkyl) substituent, each (heteroalkyl) substituent being independently selected from the group consisting of carboxylic acid, amino, thioalkyl, thiol, acetamide, and C₁-C₃ alkyl). In some embodiments, R³ is H or substituted or unsubstituted alkyl.

In some embodiments, R^{1c} is described elsewhere herein.

In some embodiments, R³ is H or unsubstituted alkyl. In some embodiments, R³ is H or C₁-C₆ alkyl. In some embodiments, R³ is H, methyl, ethyl, isopropyl, or tert-butyl. In some embodiments, R³ is H.

In some embodiments, provided herein is a compound, or a pharmaceutically acceptable salt or solvate (e.g., or a stereoisomer) thereof, having the structure of Formula (III):

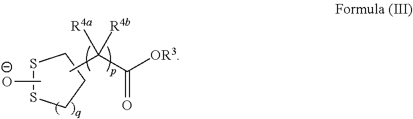

In some embodiments, each R^{4a} and R^{4b} is independently H, halogen, or substituted or unsubstituted alkyl. In some embodiments, p is an integer from 1-10. In some embodiments, q is an integer from 1-3. In some embodiments, R³ is H or substituted or unsubstituted alkyl.

In some embodiments, q, p, R^{4a}, and R^{4b} are each described elsewhere herein.

In some embodiments, the compound has the structure of Formula (IIIc):

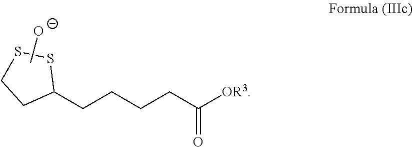

In some embodiments, R³ is H or unsubstituted alkyl. In some embodiments, R³ is H or C₁-C₆ alkyl. In some embodiments, R³ is H, methyl, ethyl, isopropyl, or tert-butyl. In some embodiments, R³ is H.

In some embodiments, the sulfoxide of any compound provided herein is racemic. In some embodiments, the sulfoxide of any compound provided herein is an enantiomer. In some embodiments, the sulfoxide of any compound provided herein is has a stereochemistry that is (R) or (S).

In some embodiments, the thiolanyl oxide ring structure is substituted with one or more substituent (e.g., each substituent being independently selected from the group consisting of halogen or unsubstituted or substituted alkyl).

In some embodiments, provided herein is a composition comprising a compound of any one of Formula (III) (e.g., Formula (III), Formula (IIIa), Formula (IIIb), Formula (IIIc), Formula (IIId), or Table 3) and lipoic acid (e.g., such that at 50% or more, 75% or more, 90% or more, or 95% or more of the composition comprises a compound of any one of Formula (III) (e.g., Formula (III), Formula (IIIa), Formula (IIIb), Formula (IIIc), Formula (IIId), or Table 3)).

In some embodiments, provided herein is a compound, or a pharmaceutically acceptable salt or solvate (e.g., or a stereoisomer) thereof, having the structure of Formula (IV):

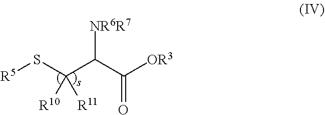

In some embodiments, R⁵ is —SR^{1c}. In some embodiments, each R^{1c} is independently substituted or unsubstituted (e.g., straight or branched) alkyl (e.g., substituted with one or more (alkyl) substituent, each (alkyl) substituent being independently selected from the group consisting of carboxylic acid, —SH, thioalkyl, acetamide, amino, oxo, optionally substituted heterocycloalkyl (e.g., N-attached pyrrolidinyl substituted with —COOH)) or substituted or unsubstituted (e.g., straight or branched) heteroalkyl (e.g., substituted with one or more (heteroalkyl) substituent, each (heteroalkyl) substituent being independently selected from the group consisting of carboxylic acid, amino, thioalkyl, thiol, acetamide, and C₁-C₃ alkyl). In some embodiments, R⁶ and R⁷ are each independently H, substituted or unsubstituted alkyl, or substituted or unsubstituted heteroalkyl. In some embodiments, each R¹⁰ and R¹¹ is independently H, halogen, C₁-C₃-alkyl, C₁-C₃-haloalkyl, C₁-C₃-alkoxy, C₃-C₅-cycloalkyl, or R¹⁰ and R¹¹ are taken together with the atoms to which they are attached to form a C₃-C₅-cycloalkyl. In some embodiments, R³ is H or substituted or unsubstituted alkyl. In some embodiments, s is an integer from 1-10.

In some embodiments, R^{1c}, R³, R⁵, R⁶, R⁷, R¹⁰, R¹¹ and s are each described elsewhere herein.

In some embodiments, the compound has the structure of Formula (IVa):

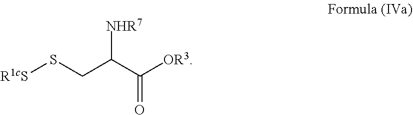

In some embodiments, R⁷ is H or —(C═O)CH₃. In some embodiments, R⁷ is H. In some embodiments, R⁷ is —(C═O)CH₃.

In some embodiments, R^{1c}, R³, and R⁷ are each described elsewhere herein.

In some embodiments, provided herein is a pharmaceutical composition comprising any compound provided herein, such as a compound represented by any structure herein, such as, for example, Formula (I), Formula (I-A), Formula (I-B), Formula (I-C), Formula (I-D), Formula (I′), Formula (Ia), Formula (Ib), Formula (Ic), Formula (Id), Formula (II), Formula (IIa), Formula (IIb), Formula (IIc), Formula (IId), Formula (III), Formula (IIIa), Formula (IIIb), Formula (IIIc), Formula (IIId), Formula (IV), Formula (IVa), Table 1, Table 2, Table 3, Table 4, or a pharmaceutically acceptable salt thereof, and at least one pharmaceutically acceptable excipient. In some embodiments, the pharmaceutical composition is suitable for ophthalmic administration. In some embodiments, the pharmaceutical composition is suitable for topical ophthalmic administration. In some embodiments, topical ophthalmic administration is administration in and/or around the eye, such as to the eyelid margin. In some embodiments, topical ophthalmic administration is administration to the ocular surface and the inner surface to the eyelid.

In some embodiments, a compound or a pharmaceutical composition comprising any compound provided herein, such as a compound of any one of Formula (I), Formula (I-A), Formula (I-B), Formula (I-C), Formula (I-D), Formula (I′), Formula (Ia), Formula (Ib), Formula (Ic), Formula (Id), Formula (II), Formula (IIa), Formula (IIb), Formula (IIc), Formula (IId), Formula (III), Formula (IIIa), Formula (IIIb), Formula (IIIc), Formula (IIId), Formula (IV), Formula (IVa), Table 1, Table 2, Table 3, Table 4, or a pharmaceutically acceptable salt thereof, is substantially hydrolytically stable (e.g., stable in an aqueous composition (e.g., solution), such as a buffer solution or ophthalmically acceptable aqueous composition). In some embodiments, the compound or the pharmaceutical composition is formulated in an aqueous vehicle. In some embodiments, the compound or the pharmaceutical composition is formulated and stored in an aqueous vehicle. In some instances, compositions or formulations provided herein are chemically and/or physically stable in an aqueous composition.

In some embodiments, a compound provided herein, such as a compound of any one of Formula (I), Formula (I-A), Formula (I-B), Formula (I-C), Formula (I-D), Formula (I′), Formula (Ia), Formula (Ib), Formula (Ic), Formula (Id), Formula (II), Formula (IIa), Formula (IIb), Formula (IIc), Formula (IId), Formula (III), Formula (IIIa), Formula (IIIb), Formula (IIIc), Formula (IIId), Formula (IV), Formula (IVa), Table 1, Table 2, Table 3, Table 4, or a pharmaceutically acceptable salt thereof, is reduced to one or more keratolytic agent (e.g., a free form of a radical of Formula (II), Formula (IIa), Formula (IIb), Formula (IIc), Formula (IId), Formula (III), Formula (IIIa), Formula (IIIb), Formula (IIIc), Formula (IIId), Formula (IV), Formula (IVa), Table 2, Table 3, or Table 4, such as wherein R is a negative charge or H) and/or hydrolyzed to an active pharmaceutical agent (e.g., a free form of a radical of Formula (I), Formula (I-A), Formula (I-B), Formula (I-C), Formula (I-D), Formula (I′), Formula (Ia), Formula (Ib), Formula (Ic), Formula (Id), or Table 1, such as wherein R is a negative charge or H). In some embodiments, the compound or pharmaceutical composition is reduced to one or more keratolytic agent in an ocular space. In some embodiments, the compound or pharmaceutical composition is reduced to one or more keratolytic agent by a reductase in an ocular space.

In some embodiments, a compound provided herein, such as a compound of any one of Formula (I), Formula (I-A), Formula (I-B), Formula (I-C), Formula (I-D), Formula (I′), Formula (Ia), Formula (Ib), Formula (Ic), Formula (Id), Table 1, or a pharmaceutically acceptable salt thereof, is hydrolyzed to an active pharmaceutical agent (e.g., a free form of a radical of Formula (I), Formula (I-A), Formula (I-B), Formula (I-C), Formula (I-D), Formula (I′), Formula (Ia), Formula (Ib), Formula (Ic), Formula (Id), or Table 1, such as wherein R is a negative charge or H) and a keratolytic agent. In some embodiments, the compound or pharmaceutical composition is hydrolyzed to an active pharmaceutical agent and a keratolytic agent in an ocular space. In some embodiments, the compound or pharmaceutical composition is hydrolyzed to an active pharmaceutical agent and a keratolytic agent by an esterase in an ocular space. In some embodiments, the active pharmaceutical agent is an anti-inflammatory agent. In some embodiments the anti-inflammatory agent is (S)-3-(4-((4-carbamoylpiperidine-1-carbonyl)oxy)phenyl)-2-((S)-4-methyl-2-(2-(o-tolyloxy)acetamido)pentanamido)propanoic acid. In some embodiments, the keratolytic agent is a carboxylic acid. In some embodiments, the carboxylic acid is selected from the group consisting of acetic acid, glycolic acid, lactic acid, lipoic acid, pivalic acid, isobutryic acid, butyric acid, propionic acid, formic acid, and carbonic acid. In some embodiments, the active keratolytic agent is a thiol.

In some embodiments, a compound or a pharmaceutical composition comprising any compound provided herein, such as a compound of any one of Formula (I), Formula (I-A), Formula (I-B), Formula (I-C), Formula (I-D), Formula (I′), Formula (Ia), Formula (Ib), Formula (Ic), Formula (Id), Formula (II), Formula (IIa), Formula (IIb), Formula (IIc), Formula (IId), Formula (III), Formula (IIIa), Formula (IIIb), Formula (IIIc), Formula (IIId), Formula (IV), Formula (IVa), Table 1, Table 2, Table 3, Table 4, or a pharmaceutically acceptable salt thereof. In certain embodiments, the composition further comprises an amount of a free form of a radical of any of Formula (I), Formula (I-A), Formula (I-B), Formula (I-C), Formula (I-D), Formula (I′), Formula (Ia), Formula (Ib), Formula (Ic), Formula (Id), Formula (II), Formula (IIa), Formula (IIb), Formula (IIc), Formula (IId), Formula (III), Formula (IIIa), Formula (IIIb), Formula (IIIc), Formula (IIId), Formula (IV), Formula (IVa), Table 1, Table 2, Table 3, Table 4, or the like (such as wherein the free form is the radical, wherein R is a negative charge or an H). In some embodiments, a composition provided herein comprises a (e.g., weight or molar) ratio of a compound provided herein to a free form of a radical of Formula (I), Formula (I-A), Formula (I-B), Formula (I-C), Formula (I-D), Formula (I′), Formula (Ia), Formula (Ib), Formula (Ic), Formula (Id), Formula (II), Formula (IIa), Formula (IIb), Formula (IIc), Formula (IId), Formula (III), Formula (IIIa), Formula (IIIb), Formula (IIIc), Formula (IIId), Formula (IV), Formula (IVa), Table 1, Table 2, Table 3, Table 4, or a pharmaceutically acceptable salt thereof (e.g., wherein R is a negative charge or an H) is about 1:99 to about 100:0 (e.g., the amount of the free form of the radical relative to the overall amount of free form of the radical plus the conjugate is between 0% (weight or molar) and 99%). In some embodiments, the relative amount of the free form of the radical is 0% to about 50%, such 0% to about 20%, 0% to about 10%, about 0.1% to about 10%, about 0.1% to about 5%, less than 5%, less than 2.5%, less than 2%, or the like (percentages being weight/weight or mole/mole percentages). In some instances, such aqueous compositions are pre-manufactured or are manufactured at the time of application in order to maintain high concentrations of the compound relative to the free form of a radical thereof. In some embodiments, such concentrations of the compound are present in the composition for at least 45 minutes in an aqueous composition (such as in an aqueous composition, e.g., a HEPES buffer, such as under the conditions described herein, such as in Tables 5 and 6). Tables 5 and 6 of the Examples illustrate good stability of the compositions provided herein and such recitations are incorporated in the disclosure hereof. Further, in some instances, compounds provided herein release free form of a radical of a compound of Formula (I), Formula (I-A), Formula (I-B), Formula (I-C), Formula (I-D), Formula (I′), Formula (Ia), Formula (Ib), Formula (Ic), Formula (Id), Formula (II), Formula (IIa), Formula (IIb), Formula (IIc), Formula (IId), Formula (III), Formula (IIIa), Formula (IIIb), Formula (IIIc), Formula (IIId), Formula (IV), Formula (IVa), Table 1, Table 2, Table 3, or Table 4 (e.g., wherein R is a negative charge or H), such as when administered to an individual (e.g., ocular (e.g., peri-ocular) or dermatological administration). In more specific instances, when administered to an individual at a location with esterases and/or reductases present, rapid release of active (free) forms of a radical of Formula (I), Formula (I-A), Formula (I-B), Formula (I-C), Formula (I-D), Formula (I′), Formula (Ia), Formula (Ib), Formula (Ic), Formula (Id), Formula (II), Formula (IIa), Formula (IIb), Formula (IIc), Formula (IId), Formula (III), Formula (IIIa), Formula (IIIb), Formula (IIIc), Formula (IIId), Formula (IV), Formula (IVa), Table 1, Table 2, Table 3, Table 4, (e.g., wherein R is a negative charge or H) (and, a keratolytic agent and/or agent that further produces active keratolytic agent(s) (e.g., by further hydrolysis and/or reduction thereof)).

In some embodiments, provided herein a compound or a pharmaceutical composition comprising any compound provided herein, such as a compound of any one of Formula (I), Formula (I-A), Formula (I-B), Formula (I-C), Formula (I-D), Formula (I′), Formula (Ia), Formula (Ib), Formula (Ic), Formula (Id), Formula (II), Formula (IIa), Formula (IIb), Formula (IIc), Formula (IId), Formula (III), Formula (IIIa), Formula (IIIb), Formula (IIIc), Formula (IIId), Formula (IV), Formula (IVa), Table 1, Table 2, Table 3, Table 4, or a pharmaceutically acceptable salt thereof, has keratolytic effects (e.g., reduces disulfide (S—S) bonds) (e.g., in any environment provided herein), such as, for example, as shown in FIG. 1.

Provided in some embodiments herein is a method of treating inflammation and/or hyperkeratosis, the method comprising administering to an individual (e.g., in need thereof) any compound provided herein (e.g., of any Formula or Table provided herein) (e.g., in a therapeutically effective amount). In specific embodiments, the inflammation and/or hyperkeratosis is inflammation and/or hyperkeratosis of the eye, periocular structures (e.g., eyelid), and/or skin.

Provided in some embodiments herein is a method of treating a dermatological or an ophthalmic disease or disorder in an individual in need of thereof, comprising administering to the individual in need thereof a composition comprising any compound provided herein, such as a compound represented by any structure herein, such as, for example, Formula (I), Formula (I-A), Formula (I-B), Formula (I-C), Formula (I-D), Formula (I′), Formula (Ia), Formula (Ib), Formula (Ic), Formula (Id), Formula (II), Formula (IIa), Formula (IIb), Formula (IIc), Formula (lid), Formula (III), Formula (IIIa), Formula (IIIb), Formula (IIIc), Formula (IIId), Formula (IV), Formula (IVa), Table 1, Table 2, Table 3, Table 4, or a pharmaceutically acceptable salt thereof. In some embodiments, the dermatological or ophthalmic disease or disorder is inflammation or hyperkeratosis of the eyes or skin (e.g., the ocular surface). In some embodiments, the dermatological or ophthalmic dermatological disease or disorder is selected from the group consisting of meibomian gland dysfunction (MGD), dry eye disease (DED), ocular manifestations of graft versus host disease, vernal keratoconjunctivitis, atopic keratoconjunctivitis, Cornelia de Lange Syndrome, evaporative eye disease, aqueous deficiency dry eye, blepharitis, and seborrheic blepharitis. In some embodiments, the dermatological or ophthalmic disease or disorder is inflammation or hyperkeratosis (e.g., of the eyes or skin), such as, for example, meibomian gland dysfunction (MGD), dry eye disease (DED), ocular manifestations of graft versus host disease, vernal keratoconjunctivitis, atopic keratoconjunctivitis, Cornelia de Lange Syndrome, evaporative eye disease, aqueous deficiency dry eye, blepharitis, seborrheic blepharitis, or any combination thereof.

In some embodiments, the ophthalmic disease or disorder is selected from dry eye, lid wiper epitheliopathy (LWE), contact lens discomfort (CLD), contact lens discomfort, dry eye syndrome, evaporative dry eye syndrome, aqueous deficiency dry eye syndrome, blepharitis, keratitis, meibomian gland dysfunction, conjunctivitis, lacrimal gland disorder, inflammation of the anterior surface of the eye, infection of the anterior surface of the eye, infection of the lid, demodex lid infestation, lid wiper epitheliopathy and autoimmune disorder of the anterior surface of the eye.

In some embodiments, provided herein is a method of treating an ocular (e.g., peri-ocular) or dermatological indication (e.g., associated with keratolytic activity, inflammation, and/or microbial infiltration), the method comprising administering a therapeutically effective amount of a compound or composition provided herein. In some embodiments, a composition provided herein (e.g., used in a method provided herein) comprises a compound provided herein in a therapeutically effective amount (e.g., at a concentration effective to treat keratosis/keratolytic activity, inflammation, and/or microbial infiltration), in the eye, surrounding tissue, or skin. In some embodiments, a (e.g., pharmaceutical and/or ophthalmic) composition provided herein comprises about 0.1 wt. % to about 10 wt. % of a compound provided herein.

In some embodiments, ocular and/or dermatological disorders include, for example, inflammatory conditions of the eyelids (e.g., hordeolum (stye), blepharitis, and chalazion), ocular surface (e.g., dry eye disease and anterior uveitis) and posterior eye (e.g., posterior and pan-uveitis), abnormalities of the peri-ocular glands (e.g., meibomian gland dysfunction (MGD)), allergic-type conditions, (e.g., eczema, atopic dermatitis, atopic keratoconjunctivitis refractory to topical steroid treatment, and vernal keratoconjunctivitis), surgical complications (e.g., corneal transplant rejection, post-corneal transplant glaucoma, cataracts secondary to phakic corneal transplant, fungal infections in keratoplasty patients, and post-LASIK dry eye and/or poor refractive outcomes), corneal abnormalities (e.g., inflammatory corneal ulceration, rheumatoid corneal ulcers, and Thygeson's superficial punctate keratitis), conjunctival abnormalities (e.g., iridocyclitis, ligneous conjunctivitis), ocular complications from systemic treatments and/or autoimmune diseases (e.g., pauciarticular juvenile rheumatoid arthritis, graft versus host disease, and sjogren's syndrome) and/or infectious disease of the anterior surface of the eye. In some embodiments, provided herein are compositions and methods for the treatment of ocular and periocular abnormalities that have multifactorial etiologies and interactions.

### BRIEF DESCRIPTION OF THE DRAWINGS

Some of the novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows free thiol radical formation for a compound provided herein.

### INCORPORATION BY REFERENCE

All publications, patents, and patent applications mentioned in this specification are herein incorporated by reference for the specific purpose identified herein.

### DETAILED DESCRIPTION

### Certain Definitions

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary from 1% to 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

The terms “treat,” “treating,” or “treatment” as used herein, include reducing, alleviating, abating, ameliorating, relieving, or lessening the symptoms associated with a disease, disease sate, or indication (e.g., addiction, such as opioid addiction, or pain) in either a chronic or acute therapeutic scenario. Also, treatment of a disease or disease state described herein includes the disclosure of use of such compound or composition for the treatment of such disease, disease state, or indication.

“Amino” refers to the —NH₂ radical.

“Cyano” refers to the —CN radical.

“Nitro” refers to the —NO₂ radical.

“Oxo” refers to the ═O radical.

“Alkyl” generally refers to an acyclic (e.g., straight or branched) or cyclic hydrocarbon (e.g., chain) radical consisting solely of carbon and hydrogen atoms, such as having from one to fifteen carbon atoms (e.g., C₁-C¹⁵ alkyl). Unless otherwise state, alkyl is saturated or unsaturated (e.g., an alkenyl, which comprises at least one carbon-carbon double bond). Disclosures provided herein of an “alkyl” are intended to include independent recitations of a saturated “alkyl,” unless otherwise stated. Alkyl groups described herein are generally monovalent, but may also be divalent (which may also be described herein as “alkylene” or “alkylenyl” groups). In certain embodiments, an alkyl comprises one to thirteen carbon atoms (e.g., C₁-C₁₃ alkyl). In certain embodiments, an alkyl comprises one to eight carbon atoms (e.g., C₁-C⁸ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. In general, alkyl groups are each independently substituted or unsubstituted. Each recitation of “alkyl” provided herein, unless otherwise stated, includes a specific and explicit recitation of an unsaturated “alkyl” group. Similarly, unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜ OR^{a} (where t is 1 or 2), —S(O)ₜR^{a} (where t is 1 or 2) and —S(O)ₜ N(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is optionally substituted as described for “alkyl” groups.

“Alkylene” or “alkylene chain” generally refers to a straight or branched divalent alkyl group linking the rest of the molecule to a radical group, such as having from one to twelve carbon atoms, for example, methylene, ethylene, propylene, i-propylene, n-butylene, and the like. Unless stated otherwise specifically in the specification, an alkylene chain is optionally substituted as described for alkyl groups herein.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system can contain hydrogen and carbon from five to eighteen carbon atoms, where at least one of the rings in the ring system is fully unsaturated, i.e., it contains a cyclic, delocalized (4n+2) π-electron system in accordance with the Hülckel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜ OR^{a} (where t is 1 or 2) and —R^{b}—S(O)ₜ N(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” or “aryl-alkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” or “cycloalkyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl or cycloalkyl is saturated (i.e., containing single C—C bonds no double or triple bonds between two carbons) or unsaturated (i.e., containing one or more double bonds or triple bonds). Examples of saturated cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}—R^{b}—C(O)N(R^{a})₂—R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜ OR^{a} (where t is 1 or 2) and —R^{b}—S(O)ₜ N(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carboxylic acid,” “COOH,” or “(C═O)OH” refers to a radical of the formula —COOH. Each recitation of “carboxylic acid,” “COOH,” or “(C═O)OH” provided herein, unless otherwise stated, includes a specific and explicit recitation of an esterified “carboxylic acid,” “COOH,” or “(C═O)OH” group (e.g., or radical thereof). In some embodiments, the esterified carboxylic acid group (or radical thereof) is (C═O)O—C₁-C₄alkyl, wherein alkyl is as defined hereinabove. In some embodiments, “carboxylic acid,” “COOH,” or “(C═O)OH” is COOH. In some embodiments, “carboxylic acid,” “COOH,” or “(C═O)OH” is (C═O)O—C₁-C₄alkyl.

“Carbocyclylalkyl” refers to a radical of the formula —R^{c}-carbocyclyl where R_{c} is an alkylene chain as defined above. The alkylene chain and the carbocyclyl radical is optionally substituted as defined above.

“Carbocyclylalkenyl” refers to a radical of the formula —R^{c}-carbocyclyl where R_{c} is an alkenylene chain as defined above. The alkenylene chain and the carbocyclyl radical is optionally substituted as defined above.

“Carbocyclylalkoxy” refers to a radical bonded through an oxygen atom of the formula−O—R^{c}-carbocyclyl where R^{c} is an alkylene chain as defined above. The alkylene chain and the carbocyclyl radical is optionally substituted as defined above.

“Halo” or “halogen” refers to fluoro, bromo, chloro, or iodo substituents.

“Haloalkyl” refers to an alkyl radical, as defined above, that is substituted by one or more halogen radicals, as defined above, for example, trihalomethyl, dihalomethyl, halomethyl, and the like. In some embodiments, the haloalkyl is a fluoroalkyl, such as, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

The term “heteroalkyl” refers to an alkyl group as defined above in which one or more skeletal carbon atoms of the alkyl are substituted with a heteroatom (with the appropriate number of substituents or valencies—for example, —CH₂— may be replaced with —NH— or —O—). For example, each substituted carbon atom is independently substituted with a heteroatom, such as wherein the carbon is substituted with a nitrogen, oxygen, sulfur, or other suitable heteroatom. In some instances, each substituted carbon atom is independently substituted for an oxygen, nitrogen (e.g. —NH—, —N(alkyl)-, or —N(aryl)- or having another substituent contemplated herein), or sulfur (e.g. —S—, —S(═O)—, or —S(═O)₂—). In some embodiments, a heteroalkyl is attached to the rest of the molecule at a carbon atom of the heteroalkyl. In some embodiments, a heteroalkyl is attached to the rest of the molecule at a heteroatom of the heteroalkyl. In some embodiments, a heteroalkyl is a C₁-C¹⁸ heteroalkyl. In some embodiments, a heteroalkyl is a C₁-C₁₂ heteroalkyl. In some embodiments, a heteroalkyl is a C₁-C₆ heteroalkyl. In some embodiments, a heteroalkyl is a C₁-C₄ heteroalkyl. Representative heteroalkyl groups include, but are not limited to —OCH₂OMe, or —CH₂CH₂OMe. In some embodiments, heteroalkyl includes alkoxy, alkoxyalkyl, alkylamino, alkylaminoalkyl, aminoalkyl, heterocycloalkyl, heterocycloalkyl, heterocyclyl, and heterocycloalkylalkyl, as defined herein. Unless stated otherwise specifically in the specification, a heteroalkyl group is optionally substituted as defined above for an alkyl group.

“Heteroalkylene” refers to a divalent heteroalkyl group defined above which links one part of the molecule to another part of the molecule. Unless stated specifically otherwise, a heteroalkylene is optionally substituted, as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, “heterocyclyl” and “heterocycloalkyl” are used interchangeably herein. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl radical is saturated (i.e., containing single C—C bonds only) or unsaturated (e.g., containing one or more double bonds or triple bonds in the ring system). In some instances, the heterocyclyl radical is saturated (e.g., dithiolanyl or dithiolanyl oxide). In some instances, the heterocyclyl radical is saturated and substituted (e.g., dithiolanyl oxide). In some instances, the heterocyclyl radical is unsaturated. The heterocyclyl is attached to the rest of the molecule through any atom of the ring(s). Examples of such heterocyclyl radicals include, but are not limited to, dithiolanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclyl alkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b} C(O)R^{a} , —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜ OR^{a} (where t is 1 or 2) and —R^{b}—S(O)ₜ N(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R_{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, b enzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahy droquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}—R^{b}—OC(O)—R^{a}, —R^{b}—OC(O)OR^{a}, —R^{b}—OC(O)—N(R^{a})₂, —R^{b}—N(R^{a})₂, —R^{b}—C(O)R^{a}, —R^{b}—C(O)O)OR^{a}, —R^{b}—C(O)N(R^{a})₂, —R^{b}—O—R^{c}—C(O)N(R^{a})₂, —R^{b}—N(Rₐ)C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜ OR^{a} (where t is 1 or 2) and —R^{b}—(O)ₜ N(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R_{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)— or (S)—. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

In general, optionally substituted groups are each independently substituted or unsubstituted. Each recitation of an optionally substituted group provided herein, unless otherwise stated, includes an independent and explicit recitation of both an unsubstituted group and a substituted group (e.g., substituted in certain embodiments, and unsubstituted in certain other embodiments). Unless otherwise stated, substituted groups may be substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜ OR^{a} (where t is 1 or 2), —S(O)ₜR^{a} (where t is 1 or 2) and —S(O)ₜ N(R^{a})₂ (where t is 1 or 2) where each IV is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Pharmaceutically acceptable salt” includes both acid and base addition salts. A pharmaceutically acceptable salt of any one of the pharmacological agents described herein is intended to encompass any and all pharmaceutically suitable salt forms. Exemplary pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

Compositions

The meibomian glands are large sebaceous glands located in the eyelids, and unlike skin, are unassociated with hair. The meibomian glands produce the lipid layer of the tear film that protects it against evaporation of the aqueous phase. The meibomian gland orifice is located on the epithelial side of the lid margin, and can be a few hundred microns from the mucosal side. The glands are located on both upper and lower eyelids, with higher amounts of the glands on the upper eyelid. A single meibomian gland is composed of clusters of secretory acini that are arranged circularly around a long central duct and connected to it by short ductules. The terminal part of the central duct is lined by an ingrowth of the epidermis that covers the free lid margin and forms a short excretory duct that opens as an orifice at the posterior part of the lid margin just anterior to the mucocutaneous junction near the inner lid border. The oily secretion composed of lipids is synthesized within the secretory acini. The lipid secretion is a liquid at near body temperature and is delivered to the skin of the lid margin as a clear fluid, called “meibum.” It forms shallow reservoirs on the upper and lower lid margins, and consists of a complex mixture of cholesterol, wax, cholesteryl esters, phospholipids, with small amounts of triglycerides, triacylglycerols, and hydrocarbons. The separate meibomian glands are arranged in parallel, and in a single row throughout the length of the tarsal plates in the upper and lower lids. The extent of the glands corresponds roughly to the dimensions of the tarsal plates.

The term “keratinized obstruction” as used herein refers to a blockage of the meibomian gland, regardless of the location of the blockage. In some embodiments, the blockage is complete, whereas in other embodiments, the blockage is partial. Regardless of the degree of blockage, such keratinized obstruction leads to meibomian gland dysfunction. In some embodiments, the keratinized obstruction is composed of keratinized material and lipids. In some embodiments, the keratinized obstruction is a blockage at the meibomian gland orifice and excretory duct. In some embodiments, the keratinized obstruction is caused by keratinization of the epithelium at the lid margin and meibomian gland. In certain instances, the keratin obstruction is influenced by the migration or aberrant differentiation of stem cells. In some embodiments, the keratinized obstruction results in reduced delivery of oil to the lid margin and tear film, and stasis inside the meibomian gland that causes increased pressure, resultant dilation, acinar atrophy, and low secretion. In certain instances, keratinization of the meibomian gland causes degenerative gland dilation and atrophy.

Ocular surface diseases is a group of diseases including, but not limited to, dry eye syndrome (including evaporative DES and/or aqueous deficiency DES), blepharitis, keratitis, meibomian gland dysfunction, conjunctivitis, lacrimal gland disorder, contact lens related conditions and inflammatory, infectious, or autoimmune diseases or disorders of the anterior surface of the eye. The term, “meibomian gland dysfunction,” as used herein, refers to chronic, diffuse abnormality of the meibomian glands, that is characterized by terminal duct obstruction or qualitative or quantitative changes in the glandular secretion, or both. MGD may result in alteration of the tear film, eye irritation symptoms, inflammation, or ocular surface disease. The most prominent aspects of MGD are obstruction of the meibomian gland orifices and terminal ducts and changes in the meibomian gland secretions.

In some instances, meibomian gland dysfunction (MGD) is a chronic, diffuse abnormality of the meibomian glands, which can be characterized by terminal duct obstruction and/or qualitative/quantitative changes in the glandular secretion. Terminal duct obstruction is caused by hyperkeratinization of the ductal epithelium (Nichols et al, Inv. Oph. & Vis. Sci. (2011); 52(4):1922-1929). These alterations in both meibum quality and expression may result in alteration of the tear film, symptoms of eye irritation, and ocular surface disease such as evaporative dry eye. The principal clinical consequence of MGD is evaporative dry eye syndrome and large population based studies (i.e., Bankok Study and the Shihpai Eye Study) estimate that over 60% of patients with dry eye symptoms also have MGD (Schaumberg et al, Investigative Ophthalmology and Visual Science. (2011); 52(4):1994-2005).

MGD is a leading contributor of dry eye syndrome. The occurrence of dry eye syndrome is widespread and affects about 20 million patients in the United States alone. Dry eye syndrome is a disorder of the ocular surface resulting from either inadequate tear production or excessive evaporation of moisture from the surface of the eye. Tears are important to corneal health because the cornea does not contain blood vessels, and relies on tears to supply oxygen and nutrients. Tears and the tear film are composed of lipids, water, and mucus, and disruption of any of these can cause dry eye. An inadequate amount of lipids flowing from the meibomian glands as caused by a keratinized obstruction, may cause excessive evaporation, thereby causing dry eye syndrome.

In some embodiments, altered meibomian gland secretion is detected by physically expressing the meibomian glands by applying digital pressure to the tarsal plates. In subjects without MGD, the meibum is a pool of clear oil. In MGD, both the quality and expressibility of the expressed material is altered. The altered meibum is also known as meibomian excreta and is made up of a mixture of altered secretions and keratinized epithelial material. In MGD, the quality of expressed lipid varies in appearance from a clear fluid, to a viscous fluid containing particulate matter and densely opaque, toothpaste-like material. The meibomian orifices may exhibit elevations above surface level of the lid, which is referred to as plugging or pouting, and is due to obstruction of the terminal ducts and extrusion of a mixture of meibomian lipid and keratinized material.

Obstructive MGD is characterized by all or some of the following: 1) chronic ocular discomfort, 2) anatomic abnormalities around the meibomian gland orifice (which is one or more of the following: vascular engorgement, anterior or posterior displacement of the mucocutaneous junction, irregularity of the lid margin) and 3) obstruction of the meibomian glands (obstructive findings of the gland orifices by slit lamp biomicroscopy (pouting, plugging or ridge), decreased meibum expression by moderate digital pressure).

Current methods for assessing and monitoring MGD symptoms include, but are not limited to patient questionnaires, meibomian gland expression, tear stability break up time, and determining the number of patent glands as seen by digital expression.

In some embodiments, the symptoms of a patient are assessed by asking the patient a series of questions. Questionnaires allow the assessment of a range of symptoms associated with ocular discomfort. In some embodiments, the questionnaire is the SPEED questionnaire. The SPEED questionnaire assesses frequency and severity of a patient's dry eye symptoms. It examines the occurrence of symptoms on the current day, past 72 hours and past three months. A SPEED score is tallied based on the patient's answers to the questions, to give a range of severity of the patient's symptoms. The SPEED questionnaire includes questions such as the following: 1) what dry eye symptoms are you experiencing, and when do they occur? 2) how frequently do you experience dryness, grittiness, or scratchiness in your eyes? 3) how often do you experience soreness or irritation of the eyes? 4) how often do you experience burning or watering of the eyes? 5) how often do you experience eye fatigue? and 6) how severe are the symptoms?

Meibomian gland expressibility is optionally determined to assess the meibomian gland function. In normal patients, meibum is a clear to light yellow oil. Meibum is excreted from the glands when digital pressure is placed on the glands. Changes in meibomian gland expressibility are one potential indicator of MGD. In some embodiments, during expression, quantifying the amount of physical force applied during expression is monitored in addition to assessing lipid volume and lipid quantity.

Tear stability break up time (TBUT) is a surrogate marker for tear stability. Tear film instability is a core mechanism in dry eye and MGD. Low TBUT implies a possibility of lipid layer compromise and MGD. TBUT is optionally measured by examining fluorescein breakup time, as defined as the time to initial breakup of the tear film after a blink. Fluorescein is optionally applied by wetting a commercially available fluorescein-impregnated strip with saline, and applied to the inferior fornix or bulbar conjuctiva. The patient is then asked to blink several times and move the eyes. The break up is then analyzed with a slit lamp, a cobalt blue filter, and a beam width of 4 mm. The patient is instructed to blink, and the time from upstroke of the last blink to the first tear film break or dry spot formation is recorded as a measurement.

Other methods for assessing MGD symptoms, include but are not limited to, Schirmer test, ocular surface staining, lid morphology analysis, meibography, meibometry, interferometry, evaporimetry, tear lipid composition analysis, fluorophotometry, meiscometry, osmolarity analysis, indices of tear film dynamics, evaporation and tear turnover.

Current treatments for MGD include lid warming, lid massage, lid hygiene, lid expression and meibomian gland probing. Pharmacological methods, prior to those described herein, have not been used.

Lid hygiene is considered the primary treatment for MGD and consists of three components: 1) application of heat, 2) mechanical massage of eyelids and 3) cleansing the eyelid. Eyelid warming procedures improve meibomian gland secretion by melting the pathologically altered meibomian lipids. Warming is achieved by warm compresses or devices. Mechanical lid hygiene includes the use of scrubs, mechanical expression and cleansing with various solutions of the eyelashes and lid margins. Lid margins are optionally also cleansed with hypoallergenic bar soap, dilute infant shampoo or commercial lid scrubs. Physical expression of meibomian glands is performed in a physician's office or is performed by the patient at home. The technique varies from gentle massage of the lids against the eyeball to forceful squeezing of the lids either against each other or between a rigid object on the inner lid surface and a finger, thumb, or rigid object (such as a glass rod, cotton swab, or metal paddle) on the outer lid surface. The rigid object on the inner lid surface protects the eyeball from forces transferred through the eyelid during expression and to offer a stable resistance, to increase the amount of force that is applied to the glands.

Eyelid warming is limited because the warming melts the lipids, but does not address movement of the keratinized material. Further, eyelid warming induces transient visual degradation due to corneal distortion. Mechanical lid hygiene is also limited because the force needed to remove an obstruction can be significant, resulting in significant pain to the patient. The effectiveness of mechanical lid hygiene is limited by the patient's ability to tolerate the associated pain during the procedure. Other treatments for MGD are limited.

Physical opening of meibomian glands obstruction by meibomian gland expression is an acceptable method to improve meibomian gland secretion and dry eye symptoms. In addition probing of the meibomian gland canal has been used to open the obstructed canal. Both methods, expression and probing, are limited, however, by the pain induced by the procedure, the possible physical insult to the gland and canal structures and their short lived effect estimated at days and weeks. Therefore, methods are needed to improve patient comfort, which will not cause harm to the meibomian glands and canals, that will reduce the dependency on frequent office visits and improve secretion of meibum.

U.S. Pat. No. 9,463,201 entitled, “Compositions and methods for the treatment of meibomian gland dysfunction” describes a method for treating meibomian gland dysfunction involving the topical administration of a therapeutically-effective amount of at least one keratolytic agent in an ophthalmically-acceptable carrier. The patent includes keratolytic agents that are inorganic selenium (Se) compounds such as selenium disulfide (SeS₂) or organoselenium compounds such as Ebselen (2-Phenyl-1,2-benzoselenazol-3-one). This agent would treat the underlying cause of MGD, but not a “plus” inflammatory disease as described by the DEWS report on MGD.

The role of inflammation in the etiology of MGD is controversial. The terms posterior blepharitis and MGD are not synonymous. Posterior blepharitis describes inflammatory conditions of the posterior lid margin and has various causes, of which MGD can be one possible cause (Nichols et al 2011). In its earliest stages, MGD is not associated with clinical signs characteristic of posterior blepharitis. As MGD progresses, an MGD-related posterior blepharitis is said to be present. MGD-related posterior blepharitis affects the meibomian glands and meibomian gland orifices. MGD-related posterior blepharitis is characterized by flora changes, esterase and lipase release, lipid changes, and eyelid inflammation. Hyperkeratinization of the meibomian gland epithelium (thickening of the lining of the glands) may lead to obstruction and a decrease in the quantity of meibomian gland secretions and may be responsible for MGD-related posterior blepharitis. Diagnosis of MGD-related posterior blepharitis includes meibomian gland expression with demonstration of an altered quality of expressed secretions, and/or by a loss of gland functionality (decreased or absent expressibility). The TFOS report on Meibomian Gland Disease specifically notes that anterior blepharitis and exacerbated inflammatory ocular surface disease are “plus” diseases to MGD which are managed by topical, ocular steroids (Nichols et al 2011). Since these “plus” conditions can be present in various levels of severity from early to late MGD there is a need for treatments and/or combinations of treatments that can target both the underlying non-inflammatory pathophysiology of MGD and inflammation associated with these comorbid conditions.

MGD-related inflammatory eye disease may comprise a different mechanism than blepharitis-related MGD. MGD-related inflammatory eye disease is characterized by an inflammatory cascade involving activation and migration of T lymphocytes to the inflamed tissue. T lymphocyte infiltration may result in lacrimal gland stimulation and upregulation of cytokines. Exemplary cytokines that may be involved in MGD-related inflammatory eye disease include, but are not limited to, interleukin-1, interleukin-4, interleukin-6, inteleukin-8, interferon gamma, macrophage inflammatory protein 1 alpha, and tumor necrosis factor alpha. Kinase pathways including the mitogen activated protein kinase (MAPK) pathway are also activated in the inflammatory cascade. The inflammatory process results in loss of mucin-producing goblet cells and destruction of the ocular surface that can lead to further damage.

Dry eye syndrome, also known as keratoconjunctivitis sicca (KCS), is considered a self-sustaining disease that is progressively disconnected from its initial cause. Dry eye syndrome is associated with inflammation at the ocular surface and periocular tissue. Inflammation is characterized by the activation and migration of T lymphocytes to the inflamed tissue including in the conjunctiva and lacrimal glands. Inflammatory cytokines, chemokines, and matrix metalloproteinase have also been identified as being increased.

Animal models of dry eye disease have been established and reviewed (Barabino, et al, (Invest. Ophthalmol. Vis. Sci. 2004, 45:1641-1646)). Barabino, et al, (Invest. Ophthalmol. Vis. Sci. 2005, 46:2766-2771) described a model wherein exposure of normal mice to a low-humidity environment in a controlled-environment chamber leads to significant alterations in tear secretion, goblet cell density, and acquisition of dry eye-related ocular surface signs. However, no single animal model adequately accounts for the immune, endocrine, neuronal and environmental factors which contribute to dry eye pathogenesis.

Anti-inflammatory agents may be used to treat ocular surface diseases or disorders including dry eye syndrome. Corticosteroids are an effective anti-inflammatory therapy in dry eye disease. For example, in a 4-week, double-masked, randomized study in 64 patients with dry eye and delayed tear clearance, loteprednol etabonate 0.5% ophthalmic suspension (Lotemax [Bausch and Lomb, Rochester, NY]), QID, was found to be more effective than its vehicle in improving some signs and symptoms (Pflugfelder et al, Am J Ophthalmol (2004); 138:444-57). The TFOS 2007 report on dry eye disease went so far as to conclude that, “In the US Federal Regulations, ocular corticosteroids receiving “class labeling” are indicated for the treatment “ . . . of steroid responsive inflammatory conditions of the palpebral and bulbar conjunctiva, cornea and anterior segment of the globe such as allergic conjunctivitis, acne rosacea, superficial punctate keratitis, herpes zoster keratitis, iritis, cyclitis, selected infective conjunctivitis, when the inherent hazard of steroid use is accepted to obtain an advisable diminution in edema and inflammation.” KCS, in some instances, is included in this list of steroid-responsive inflammatory conditions (Therapy Subcommittee of the International Dry Eye WorkShop, 2007. Management and Therapy of Dry Eye Disease: Report of the Management and Therapy Subcommittee of the International Dry Eye WorkShop (2007). 2007; 5: 163-178).” While the US FDA does not agree with this conclusion, short courses of steroids, especially Lotemax, can be commonly used to treat inflammation associated with dry eye disease.

Other anti-inflammatory agents include nonsteroidal anti-inflammatory drugs (NSAIDs). NSAIDs inhibit the activity of cyclooxygenases including cyclooxygenase-1 (COX-1) and cyclooxygenase-2 (COX-2), which are enzymes involved in the synthesis of prostaglandins and thromboxanes from arachidonic acid. Prostaglandin and thromboxane signaling are involved in inflammation and immune modulation. In some cases, NSAIDs are used for treating dry eye disease by treating the inflammation at the ocular surface.

Treatment of dry eye is also accomplished through agents that enhance tear fluid and mucin production. For example, agonists of the P2Y₂ receptor have been shown to increase tear fluid and mucin secretion. The mechanism is thought to involve P2Y₂ signaling to raise intracellular calcium and open chloride channels in the apical membrane. The P2Y₂ receptor belongs to the family of purinergic receptors, which have been classified into P1 receptors and P2 receptors on the basis of their native agonism by purine nucleosides and purine and pyrimidine nucleotides, respectively. P2 receptors are further distinguished physiologically into two types: P2X receptors and P2Y receptors. The P2Y receptors are involved in diver signaling including platelet aggregation, immunity, lipid metabolism, and bone activity. Several studies have also demonstrated the presence of P2X and P2Y receptors in ocular tissues, including the retina, ciliary body, and lens. These studies indicate that P2Y₂ receptors appear to be the main subtype of purinergic receptor located at the ocular surface. P2Y₂ receptors have also been demonstrated to be localized in ocular tissues in the conjunctival epithelial goblet and serous cells and meibomian gland acinus and ductal epithelial cells of the rhesus macaque.

### (S)-3-(4-((4-carbamoylpiperidine-1-carbonyl)oxy)phenyl)-2-((S)-4-methyl-2-(2-(o-tolyloxy)acetamido)pentanamido)propanoic acid

(S)-3-(4-((4-carbamoylpiperidine-1-carbonyl)oxy)phenyl)-2-((S)-4-methyl-2-(2-(o-tolyloxy)acetamido)pentanamido)propanoic acid has a molecular formula of C₃₁—H₄₀N₄O₈ and a molecular weight of 596.7. The structural formula is:

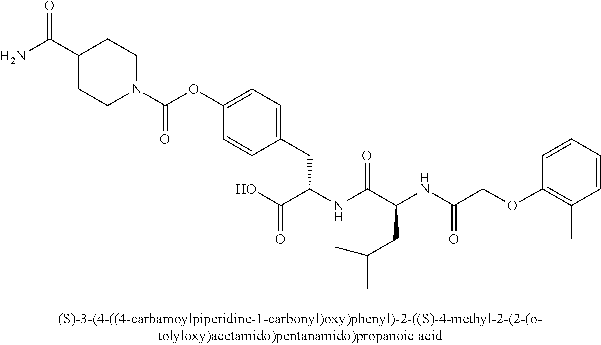 [SMILES: Cc1ccccc1OCC(=O)N[C@@H](CC(C)C)C(=O)N[C@@H](Cc1ccc(OC(=O)N2CCC(C(N)=O)CC2)cc1)C(=O)O]

(S)-3-(4-((4-carbamoylpiperidine-1-carbonyl)oxy)phenyl)-2-((S)-4-methyl-2-(2-(o-tolyloxy)acetamido)pentanamido)propanoic acid is a potent integrin a4 antagonist (Ravensberg et al, Allergy (2006) 61, 1097-1103) that has demonstrated improvements in objective signs of dry eye in the murine DS model. The potent integrin α4 antagonist was found to act locally at the level of the ocular surface, presumably by preventing the migration of antigen-presenting cells to the draining lymph nodes with a resulting interruption of the immune cycle of dry eye (Invest. Ophthalmol. Vis. Sci. (2015) 56(10), 5888-5895).

Described herein are compounds (e.g., keratolytic conjugates and/or dual acting-agents) which address simultaneously the non-inflammatory keratolytic blockage component of meibomian gland dysfunction and the inflammation associated dry eye disease including aqueous deficiency. In some embodiments, a compound provided herein is useful as either an acute therapy (e.g., by a trained specialist or physician) or as a chronic therapy (e.g., in the hands of a patient, or alternatively, by a trained specialist or physician). A compound provided herein is tested, in some embodiments, using the assays and methods described herein (e.g., as described in the examples). In some embodiments, a compound provided herein represents a significant advance in the art as the first-order metabolites obtained from metabolism of the agents are operative against both the keratolytic and the inflammatory component of dry eye disease.

In some embodiments, provided herein is a compound, having the structure of Formula (I):

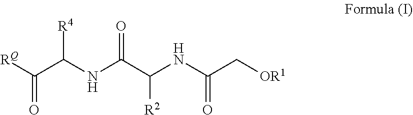

or a pharmaceutically acceptable salt or solvate (e.g., or a stereoisomer) thereof, wherein:
R¹ is aryl, cycloalkyl, heterocyclyl, or heteroaryl, wherein the aryl, cycloalkyl, heterocyclyl, or heteroaryl is optionally substituted;
R² is substituted or unsubstituted alkyl;
R⁴ is -L^{a}-R^{4x}, wherein L^{a} is a bond, alkylene, or heteroalkylene, and R^{4x} is absent, substituted or unsubstituted cycloalkyl, substituted or unsubstituted heterocycloalkyl, substituted or unsubstituted aryl, or substituted or unsubstituted heteroaryl; and
R^{Q} is -L′-D, wherein:
   D is a keratolytic agent (e.g., or a radical thereof); and
   L′ is a (e.g., hydrolyzable) linker.

In some embodiments, L^{a} is alkylene or heteroalkylene. In some embodiments, L^{a} is alkylene. In some embodiments, R^{4x} is substituted or unsubstituted aryl or substituted or unsubstituted heteroaryl. In some embodiments, R^{4x} is substituted or unsubstituted aryl. In some embodiments, R⁴ is optionally substituted alkyl-aryl. In some embodiments, L^{a} is alkyl and R^{4x} is substituted or unsubstituted aryl. In some embodiments, L^{a} is alkyl and R^{4x} is substituted or unsubstituted aryl. In some embodiments, L^{a} is alkyl and R^{4x} is substituted aryl (e.g., being substituted with substituted heteroalkyl, being substituted with optionally substituted (e.g., N-attached) heterocycloalkyl. In some embodiments, L^{a} is alkyl and R^{4x} is substituted aryl, the aryl being substituted with heteroalkyl further substituted with N-attached heterocycloalkyl further substituted with formamidyl.

In some embodiments provided herein is a compound, having the structure of Formula (I-A):

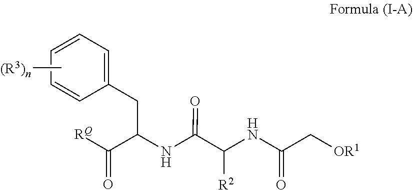

or a pharmaceutically acceptable salt or solvate (e.g., or a stereoisomer) thereof,
wherein:
   R¹ is aryl, cycloalkyl, heterocyclyl, or heteroaryl, wherein the aryl, cycloalkyl, heterocyclyl, or heteroaryl is optionally substituted;
   R² is substituted or unsubstituted alkyl;
   each R³ is independently selected from the group consisting of halogen, —CN, —NO₂, substituted or unsubstituted alkyl, substituted or unsubstituted heteroalkyl, substituted or unsubstituted alkoxy, amine, amide, ester, carboxylic acid;
   n is 0-5; and
   R^{Q} is -L′-D, wherein:
      D is a keratolytic agent; and
      L′ is a (e.g., hydrolyzable) linker.

In some embodiments, L′ comprises one or more linker groups, wherein each linker group is selected from the group consisting of a bond, —O—, —S—, alkyl (alkylenyl), heteroalkyl (heteroalkylenyl), disulfide, ester and carbonyl. In some embodiments, the keratolytic agent comprises one or more group (e.g., keratolytic group), each group (e.g., keratolytic group) being independently selected from the group consisting of thiol, disulfide, selenium (e.g., selenide, diselenide), and carboxylic acid.

In some embodiments, provided herein is a compound having the structure of Formula (I-B):

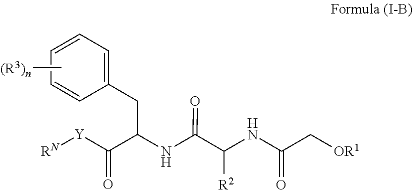

or a pharmaceutically acceptable salt or solvate (e.g., or a stereoisomer) thereof, wherein:
   R¹ is aryl, cycloalkyl, heterocyclyl, or heteroaryl, wherein the aryl, cycloalkyl, heterocyclyl, or heteroaryl is optionally substituted;
   R² is substituted or unsubstituted alkyl;
   each R³ is independently selected from the group consisting of halogen, —CN, —NO₂, substituted or unsubstituted alkyl, substituted or unsubstituted heteroalkyl, substituted or unsubstituted alkoxy, amine, amide, ester, carboxylic acid, substituted or unsubstituted cycloalkyl, or substituted or unsubstituted heterocycloalkyl;
   n is 0-5;
   Y is 0 or S; and
   R^{N} is alkyl substituted with at R^{4a}st one oxo or heteroalkyl substituted with at R^{4a}st one oxo, the alkyl or heteroalkyl being further optionally substituted.

In some embodiments, provided herein is a compound having the structure of Formula (I-C):

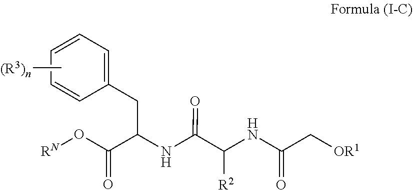

or a pharmaceutically acceptable salt or solvate (e.g., or a stereoisomer) thereof, wherein
R¹ is aryl, cycloalkyl, heterocyclyl, or heteroaryl, wherein the aryl, cycloalkyl, heterocyclyl, or heteroaryl is optionally substituted;
   R² is substituted or unsubstituted alkyl;
   each R³ is independently selected from the group consisting of halogen, —CN, —NO₂, substituted or unsubstituted alkyl, substituted or unsubstituted heteroalkyl, substituted or unsubstituted alkoxy, amine, amide, ester, carboxylic acid;
   n is 0-5; and
   R^{N} is alkyl substituted with at least one oxo or heteroalkyl substituted with at least one oxo, the alkyl or heteroalkyl being further optionally substituted.

In some embodiments, the alkyl or heteroalkyl (e.g., of R^{N}) is substituted with one or more substituent, each substituent independently selected from the group consisting of alkyl, heteroalkyl, hydroxyl, thiol, thioether, disulfide, seleno, selenol, selenide, diselenide, sulfone, amide, halo, oxo, heterocyclyl, and cycloalkyl, wherein the heterocyclyl, and cycloalkyl is optionally substituted (e.g., with one or more substituent selected from the group consisting of alkyl, heteroalkyl, hydroxyl, thiol, thioether, disulfide, selenol, selenide, diselenide, sulfone, amide, halo and oxo).

In some embodiments, R^{N} is:

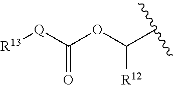

wherein:
   Q is —O— or a bond;
   R¹² is hydrogen, alkyl, heteroalkyl, or haloalkyl; and
   R¹³ is alkyl, heteroalkyl, or heterocycloalkyl, the alkyl, heteroalkyl, or heterocycloalkyl being optionally substituted.

In some embodiments, the alkyl or heteroalkyl (e.g., of R¹³) is substituted with one or more substituent, each substituent independently selected from the group consisting of alkyl, heteroalkyl, hydroxyl, thiol, thioether, disulfide, seleno, selenol, selenide, diselenide, sulfone, amide, ester, carboxylic acid, halo, oxo, heterocyclyl, and cycloalkyl, wherein the heterocyclyl, and cycloalkyl is optionally substituted (e.g., with one or more substituent selected from the group consisting of alkyl, heteroalkyl, hydroxyl, thiol, thioether, disulfide, selenol, sulfone, amide, ester halo and oxo).

In some embodiments, R¹ is optionally substituted aryl, heteroaryl, cycloalkyl, or heterocyclyl. In some embodiments, le is substituted aryl. In some embodiments, le is phenyl substituted with alkyl. In some embodiments, R¹ is phenyl substituted with methyl.

In some embodiments, R₂ is unsubstituted alkyl. In some embodiments, R₂ is C₁-C₆ alkyl. In some embodiments, R₂ is isobutyl.

In some embodiments, R³ is substituted heteroalkyl, substituted or unsubstituted cycloalkyl, or substituted or unsubstituted heterocycloalkyl. In some embodiments, R³ is substituted heteroalkyl. In some embodiments, R³ is substituted heteroalkyl, being substituted with optionally substituted (e.g., N-attached) heterocycloalkyl. In some embodiments, R³ is substituted heteroalkyl, being substituted with N-attached heterocycloalkyl substituted with formamidyl.

In some embodiments, provided herein is a compound having the structure of Formula (I-D):

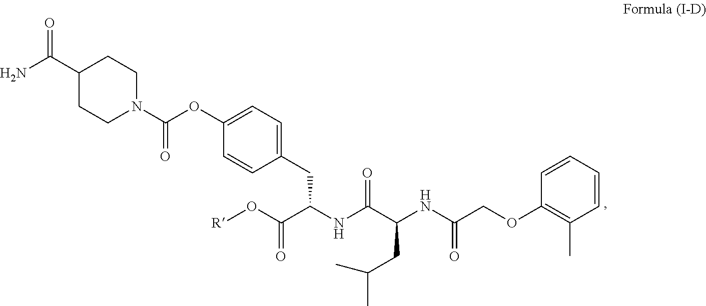

or a pharmaceutically acceptable salt or solvate (e.g., or a stereoisomer) thereof,
wherein:
   R′ is substituted alkyl (e.g., substituted with at least one oxo) or substituted or unsubstituted heteroalkyl.

In some embodiments, R′ is substituted alkyl, the alkyl being substituted with substituted alkoxy. In some embodiments, R′ is substituted alkyl, the alkyl being substituted with substituted alkoxy, the alkoxy being substituted with one or more substituent, each substituent being independently selected from the group consisting of oxo, hydroxy, optionally substituted C₁-C₆ alkoxy, thiol, optionally substituted heterocycloalkyl, acetamidyl, substituted unsaturated cycloalkyl (e.g., being substituted with one or more C₁-C₄ alkyl), and amino.

In some embodiments, R′ is substituted alkyl, the alkyl being substituted with substituted alkoxy, the alkoxy being substituted with C₁-C₆ alkoxy further substituted with one or more substituent, each substituent being independently selected from the group consisting of oxo, hydroxy, optionally substituted C₁-C₄ alkoxy (e.g., being optionally substituted with hydroxyl), optionally substituted heterocycloalkyl, and hydroxyalkyl. In some embodiments, R′ is substituted alkyl, the alkyl being substituted with substituted alkoxy, the alkoxy being substituted with C₁-C₆ alkoxy further substituted with C₁-C₄ alkoxy optionally substituted with oxo, hydroxy, C₁-C₄ alkoxy, and/or C₁-C₄ alkyl. In some embodiments, R′ is substituted alkyl, the alkyl being substituted with substituted alkoxy, the alkoxy being substituted with 1, 3-dioxane substituted with one or more methyl. In some embodiments, R′ is substituted alkyl, the alkyl being substituted with substituted alkoxy, the alkoxy being substituted with C₁-C₆ alkoxy further substituted with C₁-C₆ alkyl-acyl optionally substituted with hydroxyl.

In some embodiments, R′ is substituted alkyl, the alkyl being substituted with substituted alkoxy, the alkoxy being substituted with substituted unsaturated cycloalkyl (e.g., being substituted with one or more C₁-C₄ alkyl),

In some embodiments, R′ is substituted alkyl, the alkyl being substituted with substituted alkoxy, the alkoxy being substituted with heterocycloalkyl (e.g., dithiolanyl or dithiolanyl oxide). In some embodiments, R′ is substituted alkyl, the alkyl being substituted with substituted alkoxy, the alkoxy being substituted with dithiolanyl or dithiolanyl oxide.

In some embodiments, R′ is substituted alkyl, the alkyl being substituted with substituted alkoxy, the substituted alkoxy being substituted with oxo and one or more other substituent, each other substituent being independently selected from the group consisting of optionally substituted C₁-C₆ alkoxy (e.g., being substituted with one or more substituent, each substituent being independently selected from the group consisting of oxo, hydroxy, optionally substituted C₁-C₄ alkoxy (e.g., being optionally substituted with oxo, hydroxy, C₁-C₄ alkoxy, and/or C₁-C₄ alkyl), optionally substituted heterocycloalkyl (e.g., 1, 3-dioxane substituted with one or more methyl), hydroxyalkyl, optionally substituted C₁-C₆ alkyl-acyl (e.g., the acyl being optionally substituted with hydroxy)), hydroxy, thiol, heterocycloalkyl (e.g., dithiolanyl or dithiolanyl oxide), acetamide, and amino. In some embodiments, the C₁-C₆ alkoxy is substituted with one or more substituent, each substituent being independently selected from the group consisting of oxo, hydroxy, optionally substituted C₁-C₄ alkoxy (e.g., being optionally substituted with oxo, hydroxy, C₁-C₄ alkoxy, and/or C₁-C₄ alkyl), optionally substituted heterocycloalkyl (e.g., 1, 3-dioxane substituted with one or more methyl), hydroxyalkyl, optionally substituted C₁-C₆ alkyl-acyl (e.g., the acyl being optionally substituted with hydroxy). In some embodiments, the heterocycloalkyl is dithiolanyl or dithiolanyl oxide.

In some embodiments, R′ is substituted or unsubstituted (e.g., linear or branched) heteroalkyl comprising one or more —C—O—C— (e.g., within the (e.g., linear or branched) heteroalkyl chain).

In some embodiments, R′ is substituted or unsubstituted (e.g., linear or branched) heteroalkyl comprising one or more ester, one or more carbonate, one or more amide, and/or one or more disulfide (e.g., within the (e.g., linear or branched) heteroalkyl chain).

In some embodiments, R′ is substituted or unsubstituted (e.g., linear or branched) heteroalkyl comprising one carbonate (e.g., within the (e.g., linear or branched) heteroalkyl chain).

In some embodiments, R′ is substituted or unsubstituted (e.g., linear or branched) heteroalkyl comprising one or two ester (e.g., within the (e.g., linear or branched) heteroalkyl chain).

In some embodiments, R′ is substituted or unsubstituted (e.g., linear or branched) heteroalkyl comprising one ester (e.g., within the (e.g., linear or branched) heteroalkyl chain).

In some embodiments, R′ is substituted or unsubstituted (e.g., linear or branched) heteroalkyl comprising one ester and one carbonate (e.g., within the (e.g., linear or branched) heteroalkyl chain).

In some embodiments, R′ is substituted or unsubstituted (e.g., linear or branched) heteroalkyl comprising one or two ester and one amide (e.g., within the (e.g., linear or branched) heteroalkyl chain).

In some embodiments, R′ is substituted or unsubstituted (e.g., linear or branched) heteroalkyl comprising one ester and one amide (e.g., within the (e.g., linear or branched) heteroalkyl chain).

In some embodiments, R′ is substituted or unsubstituted (e.g., linear or branched) heteroalkyl comprising one or two amide (e.g., within the (e.g., linear or branched) heteroalkyl chain).

In some embodiments, R′ is substituted or unsubstituted (e.g., linear or branched) heteroalkyl comprising one or two disulfide (e.g., within the (e.g., linear or branched) heteroalkyl chain).

In some embodiments, R′ is substituted or unsubstituted (e.g., linear or branched) heteroalkyl comprising one or two disulfide and one ester (e.g., within the (e.g., linear or branched) heteroalkyl chain).

In some embodiments, R′ is substituted (e.g., linear or branched) heteroalkyl, the (e.g., linear or branched) heteroalkyl being substituted with one or more (heteroalkyl) substituent, each (heteroalkyl) substituent being independently selected from the group consisting of optionally substituted C₁-C₆ alkyl, acetamide, hydroxy, heterocycloalkyl, thiol, thioalkyl, amino, and carboxylic acid.

In some embodiments, R′ is substituted (e.g., linear or branched) heteroalkyl, the (e.g., linear or branched) heteroalkyl being substituted with substituted C₁-C₆ alkyl, the C₁-C₆ alkyl being substituted with heteroalkyl being further optionally substituted with one or more other substituent, each other substituent being independently selected from the group consisting of hydroxy, carboxylic acid, optionally substituted N-substituted pyrrolidinyl (e.g., optionally substituted with carboxylic acid)). In some embodiments, R′ is substituted (e.g., linear or branched) heteroalkyl, the (e.g., linear or branched) heteroalkyl being substituted with acetamide and carboxylic acid.

In some embodiments, R′ is substituted (e.g., linear or branched) heteroalkyl, the (e.g., linear or branched) heteroalkyl being substituted with heterocycloalkyl. In some embodiments, R′ is substituted (e.g., linear or branched) heteroalkyl, the (e.g., linear or branched) heteroalkyl being substituted with 1,2-dithiolane, 1,2-dithiolane oxide, optionally substituted dioxane (e.g., optionally substituted with one or more C₁-C₆ alkyl), (e.g., N-substituted) pyrrolidine (e.g., substituted with alkyl (e.g., further substituted with oxo, thiol, and C₁-C₃ alkyl)), or substituted (e.g., N-attached) pyrrolidine (e.g., substituted with carboxylic acid).

In some embodiments, R′ is substituted branched heteroalkyl.

In some embodiments, R′ is:

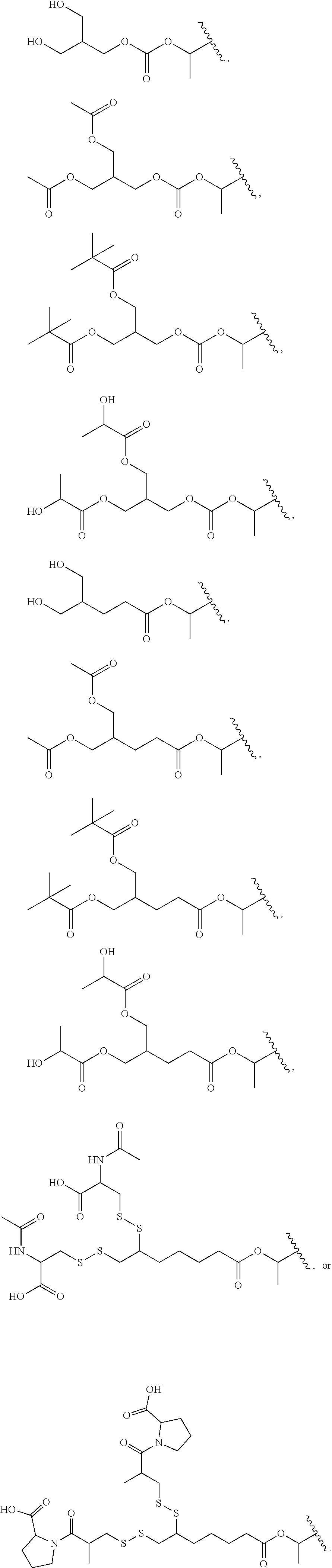

In some embodiments, R′ is:

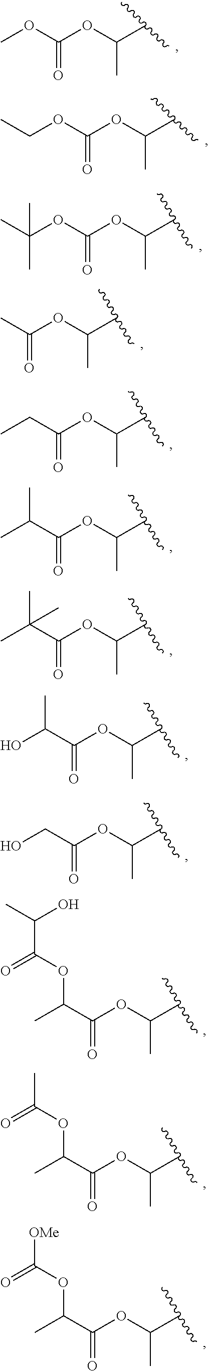

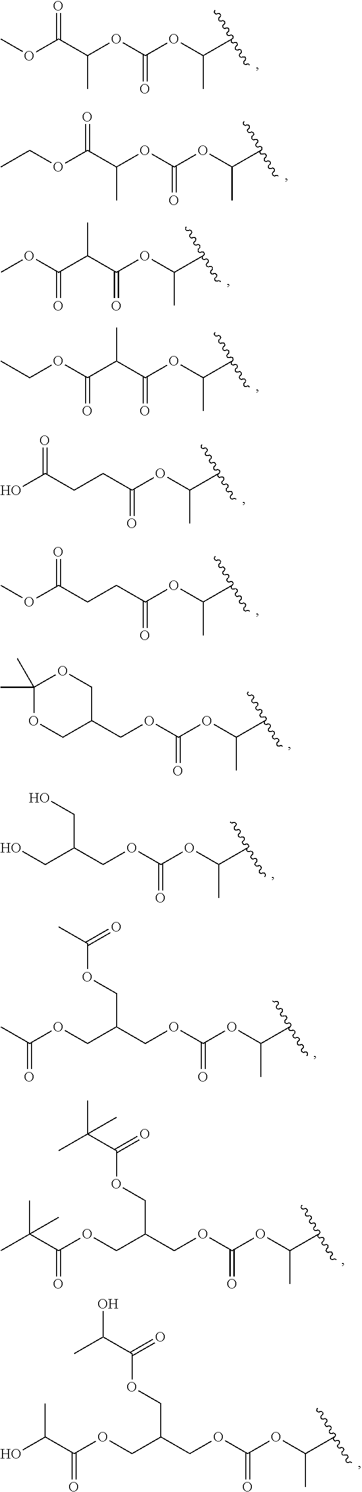

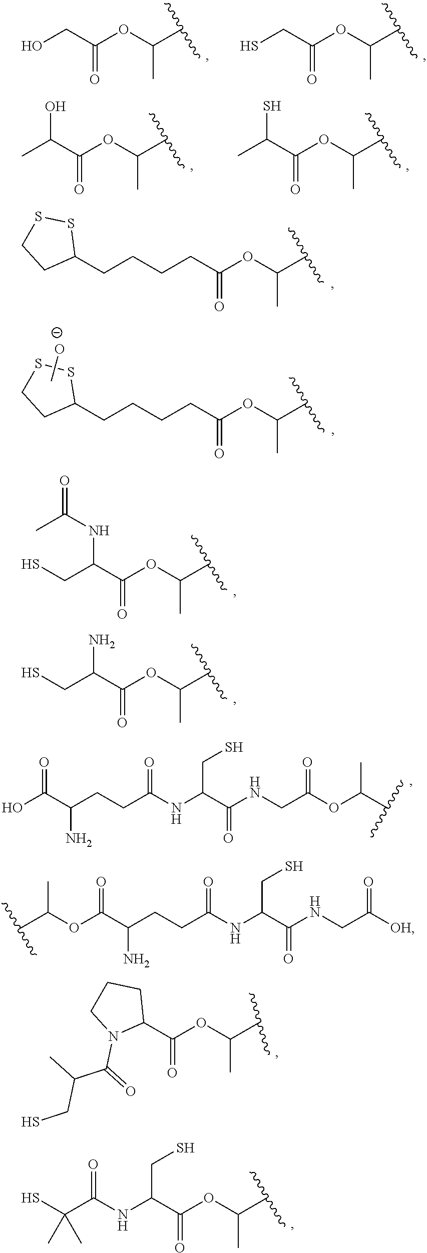

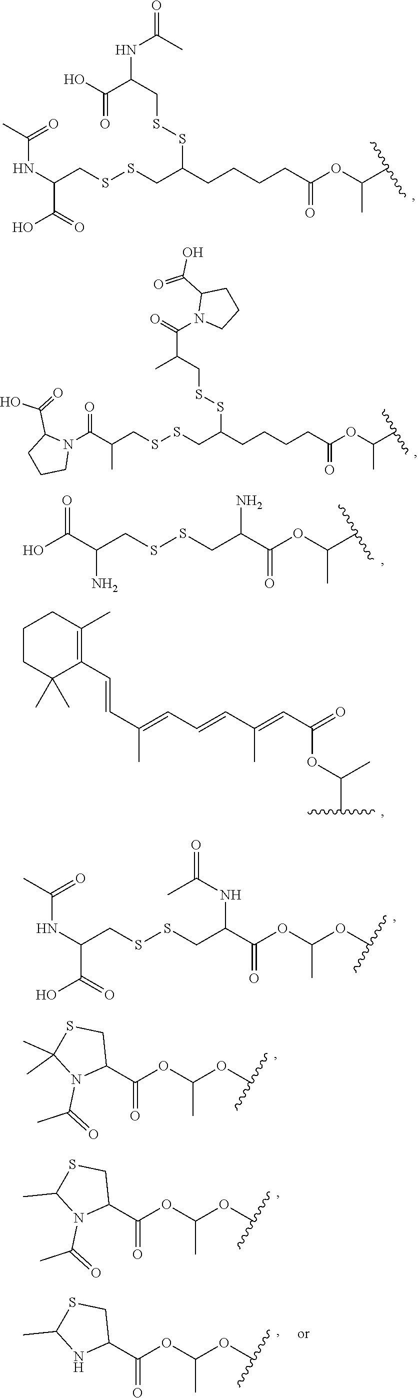

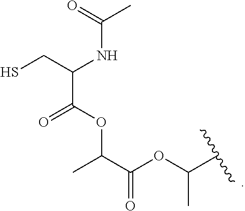 [SMILES: *C(C)OC(=O)C(C)OC(=O)C(CS)NC(C)=O]

In some embodiments, R′ is:

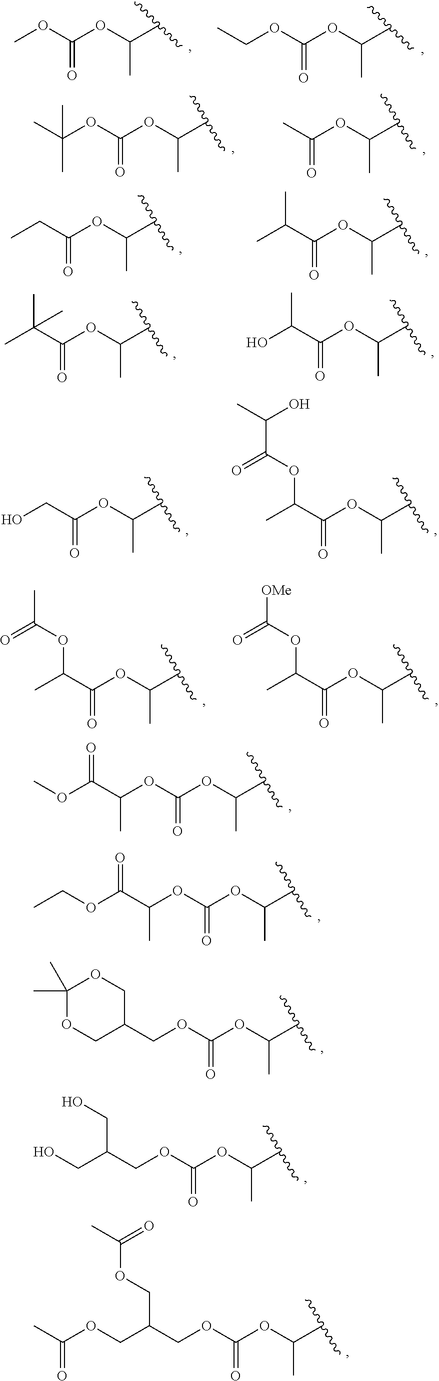

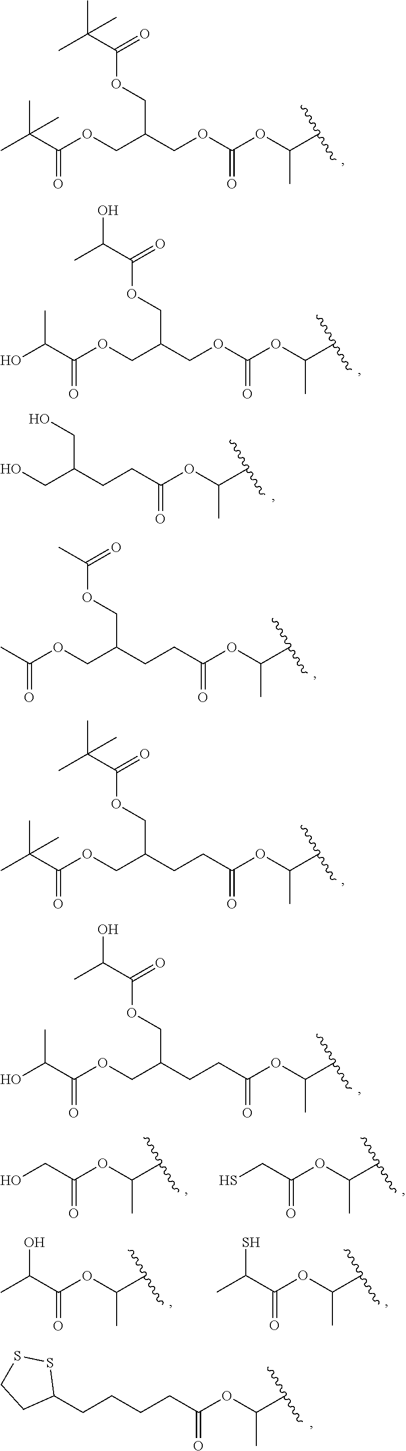

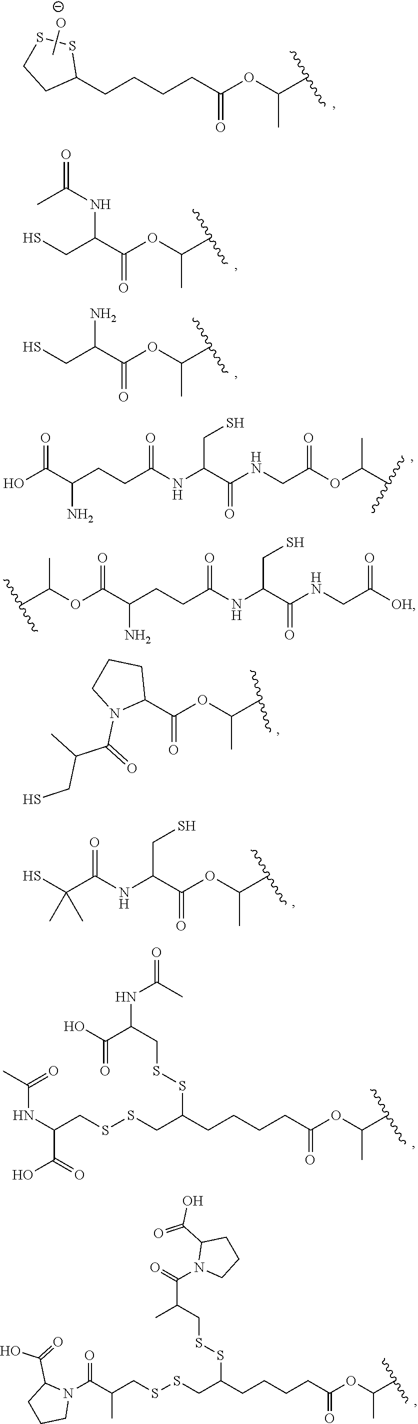

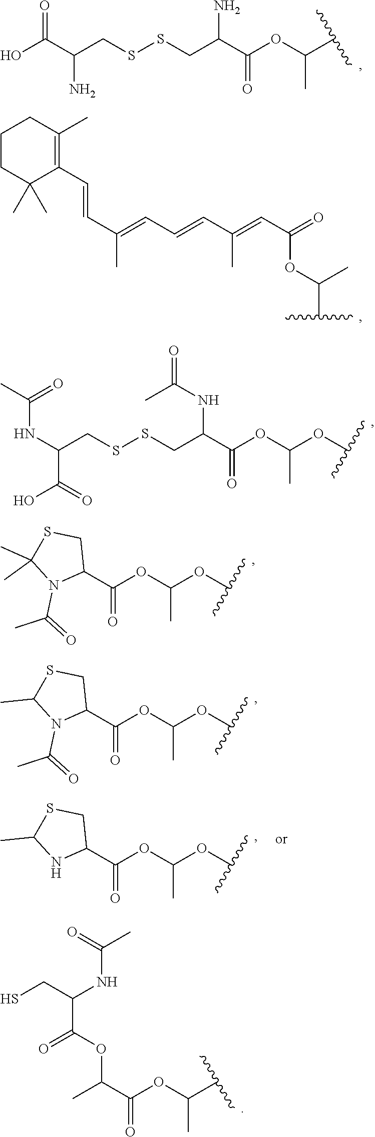

In some embodiments, provided herein is a compound having the structure of Formula

(Ia):

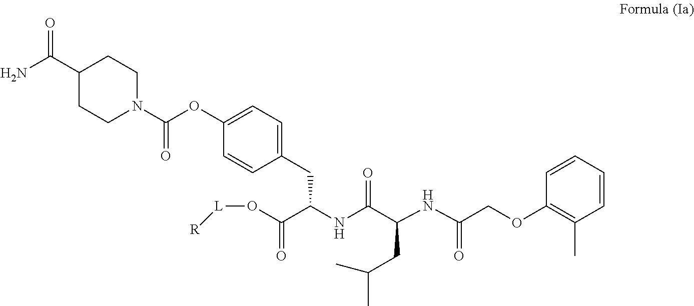

or a pharmaceutically acceptable salt or solvate (e.g., or a stereoisomer) thereof,
wherein:
   L is bond, —O(C═O)(OCR⁸R⁹)_{z}—, or —(C═O)(OCR⁸R⁹)_{z}—;
      each R⁸ and R⁹ is independently H, halogen, C₁-C₃-alkyl, C₁-C₃-haloalkyl, C₁-C₃-alkoxy, C₃-C₅-cycloalkyl, or R⁸ and R⁹ are taken together with the atoms to which they are attached to form a C₃-C₅-cycloalkyl;
      z is 1-6; and
   R is substituted or unsubstituted (e.g., straight or branched) alkyl, substituted or unsubstituted (e.g., straight or branched) heteroalkyl, or substituted or unsubstituted heterocycloalkyl (e.g., (N—) substituted with alkyl (e.g., further substituted with oxo and thiol)).

In some embodiments, L is bond. In some embodiments, L is —(C═O)(OCR⁸R⁹)_{z}—. In some embodiments, L is —O(C═O)(OCR⁸R⁹)_{z}—. In some embodiments z is 1-3. In some embodiments, z is 1. In some embodiments, each R⁸ and R⁹ is independently H or C₁-C₃-alkyl. In some embodiments, each R⁸ is H and each R⁹ is C₁-C₃-alkyl. In some embodiments, each R⁸ is H and each R⁹ is CH₃. In some embodiments, R⁸ and R⁹ are H.

In some embodiments, L is —(C═O)OCH(CH₃)—.

In some embodiments, L is —O(C═O)OCH(CH₃)—.

In some embodiments, R is substituted (e.g., straight or branched) alkyl, the (e.g., straight or branched) alkyl being substituted with one or more (alkyl) substituent, each (alkyl) substituent being independently selected from the group consisting of hydroxy, optionally substituted alkoxy (e.g., optionally substituted with oxo and hydroxy or oxo and C₁-C₃ alkoxy)), oxo, optionally substituted alkyl (e.g., optionally substituted with alkoxy further optionally substituted with oxo, C₁-C₄ alkyl, and/or hydroxy), optionally substituted heterocycloalkyl (e.g., optionally substituted dioxane (e.g., 1,3 dioxanyl optionally substituted with methyl), dithiolanyl, or dithiolanyl oxide), hydroxyalkyl, thiol, acetamide, substituted unsaturated cycloalkyl (e.g., being substituted with one or more C₁-C₄ alkyl), and amino.

In some embodiments, R is:

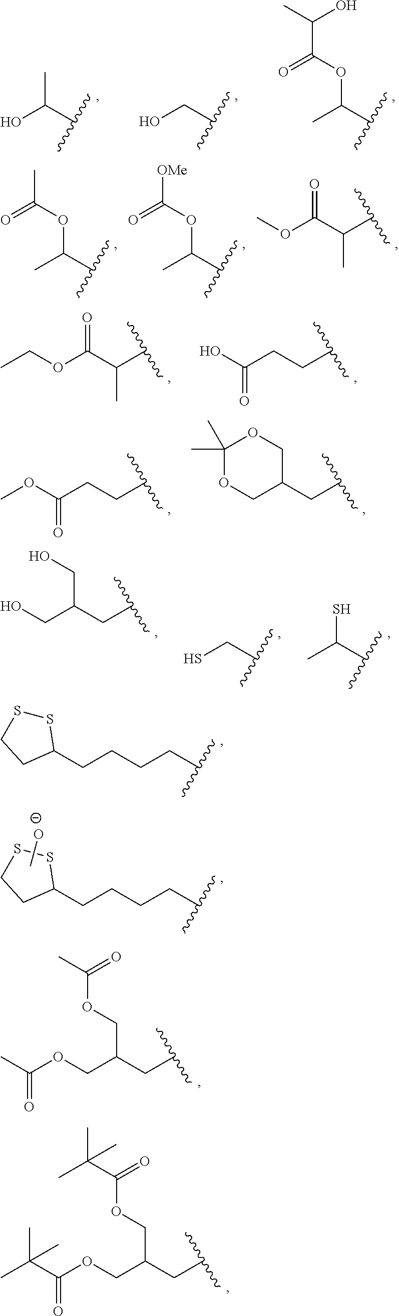

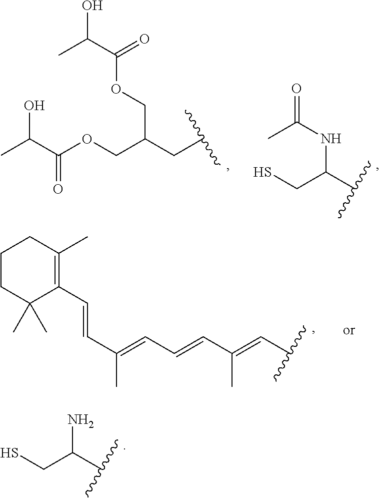

In some embodiments, R is:

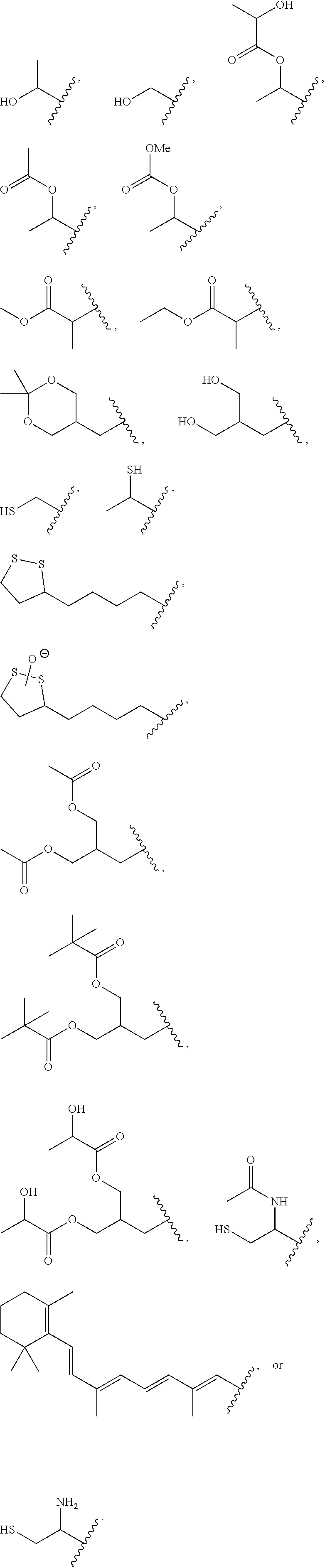

In some embodiments, R is substituted (e.g., linear or branched) heteroalkyl, the (e.g., linear or branched) heteroalkyl being substituted with one or more (heteroalkyl) substituent, each (heteroalkyl) substituent being independently selected from the group consisting of hydroxy, C₁-C₆ alkoxy, thioalkyl, amino, carboxylic acid, C₁-C₆ alkyl, acetamide, thiol, oxo, and optionally substituted (e.g., N-attached) heterocycloalkyl (e.g., optionally substituted with carboxylic acid).

In some embodiments, R is:

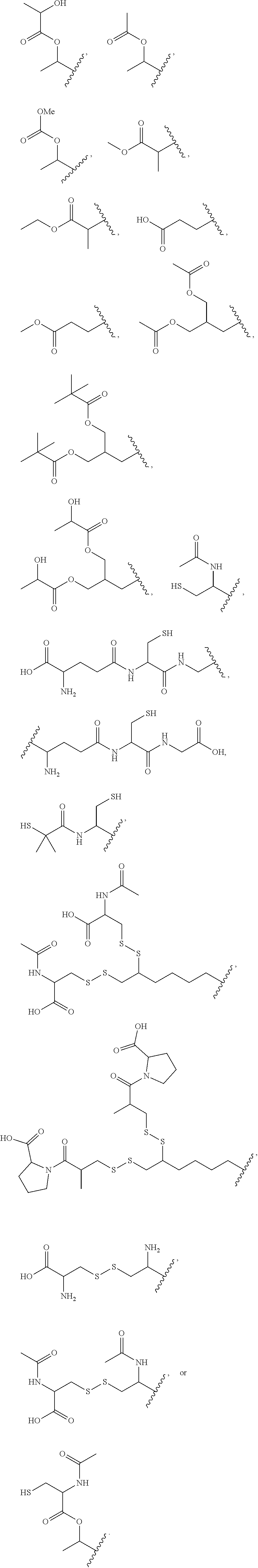

In some embodiments, R is:

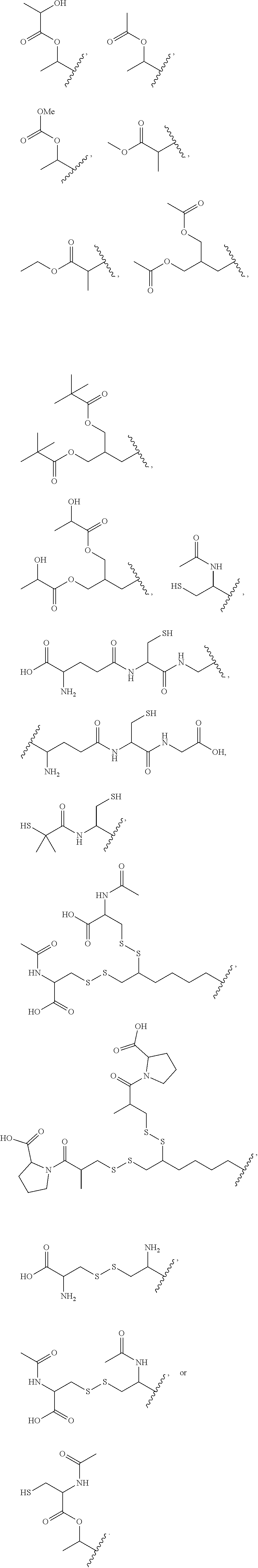

In some embodiments, R is substituted heterocycloalkyl (e.g., N-substituted with alkyl (e.g., further substituted with oxo and thiol)).

In some embodiments, R is:

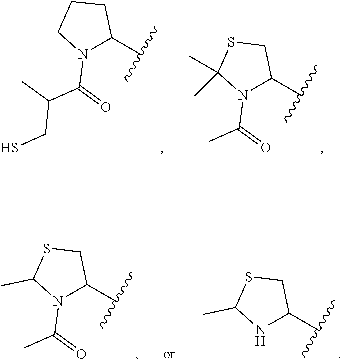

In some embodiments, R is unsubstituted alkyl (e.g., methyl, ethyl, isopropyl, or t-butyl). In some embodiments, L is —O(C═O)OCH(CH₃)— and R is unsubstituted alkyl (e.g., methyl, ethyl, isopropyl, or t-butyl).

In some embodiments, R is:

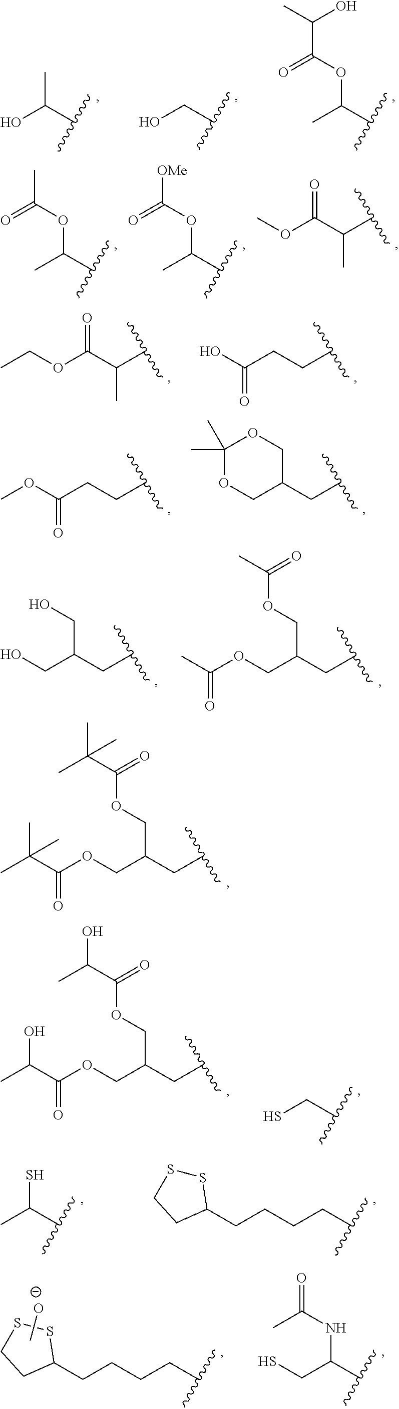

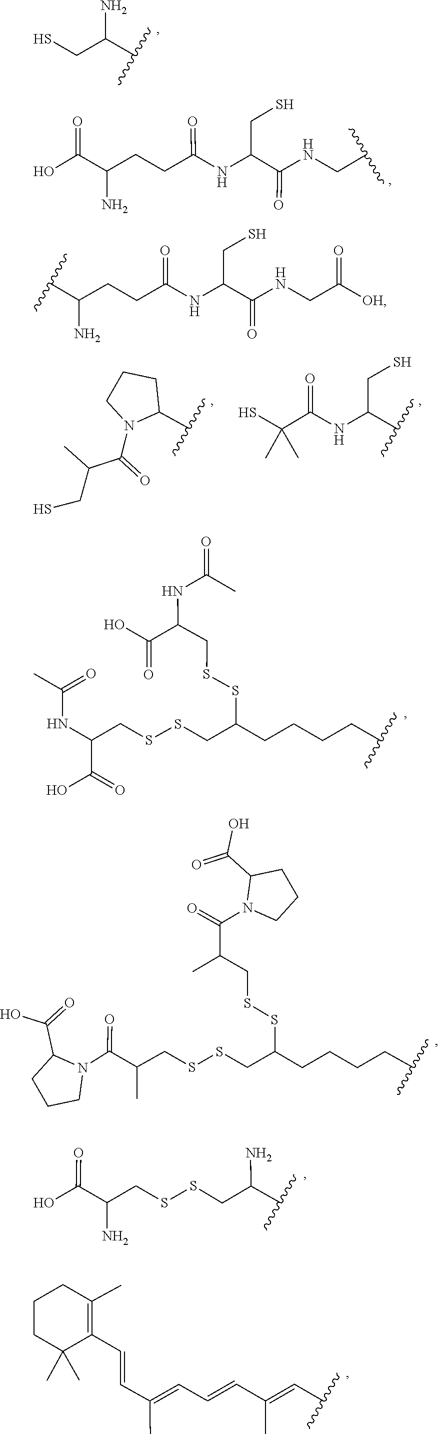

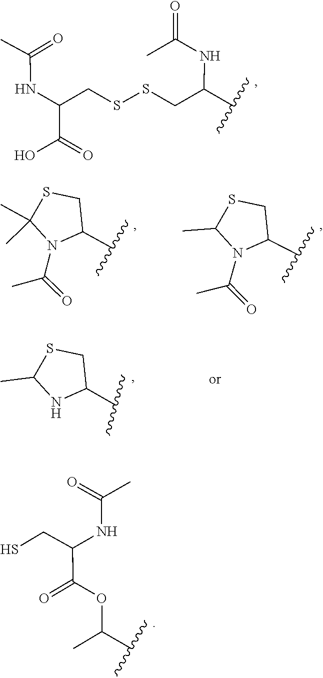

In some embodiments, R is:

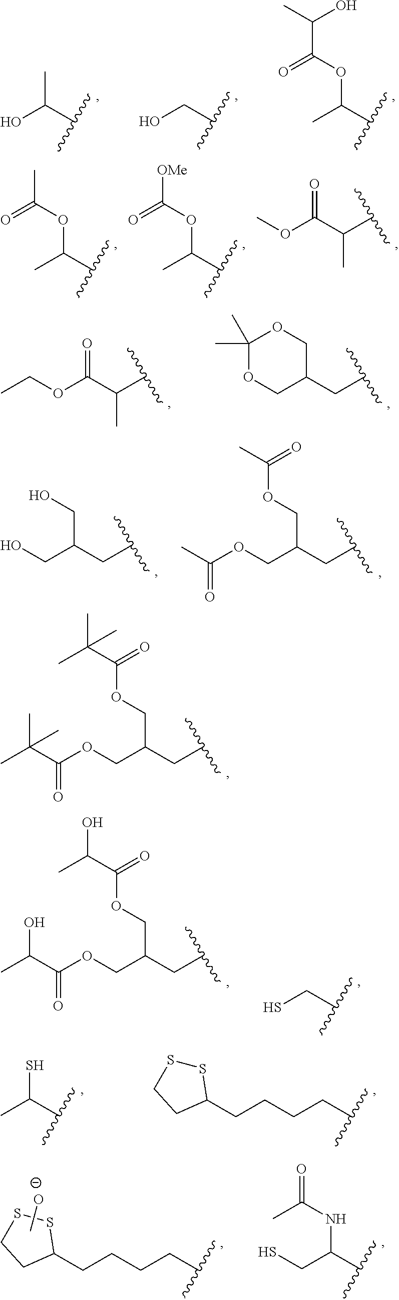

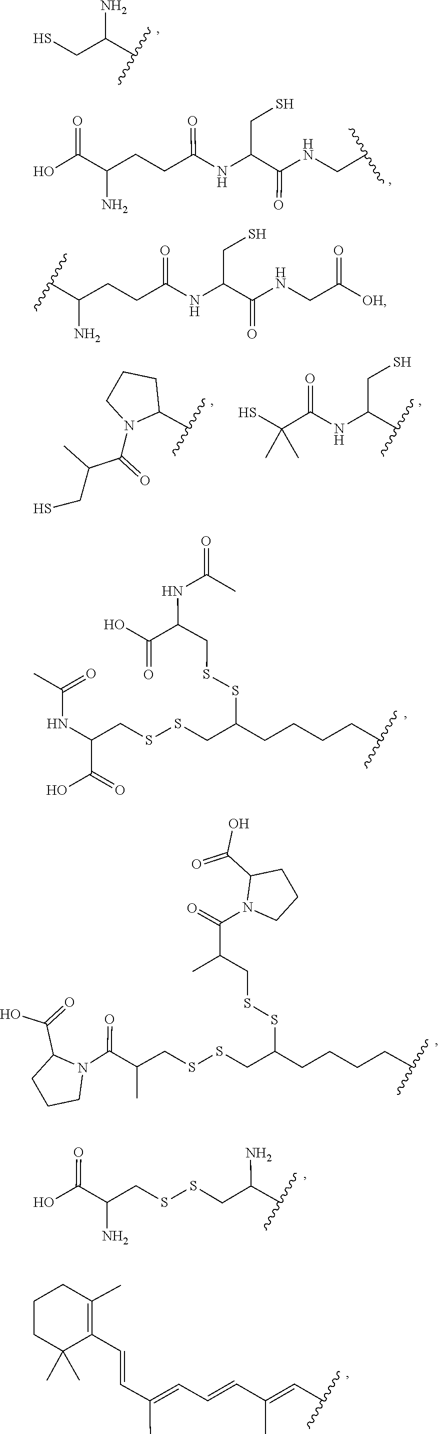

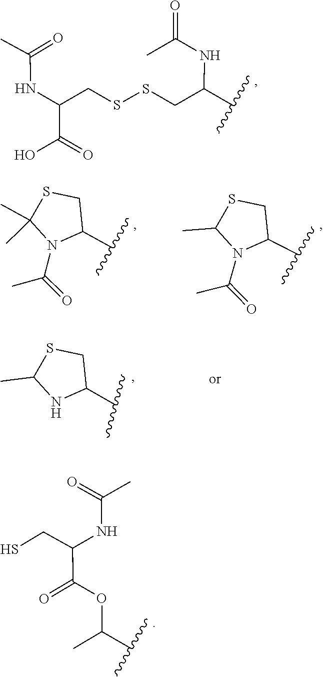

In some embodiments, R is the radical recited in Compound 1.

In some embodiments, R is the radical recited in Compound 2.

In some embodiments, R is the radical recited in Compound 3.

In some embodiments, R is the radical recited in Compound 4.

In some embodiments, R is the radical recited in Compound 5.

In some embodiments, R is the radical recited in Compound 6.

In some embodiments, R is the radical recited in Compound 7.

In some embodiments, R is the radical recited in Compound 8.

In some embodiments, R is the radical recited in Compound 9.

In some embodiments, R is the radical recited in Compound 10.

In some embodiments, R is the radical recited in Compound 11.

In some embodiments, R is the radical recited in Compound 11A.

In some embodiments, R is the radical recited in Compound 12.

In some embodiments, R is the radical recited in Compound 12A.

In some embodiments, R is the radical recited in Compound 13.

In some embodiments, R is the radical recited in Compound 13A.

In some embodiments, R is the radical recited in Compound 14.

In some embodiments, R is the radical recited in Compound 15.

In some embodiments, R is the radical recited in Compound 16.

In some embodiments, R is the radical recited in Compound 17.

In some embodiments, R is the radical recited in Compound 18.

In some embodiments, R is the radical recited in Compound 19.

In some embodiments, R is the radical recited in Compound 20.

In some embodiments, R is the radical recited in Compound 24.

In some embodiments, R is the radical recited in Compound 25.

In some embodiments, R is the radical recited in Compound 26.

In some embodiments, R is the radical recited in Compound 27.

In some embodiments, R is the radical recited in Compound 28.

In some embodiments, R is the radical recited in Compound 29.

In some embodiments, R is the radical recited in Compound 30.

In some embodiments, R is the radical recited in Compound 31.

In some embodiments, R is the radical recited in Compound 32.

In some embodiments, R is the radical recited in Compound 33.

In some embodiments, R is the radical recited in Compound 34.

In some embodiments, R is the radical recited in Compound 35.

In some embodiments, R is the radical recited in Compound 36.

In some embodiments, R is the radical recited in Compound 37.

In some embodiments, R is the radical recited in Compound 38.

In some embodiments, R is the radical recited in Compound 39.

In some embodiments, R is the radical recited in Compound 40.

In some embodiments, R is the radical recited in Compound 41.

In some embodiments, R is the radical recited in Compound 42.

In some embodiments, R is the radical recited in Compound 46.

In some embodiments, R is the radical recited in Compound 47.

In some embodiments, R is the radical recited in Compound 48.

In some embodiments, R is the radical recited in Compound 49.

In some embodiments, R is the radical recited in Compound 60.

In some embodiments, R is the radical recited in Compound 61.

In some embodiments, R is the radical recited in Compound 62.

In some embodiments, R is the radical recited in Compound 63.

In some embodiments, R is the radical recited in Compound 64.

In some embodiments, R is the radical recited in Compound 65.

In some embodiments, R is the radical recited in Compound 66.

In some embodiments, R is the radical recited in Compound 67.

In some embodiments, R is the radical recited in Compound 68.

In some embodiments, R is the radical recited in Compound 69.

In some embodiments, R is the radical recited in Compound 70.

In some embodiments, the compound is other than a compound having the structure:

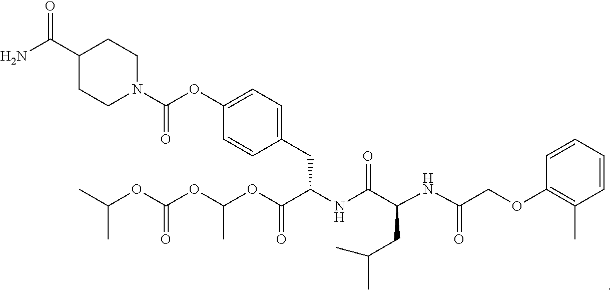 [SMILES: Cc1ccccc1OCC(=O)N[C@@H](CC(C)C)C(=O)N[C@@H](Cc1ccc(OC(=O)N2CCC(C(N)=O)CC2)cc1)C(=O)OC(C)OC(=O)OC(C)C]

In some embodiments, provided herein is a compound having the structure of Formula

(%):

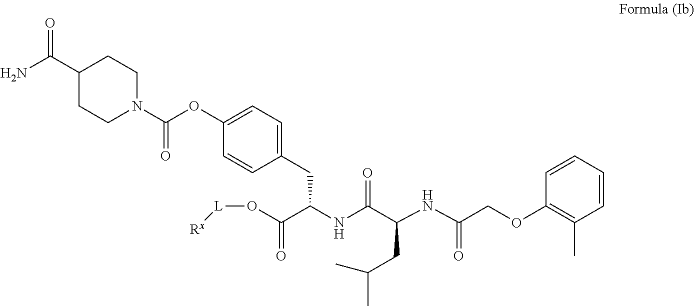

or a pharmaceutically acceptable salt or solvate (e.g., or a stereoisomer) thereof,
wherein:
   L is bond, —O(C═O)(OCR⁸R⁹)_{z}—, or —(C═O)(OCR⁸R⁹)_{z}—;
      each R⁸ and R⁹ is independently H, halogen, C₁-C₃-alkyl, C₁-C₃-haloalkyl, C₁-C₃-alkoxy, C₃-C₅-cycloalkyl, or R⁸ and R⁹ are taken together with the atoms to which they are attached to form a C₃-C₅-cycloalkyl;
      z is 1-6; and
   R^{X} is:

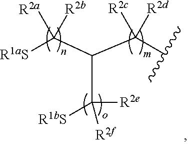

R^{1a} and R^{1b} are each independently —H or SR^{1c};
   each R^{1c} is independently substituted or unsubstituted (e.g., straight or branched) alkyl (e.g., substituted with one or more (alkyl) substituent, each (alkyl) substituent being independently selected from the group consisting of carboxylic acid, —SH, thioalkyl, acetamide, amino, oxo, optionally substituted heterocycloalkyl (e.g., N-attached pyrrolidinyl substituted with —COOH)) or substituted or unsubstituted (e.g., straight or branched) heteroalkyl (e.g., substituted with one or more (heteroalkyl) substituent, each (heteroalkyl) substituent being independently selected from the group consisting of carboxylic acid, amino, thioalkyl, thiol, acetamide, and C₁-C₃ alkyl);
each R^{2a}, R^{2b}, R^{2c}, R^{2d}, R^{2e}, and R^{2f} is independently H, halogen, C₁-C₃-alkyl, C₁-C₃-haloalkyl, C₁-C₃-alkoxy, C₃-C₅-cycloalkyl, or any one of R^{2a} and R^{2b}, R^{2c} and R^{2d}, or R^{2e} and R^{2f} are taken together with the atoms to which they are attached to form a C₃-C₅-cycloalkyl;
m is an integer from 1-10; and
n and o are each independently an integer from 0-3.

In some embodiments, o is 0.

In some embodiments, o is 0, and R^{x} is:

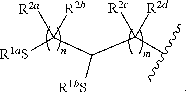

In some embodiments, o is 0 and n is 1.

In some embodiments, o is 0, n is 1, and R^{x} is:

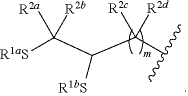

In some embodiments, m is an integer from 3-5.

In some embodiments, each R^{2a}, R^{2b}, R^{2c}, R^{2d}, R^{2e}, and R^{2f} is independently H, halogen,

C₁-C₃alkyl, or C₁-C₃haloalkyl. In some embodiments, each R^{2a}, R^{2b}, R^{2c}, R^{2d}, R^{2e}, and R^{2f} is H.

In some embodiments, R^{x} is:

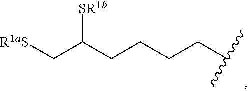

wherein:
   R^{1a} and R^{1b} are each independently —H or —SR^{1c}; and
      each R^{1c} is independently substituted or unsubstituted (e.g., straight or branched) alkyl (e.g., substituted with one or more (alkyl) substituent, each (alkyl) substituent being independently selected from the group consisting of carboxylic acid, —SH, thioalkyl, acetamide, amino, oxo, optionally substituted heterocycloalkyl (e.g., N-attached pyrrolidinyl substituted with —COOH)) or substituted or unsubstituted (e.g., straight or branched) heteroalkyl (e.g., substituted with one or more (heteroalkyl) substituent, each (heteroalkyl) substituent being independently selected from the group consisting of carboxylic acid, amino, thioalkyl, thiol, acetamide, and C₁-C₃ alkyl).

In some embodiments, R^{1a} is —H or —SR^{1c} and R^{1b} is —SR^{1c}, or R^{1a} is —SR^{1c} and R^{1b} is —H or —SR^{1c}. In some embodiments, R^{1a} and R^{1b} are each —SR^{1c}.

In some embodiments, R^{1a} and R^{1b} each independently comprise a radical of one or more keratolytic group (e.g., each radical of the one or more keratolytic group being independently selected from the group consisting of a radical of glycolic acid (GA), a radical of thioglycolic acid (TGA), a radical of lactic acid (Lac), a radical of thiolactic acid (TLac), a radical of lipoic acid (Lip), a radical of lipoic acid sulfoxide (Lipox), a radical of dihydrolipoic acid (diHLip), a radical of N-acetyl cysteine (NAC), a radical of cysteine (Cys), a radical of glutathione (GSH), a radical of captopril (Cap), and a radical of bucillamine (Buc)).

In some embodiments, R^{1a} and R^{1b} are each independently a radical of one or more keratolytic group, each radical of the one or more keratolytic group being independently selected from the group consisting of a radical of glycolic acid (GA), a radical of thioglycolic acid (TGA), a radical of lactic acid (Lac), a radical of thiolactic acid (TLac), a radical of lipoic acid (Lip), a radical of lipoic acid sulfoxide (Lipox), a radical of dihydrolipoic acid (diHLip), a radical of N-acetyl cysteine (NAC), a radical of cysteine (Cys), a radical of glutathione (GSH), a radical of captopril (Cap), and a radical of bucillamine (Buc).

In some embodiments, R^{1a} and R^{1b} each independently comprise a (thiol) radical of one or more keratolytic group, each (thiol) radical of the one or more keratolytic group being independently selected from the group consisting of a (thiol) radical of thioglycolic acid (TGA), a (thiol) radical of thiolactic acid (TLac), a (thiol) radical of dihydrolipoic acid (diHLip), a (thiol) radical of N-acetyl cysteine (NAC), a (thiol) radical of cysteine (Cys), a (thiol) radical of glutathione (GSH), a (thiol) radical of captopril (Cap), and a (thiol) radical of bucillamine (Buc).

In some embodiments, R^{1a} and R^{1b} are each independently a thiol radical of one or more keratolytic group, each thiol radical of the one or more keratolytic group being independently selected from the group consisting of a thiol radical of thioglycolic acid (TGA), a thiol radical of thiolactic acid (TLac), a thiol radical of dihydrolipoic acid (diHLip), a thiol radical of N-acetyl cysteine (NAC), a thiol radical of cysteine (Cys), a thiol radical of glutathione (GSH), a thiol radical of captopril (Cap), and a thiol radical of bucillamine (Buc).

In some embodiments, the (e.g., thiol) radical of the keratolytic agent comprises a (e.g., thiol) radical of one or more keratolytic group, each (e.g., thiol) radical of the one or more keratolytic group being independently selected from the group consisting of [Lac-Lac]·, [Lac-NAC]·, [Cys-Cys]·, [diHLip-NAC-NAC]·, [diHLip-NAC]·, [diHLip-Cap-Cap]·, [diHLip-Cap]·, [diHLip-Cys-Cys]·, [diHLip-Cys]·, [diHLip-Lipox-Lipox]·, and [diHLip-Lipox]·.

Unless stated otherwise, a radical (or·) is molecule having unpaired electrons. In some embodiments, the radical is a radical of a heteroatom (e.g., —O·, —N·, or —S). In some embodiments, the radical (e.g., the molecule having unpaired electron) is paired with another unpaired electron of another molecule to form paired electrons. In some embodiments, a radical of a keratolytic agent provided herein is paired with any compound provided herein. In some embodiments, a first radical of a keratolytic agent provided herein is paired with a second radical of a keratolytic provided herein.

In some embodiments, the thiol radical of the keratolytic group is the point of attachment of R^{1a} and/or R^{1b} to the rest of the molecule. In some embodiments, R^{1a} and/or R^{1b} attach to the rest of the molecule to form a disulfide bond.

In some embodiments, R^{1a} and R^{1b} are each independently —H or:

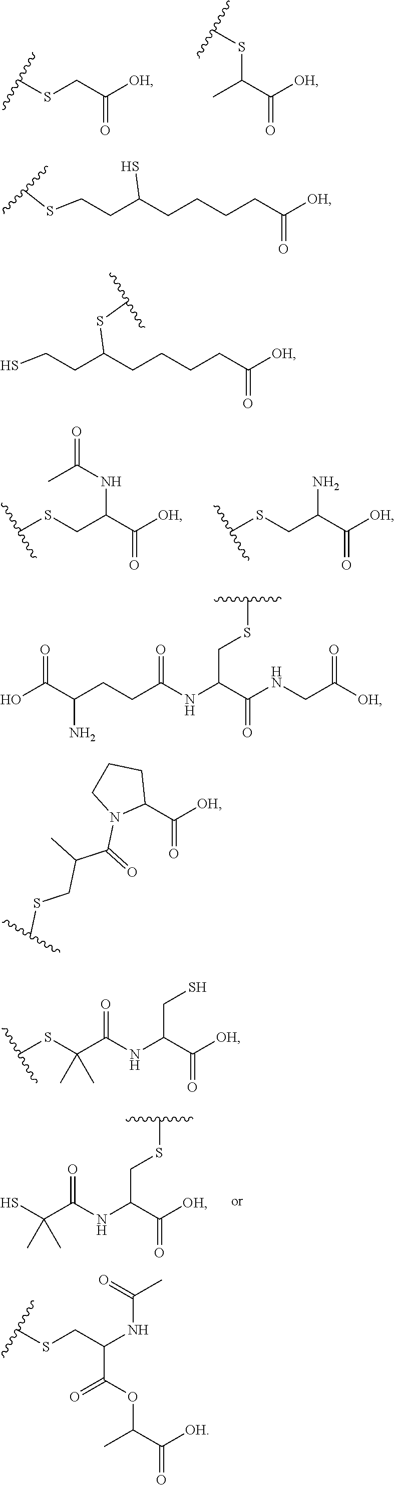

In some embodiments, R^{1a} and R^{1b} are the same. In some embodiments, R^{1a} and R^{1b} are different.

In some embodiments, R^{x} is:

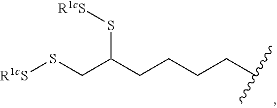

wherein:
   each R^{1c} is independently substituted or unsubstituted (e.g., straight or branched) alkyl (e.g., substituted with one or more (alkyl) substituent, each (alkyl) substituent being independently selected from the group consisting of carboxylic acid, —SH, thioalkyl, acetamide, amino, oxo, optionally substituted heterocycloalkyl (e.g., N-attached pyrrolidinyl substituted with —COOH)) or substituted or unsubstituted (e.g., straight or branched) heteroalkyl (e.g., substituted with one or more (heteroalkyl) substituent, each (heteroalkyl) substituent being independently selected from the group consisting of carboxylic acid, amino, thioalkyl, thiol, acetamide, and C₁-C₃ alkyl).

In some embodiments, each R^{1c} is independently substituted or unsubstituted (e.g., straight or branched) alkyl or substituted or unsubstituted (e.g., straight or branched) heteroalkyl. In some embodiments, each R^{1c} is independently substituted (e.g., straight or branched) alkyl or substituted (e.g., straight or branched) heteroalkyl.

In some embodiments, each R^{1c} is independently substituted (e.g., straight or branched) alkyl, the substituted alkyl being substituted with one or more (alkyl) substituent, each (alkyl) substituent being independently selected from the group consisting of carboxylic acid, —SH, thioalkyl, acetamide, amino, oxo, and optionally substituted heterocycloalkyl (e.g., N-attached pyrrolidinyl substituted with —COOH).

In some embodiments, each R^{1c} is independently substituted (e.g., straight or branched) heteroalkyl, the substituted heteroalkyl being substituted with one or more (heteroalkyl) substituent, each (heteroalkyl) substituent being independently selected from the group consisting of carboxylic acid, amino, thioalkyl, thiol, acetamide, and C₁-C₃ alkyl.

In some embodiments, R^{1a}, R^{1b} and each R^{1c} each independently comprise one or more substituent that is a carboxylic acid or an ester. In some embodiments, R^{1a}, R^{1b}, and each R^{1c} each independently comprise one or more substituent that is a carboxylic acid (e.g., —(C═O)OH). In some embodiments, R^{1a} comprises one or more substituent that is a carboxylic acid (e.g., —(C═O)OH). In some embodiments, R^{1b} comprises one or more substituent that is a carboxylic acid (e.g., —(C═O)OH). In some embodiments, each R^{1c} independently comprises one or more substituent that is a carboxylic acid (e.g., —(C═O)OH). In some embodiments, R^{1a}, R^{1b} and each R^{1c} each independently comprise one or more substituent that is an ester (e.g., —(C═O)O—C₁-C₄alkyl). In some embodiments, R^{1a} comprises one or more substituent that is an ester (e.g., —(C═O)O—C₁-C₄alkyl). In some embodiments, R^{1b} comprises one or more substituent that is an ester (e.g., —(C═O)O—C₁-C₄alkyl). In some embodiments, each R^{1c} independently comprises one or more substituent that is an ester (e.g., —(C═O)O—C₁-C₄alkyl).

In some embodiments, the —(C═O)OH of R^{1a}, R^{1b}, and/or R^{1c} is optionally esterified (e.g., —(C═O)OH or —(C═O)O—C₁-C₄alkyl).

In some embodiments, provided herein is a compound having the structure of Formula (IC):

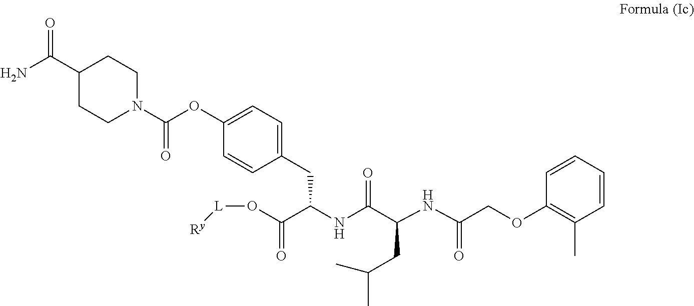

or a pharmaceutically acceptable salt or solvate (e.g., or a stereoisomer) thereof,
wherein:
   L is bond, —O(C═O)(OCR⁸R⁹)_{z}—, or —(C═O)(OCR⁸R⁹)_{z}—;
      each R⁸ and R⁹ is independently H, halogen, C₁-C₃-alkyl, C₁-C₃-haloalkyl, C₁-C₃-alkoxy, C₃-C₅-cycloalkyl, or R⁸ and R⁹ are taken together with the atoms to which they are attached to form a C₃-C₅-cycloalkyl;
      z is 1-6; and
   R^{y} is:

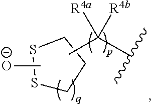

each R^{4a} and R^{4b} is independently H, halogen, or substituted or unsubstituted alkyl;
p is an integer from 1-10; and
q is an integer from 1-3.

In some embodiments, q is 1.

In some embodiments, p is an integer from 3-5. In some embodiments, p is 4. In some embodiments, q is 1 and p is 4.

In some embodiments, each R^{4a} and R^{4b} is independently H or substituted or unsubstituted alkyl. In some embodiments, each R^{4a} and R^{4b} is independently H, halogen, C₁-C₃alkyl, or C₁-C₃haloalkyl. In some embodiments, each R^{4a} and R^{4b} is H.

In some embodiments, R^{y} is:

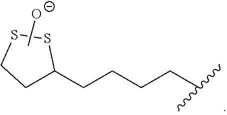

In some embodiments, provided herein is a compound having the structure of Formula

(Id):

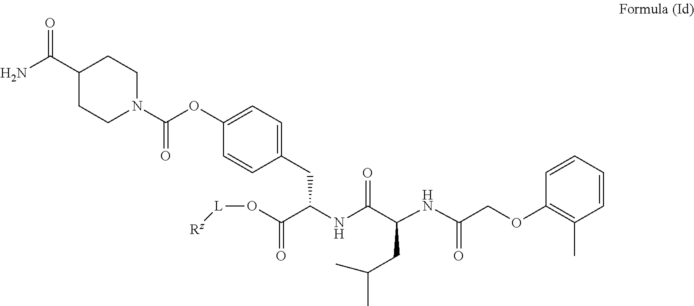

or a pharmaceutically acceptable salt or solvate (e.g., or a stereoisomer) thereof,
wherein:
   L is bond, —O(C═O)(OCR⁸R⁹)_{z}—, or —(C═O)(OCR⁸R⁹)_{z}—;
      each R⁸ and R⁹ is independently H, halogen, C₁-C₃-alkyl, C₁-C₃-haloalkyl, C₁-C₃-alkoxy, C₃-C₅-cycloalkyl, or R⁸ and R⁹ are taken together with the atoms to which they are attached to form a C₃-C₅-cycloalkyl;
      z is 1-6; and
   R^{Z} is:

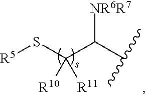

R⁵ is —SR^{1c};
   R^{1c} is substituted or unsubstituted (e.g., straight or branched) alkyl (e.g., substituted with one or more (alkyl) substituent, each (alkyl) substituent being independently selected from the group consisting of carboxylic acid, —SH, thioalkyl, acetamide, amino, oxo, optionally substituted heterocycloalkyl (e.g., N-attached pyrrolidinyl substituted with —COOH)) or substituted or unsubstituted (e.g., straight or branched) heteroalkyl (e.g., substituted with one or more (heteroalkyl) substituent, each (heteroalkyl) substituent being independently selected from the group consisting of carboxylic acid, amino, thioalkyl, thiol, acetamide, and C₁-C₃ alkyl);
R⁶ and R⁷ are each independently H, substituted or unsubstituted alkyl, or substituted or unsubstituted heteroalkyl;
each R¹⁰ and R¹¹ is independently H, halogen, C₁-C₃-alkyl, C₁-C₃-haloalkyl, C₁-C₃-alkoxy, C₃-C₅-cycloalkyl, or two of R¹⁰ and R¹¹ are taken together with the atoms to which they are attached to form a C₃-C₅-cycloalkyl; and
s is an integer from 1-10.

In some embodiments, R⁶ and R⁷ are each independently H or substituted or unsubstituted alkyl (e.g., C₁-C₃ alkyl optionally substituted with oxo). In some embodiments, R⁶ and R⁷ are each independently H or C₁-C₃ alkyl optionally substituted with oxo. In some embodiments, R⁶ and R⁷ are each independently H or —(C═O)CH₃. In some embodiments, R⁶ is H and R⁷ is H or —(C═O)CH₃. In some embodiments, R⁶ is H and R⁷ is —(C═O)CH₃. In some embodiments, R⁶ and R⁷ are H.

In some embodiments, each R¹⁰ and R¹¹ is independently H, halogen, C₁-C₃alkyl, or C₁-C₃haloalkyl. In some embodiments, each R¹⁰ and R¹¹ is H.

In some embodiments, s is 1-3. In some embodiments, s is 1. In some embodiments, s is 1 and R¹⁰ and R¹¹ are H.

In some embodiments, R⁵ comprises a radical of one or more keratolytic group (e.g., each radical of the one or more keratolytic group being independently selected from the group consisting of a radical of glycolic acid (GA), a radical of thioglycolic acid (TGA), a radical of lactic acid (Lac), a radical of thiolactic acid (TLac), a radical of lipoic acid (Lip), a radical of lipoic acid sulfoxide (Lipox), a radical of dihydrolipoic acid (diHLip), a radical of N-acetyl cysteine (NAC), a radical of cysteine (Cys), a radical of glutathione (GSH), a radical of captopril (Cap), and a radical of bucillamine (Buc)).

In some embodiments, R⁵ is a radical of one or more keratolytic group, each radical of the one or more keratolytic group being independently selected from the group consisting of a radical of glycolic acid (GA), a radical of thioglycolic acid (TGA), a radical of lactic acid (Lac), a radical of thiolactic acid (TLac), a radical of lipoic acid (Lip), a radical of lipoic acid sulfoxide (Lipox), a radical of dihydrolipoic acid (diHLip), a radical of N-acetyl cysteine (NAC), a radical of cysteine (Cys), a radical of glutathione (GSH), a radical of captopril (Cap), and a radical of bucillamine (Buc).

In some embodiments, R⁵ comprises a (thiol) radical of one or more keratolytic group, each (thiol) radical of the one or more keratolytic group being independently selected from the group consisting of a (thiol) radical of thioglycolic acid (TGA), a (thiol) radical of thiolactic acid (TLac), a (thiol) radical of dihydrolipoic acid (diHLip), a (thiol) radical of N-acetyl cysteine

(NAC), a (thiol) radical of cysteine (Cys), a (thiol) radical of glutathione (GSH), a (thiol) radical of captopril (Cap), and a (thiol) radical of bucillamine (Buc).

In some embodiments R⁵ is a thiol radical of one or more keratolytic group, each thiol radical of the one or more keratolytic group being independently selected from the group consisting of a thiol radical of thioglycolic acid (TGA), a thiol radical of thiolactic acid (TLac), a thiol radical of dihydrolipoic acid (diHLip), a thiol radical of N-acetyl cysteine (NAC), a thiol radical of cysteine (Cys), a thiol radical of glutathione (GSH), a thiol radical of captopril (Cap), and a thiol radical of bucillamine (Buc).

In some embodiments, the (e.g., thiol) radical of the keratolytic agent comprises a (e.g., thiol) radical of one or more keratolytic group, each (e.g., thiol) radical of the one or more keratolytic group being independently selected from the group consisting of [Lac-Lac]·, [Lac-NAC]·, [Cys-Cys]·, [diHLip-NAC-NAC]·, [diHLip-NAC]·, [diHLip-Cap-Cap]·, [diHLip-Cap]·, [diHLip-Cys-Cys]·, [diHLip-Cys]·, [diHLip-Lipox-Lipox]·, and [diHLip-Lipox]·.

In some embodiments, the thiol radical of the keratolytic group is the point of attachment of R⁵ to the rest of the molecule. In some embodiments, R⁵ attaches to the rest of the molecule to form a disulfide bond.

In some embodiments, R⁵ is:

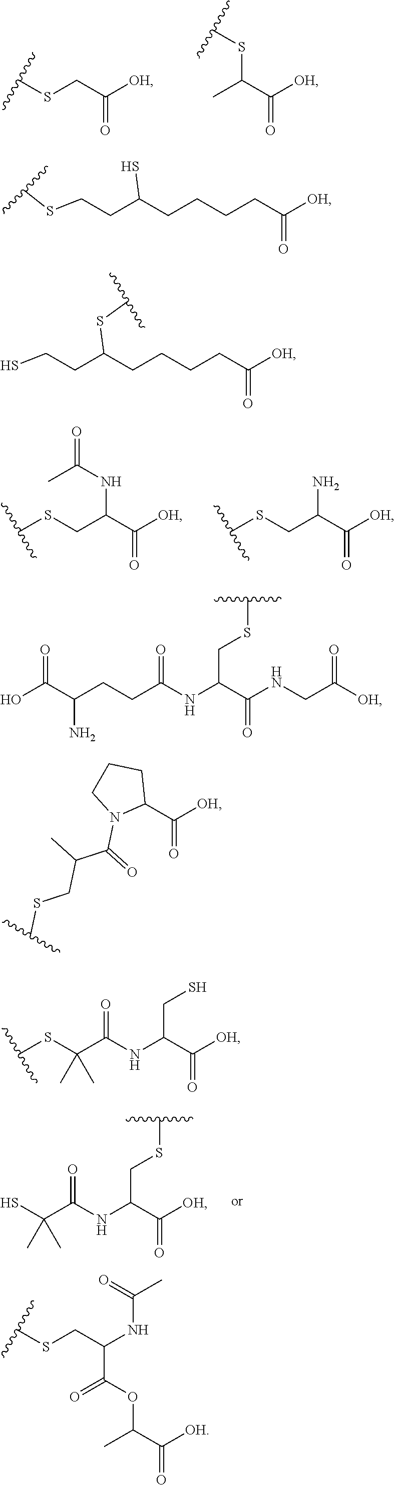

In some embodiments, R^{z} is:

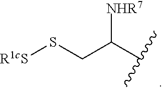

In some embodiments, R⁷ is H or —(C═O)CH₃. In some embodiments, R⁷ is H. In some embodiments, R⁷ is —(C═O)CH₃.

In some embodiments, each R^{1c} is independently substituted or unsubstituted (e.g., straight or branched) alkyl or substituted or unsubstituted (e.g., straight or branched) heteroalkyl. In some embodiments, each R^{1c} is independently substituted (e.g., straight or branched) alkyl or substituted (e.g., straight or branched) heteroalkyl.

In some embodiments, each R^{1c} is independently substituted (e.g., straight or branched) alkyl, the substituted alkyl being substituted with one or more (heteroalkyl) substituent, each (heteroalkyl) substituent being independently selected from the group consisting of carboxylic acid, —SH, thioalkyl, acetamide, amino, oxo, and optionally substituted heterocycloalkyl (e.g., N-attached pyrrolidinyl substituted with —COOH).

In some embodiments, each R^{1c} is independently substituted (e.g., straight or branched) heteroalkyl, the substituted heteroalkyl being substituted with one or more (heteroalkyl) substituent, each (heteroalkyl) substituent being independently selected from the group consisting of carboxylic acid, amino, thioalkyl, thiol, acetamide, and C₁-C₃ alkyl.

In some embodiments, R⁵ and each R^{1c} each independently comprise one or more substituent that is a carboxylic acid or an ester. In some embodiments, R⁵ and each R^{1c} each independently comprise one or more substituent that is a carboxylic acid (e.g., —(C═O)OH). In some embodiments, R⁵ and each R^{1c} each independently comprise one or more substituent that is an ester (e.g., —(C═O)O—C₁-C₄alkyl).

In some embodiments, the —(C═O)OH of R⁵ and/or R^{1c} is optionally esterified (e.g., —(C═O)OH or —(C═O)O—C₁-C₄alkyl). In some embodiments, the C₁-C₄alkyl is methyl, ethyl, propyl, isopropyl, butyl, or t-butyl.

Provided in some embodiments herein is a compound, a stereoisomer thereof, or a pharmaceutically acceptable salt of the compound or the stereoisomer, having a structure provided in Table 1.

TABLE 1

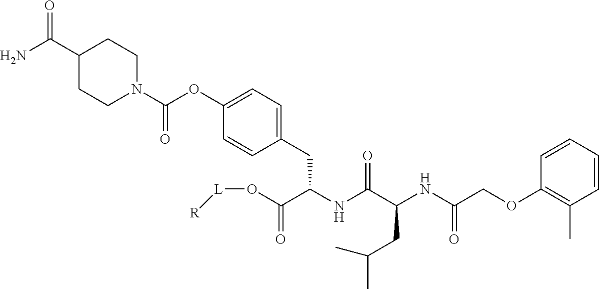

| Compound | R | L |
|---|---|---|
| 1 | 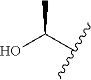 [SMILES: *[C@H](C)O] | —(C═O)OCH(CH₃)— |
| 2 | 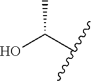 [SMILES: *[C@@H](C)O] | —(C═O)OCH(CH₃)— |
| 3 | 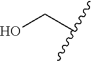 [SMILES: *CO] | —(C═O)OCH(CH₃)— |
| 4 | 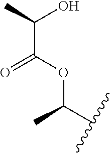 [SMILES: *[C@@H](C)OC(=O)[C@@H](C)O] | —(C═O)OCH(CH₃)— |
| 5 | 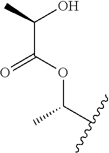 [SMILES: *[C@H](C)OC(=O)[C@@H](C)O] | —(C═O)OCH(CH₃)— |
| 6 | 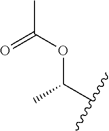 [SMILES: *[C@H](C)OC(C)=O] | —(C═O)OCH(CH₃)— |
| 7 | 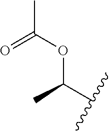 [SMILES: *[C@@H](C)OC(C)=O] | —(C═O)OCH(CH₃)— |
| 8 | 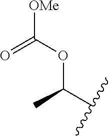 [SMILES: *[C@@H](C)OC(=O)OC] | —(C═O)OCH(CH₃)— |
| 9 | 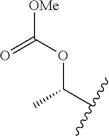 [SMILES: *[C@H](C)OC(=O)OC] | —(C═O)OCH(CH₃)— |
| 10 | 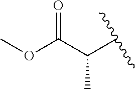 [SMILES: *[C@H](C)C(=O)OC] | —(C═O)OCH(CH₃)— |
| 11 | 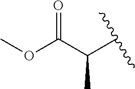 [SMILES: *[C@@H](C)C(=O)OC] | —(C═O)OCH(CH₃)— |
| 11A | 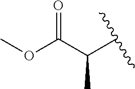 [SMILES: *[C@@H](C)C(=O)OC] | —O(C═O)OCH(CH₃)— |
| 12 | 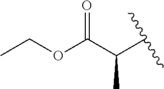 [SMILES: *[C@@H](C)C(=O)OCC] | —(C═O)OCH(CH₃)— |
| 12A | 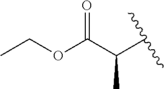 [SMILES: *[C@@H](C)C(=O)OCC] | —O(C═O)OCH(CH₃)— |
| 13 | 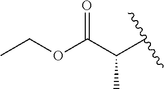 [SMILES: *[C@H](C)C(=O)OCC] | —(C═O)OCH(CH₃)— |
| 13A | 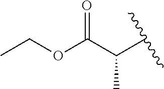 [SMILES: *[C@H](C)C(=O)OCC] | —O(C═O)OCH(CH₃)— |
| 14 | 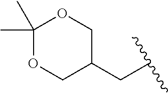 [SMILES: *CC1COC(C)(C)OC1] | —(C═O)OCH(CH₃)— |
| 15 | 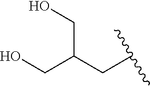 [SMILES: *CC(CO)CO] | —(C═O)OCH(CH₃)— |
| 16 | 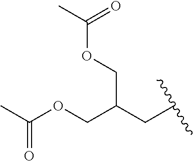 [SMILES: *CC(COC(C)=O)COC(C)=O] | —(C═O)OCH(CH₃)— |
| 17 | 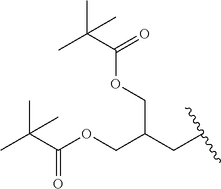 [SMILES: *CC(COC(=O)C(C)(C)C)COC(=O)C(C)(C)C] | —(C═O)OCH(CH₃)— |
| 18 | 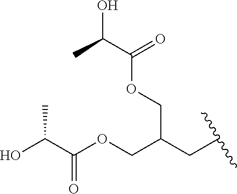 [SMILES: *CC(COC(=O)[C@@H](C)O)COC(=O)[C@@H](C)O] | —(C═O)OCH(CH₃)— |
| 19 | 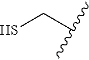 [SMILES: *CS] | —(C═O)OCH(CH₃)— |
| 20 | 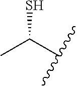 [SMILES: *[C@H](C)S] | —(C═O)OCH(CH₃)— |
| 21 | 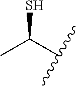 [SMILES: *[C@@H](C)S] | —(C═O)OCH(CH₃)— |
| 22 | 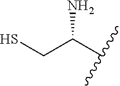 [SMILES: *[C@@H](N)CS] | —(C═O)OCH(CH₃)— |
| 23 | 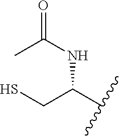 [SMILES: *[C@H](CS)NC(C)=O] | —(C═O)OCH(CH₃)— |
| 24 | 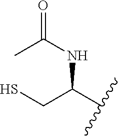 [SMILES: *[C@@H](CS)NC(C)=O] | —(C═O)OCH(CH₃)— |
| 25 | 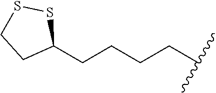 [SMILES: *CCCC[C@H]1CCSS1] | —(C═O)OCH(CH₃)— |
| 26 | 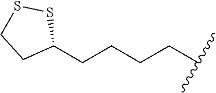 [SMILES: *CCCC[C@@H]1CCSS1] | —(C═O)OCH(CH₃)— |
| 27 | 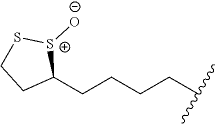 | —(C═O)OCH(CH₃)— |
| 28 | 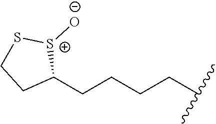 | —(C═O)OCH(CH₃)— |
| 29 | 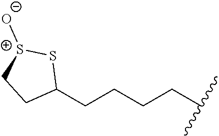 | —(C═O)OCH(CH₃)— |
| 30 | 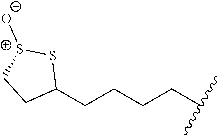 | —(C═O)OCH(CH₃)— |
| 31 | 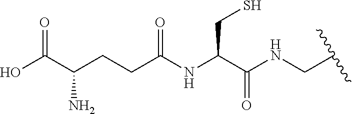 [SMILES: *CNC(=O)[C@H](CS)NC(=O)CC[C@H](N)C(=O)O] | —(C═O)OCH(CH₃)— |
| 32 | 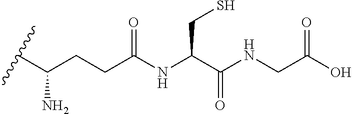 [SMILES: *[C@@H](N)CCC(=O)N[C@@H](CS)C(=O)NCC(=O)O] | —(C═O)OCH(CH₃)— |
| 33 | 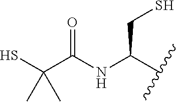 [SMILES: *[C@H](CS)NC(=O)C(C)(C)S] | —(C═O)OCH(CH₃)— |
| 34 | 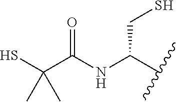 [SMILES: *[C@@H](CS)NC(=O)C(C)(C)S] | —(C═O)OCH(CH₃)— |
| 35 | 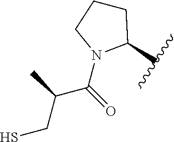 [SMILES: *[C@@H]1CCCN1C(=O)[C@H](C)CS] | —(C═O)OCH(CH₃)— |
| 36 | 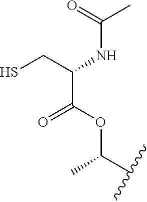 [SMILES: *[C@H](C)OC(=O)[C@H](CS)NC(C)=O] | —(C═O)OCH(CH₃)— |
| 37 | 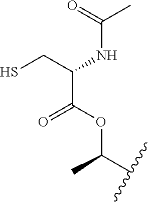 [SMILES: *[C@@H](C)OC(=O)[C@H](CS)NC(C)=O] | —(C═O)OCH(CH₃)— |
| 38 | 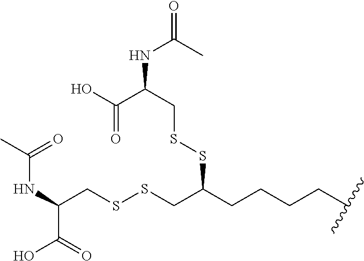 [SMILES: *CCCC[C@@H](CSSC[C@H](NC(C)=O)C(=O)O)SSC[C@H](NC(C)=O)C(=O)O] | —(C═O)OCH(CH₃)— |
| 39 | 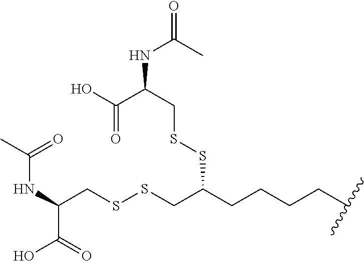 [SMILES: *CCCC[C@H](CSSC[C@H](NC(C)=O)C(=O)O)SSC[C@H](NC(C)=O)C(=O)O] | —(C═O)OCH(CH₃)— |
| 40 | 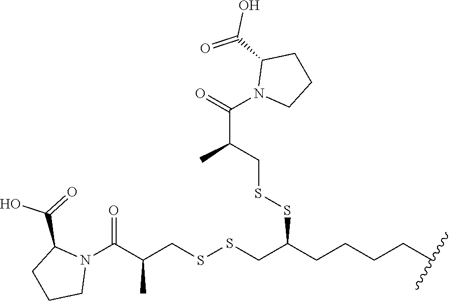 [SMILES: *CCCC[C@@H](CSSC[C@@H](C)C(=O)N1CCC[C@H]1C(=O)O)SSC[C@@H](C)C(=O)N1CCC[C@H]1C(=O)O] | —(C═O)OCH(CH₃)— |
| 41 | 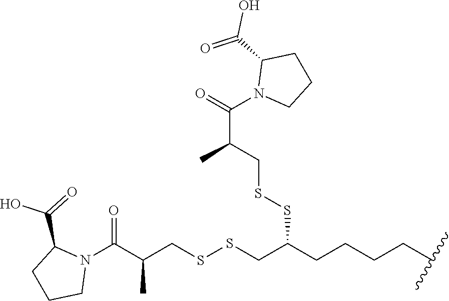 [SMILES: *CCCC[C@H](CSSC[C@@H](C)C(=O)N1CCC[C@H]1C(=O)O)SSC[C@@H](C)C(=O)N1CCC[C@H]1C(=O)O] | —(C═O)OCH(CH₃)— |
| 42 | 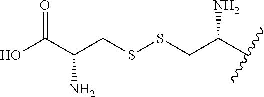 [SMILES: *[C@@H](N)CSSC[C@H](N)C(=O)O] | —(C═O)OCH(CH₃)— |
| 43 | 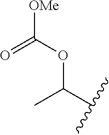 [SMILES: *C(C)OC(=O)OC] | bond |
| 44 | 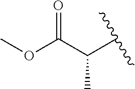 [SMILES: *[C@H](C)C(=O)OC] | —O(C═O)OCH(CH₃)— |
| 45 | 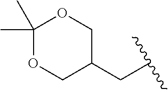 [SMILES: *CC1COC(C)(C)OC1] | —O(C═O)OCH(CH₃)— |
| 46 | —CH₃ | —(C═O)OCH(CH₃)— |
| 47 | —CH₂CH₃ | —(C═O)OCH(CH₃)— |
| 48 | —CH(CH₃)CH₃ | —(C═O)OCH(CH₃)— |
| 49 | —C(CH₃)₃ | —(C═O)OCH(CH₃)— |
| 50 | —CH₃ | —O(C═O)OCH(CH₃)— |
| 51 | —CH₂CH₃ | —O(C═O)OCH(CH₃)— |
| 52 | —CH(CH₃)CH₃ | —O(C═O)OCH(CH₃)— |
| 53 | —C(CH₃)₃ | —O(C═O)OCH(CH₃)— |
| 54 | 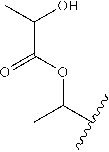 [SMILES: *C(C)OC(=O)C(C)O] | bond |
| 55 | 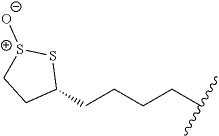 [SMILES: *CCCC[C@@H]1CC[S+]([O-])S1] | —(C═O)OCH(CH₃)— |
| 56 | 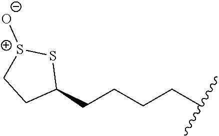 [SMILES: *CCCC[C@H]1CC[S+]([O-])S1] | —(C═O)OCH(CH₃)— |
| 57 | 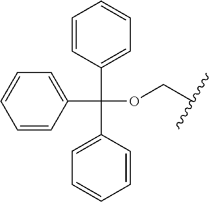 [SMILES: *COC(c1ccccc1)(c1ccccc1)c1ccccc1] | —(C═O)OCH(CH₃)— |
| 58 | 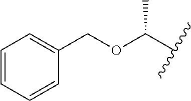 [SMILES: *[C@@H](C)OCc1ccccc1] | —(C═O)OCH(CH₃)— |
| 59 | 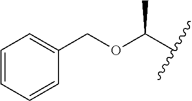 [SMILES: *[C@H](C)OCc1ccccc1] | —(C═O)OCH(CH₃)— |
| 60 | 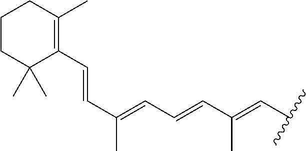 [SMILES: */C=C(C)/C=C/C=C(C)/C=C/C1=C(C)CCCC1(C)C] | —(C═O)OCH(CH₃)— |
| 61 | 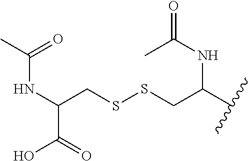 [SMILES: *C(CSSCC(NC(C)=O)C(=O)O)NC(C)=O] | —(C═O)OCH(CH₃)— |
| 62 | 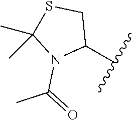 [SMILES: *C1CSC(C)(C)N1C(C)=O] | —(C═O)OCH(CH₃)— |
| 63 | 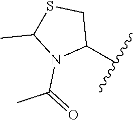 [SMILES: *C1CSC(C)N1C(C)=O] | —(C═O)OCH(CH₃)— |
| 64 | 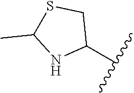 [SMILES: *C1CSC(C)N1] | —(C═O)OCH(CH₃)— |
| 65 | 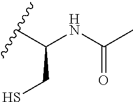 [SMILES: *[C@H](CS)NC(C)=O] | —CH(CH₃)O(C═O)O(CH₂CH₂O)₄(C═O)— |
| 66 | 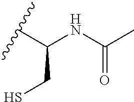 [SMILES: *[C@H](CS)NC(C)=O] | —CH(CH₃)O(C═O)O(CH₂CH₂O)₈(C═O)— |
| 67 | 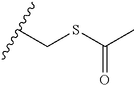 [SMILES: *CSC(C)=O] | —(C═O)OCH(CH₃)— |
| 68 | —CH₃ | bond |
| 69 | 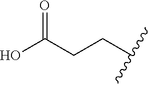 [SMILES: *CCC(=O)O] | —(C═O)OCH(CH₃)— |
| 70 | 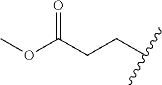 [SMILES: *CCC(=O)OC] | —(C═O)OCH(CH₃)— |

In some embodiments, provided herein is a compound having the structure of Formula

(II):

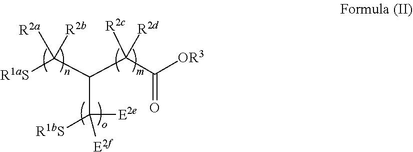

or a pharmaceutically acceptable salt or solvate (e.g., or a stereoisomer) thereof,
wherein:
   R^{1a} and R^{1b} are each independently —H or —SR^{1c}, either or both of R^{1a} and R^{1b} being —SR^{1c};
      each R^{1c} is independently substituted or unsubstituted (e.g., straight or branched) alkyl (e.g., substituted with one or more (alkyl) substituent, each (alkyl) substituent being independently selected from the group consisting of carboxylic acid, —SH, thioalkyl, acetamide, amino, oxo, optionally substituted heterocycloalkyl (e.g., N-attached pyrrolidinyl substituted with —COOH)) or substituted or unsubstituted (e.g., straight or branched) heteroalkyl (e.g., substituted with one or more (heteroalkyl) substituent, each (heteroalkyl) substituent being independently selected from the group consisting of carboxylic acid, amino, thioalkyl, thiol, acetamide, and C₁-C₃ alkyl);
   each R^{2a}, R^{2b}, R^{2c}, R^{2d}, R^{2e}, and R^{2f} is independently h, halogen, C₁-C₃-alkyl, C₁-C₃-haloalkyl, C₁-C₃-alkoxy, C₃-C₅-cycloalkyl, or two of R^{2a} and, R^{2b}, R^{2c} and R^{2d}, or R^{2e} and R^{2f} are taken together with the atoms to which they are attached to form a C₃-C₅-cycloalkyl;
   R³ is H or substituted or unsubstituted alkyl;
   m is an integer from 1-10; and
   n and o are each independently an integer from 0-3.

In some embodiments, o is 0.

In some embodiments, the compound has the structure of Formula (IIa):

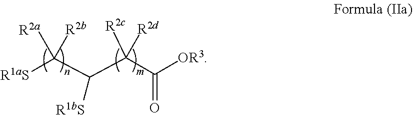

In some embodiments, o is 0 and n is 1.

In some embodiments, the compound has the structure of Formula (IIb):

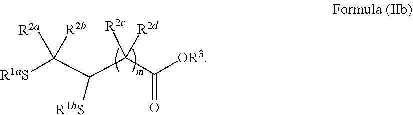

In some embodiments, m is an integer from 3-5.

In some embodiments, each R^{2a}, R^{2b}, R^{2c}, R^{2e}, and R^{2f} is independently H, halogen, C₁-C₃alkyl, or C₁-C₃haloalkyl. In some embodiments, each R^{2a}, R^{2b}, R^{2c}, R^{2d}, R^{2e}, and R^{2f} is H.

In some embodiments, the compound has the structure of Formula (IIc):

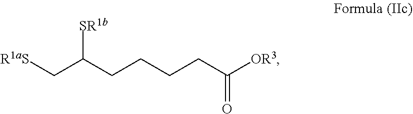

or a pharmaceutically acceptable salt or solvate (e.g., or a stereoisomer) thereof,
wherein:
   R^{1a} and R^{1b} are each independently —H or SR^{1c}; and
      each R^{1c} is independently substituted or unsubstituted (e.g., straight or branched) alkyl (e.g., substituted with one or more (alkyl) substituent, each (alkyl) substituent being independently selected from the group consisting of carboxylic acid, —SH, thioalkyl, acetamide, amino, oxo, optionally substituted heterocycloalkyl (e.g., N-attached pyrrolidinyl substituted with —COOH)) or substituted or unsubstituted (e.g., straight or branched) heteroalkyl (e.g., substituted with one or more (heteroalkyl) substituent, each (heteroalkyl) substituent being independently selected from the group consisting of carboxylic acid, amino, thioalkyl, thiol, acetamide, and C₁-C₃ alkyl); and
   R³ is H or substituted or unsubstituted alkyl.

In some embodiments, R^{1a} is —H or SR^{1c} and R^{1b} is —SR^{1c}, or R^{1a} is —SR^{1c} and R^{1b} is —H or —SR^{1c}. In some embodiments, R^{1a} and R^{1b} are each —SR^{1c}.

In some embodiments, R^{1a} and R^{1b} each independently comprise a radical of one or more keratolytic group (e.g., each radical of the one or more keratolytic group being independently selected from the group consisting of a radical of glycolic acid (GA), a radical of thioglycolic acid (TGA), a radical of lactic acid (Lac), a radical of thiolactic acid (TLac), a radical of lipoic acid (Lip), a radical of lipoic acid sulfoxide (Lipox), a radical of dihydrolipoic acid (diHLip), a radical of N-acetyl cysteine (NAC), a radical of cysteine (Cys), a radical of glutathione (GSH), a radical of captopril (Cap), and a radical of bucillamine (Buc)).

In some embodiments, R^{1a} and R^{1b} are each independently a radical of one or more keratolytic group, each radical of the one or more keratolytic group being independently selected from the group consisting of a radical of glycolic acid (GA), a radical of thioglycolic acid (TGA), a radical of lactic acid (Lac), a radical of thiolactic acid (TLac), a radical of lipoic acid (Lip), a radical of lipoic acid sulfoxide (Lipox), a radical of dihydrolipoic acid (diHLip), a radical of N-acetyl cysteine (NAC), a radical of cysteine (Cys), a radical of glutathione (GSH), a radical of captopril (Cap), and a radical of bucillamine (Buc).

In some embodiments, R^{1a} and R^{1b} each independently comprise a (thiol) radical of one or more keratolytic group, each (thiol) radical of the one or more keratolytic group being independently selected from the group consisting of a (thiol) radical of thioglycolic acid (TGA), a (thiol) radical of thiolactic acid (TLac), a (thiol) radical of dihydrolipoic acid (diHLip), a (thiol) radical of N-acetyl cysteine (NAC), a (thiol) radical of cysteine (Cys), a (thiol) radical of glutathione (GSH), a (thiol) radical of captopril (Cap), and a (thiol) radical of bucillamine (Buc).

In some embodiments, R^{1a} and R^{1b} are each independently a thiol radical of one or more keratolytic group, each thiol radical of the one or more keratolytic group being independently selected from the group consisting of a thiol radical of thioglycolic acid (TGA), a thiol radical of thiolactic acid (TLac), a thiol radical of dihydrolipoic acid (diHLip), a thiol radical of N-acetyl cysteine (NAC), a thiol radical of cysteine (Cys), a thiol radical of glutathione (GSH), a thiol radical of captopril (Cap), and a thiol radical of bucillamine (Buc).

In some embodiments, the (e.g., thiol) radical of the keratolytic agent comprises a (e.g., thiol) radical of one or more keratolytic group, each (e.g., thiol) radical of the one or more keratolytic group being independently selected from the group consisting of [Lac-Lac]·, [Lac-NAC]·, [Cys-Cys]·, [diHLip-NAC-NAC]·, [difilLip-Cap-Cap]·, [diHLip-Cys-Cys]·, [diHLip-Cys]·, [diHLip-Lipox-Lipox]·, and [diHLip-Lipox]·.

In some embodiments, the thiol radical of the keratolytic group is the point of attachment of R^{1a} and/or R^{1b} to the rest of the molecule. In some embodiments, R^{1a} and/or R^{1b} attach to the rest of the molecule to form a disulfide bond.

In some embodiments, R^{1a} and R^{1b} are each independently —H or:

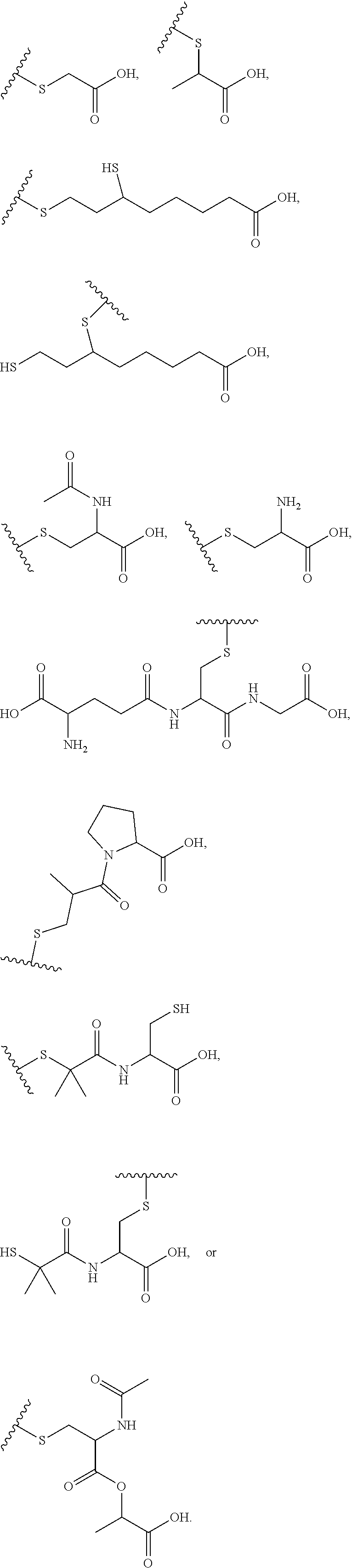

In some embodiments, R^{1a} and R^{1b} are the same. In some embodiments, R^{1a} and R^{1b} are different.

In some embodiments, the compound has the structure of Formula (IId):

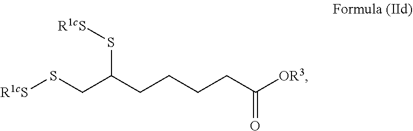

or a pharmaceutically acceptable salt or solvate (e.g., or a stereoisomer) thereof,
wherein:
   each R^{1c} is independently substituted or unsubstituted (e.g., straight or branched) alkyl (e.g., substituted with one or more (alkyl) substituent, each (alkyl) substituent being independently selected from the group consisting of carboxylic acid, —SH, thioalkyl, acetamide, amino, oxo, optionally substituted heterocycloalkyl (e.g., N-attached pyrrolidinyl substituted with —COOH)) or substituted or unsubstituted (e.g., straight or branched) heteroalkyl (e.g., substituted with one or more (heteroalkyl) substituent, each (heteroalkyl) substituent being independently selected from the group consisting of carboxylic acid, amino, thioalkyl, thiol, acetamide, and C₁-C₃ alkyl); and
   R³ is H or substituted or unsubstituted alkyl.

In some embodiments, each R^{1c} is independently substituted or unsubstituted (e.g., straight or branched) alkyl or substituted or unsubstituted (e.g., straight or branched) heteroalkyl. In some embodiments, each R^{1c} is independently substituted (e.g., straight or branched) alkyl or substituted (e.g., straight or branched) heteroalkyl.

In some embodiments, each R^{1c} is independently substituted (e.g., straight or branched) alkyl, the substituted alkyl being substituted with one or more (alkyl) substituent, each (alkyl) substituent being independently selected from the group consisting of carboxylic acid, —SH, thioalkyl, acetamide, amino, oxo, and optionally substituted heterocycloalkyl (e.g., N-attached pyrrolidinyl substituted with —COOH).

In some embodiments, each R^{1c} is independently substituted (e.g., straight or branched) heteroalkyl, the substituted heteroalkyl being substituted with one or more (heteroalkyl) substituent, each (heteroalkyl) substituent being independently selected from the group consisting of carboxylic acid, amino, thioalkyl, thiol, acetamide, and C₁-C₃ alkyl.

In some embodiments, each R^{1c} is the same. In some embodiments, each R^{1c} is different.

In some embodiments, R³ is H or unsubstituted alkyl. In some embodiments, R³ is H or C₁-C₆ alkyl. In some embodiments, R³ is H, methyl, ethyl, isopropyl, or tert-butyl. In some embodiments, R³ is H.

In some embodiments, R^{1a}, R^{1b} and each R^{1c} each independently comprise one or more substituent that is a carboxylic acid or an ester. In some embodiments, R^{1a}, R^{1b}, and each R^{1c} each independently comprise one or more substituent that is a carboxylic acid (e.g., —(C═O)OH). In some embodiments, R^{1a} comprises one or more substituent that is a carboxylic acid (e.g., —(C═O)OH). In some embodiments, R^{1b} comprises one or more substituent that is a carboxylic acid (e.g., —(C═O)OH). In some embodiments, each R^{1c} independently comprises one or more substituent that is a carboxylic acid (e.g., —(C═O)OH). In some embodiments, R^{1a}, R^{1b}, and each R^{1c} each independently comprise one or more substituent that is an ester (e.g., —(C═O)O—C₁-C₄alkyl). In some embodiments, R^{1a} comprises one or more substituent that is an ester (e.g., —(C═O)O—C₁-C₄alkyl). In some embodiments, R^{1b} comprises one or more substituent that is an ester (e.g., —(C═O)O—C₁-C₄alkyl). In some embodiments, each R^{1c} independently comprises one or more substituent that is an ester (e.g., —(C═O)O—C₁-C₄alkyl).

In some embodiments, the —(C═O)OH of R^{1a}, R^{1b}, and/or R^{1c} is optionally esterified (e.g., —(C═O)OH or —(C═O)O—C₁-C₄alkyl). In some embodiments, the C₁-C₄alkyl is methyl, ethyl, propyl, isopropyl, butyl, or t-butyl.

Provided in some embodiments herein is a compound, or a pharmaceutically acceptable salt thereof, having a structure provided in Table 2.

TABLE 2

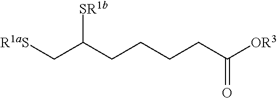

| Chemistry | | | |
|---|---|---|---|
| Example | R^{1a} | R^{1b} | R³ |
| 1-A | 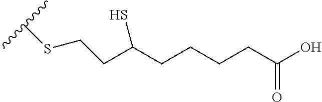 [SMILES: *SCCC(S)CCCCC(=O)O] | 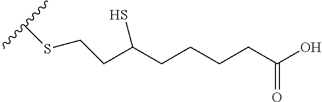 [SMILES: *SCCC(S)CCCCC(=O)O] | H |
| 2-A | 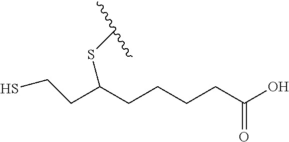 [SMILES: *SC(CCS)CCCCC(=O)O] | 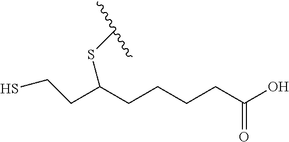 [SMILES: *SC(CCS)CCCCC(=O)O] | H |
| 3-A | 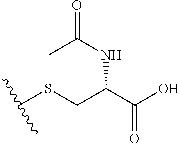 [SMILES: *SC[C@H](NC(C)=O)C(=O)O] | 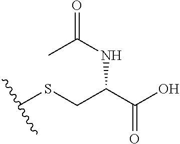 [SMILES: *SC[C@H](NC(C)=O)C(=O)O] | H |
| 4-A | 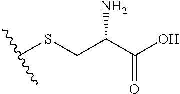 [SMILES: *SC[C@H](N)C(=O)O] | 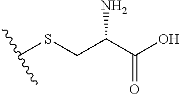 [SMILES: *SC[C@H](N)C(=O)O] | H |
| 5-A | 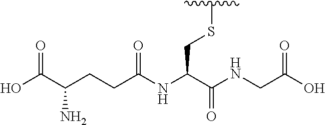 [SMILES: *SC[C@H](NC(=O)CC[C@H](N)C(=O)O)C(=O)NCC(=O)O] | 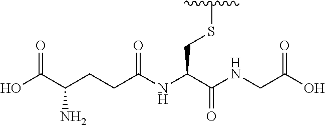 [SMILES: *SC[C@H](NC(=O)CC[C@H](N)C(=O)O)C(=O)NCC(=O)O] | H |
| 6-A | 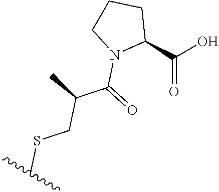 [SMILES: *SC[C@@H](C)C(=O)N1CCC[C@H]1C(=O)O] | 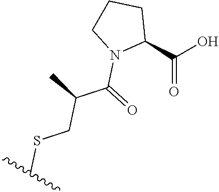 [SMILES: *SC[C@@H](C)C(=O)N1CCC[C@H]1C(=O)O] | H |
| 7-A | 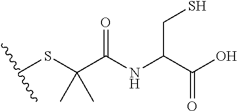 [SMILES: *SC(C)(C)C(=O)NC(CS)C(=O)O] | 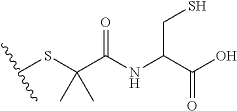 [SMILES: *SC(C)(C)C(=O)NC(CS)C(=O)O] | H |
| 8-A | 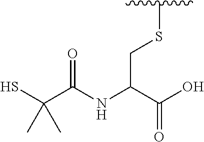 [SMILES: *SCC(NC(=O)C(C)(C)S)C(=O)O] | 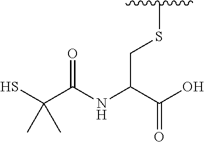 [SMILES: *SCC(NC(=O)C(C)(C)S)C(=O)O] | H |
| 9-A | 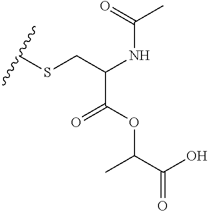 [SMILES: *SCC(NC(C)=O)C(=O)OC(C)C(=O)O] | 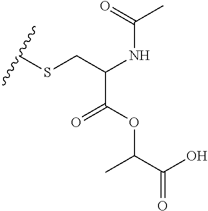 [SMILES: *SCC(NC(C)=O)C(=O)OC(C)C(=O)O] | H |
| 10-A | 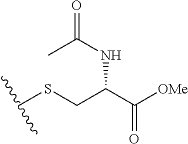 [SMILES: *SC[C@H](NC(C)=O)C(=O)OC] | 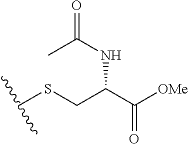 [SMILES: *SC[C@H](NC(C)=O)C(=O)OC] | H |
| 11-A | 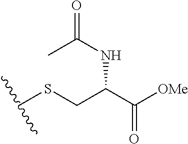 [SMILES: *SC[C@H](NC(C)=O)C(=O)OC] | 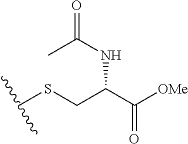 [SMILES: *SC[C@H](NC(C)=O)C(=O)OC] | CH₃ |
| 12-A | 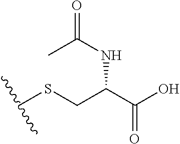 [SMILES: *SC[C@H](NC(C)=O)C(=O)O] | 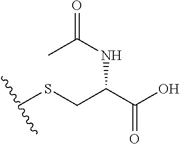 [SMILES: *SC[C@H](NC(C)=O)C(=O)O] | CH₃ |
| 13-A | 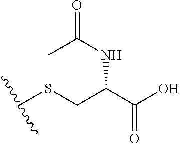 [SMILES: *SC[C@H](NC(C)=O)C(=O)O] | H | H |
| 14-A | H | 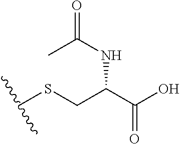 [SMILES: *SC[C@H](NC(C)=O)C(=O)O] | H |

In some embodiments, provided herein is a compound having the structure of Formula (III):

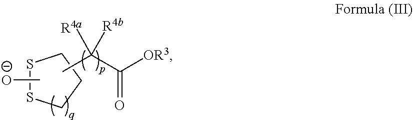

or a pharmaceutically acceptable salt or solvate (e.g., or a stereoisomer) thereof, wherein,

each R^{4a} and R^{4b} is independently H, halogen, or substituted or unsubstituted alkyl;

p is an integer from 1-10;
q is an integer from 1-3; and

R³ is H or substituted or unsubstituted alkyl.

In some embodiments, q is 1.

In some embodiments, the compound has the structure of Formula (IIIa):

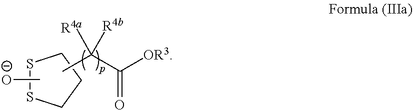

In some embodiments, p is an integer from 3-5. In some embodiments, p is 4. In some embodiments, q is 1 and p is 4.

In some embodiments, each R^{4a} and R^{4b} is independently H or substituted or unsubstituted alkyl. In some embodiments, each R^{4a} and R^{4b} is independently H, halogen, C₁-C₃alkyl, or C₁-C₃haloalkyl. In some embodiments, each R^{4a} and R^{4b} is H.

In some embodiments, the compound has the structure of Formula (IIIb):

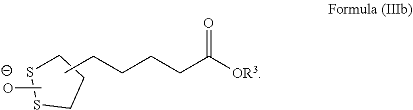

In some embodiments, the compound has the structure of Formula (IIIc):

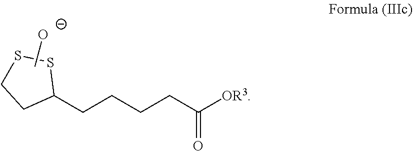

In some embodiments, R³ is H or unsubstituted alkyl. In some embodiments, R³ is H or C₁-C₆ alkyl. In some embodiments, R³ is H, methyl, ethyl, isopropyl, or tert-butyl. In some embodiments, R³ is H.

In some embodiments, the thiolanyl oxide ring structure is substituted with one or more substituent (e.g., each substituent being independently selected from the group consisting of halogen or unsubstituted or substituted alkyl).

In some embodiments, the compound has the structure of Formula (IIId):

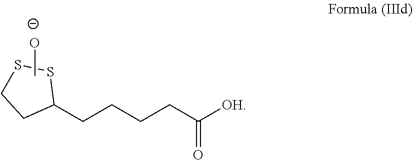

Each recitation of

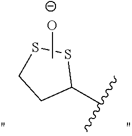

provided herein, unless otherwise stated, includes a specific

and explicit recitation of:

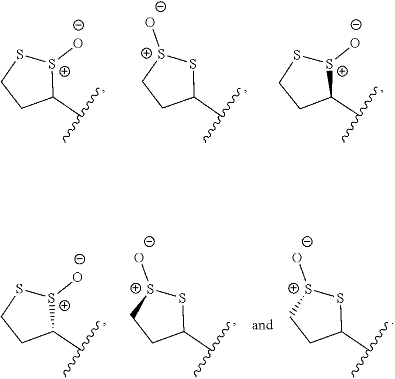

Provided in some embodiments herein is a compound, or a pharmaceutically acceptable salt thereof, having a structure provided in Table 3.

TABLE 3

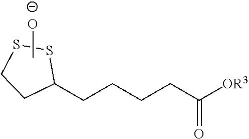

| Chemistry | | |
|---|---|---|
| Example | Ring Structure | R³ |
| 1-B | 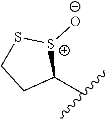 | H |
| 2-B | 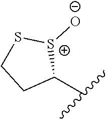 | H |
| 3-B | 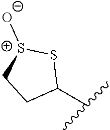 | H |
| 4-B | 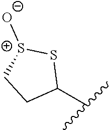 | H |
| 5-B | 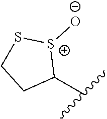 [SMILES: *C1CCS[S+]1[O-]] | CH₃ |
| 6-B | 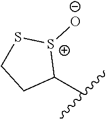 [SMILES: *C1CCS[S+]1[O-]] | CH₂CH₃ |
| 7-B | 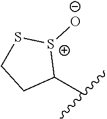 [SMILES: *C1CCS[S+]1[O-]] | CH(CH₃)CH₃ |
| 8-B | 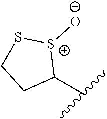 [SMILES: *C1CCS[S+]1[O-]] | C(CH₃)₃ |
| 9-B | 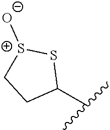 [SMILES: *C1CC[S+]([O-])S1] | CH₃ |
| 10-B | 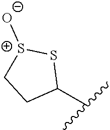 [SMILES: *C1CC[S+]([O-])S1] | CH₂CH₃ |
| 11-B | 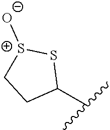 [SMILES: *C1CC[S+]([O-])S1] | CH(CH₃)CH₃ |
| 12-B | 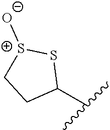 [SMILES: *C1CC[S+]([O-])S1] | C(CH₃)₃ |

In some embodiments, provided herein is a composition comprising a compound of any one of Formula (III) (e.g., Formula (III), Formula (IIIa), Formula (IIIb), Formula (IIIc), Formula (IIId), or Table 3) and lipoic acid (e.g., such that at R^{4a}st 50% or more, 75% or more, 90% or more, or 95% or more of the composition comprises a compound of any one of Formula (III) (e.g., Formula (III), Formula (IIIa), Formula (IIIb), Formula (IIIc), Formula (IIId), or Table 3)).

In some embodiments, provided herein is a compound having the structure of Formula

(IV):

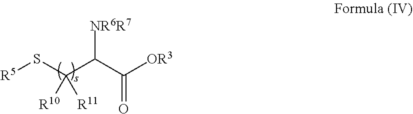

or a pharmaceutically acceptable salt or solvate (e.g., or a stereoisomer) thereof,
wherein:
   R⁵ is —SR^{1c};
      each R^{1c} is independently substituted or unsubstituted (e.g., straight or branched) alkyl (e.g., substituted with one or more (alkyl) substituent, each (alkyl) substituent being independently selected from the group consisting of carboxylic acid, —SH, thioalkyl, acetamide, amino, oxo, optionally substituted heterocycloalkyl (e.g., N-attached pyrrolidinyl substituted with —COOH)) or substituted or unsubstituted (e.g., straight or branched) heteroalkyl (e.g., substituted with one or more (heteroalkyl) substituent, each (heteroalkyl) substituent being independently selected from the group consisting of carboxylic acid, amino, thioalkyl, thiol, acetamide, and C₁-C₃ alkyl);
   R⁶ and R⁷ are each independently H, substituted or unsubstituted alkyl, or substituted or unsubstituted heteroalkyl;
   each R¹⁰ and R¹¹ is independently H, halogen, C₁-C₃-alkyl, C₁-C₃-haloalkyl, C₁-C₃-alkoxy, C₃-C₅-cycloalkyl, or two of R¹⁰ and R¹¹ are taken together with the atoms to which they are attached to form a C₃-C₅-cycloalkyl;
   R³ is H or substituted or unsubstituted alkyl; and
   s is an integer from 1-10.

In some embodiments, R⁶ and R⁷ are each independently H or substituted or unsubstituted alkyl (e.g., C₁-C₃ alkyl optionally substituted with oxo). In some embodiments, R⁶ and R⁷ are each independently H or C₁-C₃ alkyl optionally substituted with oxo. In some embodiments, R⁶ and R⁷ are each independently H or —(C═O)CH₃. In some embodiments, R⁶ is H and R⁷ is H or —(C═O)CH₃. In some embodiments, R⁶ is H and R⁷ is —(C═O)CH₃. In some embodiments, R⁶ and R⁷ are H.

In some embodiments, each R¹⁰ and R¹¹ is independently H, halogen, C₁-C₃alkyl, or C₁-C₃haloalkyl. In some embodiments, each R¹⁰ and R¹¹ is H.

In some embodiments, s is 1-3. In some embodiments, s is 1. In some embodiments, s is 1 and R¹⁰ and R¹¹ are H.

In some embodiments, R⁵ comprises a radical of one or more keratolytic group (e.g., each radical of the one or more keratolytic group being independently selected from the group consisting of a radical of glycolic acid (GA), a radical of thioglycolic acid (TGA), a radical of lactic acid (Lac), a radical of thiolactic acid (TLac), a radical of lipoic acid (Lip), a radical of lipoic acid sulfoxide (Lipox), a radical of dihydrolipoic acid (diHLip), a radical of N-acetyl cysteine (NAC), a radical of cysteine (Cys), a radical of glutathione (GSH), a radical of captopril (Cap), and a radical of bucillamine (Buc)).

In some embodiments, R⁵ is a radical of one or more keratolytic group, each radical of the one or more keratolytic group being independently selected from the group consisting of a radical of glycolic acid (GA), a radical of thioglycolic acid (TGA), a radical of lactic acid (Lac), a radical of thiolactic acid (TLac), a radical of lipoic acid (Lip), a radical of lipoic acid sulfoxide (Lipox), a radical of dihydrolipoic acid (diHLip), a radical of N-acetyl cysteine (NAC), a radical of cysteine (Cys), a radical of glutathione (GSH), a radical of captopril (Cap), and a radical of bucillamine (Buc).

In some embodiments, R⁵ comprises a (thiol) radical of one or more keratolytic group, each (thiol) radical of the one or more keratolytic group being independently selected from the group consisting of a (thiol) radical of thioglycolic acid (TGA), a (thiol) radical of thiolactic acid (TLac), a (thiol) radical of dihydrolipoic acid (diHLip), a (thiol) radical of N-acetyl cysteine (NAC), a (thiol) radical of cysteine (Cys), a (thiol) radical of glutathione (GSH), a (thiol) radical of captopril (Cap), and a (thiol) radical of bucillamine (Buc).

In some embodiments R⁵ is a thiol radical of one or more keratolytic group, each thiol radical of the one or more keratolytic group being independently selected from the group consisting of a thiol radical of thioglycolic acid (TGA), a thiol radical of thiolactic acid (TLac), a thiol radical of dihydrolipoic acid (diHLip), a thiol radical of N-acetyl cysteine (NAC), a thiol radical of cysteine (Cys), a thiol radical of glutathione (GSH), a thiol radical of captopril (Cap), and a thiol radical of bucillamine (Buc).

In some embodiments, the (e.g., thiol) radical of the keratolytic agent comprises a (e.g., thiol) radical of one or more keratolytic group, each (e.g., thiol) radical of the one or more keratolytic group being independently selected from the group consisting of [Lac-Lac]·, [Lac-NAC]·, [Cys-Cys]·, [diHLip-NAC-NAC]·, [diHLip-NAC]19, [diHLip-Cap-Cap]·, [diHLip-Cap]·, [diHLip-Cys-Cys]·, [diHLip-Cys]·, [diHLip-Lipox-Lipox]·, and [diHLip-Lipox]·.

In some embodiments, the thiol radical of the keratolytic group is the point of attachment of R⁵ to the rest of the molecule. In some embodiments, R⁵ attaches to the rest of the molecule to form a disulfide bond.

In some embodiments, R⁵ is:

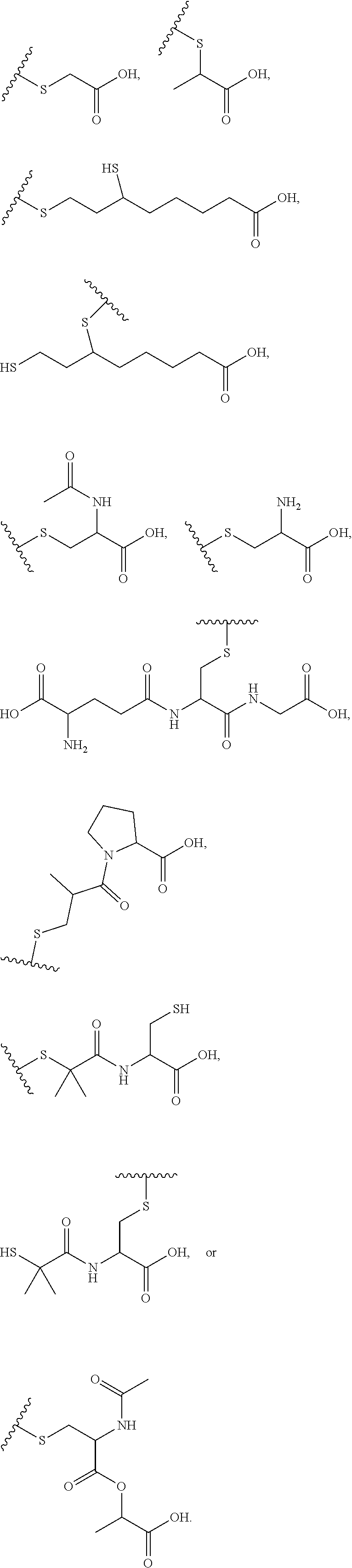

In some embodiments, the compound has the structure of Formula (IVa):

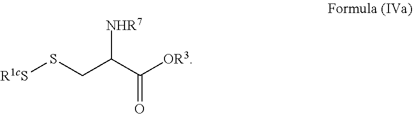

In some embodiments, R⁷ is H or —(C═O)CH₃. In some embodiments, R⁷ is H. In some embodiments, R⁷ is —(C═O)CH₃.

In some embodiments, each R^{1c} is independently substituted or unsubstituted (e.g., straight or branched) alkyl or substituted or unsubstituted (e.g., straight or branched) heteroalkyl. In some embodiments, each R^{1c} is independently substituted (e.g., straight or branched) alkyl or substituted (e.g., straight or branched) heteroalkyl.

In some embodiments, each R^{1c} is independently substituted (e.g., straight or branched) alkyl, the substituted alkyl being substituted with one or more (alkyl) substituent, each (alkyl) substituent being independently selected from the group consisting of carboxylic acid, —SH, thioalkyl, acetamide, amino, oxo, and optionally substituted heterocycloalkyl (e.g., N-attached pyrrolidinyl substituted with —COOH).

In some embodiments, each R^{1c} is independently substituted (e.g., straight or branched) heteroalkyl, the substituted heteroalkyl being substituted with one or more (heteroalkyl) substituent, each (heteroalkyl) substituent being independently selected from the group consisting of carboxylic acid, amino, thioalkyl, thiol, acetamide, and C₁-C₃ alkyl.

In some embodiments, R³ is H or unsubstituted alkyl. In some embodiments, R³ is H or C₁-C₆ alkyl. In some embodiments, R³ is H, methyl, ethyl, isopropyl, or tert-butyl. In some embodiments, R³ is H.

In some embodiments, R⁵ and each R^{1c} each independently comprise one or more substituent that is a carboxylic acid or an ester. In some embodiments, R⁵ and each R^{1c} each independently comprise one or more substituent that is a carboxylic acid (e.g., —(C═O)OH). In some embodiments, R⁵ and each independently comprise one or more substituent that is an ester (e.g., —(C═O)O—C₁-C₄alkyl).

In some embodiments, the —(C═O)OH of R⁵ and/or R^{1c} is optionally esterified (e.g., —(C═O)OH or —(C═O)O—C₁-C₄alkyl). In some embodiments, the C₁-C₄alkyl is methyl, ethyl, propyl, isopropyl, butyl, or t-butyl.

Provided in some embodiments herein is a compound, or a pharmaceutically acceptable salt thereof, having a structure provided in Table 4.

TABLE 4

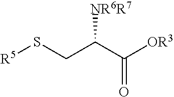

| Chemistry | | | | |
|---|---|---|---|---|
| Example | R⁵ | R⁶ | R⁷ | R³ |
| 1-C | 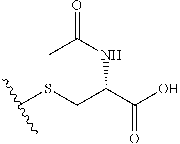 [SMILES: *SC[C@H](NC(C)=O)C(=O)O] | H | H | H |
| 2-C | 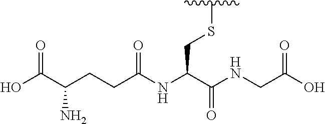 [SMILES: *SC[C@H](NC(=O)CC[C@H](N)C(=O)O)C(=O)NCC(=O)O] | H | H | H |
| 3-C | 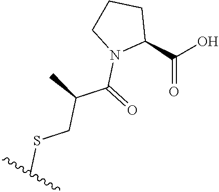 [SMILES: *SC[C@@H](C)C(=O)N1CCC[C@H]1C(=O)O] | H | H | H |
| 4-C | 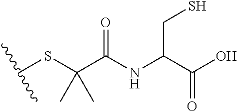 [SMILES: *SC(C)(C)C(=O)NC(CS)C(=O)O] | H | H | H |
| 5-C | 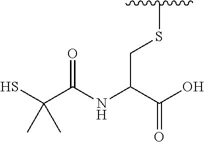 [SMILES: *SCC(NC(=O)C(C)(C)S)C(=O)O] | H | H | H |
| 6-C | 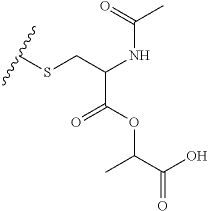 [SMILES: *SCC(NC(C)=O)C(=O)OC(C)C(=O)O] | H | H | H |
| 7-C | 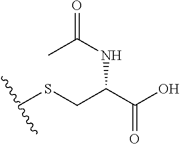 [SMILES: *SC[C@H](NC(C)=O)C(=O)O] | —(C═O)CH₃ | H | H |
| 8-C | 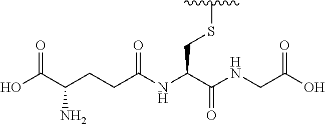 [SMILES: *SC[C@H](NC(=O)CC[C@H](N)C(=O)O)C(=O)NCC(=O)O] | —(C═O)CH₃ | H | H |
| 9-C | 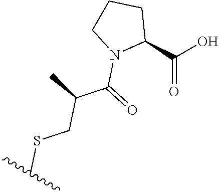 [SMILES: *SC[C@@H](C)C(=O)N1CCC[C@H]1C(=O)O] | —(C═O)CH₃ | H | H |
| 10-C | 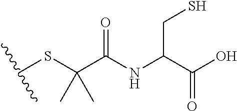 [SMILES: *SC(C)(C)C(=O)NC(CS)C(=O)O] | —(C═O)CH₃ | H | H |
| 11-C | 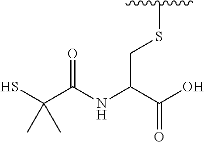 [SMILES: *SCC(NC(=O)C(C)(C)S)C(=O)O] | —(C═O)CH₃ | H | H |
| 12-C | 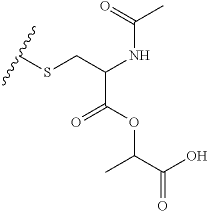 [SMILES: *SCC(NC(C)=O)C(=O)OC(C)C(=O)O] | —(C═O)CH₃ | H | H |
| 13-C | 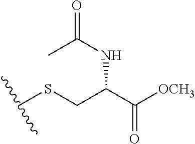 [SMILES: *SC[C@H](NC(C)=O)C(=O)OC] | H | H | H |
| 14-C | 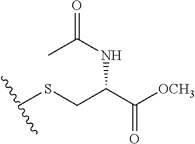 [SMILES: *SC[C@H](NC(C)=O)C(=O)OC] | H | H | CH₃ |
| 15-C | 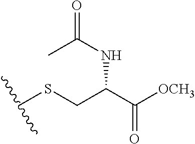 [SMILES: *SC[C@H](NC(C)=O)C(=O)OC] | —(C═O)CH₃ | H | H |
| 16-C | 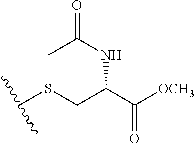 [SMILES: *SC[C@H](NC(C)=O)C(=O)OC] | —(C═O)CH₃ | H | CH₃ |
| 17-C | 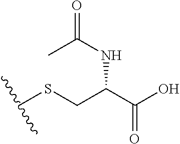 [SMILES: *SC[C@H](NC(C)=O)C(=O)O] | H | H | CH₃ |
| 18-C | 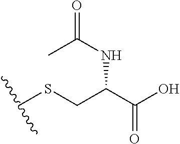 [SMILES: *SC[C@H](NC(C)=O)C(=O)O] | —(C═O)CH₃ | H | CH₃ |

The compounds used in the reactions described herein are made according to organic synthesis techniques starting from commercially available chemicals and/or from compounds described in the chemical literature or present disclosure. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2n d Edition (1999) Wiley-VCH, ISBN: O-471-19031-4; March, J. “Advanced Organic Chemistry: Reactions, Mechanisms, and Structure” 4th Edition (1992) John Wiley & Sons, ISBN: O-471-60180-2; Otera, J. (editor) “Modern Carbonyl Chemistry” (2000) Wiley-VCH, ISBN: 3-527-2987 1−1; Patai, S. “Patai's 1992 Guide to the Chemistry of Functional Groups” (1992) Interscience ISBN: O-471-93022-9; Solomons, T. W. G. “Organic Chemistry” 7th Edition (2000) John Wiley & Sons, ISBN: O-471-19095-O; Stowell, J. C., “Intermediate Organic Chemistry” 2nd Edition (1993) Wiley-Interscience, ISBN: O-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of chemicals prepared by the Chemical Abstract Service of the American Chemical Society, which are available in most public and university libraries, as well as through on-line databases (contact the American Chemical Society, Washington, D.C. for more details). Chemicals not commercially available in catalogs are optionally prepared by custom chemical synthesis houses, where many of the standard chemical supply houses (e.g., those listed above) provide custom synthesis services. A reference for the preparation and selection of pharmaceutical salts of the keratolytic conjugate described herein is P. H. Stahl & C. G. Wermuth “Handbook of Pharmaceutical Salts”, Verlag Helvetica Chimica Acta, Zurich, 2002.

In some embodiments, a compound provided herein is a compound represented by any structure herein, such as, for example, Formula (I), Formula (I-A), Formula (I-B), Formula (I-C), Formula (I-D), Formula (I′), Formula (Ia), Formula (Ib), Formula (Ic), Formula (Id), Formula (II), Formula (IIa), Formula (IIb), Formula (IIc), Formula (IId), Formula (III), Formula (IIIa), Formula (IIIb), Formula (IIIc), Formula (IIId), Formula (IV), Formula (IVa), Table 1, Table 2, Table 3, or Table 4. In some embodiments, a compound provided herein is administered as a pure chemical. In other embodiments, a compound provided herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21st Ed. Mack Pub. Co., Easton, PA (2005)).

Provided in some embodiments herein is a pharmaceutical composition comprising at least one keratolytic conjugate together with one or more pharmaceutically acceptable carriers. The carrier(s) (or excipient(s)) is acceptable or suitable if the carrier is compatible with the other ingredients of the composition and not deleterious to the recipient (i.e., the subject) of the composition.

In some embodiments, a compound provided herein (e.g., a compound represented by any structure herein, such as, for example, Formula (I), Formula (I-A), Formula (I-B), Formula (I-C), Formula (I-D), Formula (I′), Formula (Ia), Formula (Ib), Formula (Ic), Formula (Id), Formula (II), Formula (IIa), Formula (IIb), Formula (IIc), Formula (Hd), Formula (III), Formula (IIIa), Formula (IIIb), Formula (IIIc), Formula (IIId), Formula (IV), Formula (IVa), Table 1, Table 2, Table 3, or Table 4) is substantially pure, in that it contains less than, for example, about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21st Ed. Mack Pub. Co., Easton, PA (2005)).

In some embodiments provided herein is a pharmaceutical composition comprising a compound provided herein (e.g., a compound represented by any structure herein, such as, for example, Formula (I), Formula (I-A), Formula (I-B), Formula (I-C), Formula (I-D), Formula (I′), Formula (Ia), Formula (Ib), Formula (Ic), Formula (Id), Formula (II), Formula (IIa), Formula (IIb), Formula (IIc), Formula (Hd), Formula (III), Formula (IIIa), Formula (IIIb), Formula (IIIc), Formula (IIId), Formula (IV), Formula (IVa), Table 1, Table 2, Table 3, or Table 4) and at least one pharmaceutically acceptable excipient. In some embodiments, the pharmaceutical composition is suitable for ophthalmic administration. In some embodiments, the pharmaceutical composition is suitable for topical ophthalmic administration. In some embodiments, topical ophthalmic administration is administration in and/or around the eye, such as to the eyelid margin. In some embodiments, topical ophthalmic administration is administration to the ocular surface and the inner surface to the eyelid.

In some embodiments, a keratolytic conjugate provided herein (e.g., a compound represented by any structure herein, such as, for example, Formula (I), Formula (I-A), Formula (I-B), Formula (I-C), Formula (I-D), Formula (I′), Formula (Ia), Formula (Ib), Formula (Ic), Formula (Id), Formula (II), Formula (IIa), Formula (IIb), Formula (IIc), Formula (IId), Formula (III), Formula (IIIa), Formula (IIIb), Formula (IIIc), Formula (IIId), Formula (IV), Formula (IVa), Table 1, Table 2, Table 3, or Table 4) is formulated as a solution or suspension for topical administration to the eye.

In some embodiments, a keratolytic conjugate provided herein (e.g., a compound represented by any structure herein, such as, for example, Formula (I), Formula (I-A), Formula (I-B), Formula (I-C), Formula (I-D), Formula (I′), Formula (Ia), Formula (Ib), Formula (Ic), Formula (Id), Formula (II), Formula (IIa), Formula (IIb), Formula (IIc), Formula (IId), Formula (III), Formula (IIIa), Formula (IIIb), Formula (IIIc), Formula (IIId), Formula (IV), Formula (IVa), Table 1, Table 2, Table 3, or Table 4) is formulated for administration by injection. In some instances, the injection formulation is an aqueous formulation. In some instances, the injection formulation is a non-aqueous formulation. In some instances, the injection formulation is an oil-based formulation, such as sesame oil, or the like.

In some embodiments, the dose of the composition comprising at least one keratolytic conjugate as provided herein differ, depending upon the patient's (e.g., human) condition, that is, general health status, age, and other factors.

Pharmaceutical compositions provided in some embodiments herein are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity). Optimal doses are generally determined using experimental models and/or clinical trials. The optimal dose depends upon the body mass, weight, or blood volume of the patient.

In other embodiments, the topical compositions described herein are combined with a pharmaceutically suitable or acceptable carrier (e.g., a pharmaceutically suitable (or acceptable) excipient, physiologically suitable (or acceptable) excipient, or physiologically suitable (or acceptable) carrier). Exemplary excipients are described, for example, in Remington: The Science and Practice of Pharmacy (Gennaro, 21st Ed. Mack Pub. Co., Easton, PA (2005)).

Methods of Treatment Utilizing Keratolytic Conjugates

In some embodiments provided herein is a method of treating a dermatological or ophthalmic disease or disorder in a patient in need of thereof, comprising administering to the patient any compound provided herein, or a pharmaceutically acceptable salt thereof, or a (e.g., pharmaceutical) composition comprising any compound provided herein, or a pharmaceutically acceptable salt thereof, such as a compound represented by any structure herein, such as, for example, Formula (I), Formula (I-A), Formula (I-B), Formula (I-C), Formula (I-D), Formula (I′), Formula (Ia), Formula (Ib), Formula (Ic), Formula (Id), Formula (II), Formula (IIa), Formula (IIb), Formula (IIc), Formula (IId), Formula (III), Formula (IIIa), Formula (IIIb), Formula (IIIc), Formula (IIId), Formula (IV), Formula (IVa), Table 1, Table 2, Table 3, or Table 4. In some embodiments provided herein the pharmaceutical composition is in the form of a solution or suspension suitable for topical ophthalmic administration. In some embodiments, topical ophthalmic administration is administration in and/or around the eye, such as to the eyelid margin. In some embodiments, topical ophthalmic administration is administration to the ocular surface and the inner surface to the eyelid. In some embodiments, the dermatological or ophthalmic disease or disorder is inflammation or hyperkeratosis (e.g., of the eyes or skin). In some embodiments, the dermatological or ophthalmic disease or disorder is inflammation or hyperkeratosis of the eyes or skin (e.g., the ocular surface). In some embodiments, the dermatological or ophthalmic dermatological disease or disorder is selected from the group consisting of meibomian gland dysfunction (MGD), dry eye disease (DED), ocular manifestations of graft versus host disease, vernal keratoconjunctivitis, atopic keratoconjunctivitis, Cornelia de Lange Syndrome, evaporative eye disease, aqueous deficiency dry eye, blepharitis, and seborrheic blepharitis. In some embodiments, the dermatological or ophthalmic disease or disorder is inflammation or hyperkeratosis (e.g., of the eyes or skin), such as, for example, meibomian gland dysfunction (MGD), dry eye disease (DED), ocular manifestations of graft versus host disease, vernal keratoconjunctivitis, atopic keratoconjunctivitis, Cornelia de Lange Syndrome, evaporative eye disease, aqueous deficiency dry eye, blepharitis, seborrheic blepharitis, or any combination thereof.

In some embodiments, the ophthalmic disease or disorder is selected from the group consisting of dry eye, lid wiper epitheliopathy (LWE), contact lens discomfort (CLD), dry eye syndrome, evaporative dry eye syndrome, aqueous deficiency dry eye syndrome, blepharitis, keratitis, meibomian gland dysfunction, conjunctivitis, lacrimal gland disorder, contact lens related conditions and inflammation of the anterior surface of the eye, infection of the anterior surface of the eye, and autoimmune disorder of the anterior surface of the eye.

Provided herein is a method for treating an ocular surface disorder in an individual in need thereof comprising topical administration of a keratolytic conjugate to the individual in need thereof. In some embodiments, administration occurs with the assistance of a health-care provider (e.g., this category includes both acute and maintenance uses of the keratolytic conjugate). An acute use, in some embodiments, requires a stronger keratolytic conjugate (either in terms of concentration of the agent or the inherent activity of the agent). A maintenance use, in some embodiments, allows for the use of lower concentrations of the agent, or agents with lower inherent activity. A maintenance use, in some embodiments, involves a patient at a routine visit to the health care provider. Both acute uses and maintenance uses optionally involve use of an eye-protecting device or apparatus. In some embodiments, the acute use is performed by the health care provider, and the maintenance use is performed by the patient or non-health care provider. In some embodiments, administration does not occur with the active assistance of a health care provider (e.g., but rather involves the patient applying the keratolytic conjugate to his/her own eyelid margin). In some embodiments, such administration occurs over an extended period of time (e.g., one way of describing this patient-administered multi-administration mode is as a chronic use). In some embodiments, different or second formulations of the keratolytic conjugate are used for chronic or patient-administered uses. In some embodiments the different or second formulation utilizes a lower concentration of the keratolytic conjugate. In some embodiments, the second or different formulation utilizes a keratolytic conjugate that has a lower activity than the first formulation.

It should be understood that the present methods also include the physical removal of an obstruction in an meibomian gland (e.g., followed by chronic and/or maintenance administration of a keratolytic conjugate provided herein).

In some embodiments provided herein is a method for treating meibomian gland dysfunction in a patient in need thereof, comprising topically administering to the patient a composition comprising a therapeutically-effective amount of at least one keratolytic conjugate in an ophthalmically-acceptable carrier. In some embodiments, the topical administration of the composition comprising a therapeutically-effective amount of at least one keratolytic conjugate in an ophthalmically-acceptable carrier results in enhanced meibum production.

In some embodiments, the topical administration of the composition comprising a therapeutically-effective amount of at least one keratolytic conjugate in an ophthalmically-acceptable carrier occurs until the keratinized obstruction is relieved. In some embodiments, the topical administration of the composition comprising a therapeutically-effective amount of at least one keratolytic conjugate in an ophthalmically-acceptable carrier occurs periodically after relieving of the keratinized obstruction. In some embodiments, the topical administration of the composition comprising a therapeutically-effective amount of at least one keratolytic conjugate in an ophthalmically-acceptable carrier is a single administration. In some embodiments, the topical administration of the composition comprising a therapeutically-effective amount of at least one keratolytic conjugate in an ophthalmically-acceptable carrier is a periodic administration. In some embodiments, the topical administration of the composition comprising a therapeutically-effective amount of at least one keratolytic conjugate in an ophthalmically-acceptable carrier occurs once a day. In some embodiments, the topical administration of the composition comprising a therapeutically-effective amount of at least one keratolytic conjugate in an ophthalmically-acceptable carrier occurs twice a day. In some embodiments, the topical administration of the composition comprising a therapeutically-effective amount of at least one keratolytic conjugate in an ophthalmically-acceptable carrier occurs more than twice a day.

In some embodiments, the composition for topical administration comprises a therapeutically-effective amount of at least one keratolytic conjugate in an ophthalmically-acceptable carrier is a solution. In some embodiments, the composition for topical administration comprises a therapeutically-effective amount of at least one keratolytic conjugate in an ophthalmically-acceptable carrier is a solution suitable for topical administration as eye drops. In some embodiments, the composition for topical administration comprises a therapeutically-effective amount of at least one keratolytic conjugate in an ophthalmically-acceptable carrier is a gel, ocular insert, spray, or other topical ocular delivery method. In some embodiments, the composition for topical administration comprises a therapeutically-effective amount of at least one keratolytic conjugate in an ophthalmically-acceptable carrier is a semi-solid. In some embodiments, the composition for topical administration comprises a therapeutically-effective amount of at least one keratolytic conjugate in an ophthalmically-acceptable carrier is homogenous. In some embodiments, the composition for topical administration comprises a therapeutically-effective amount of at least one keratolytic conjugate in an ophthalmically-acceptable carrier is a dispersion. In some embodiments, the composition for topical administration comprises a therapeutically-effective amount of at least one keratolytic conjugate in an ophthalmically-acceptable carrier is hydrophilic. In some embodiments, the composition for topical administration comprises a therapeutically-effective amount of at least one keratolytic conjugate in an ophthalmically-acceptable carrier and an or oleaginous base. In some embodiments, the composition for topical administration comprises a therapeutically-effective amount of at least one keratolytic conjugate in an ophthalmically-acceptable carrier and at least one ophthalmically-acceptable excipient.

In some embodiments provided herein is a method for treating MGD in a patient in need thereof comprising topical administration of a composition comprising a keratolytic conjugate. In some embodiments, the topical administration of the composition comprising a keratolytic conjugate occurs once a week. In some embodiments, the topical administration of the composition comprising a keratolytic conjugate occurs twice a week. In some embodiments, the topical administration of the composition comprising a keratolytic conjugate occurs every other day. In some embodiments, the topical administration of the composition comprises a keratolytic conjugate occurs every day. In some embodiments, the topical administration of the composition comprises a keratolytic conjugate occurs several times a day.

In some embodiments, the method comprises administering a compound or formulation provided herein in an acute treatment scenario. In some embodiments, the method comprises treatment of a patient naïve to treatment. In some embodiments, the method comprises administering a compound or formulation provided herein in a chronic treatment scenario. In some embodiments, the method comprises administering a compound or formulation provided herein in a maintenance therapy scenario. In an acute treatment scenario, the administered dosage of keratolytic conjugate may be higher than the administered dosage of keratolytic conjugate employed in a chronic treatment scenario or a maintenance therapy scenario. In an acute treatment scenario, the keratolytic conjugate may be different from the keratolytic conjugate employed in a chronic treatment scenario. In some embodiments, the course of therapy begins in the initial phase of therapy as an acute treatment scenario and later transitions into a chronic treatment scenario or a maintenance therapy scenario. In some embodiments, the meibomian gland opening pharmacological agent administered in the acute treatment scenario is a keratolytic agent and/or keratoplastic agent, and the pharmacological agent administered in the chronic treatment scenario or a maintenance therapy scenario is a keratolytic conjugate.

In some embodiments, an initial treatment is administered (e.g., by a physician or healthcare professional) to an individual to initially open a blockage of the meibomian gland, such as by placing a more highly concentrated formulation of one of the keratolytic conjugate provided herein. In the event the higher concentration formulations are required, the application thereof may require ocular shielding or other activity to minimize the impact of irritation or disruption of the ocular surface or surrounding tissues. Following such a procedure, a patient may be given a different formulation of keratolytic conjugate to take home to apply periodically to the lid margin to maintain the patency of the meibomian gland. Such application may occur twice daily, once a day, weekly or monthly, depending on the formulation activity and the therapeutic product profile of the formulation.

Provided in some embodiments of the methods of treatment described herein is the location of the topical administration of the composition. In some embodiments, the composition comprising a keratolytic conjugate is administered such that no irritation to eye occurs. In some embodiments, the composition comprising a keratolytic conjugate is administered to the eye lid margin.

In some embodiments of the methods of treatment provided herein is the use of a protective element provided to the eye to avoid irritation to the eye. Although the formulations described herein are generally non-irritating, in some embodiments (e.g., high concentration of agent or when used on a sensitive eye) a protective element provides an additional layer of safety and comfort for the patient. In some embodiments, the composition comprising a keratolytic conjugate is administered while an eye shield is placed on the eye to reduce contact of the pharmacological agent with the cornea and/or conjunctiva such that reduced irritation to eye occurs. In some embodiments, the eye shield is a contact lens or an eye covering. In some embodiments, the eye covering comprises a self-adhesive. In some embodiments, the composition comprising a keratolytic conjugate is administered while the lid is pulled away from the globe to reduce contact of the pharmacological agent with the cornea and/or conjunctiva such that reduced irritation to eye occurs.

While exemplary embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Examples

### I. Chemical Synthesis

Solvents, reagents and starting materials were purchased from commercial vendors and used as received unless otherwise described. All reactions were performed at room temperature unless otherwise stated. Starting materials were purchased from commercial sources or synthesized according to the methods described herein or using literature procedures or the present disclosure. (2 S)-3-[4-carbamoylpiperidine-1-carbonyl)oxyphenyl]-2-[[(2 S)-4-methyl-2-[[2-(2-methylphenoxy)acetyl]amino]pentanoyl]amino]propanoic acid was prepared according the literature procedures, see WO0037444 A1 for details.

### Abbreviations

The following abbreviations are used in the Examples and other parts of the examples:
AcOH: acetic acid
CDCl₃: deuterated chloroform
DCM: dichloromethane
DIPEA: N,N-diisopropylethylamine
DMF: N,N-dimethylformamide
DMSO-d₆: dimethyl sulfoxide-d₆
EtOAc: ethyl acetate
h: hours
H₂O: water
HPLC: high performance liquid chromatography
MeCN: acetonitrile
MeOH: methanol
MgSO₄: magnesium sulfate
min.: minute(s)
N₂: nitrogen
NaHCO₃: sodium bicarbonate
prep: prepartive
Rt: retention time
r.t.: room temperature
sat.: saturated
TFA: trifluoroacetic acid
THF: tetrahydrofuran
wt: weight

### Analytical Methods:

Method A: Phenomenex Gemini NX C18 5 μm, 150×4.6 mm; A=water+0.1% formic acid; B=MeOH+0.1% formic acid; 40° C.; % B: 0.0 min 5%, 0.5 min 5%, 7.5 min 95%, 10.0 min 95%, 10.1 min 5%, 13.0 min 5%; 1.5 mL/min.

Method B: Waters Sunfire C18 3.5 μm, 50×4.6 mm; A=water+0.1% formic acid; B=MeCN; 40° C.; % B: 0.0 min 5%, 1.0 min 37.5%, 3.0 min 95%, 3.5 min 95%, 3.51 min 5%, 4.0 min 5%; 2.25 mL/min.

Method C: Phenomenex Gemini NX C18 5 μm, 150×4.6 mm; A=water+0.1% formic acid; B=MeCN; 40° C.; % B: 0.0 min 5%, 0.5 min 5%, 7.5 min 95%, 10.0 min 95%, 10.1 min 5%, 13.0 min 5%; 1.5 mL/min.

### Chemical Synthesis Example 1

### 4-((2S)-3-(1-((Isopropoxycarbonyl)oxy)ethoxy)-2-((S)-4-methyl-2-(2-(o-tolyloxy)acetamido)pentanamido)-3-oxopropyl)phenyl 4-carbamoylpiperidine-1-carboxylate

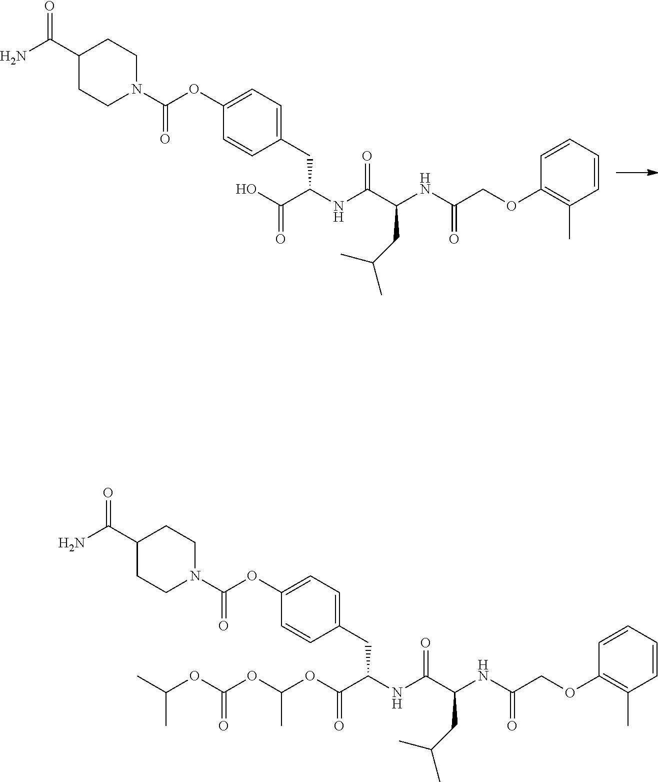 [SMILES: Cc1ccccc1OCC(=O)N[C@@H](CC(C)C)C(=O)N[C@@H](Cc1ccc(OC(=O)N2CCC(C(N)=O)CC2)cc1)C(=O)O ; Cc1ccccc1OCC(=O)N[C@@H](CC(C)C)C(=O)N[C@@H](Cc1ccc(OC(=O)N2CCC(C(N)=O)CC2)cc1)C(=O)OC(C)OC(=O)OC(C)C]

3-[4-(4-(4-Carbamoylpiperidine-1-carbonyl)oxyphenyl]-2-[[(2 S)-4-methyl-2-[[2-(2-methylphenoxy)acetyl]amino]pentanoyl]amino]propanoic acid (80 mg, 0.134 mmol) was dissolved in anhydrous DMF (5.0 mL). 1-Chloroethyl isopropyl carbonate (50 μL 0.327 mmol) was added and the mixture stirred at 60° C. for 24 hours. DIPEA (80 μL, 0.459 mmol) and 1-chloroethyl isopropyl carbonate (50 μL 0.327 mmol) were added and the mixture stirred at 60° C. for 2 hours. The solvent was evaporated in vacuo, and the residue partitioned between EtOAc (40 mL) and sat. NaHCO_{3(aq)} (20 mL). The layers were separated, and the organic phase washed with sat. brine solution (20 mL), dried (MgSO₄), filtered, and the solvent was evaporated in vacuo. The crude product was purified by flash chromatography (Biotage SP1; 10 g SNAP cartridge) eluting with EtOAc→20% acetone-EtOAc to yield[4-[3-(1-isopropoxycarbonyloxyethoxy)-2-[[(2S)-4-methyl-2-[[2-(2-methylphenoxy)acetyl]amino]pentanoyl]amino]-3-oxo-propyl]phenyl]4-carbamoylpiperidine-1-carboxylate (58 mg, 60%) as an off-white solid. LCMS (Method A): Rt=8.36 mins; [M+H]⁺=727.6.

### Chemical Synthesis Example 2

### 1-Chloroethyl 2-(trityloxy)acetate

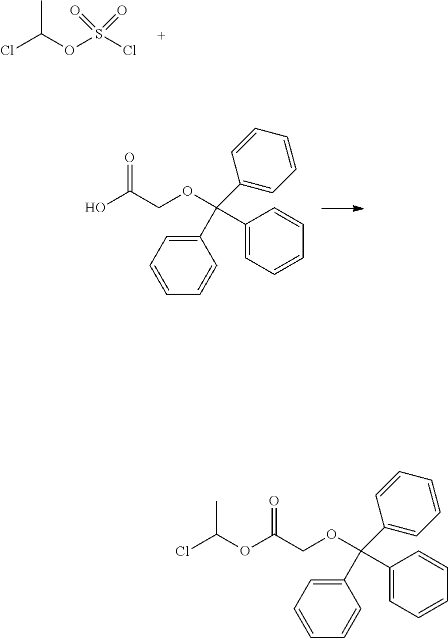

A mixture of 1-chloroethyl sulfochloridate (309 mg, 1.73 mmol), 2-(trityloxy)acetic acid (500 mg, 1.57 mmol) tetrabutylammonium hydrogen sulfate (53 mg, 0.157 mmol) and sodium bicarbonate (528 mg, 6.28 mmol) in DCM (15 mL) and water (15 mL) were stirred vigorously at r.t. for 6 h. The reaction mixture was passed through a phase separator and the filtrate was evaporated in vacuo to yield a viscous oil. The product was purified by flash chromatography eluting with iso-hexane→40% EtOAc-isohexane and then further purified by flash chromatography eluting with 20% DCM-isohexane→80% DCM-isohexane to yield 1-chloroethyl 2-(trityloxy)acetate as a colourless oil (170 mg, 28%). LCMS (Method B): Rt=3.45 min; [M-C₄H₆ClO₃]⁺=243.2. ¹HNMR (400 MHz, CDCl₃) δ 7.46-7.49 (m, 6H), 7.24-7.34 (m, 9H), 6.52 (q, J=5.8 Hz, 1H), 3.85 (s, 2H), 1.73 (d, J=6.0 Hz, 3H).

### 4-((2S)-2-((S)-4-Methyl-2-(2-(o-tolyloxy)acetamido)pentanamido)-3-oxo-3-(1-(2-(trityloxy)acetoxy)ethoxy)propyl)phenyl 4-carbamoylpiperidine-1-carboxylate

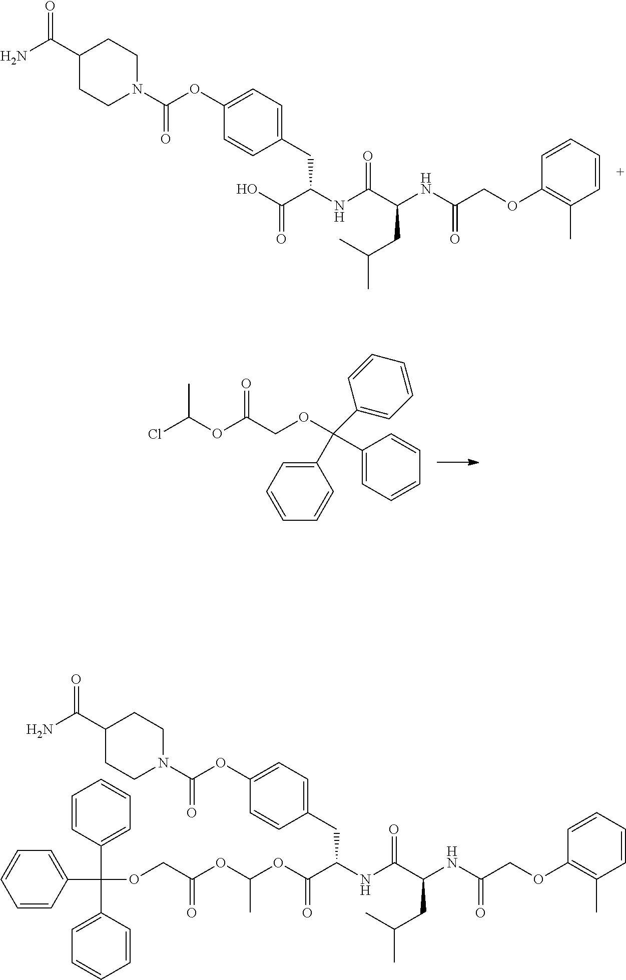 [SMILES: Cc1ccccc1OCC(=O)N[C@@H](CC(C)C)C(=O)N[C@@H](Cc1ccc(OC(=O)N2CCC(C(N)=O)CC2)cc1)C(=O)O ; CC(Cl)OC(=O)COC(c1ccccc1)(c1ccccc1)c1ccccc1 ; Cc1ccccc1OCC(=O)N[C@@H](CC(C)C)C(=O)N[C@@H](Cc1ccc(OC(=O)N2CCC(C(N)=O)CC2)cc1)C(=O)OC(C)OC(=O)COC(c1ccccc1)(c1ccccc1)c1ccccc1]

A mixture of (S)-3-(4-((4-carbamoylpiperidine-1-carbonyl)oxy)phenyl)-2-((S)-4-methyl-2-(2-(o-tolyloxy)acetamido)pentanamido)propanoic acid (100 mg, 0.168 mmol), 1-chloroethyl 2-(trityloxy)acetate (77 mg, 0.201 mmol), DIPEA (58 μL, 0.335 mmol) in DMF (3.0 mL) were heated under an atmosphere of nitrogen at 50° C. for 48 h. The mixture was cooled to r.t. and diluted with EtOAc (30 mL), sequentially washed with sat. aqueous NaHCO₃ (30 mL), water (30 mL) and sat. brine solution (30 mL), dried (MgSO₄), filtered and the solvent evaporated in vacuo. The crude product was purified by flash chromatography eluting with isohexane→EtOAc—→10% MeOH-EtOAc to yield 4-((2S)-2-((S)-4-methyl-2-(2-(o-tolyloxy)acetamido)pentanamido)-3-oxo-3-(1-(2-(trityloxy)acetoxy)ethoxy)propyl)phenyl 4-carbamoylpiperidine-1-carboxylate as a colourless oil (19.3 mg, 12%). LCMS (Method B): Rt=3.34 min; [M+NH₄]⁺=958.5.

### 4-((2S)-3-(1-(2-Hydroxyacetoxy)ethoxy)-2-((S)-4-methyl-2-(2-(o-tolyloxy)acetamido)pentanamido)-3-oxopropyl)phenyl 4-carbamoylpiperidine-1-carboxylate

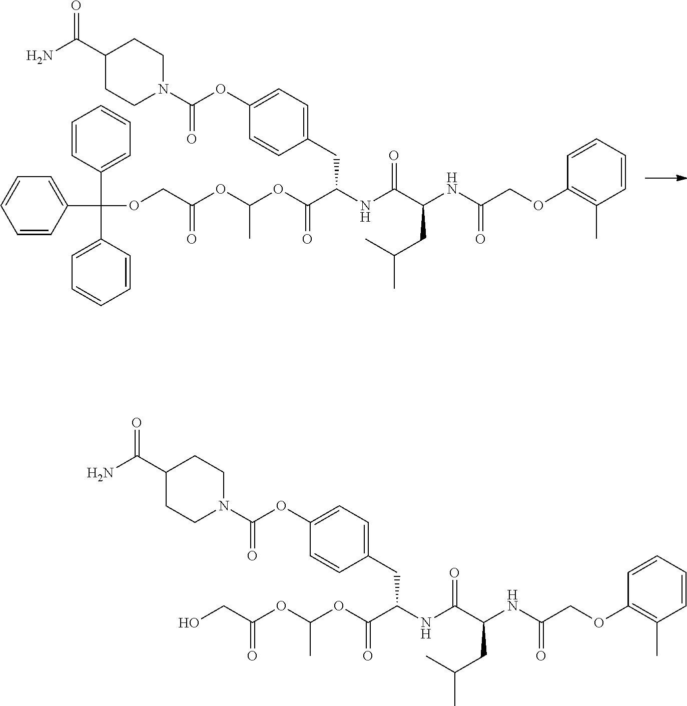 [SMILES: Cc1ccccc1OCC(=O)N[C@@H](CC(C)C)C(=O)N[C@@H](Cc1ccc(OC(=O)N2CCC(C(N)=O)CC2)cc1)C(=O)OC(C)OC(=O)COC(c1ccccc1)(c1ccccc1)c1ccccc1 ; Cc1ccccc1OCC(=O)N[C@@H](CC(C)C)C(=O)N[C@@H](Cc1ccc(OC(=O)N2CCC(C(N)=O)CC2)cc1)C(=O)OC(C)OC(=O)CO]

To a solution of 4-((2S)-2-((S)-4-methyl-2-(2-(o-tolyloxy)acetamido)pentanamido)-3-oxo-3-(1-(2-(trityloxy)acetoxy)ethoxy)propyl)phenyl 4-carbamoylpiperidine-1-carboxylate (19 mg, 20.2 μmol) in DCM (2.0 mL) was added triethylsilane (3.3 μL, 20.5 μmol) followed by a solution of TFA (0.2 mL) in DCM (2.0 mL). The reaction mixture was stirred at r.t. for 45 min. The reaction mixture was diluted with DCM (10 mL), washed with sat. aqueous NaHCO₃ (10 mL), passed through a phase separator, the filtrate evaporated in vacuo and the resultant residue dissolved in DMSO and the crude product purified by preparative reversed-phase HPLC. The appropriate fractions were combined and evaporated in vacuo (lyophilisation) to yield a sticky solid. The residue was dissolved in 1:1 MeCN—H₂O and the solution frozen (−78° C.). The solvent was evaporated in vacuo (lyophilisation) to yield 4-((2S)-3-(1-(2-hydroxyacetoxy)ethoxy)-2-((S)-4-methyl-2-(2-(o-tolyloxy)acetamido)pentanamido)-3-oxopropyl)phenyl 4-carbamoylpiperidine-1-carboxylate as a white solid (3.6 mg, 25%). LCMS (Method A): Rt=7.84 min; [M+H]⁺=699.7.

### Chemical Synthesis Example 3

### 1-Chloroethyl (2R)-2-(benzyloxy)propanoate

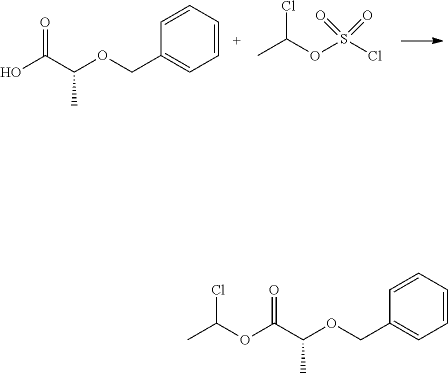

To a solution of (R)-2-(benzyloxy)propanoic acid (100 mg, 0.530 mmol) and 1-chloroethyl sulfurochloridate (132 mg, 7.45 mmol) in DCM (10 mL) was added a solution of tetrabutylammonium hydrogen sulfate (18 mg, 0.0530 mmol) and sodium bicarbonate (177 mg, 2.11 mmol) in water (10 mL) and the reaction mixture stirred vigorously at r.t. for 16 hours. The reaction mixture was passed through a phase separator and the filtrate evaporated in vacuo to give 1-chloroethyl (2R)-2-(benzyloxy)propanoate as a clear oil (100 mg, 78%). LCMS (Method B): Rt=2.91 min; [M+H]⁺=242.4.

### 4-((2S)-3-(1-(((R)-2-(Benzyloxy)propanoyl)oxy)ethoxy)-2-((S)-4-methyl-2-(2-(o-tolyloxy)acetamido)pentanamido)-3-oxopropyl)phenyl 4-carbamoylpiperidine-1-carboxylate

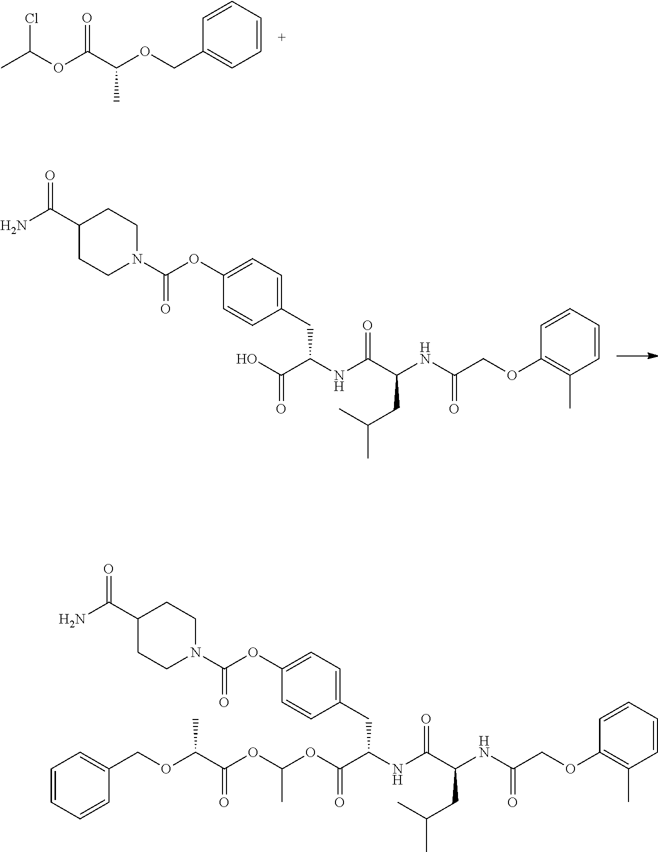 [SMILES: CC(Cl)OC(=O)[C@@H](C)OCc1ccccc1 ; Cc1ccccc1OCC(=O)N[C@@H](CC(C)C)C(=O)N[C@@H](Cc1ccc(OC(=O)N2CCC(C(N)=O)CC2)cc1)C(=O)O ; Cc1ccccc1OCC(=O)N[C@@H](CC(C)C)C(=O)N[C@@H](Cc1ccc(OC(=O)N2CCC(C(N)=O)CC2)cc1)C(=O)OC(C)OC(=O)[C@@H](C)OCc1ccccc1]

A stirred solution of (S)-3-(4-((4-carbamoylpiperidine-1-carbonyl)oxy)phenyl)-2-((S)-4-methyl-2-(2-(o-tolyloxy)acetamido)pentanamido)propanoic acid (148 mg, 0.250 mmol), 1-chloroethyl (2R)-2-(benzyloxy)propanoate (50 mg, 0.206 mmol) and DIPEA (72 μL, 0.412 mmol) in DMF (10 mL) were stirred at 60° C. for 24 h. The solvent was evaporated in vacuo and the resulting crude product purified by preparative reversed-phase HPLC to yield 4-((2S)-3-(1-(((R)-2-(benzyloxy)propanoyl)oxy)ethoxy)-2-((S)-4-methyl-2-(2-(o-tolyloxy)acetamido)pentanamido)-3-oxopropyl)phenyl 4-carbamoylpiperidine-1-carboxylate as a white solid (55 mg, 33%). LCMS (Method B): Rt=2.98 min; [M+H]⁺=803.5.

### 4-((2S)-3-(1-(((R)-2-Hydroxypropanoyl)oxy)ethoxy)-2-((S)-4-methyl-2-(2-(o-tolyloxy)acetamido)pentanamido)-3-oxopropyl)phenyl 4-carbamoylpiperidine-1-carboxylate

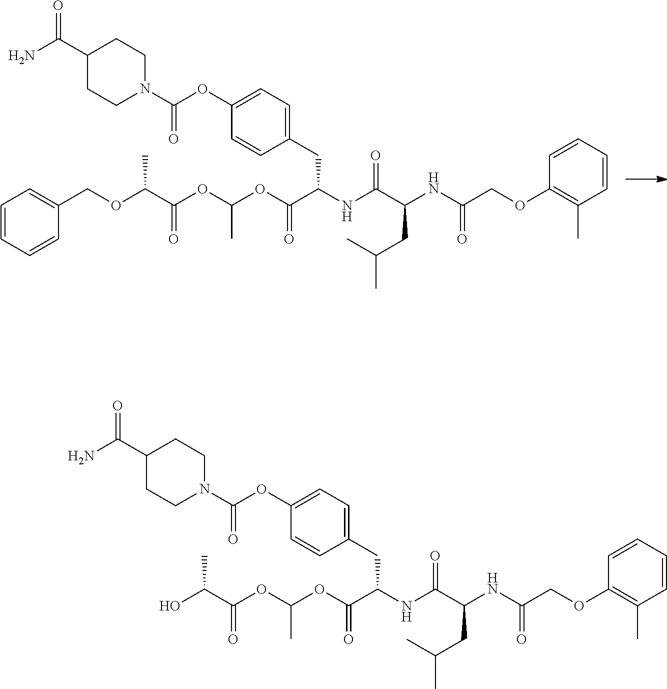 [SMILES: Cc1ccccc1OCC(=O)N[C@@H](CC(C)C)C(=O)N[C@@H](Cc1ccc(OC(=O)N2CCC(C(N)=O)CC2)cc1)C(=O)OC(C)OC(=O)[C@@H](C)OCc1ccccc1 ; Cc1ccccc1OCC(=O)N[C@@H](CC(C)C)C(=O)N[C@@H](Cc1ccc(OC(=O)N2CCC(C(N)=O)CC2)cc1)C(=O)OC(C)OC(=O)[C@@H](C)O]

To a mixture of 4-((2S)-3-(1-(((R)-2-(benzyloxy)propanoyl)oxy)ethoxy)-2-((S)-4-methyl-2-(2-(o-tolyloxy)acetamido)pentanamido)-3-oxopropyl)phenyl 4-carbamoylpiperidine-1-carboxylate (55 mg, 68.0 μmol) and ammonium formate (43.2 mg, 0.69 mmol) in EtOAc (10 mL) under a nitrogen atmosphere was added palladium on carbon (3 mg, 28 μmol, 5% wt) and the reaction mixture stirred at 60° C. for 2 h. The reaction mixture was filtered through celite washing with EtOAc and the filtrate evaporated in vacuo. The crude product was purified by preparative reversed-phase HPLC to yield 4-((2S)-3-(1-(((R)-2-hydroxypropanoyl)oxy)ethoxy)-2-((S)-4-methyl-2-(2-(o-tolyloxy)acetamido)pentanamido)-3-oxopropyl)phenyl 4-carbamoylpiperidine-1-carboxylate as a white solid (33.8 mg, 69%). LCMS (Method A): Rt=8.00 min; [M+H]⁺=713.7. ¹HNMR (400 MHz, CDCl₃) δ7.08-7.17 (m, 4H), 6.87-7.02 (m, 4H), 6.76 (t, J=7.6 Hz, 1H), 5.70-6.00 (m, 2H), 4.76-4.84 (m, 1H), 4.42-4.59 (m, 4H), 4.27 (q, J=6.9 Hz, 1H), 4.20 (s, 2H), 2.95-3.17 (m, 2H), 2.86 (s, 1H), 2.30-2.37 (m, 2H), 2.28 (d, J=3.7 Hz, 3H), 1.87 (d, J=12.8 Hz, 2H), 1.64-1.70 (m, 4H), 1.58 (q, J=6.1 Hz, 2H), 1.52 (d, J=5.0 Hz, 2H), 1.47 (d, J=5.5 Hz, 2H), 1.39 (t, J=7.1 Hz, 3H), 0.88 (d, J=4.1 Hz, 3H), 0.87 (d, J=4.1 Hz, 3H).

### Chemical Synthesis Example 4

### 1-Chloroethyl 5—((R)-1,2-dithiolan-3-yl)pentanoate

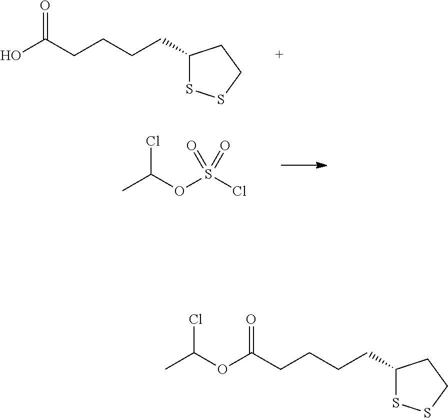

To a stirred mixture of lipoic acid (166 mg, 0.788 mmol), tetrabutylammonium hydrogen sulfate (27 mg, 78.0 μmol) and sodium bicarbonate (241 mg, 3.15 mmol) in water (5 mL) and DCM (3 mL) at r.t. was added a solution of 1-chloroethyl sulfurochloridate (200 mg, 1.12 mmol) in DCM (2 mL) dropwise over 5 min. The mixture was stirred at r.t. for 20 h. The reaction mixture was diluted with H₂O (5 mL) and DCM (10 mL) and the layers separated. The organic phase was washed successively with sat. NaHCO₃(aq) (2×10 mL) and sat. brine solution (10 mL). The organic phase was dried (MgSO₄), filtered and the solvent evaporated in vacuo to yield 1-chloroethyl 5—((R)-1,2-dithiolan-3-yl)pentanoate (60 mg, 28%) as a colourless oil. 1-H-NMR (400 MHz, CDCl₃): δ6.55 (q, J=5.8 Hz, 1H), 3.57 (dt, J=14.3, 6.3 Hz, 1H), 3.34-3.38 (m, 1H), 3.07-3.22 (m, 2H), 2.33-2.51 (m, 4H), 1.87-1.96 (m, 1H), 1.75-1.79 (m, 3H), 1.63-1.73 (m, 6H), 1.40-1.62 (m, 4H).

### 4-((2S)-3-(1-((5—((R)-1,2-Dithiolan-3-yl)pentanoyl)oxy)ethoxy)-2-((S)-4-methyl-2-(2-(o-tolyloxy)acetamido)pentanamido)-3-oxopropyl)phenyl 4-carbamoylpiperidine-1-carboxylate

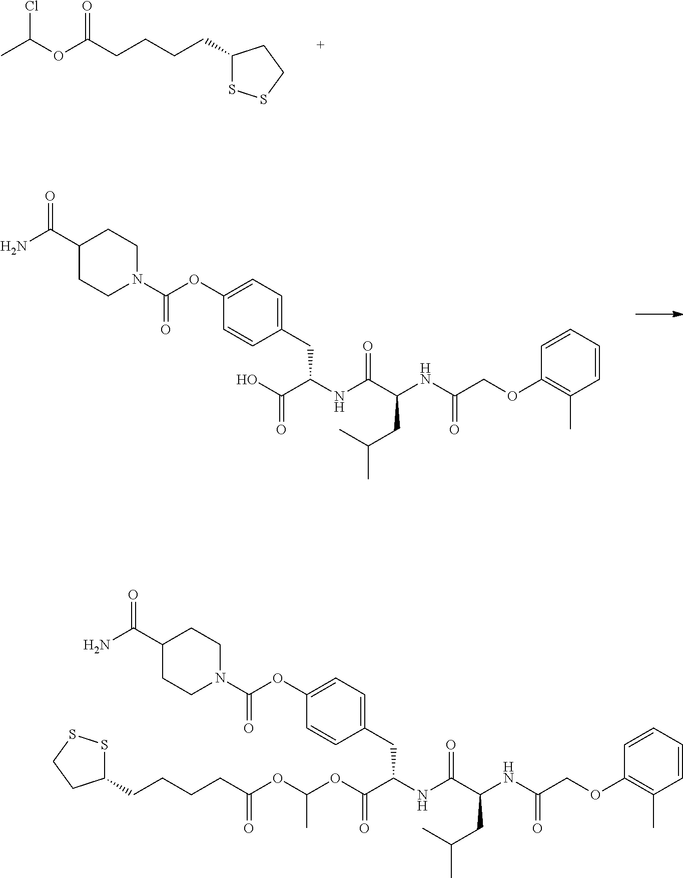 [SMILES: CC(Cl)OC(=O)CCCC[C@@H]1CCSS1 ; Cc1ccccc1OCC(=O)N[C@@H](CC(C)C)C(=O)N[C@@H](Cc1ccc(OC(=O)N2CCC(C(N)=O)CC2)cc1)C(=O)O ; Cc1ccccc1OCC(=O)N[C@@H](CC(C)C)C(=O)N[C@@H](Cc1ccc(OC(=O)N2CCC(C(N)=O)CC2)cc1)C(=O)OC(C)OC(=O)CCCC[C@@H]1CCSS1]

(S)-3-(4-((4-Carbamoylpiperidine-1-carbonyl)oxy)phenyl)-2-((S)-4-methyl-2-(2-(o-tolyloxy)acetamido)pentanamido)propanoic acid (55 mg, 92 μmol) and 1-chloroethyl 5—((R)-1,2-dithiolan-3-yl)pentanoate (60 mg, 0.225 mmol) were dissolved in anhydrous DMF (3.4 mL). DIPEA (55 mL, 0.316 mmol) was added and the mixture stirred at 50° C. for 18 h. The solvent was evaporated in vacuo and the resulting residue dissolve in EtOAc (20 mL) and sat. NaHCO₃ (aq) (15 mL). The layers were separated, and the organic layer washed with sat. brine solution (15 mL), dried (MgSO₄), filtered and the solvent evaporated in vacuo. The crude product was purified by flash chromatography eluting with isohexane→EtOAc→20% acetone-EtOAc to yield the desired product in crude form. This material was further purified by preparative reversed-phase HPLC and the appropriate fractions were combined and the solution frozen (−78° C.). The solvent was evaporated in vacuo (lyophilisation) to yield 4-((2S)-3-(1-((5—((R)-1,2-dithiolan-3-yl)pentanoyl)oxy)ethoxy)-2-((S)-4-methyl-2-(2-(o-tolyloxy)acetamido)pentanamido)-3-oxopropyl)phenyl 4-carbamoylpiperidine-1-carboxylate (2.1 mg, 3%) as a white solid. LCMS (Method C): Rt=7.62 min; [M+H]³⁰=829.5. ¹HNMR (400 MHz, Acetone-D₆): δ7.64 (dd, J=20.6, 7.8 Hz, 1H), 7.32 (d, J=8.7 Hz, 1H), 7.14-7.23 (m, 4H), 7.02 (q, J=4.0 Hz, 2H), 6.83-6.91 (m, 3H), 4.46-4.72 (m, 4H), 4.22 (s, 2H), 3.58-3.74 (m, 1H), 2.91-3.19 (m, 7H), 2.19-2.51 (m, 10H), 1.84-1.90 (m, 2H), 1.54-1.75 (m, 7H), 1.28-1.56 (m, 6H), 0.86-0.90 (m, 6H).

### Chemical Synthesis Example 5

### 5-((3R)-2-Oxido-1,2-dithiolan-3-yl)pentanoic acid and 5-((3R)-1-Oxido-1,2-dithiolan-3-yl)pentanoic acid

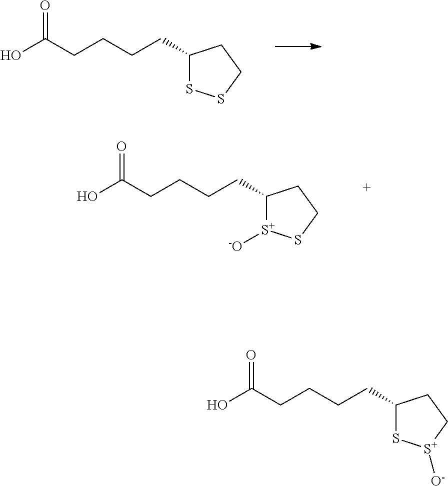

Lipoic acid (350 mg, 1.70 mmol) was dissolved in acetone (1.7 mL) and hydrogen peroxide (0.35 mL, 3.39 mmol, 30 wt. % in water) was added dropwise. The mixture was stirred at r.t. for 24 h. The resulting solution was concentrated in vacuo to remove most of the acetone, then water (10 mL), sat. brine solution (5 mL) and EtOAc (10 mL) added. The phases were separated, and the organic phase was washed with sat. brine solution, dried (MgSO₄), filtered and the solvent evaporated in vacuo to yield a mixture of 5-((3R)-2-oxido-1,2-dithiolan-3-yl)pentanoic acid and 5-((3R)-1-oxido-1,2-dithiolan-3-yl)pentanoic acid (376 mg, 98%) as a mixture of isomers, as a colourless oil. LCMS (Method A): Rt=1.49 min; [M+H]⁺=223.2.

### Chemical Synthesis Example 6

### 1-Chloroethyl 5-((3R)-2-oxido-1,2-dithiolan-3-yl)pentanoate and 1-Chloroethyl 5-((3R)-1-oxido-1,2-dithiolan-3-yl)pentanoate

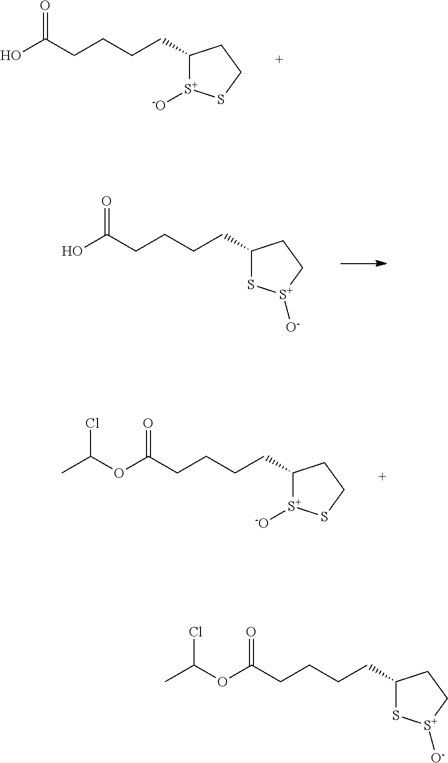

To a stirred mixture of 5-((3R)-2-oxido-1,2-dithiolan-3-yl)pentanoic acid and 5-((3R)-1-oxido-1,2-dithiolan-3-yl)pentanoic acid (130 mg, 0.585 mmol), tetrabutylammonium hydrogen sulfate (20 mg, 58.0 μmol) and sodium bicarbonate (179 mg, 2.34 mmol) in water (4.0 mL) and DCM (2.0 mL) at r.t. was added a solution of 1-chloroethyl sulfurochloridate (148 mg, 0.829 mmol) in DCM (2.0 mL) dropwise over 5 min. The mixture was stirred at r.t. for 20 h, passed through a phase separator and the filtrate evaporated in vacuo. The reaction mixture was dissolved in EtOAc and washed with sat. NaHCO₃ solution, dried (MgSO₄), filtered and the solvent evaporated in vacuo to yield a mixture of 1-chloroethyl 5-((3R)-2-oxido-1,2-dithiolan-3-yl)pentanoate and 1-chloroethyl 5-((3R)-1-oxido-1,2-dithiolan 3-yl)pentanoate (17 mg, 10%) as a mixture of isomers, as a colourless oil. LCMS (Method B): Rt=2.34 min; [M+H]⁺=285.1.

### 4-((2S)-2-((S)-4-Methyl-2-(2-(o-tolyloxy)acetamido)pentanamido)-3-(1-((5-((3R)-2-oxido-1,2-dithiolan-3-yl)pentanoyl)oxy)ethoxy)-3-oxopropyl)phenyl 4-carbamoylpiperidine-1-carboxylate and 4-((2S)-2-((S)-4-Methyl-2-(2-(o-tolyloxy)acetamido)pentanamido)-3-(1-((5-((3R)-1-oxido-1,2-dithiolan-3-yl)pentanoyl)oxy)ethoxy)-3-oxopropyl)phenyl carbamoylpiperidine-1-carboxylate

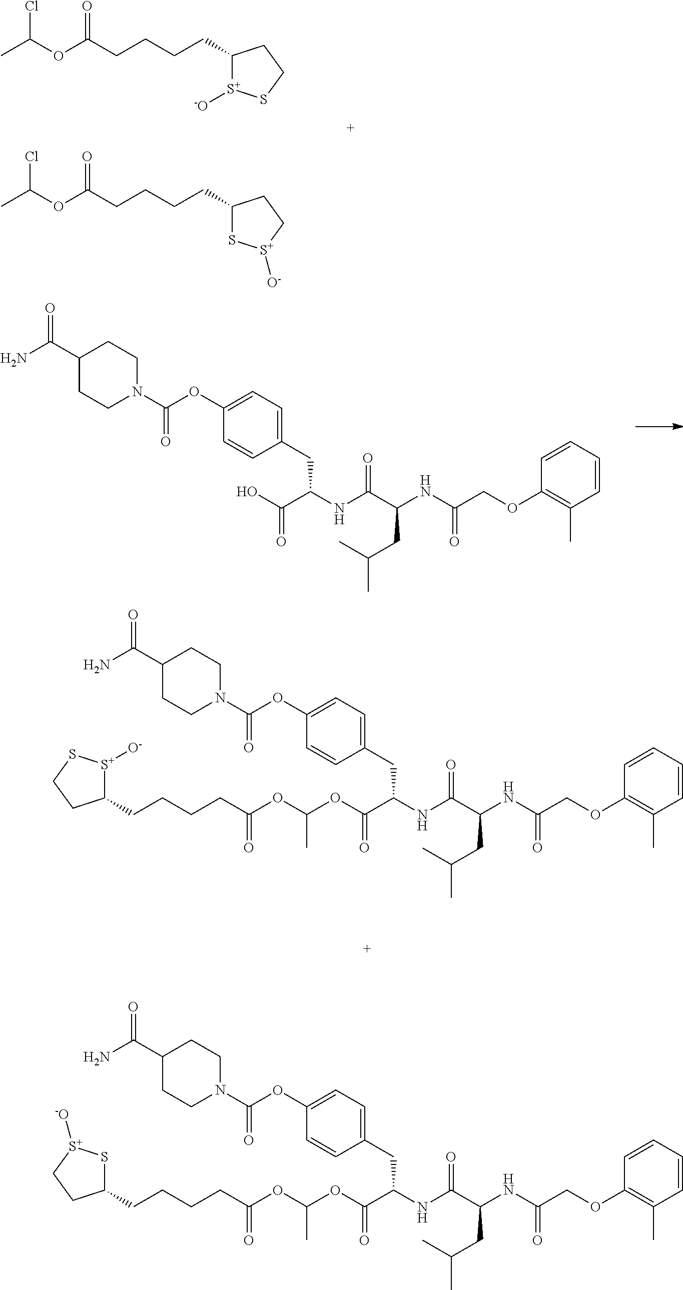

To a solution of (S)-3-(4-((4-carbamoylpiperidine-1-carbonyl)oxy)phenyl)-2-((S)-4-methyl-2-(2-(o-tolyloxy)acetamido)pentanamido)propanoic acid (25 mg, 42.0 μmol) in DMF (1.6 mL) was added a mixture of 1-chloroethyl 5-((3R)-2-oxido-1,2-dithiolan-3-yl)pentanoate and 1-chloroethyl 5-((3R)-1-oxido-1,2-dithiolan-3-yl)pentanoate (17 mg, 60 μmol) and DMF (1.6 mL) and the reaction mixture stirred at 50° C. for 48 h. The reaction mixture was partitioned between water and EtOAc. The layers were separated, and the organic phase washed successively with sat. NaHCO₃ solution and sat. brine solution, dried (MgSO₄), filtered and the solvent evaporated in vacuo. The product was purified by preparative reversed-phase HPLC and the appropriate fractions were combined and the solvent evaporated in vacuo (lyophilisation) to yield a mixture of 4-((2 S)-2-((S)-4-methyl-2-(2-(o-tolyloxy)acetamido)pentanamido)-3-(1-((5-((3R)-2-oxido-1,2-dithiol an 3-yl)pentanoyl)oxy)ethoxy)-3-oxopropyl)phenyl 4-carbamoylpiperidine-1-carboxylate and 4-((2 S)-2-((S)-4-methyl-2-(2-(o-tolyloxy)acetamido)pentanamido)-3-(1-((5-((3R)-1-oxido-1,2-dithiolan-3-yl)pentanoyl)oxy)ethoxy)-3-oxopropyl)phenyl 4-carbamoylpiperidine-1-carboxylate (0.21 mg, 0.6%) as a mixture of isomers, as a white solid. LCMS (Method B): Rt=2.65 min; [M+H]⁺=845.6.

### Chemical Synthesis Example 7

### N-Acetyl-N-(tert-butoxycarbonyl)-S-trityl-L-cysteine

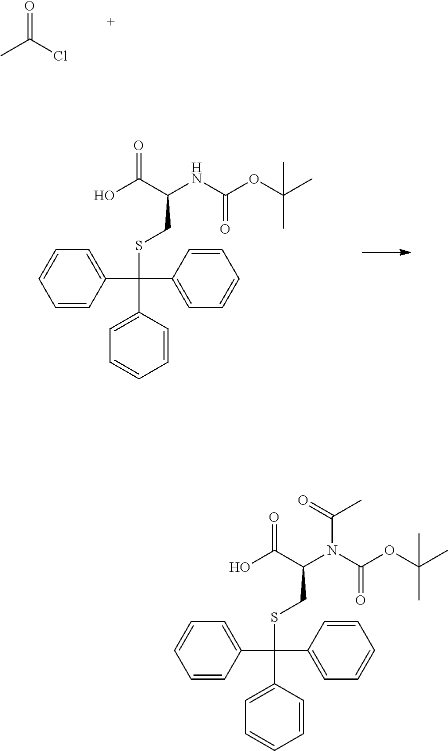

To a stirred solution of N-(tert-butoxycarbonyl)-S-trityl-L-cysteine (2.50 g, 5.39 mmol) in anhydrous acetone (50 mL) at 0° C. under an atmosphere of nitrogen was added over approx. one minute DIPEA (5.6 mL, 32.1 mmol), followed by addition of acetyl chloride (0.77 mL, 10.8 mmol) over 10 min maintaining an internal temperature between 5-1° C. The stirred reaction mixture was allowed to warm to r.t. over 2 h and stirring was continued at r.t. for 5 days. The reaction mixture was quenched with water (400 mL), vigorously stirred for 10 minutes and extracted with EtOAc (2×200 mL). The combined organics were successively washed with water (150 mL), sat. brine solution (150 mL), dried (MgSO₄), filtered and the solvent evaporated in vacuo onto silica. The crude product was purified by flash chromatography eluting with 10% EtOAc-isohexane (0.5% AcOH)→30% EtOAc-isohexane (0.5% AcOH). The combined fractions were concentrated in vacuo to about 100 mL, washed successively with water (100 mL) and sat. brine solution (100 mL), dried (MgSO₄), filtered and the solvent evaporated in vacuo to yield N-acetyl-N-(tert-butoxycarbonyl)-S-trityl-L-cysteine (1.44 g, 53%) as an off-white foam/gum. LCMS (Method B): Rt=3.25 min; EM—Hr=504.4. ¹H-MNR (400 MHz, CDCl₃) δ7.36-7.39 (m, 6H), 7.18-7.30 (m, 9H), 5.31 (dd, J=9.6, 5.0 Hz, 1H), 2.82 (dd, J=12.8, 5.0 Hz, 1H), 2.74 (dd, J=12.8, 9.6 Hz, 1H), 2.49 (s, 3H), 1.41 (s, 9H).

### 1-Chloroethyl N-acetyl-N-(tert-butoxycarbonyl)-S-trityl-L-cysteinate

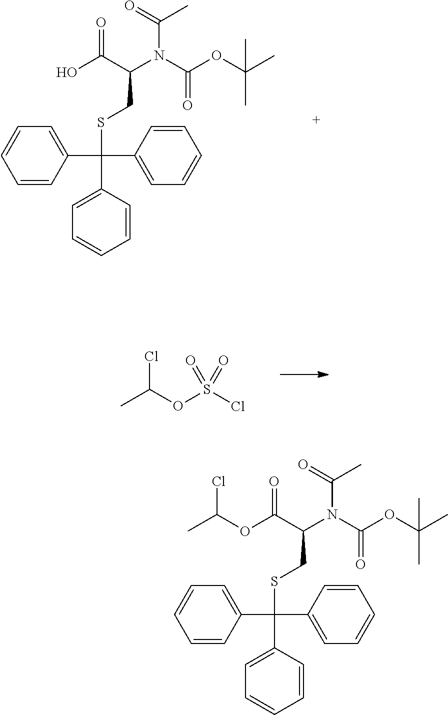

To a stirred mixture of N-acetyl-N-(tert-butoxycarbonyl)-S-trityl-L-cysteine (1.40 g, 2.77 mmol), tetrabutylammonium hydrogen sulfate (94 mg, 0.277 mmol) and sodium bicarbonate (930 mg, 11.1 mmol) in water (35 mL) and DCM (25 mL) at r.t. was added over 5 min a solution of 1-chloroethyl sulfurochloridate (694 mg, 3.88 mmol) in DCM (10 mL). The reaction mixture was stirred vigorously at r.t. for 18 h. The reaction mixture was passed through a phase separator, the filtrate evaporated in vacuo onto silica. The crude product was purified by flash chromatography eluting with isohexane EtOAc to yield 1-chloroethyl N-acetyl-N-(tert-butoxycarbonyl)-S-trityl-L-cysteinate (665 mg, 42%) as a yellow oil. LCMS (Method B): Rt=3.65 min; [M+Na]⁺=590.1. ¹H NMR (400 MHz, CDCl₃): δ 7.38-7.47 (m, 7H), 7.19-7.30 (m, 8H, overlapping with CHCl₃), 6.36-6.45 (m, 1H), 5.24-5.39 (m, 1H), 2.67-2.85 (m, 2H), 2.52 (dd, J=14.7, 3.2 Hz, 3H), 1.66-1.76 (m, 3H), 1.37-1.51 (m, 9H).

### 4-((6R,12S)-5-Acetyl-2,2,9-trimethyl-12-((S)-4-methyl-2-(2-(o-tolyloxy)acetamido)pentanamido)-4,7,11-trioxo-6-((tritylthio)methyl)-3,8,10-trioxa-5-azatridecan-13-yl)phenyl 4-carbamoylpiperidine-1-carboxylate

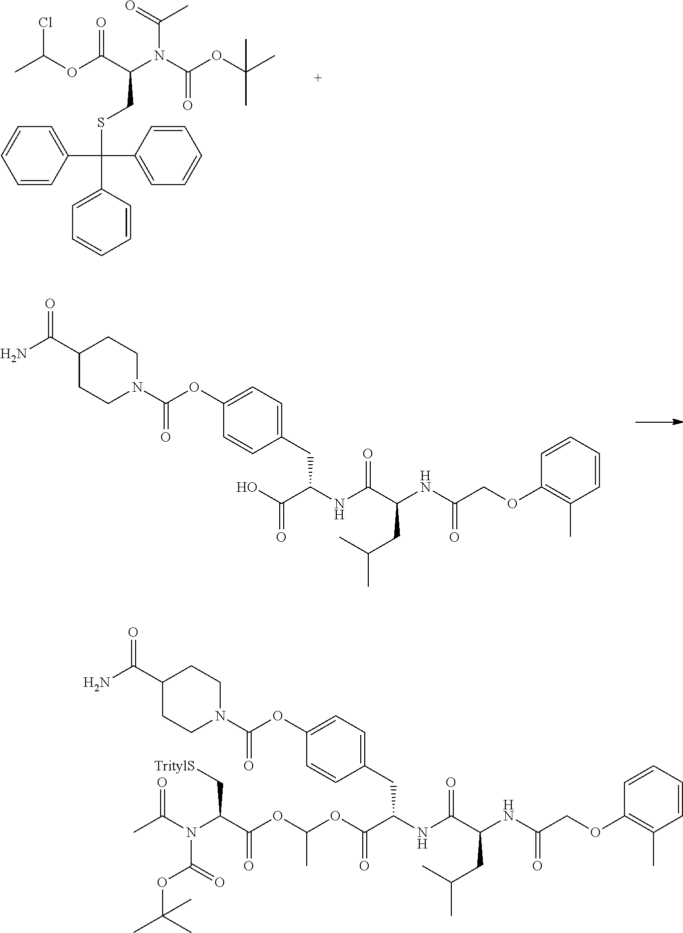

To a stirred solution of (S)-3-(4-((4-carbamoylpiperidine-1-carbonyl)oxy)phenyl)-2-((S)-4-methyl-2-(2-(o-tolyloxy)acetamido)pentanamido)propanoic acid (310 mg, 0.520 mmol) and 1-chloroethyl N-acetyl-N-(tert-butoxycarbonyl)-S-trityl-L-cysteinate (620 mg, 1.09 mmol) in anhydrous DMF (19 mL) was added DIPEA (0.33 mL, 1.89 mmol) and the mixture stirred at 50° C. for 18 h. The solvent was evaporated in vacuo and the residue partitioned between EtOAc and water. The layers were separated, and the organic phase washed successively with sat. NaHCO₃ solution and sat. brine solution, dried (MgSO₄), filtered and the solvent evaporated in vacuo. The crude product was purified by flash chromatography eluting with isohexane→acetone to yield 4-((6R,12 S)-5-acetyl-2,2,9-trim ethyl-12-((S)-4-methyl-2-(2-(o-tolyl oxy)acetamido)pentanamido)-4,7,11-tri oxo-6-((tritylthio)methyl)-3,8,10-tri oxa-5-azatridecan-13-yl)phenyl 4-carbamoylpiperidine-1-carboxylate (15 mg) as a yellow gum. This material was used without further purification. LCMS (Method B): Rt=3.58 min; [M+H]⁺=1128.9.

### 4-((2S)-3-(1-((Acetyl-L-cysteinyl)oxy)ethoxy)-2-((S)-4-methyl-2-(2-(o-tolyloxy)acetamido)pentanamido)-3-oxopropyl)phenyl 4-carbamoylpiperidine-1-carboxylate

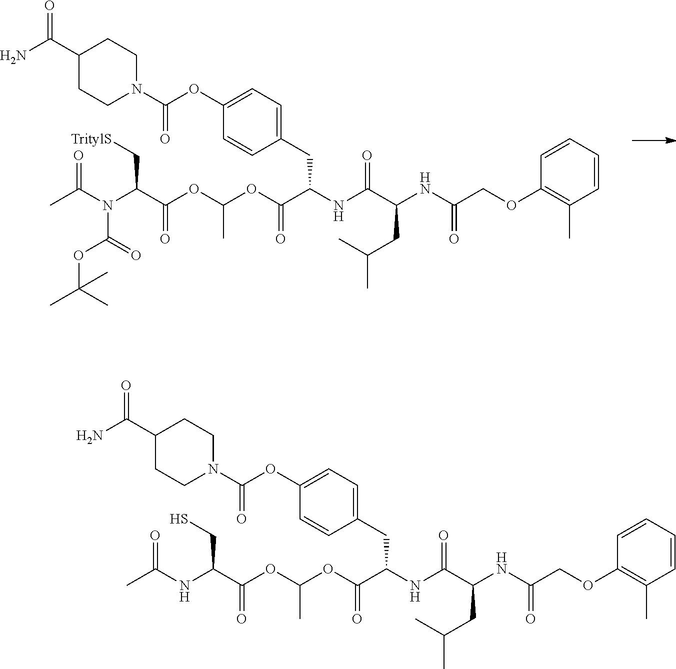

To a solution of 4-((6R,12S)-5-acetyl-2,2,9-trimethyl-12-((S)-4-methyl-2-(2-(o-tolyloxy)acetamido)pentanamido)-4,7,11-tri oxo-6-((tritylthio)methyl)-3,8,10-tri oxa-5-azatridecan-13-yl)phenyl 4-carbamoylpiperidine-1-carboxylate (17 mg, 15 μmol) in DCM (1.5 mL) was added a solution of triethylsilane (12 μL, 75 μmol) in DCM (0.5 mL) followed by a solution of 10% TFA-DCM (2.0 mL). The mixture was stirred at r.t. for 18 h. The reaction mixture was evaporated in vacuo and the resulting residue dissolved in DMSO and the crude product purified by preparative reversed-phase HPLC. The appropriate fractions were combined and the solution frozen (−78° C.). The solvent was evaporated in vacuo (lyophilisation) to yield 4-((2S)-3-(1-((acetyl-L-cysteinyl)oxy)ethoxy)-2-((S)-4-methyl-2-(2-(o-tolyloxy)acetamido)pentanamido)-3-oxopropyl)phenyl 4-carbamoylpiperidine-1-carboxylate (1.4 mg, 12%) as a white solid. LCMS (Method B): Rt=2.43 min[M+H]⁺=786.5.

### Chemical Synthesis Example 8

### 1-Chloroethyl propionate

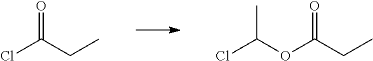

A 25 mL flask was charged with propionyl chloride (1.5 mL, 17.2 mmol) under an atmosphere of nitrogen and cooled to 0° C. Zinc chloride (0.25 mL, 0.172 mmol, 0.7 M in THF) was added followed by acetaldehyde (1.2 mL, 20.6 mmol). The reaction mixture was stirred at 0° C. for 2 h. The reaction mixture was filtered through a silica plug washing with isohexane. The filtrate was evaporated in vacuo to yield an orange oil. The crude oil was re-dissolved in isohexane (10 mL) and washed successively with saturated aqueous sodium bicarbonate solution (10 mL), water (10 mL) and sat. brine solution (10 mL), dried (MgSO₄), filtered and the solvent evaporated in vacuo to yield 1-chloroethyl propionate (1.13 g, 48%) as an orange oil which was used without further purification.

### 4-((2S)-3-(1-(Isobutyryloxy)ethoxy)-2-((S)-4-methyl-2-(2-(o-tolyloxy)acetamido)pentanamido)-3-oxopropyl)phenyl 4-carbamoylpiperidine-1-carboxylate

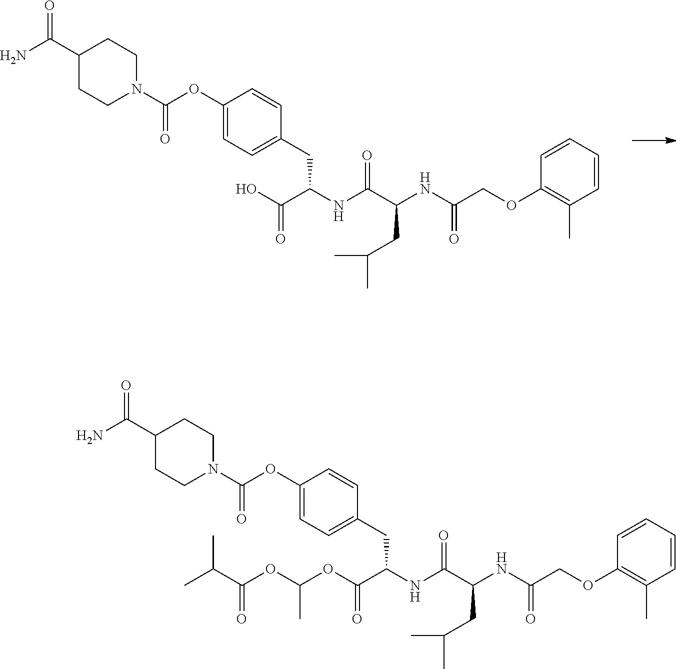 [SMILES: Cc1ccccc1OCC(=O)N[C@@H](CC(C)C)C(=O)N[C@@H](Cc1ccc(OC(=O)N2CCC(C(N)=O)CC2)cc1)C(=O)O ; Cc1ccccc1OCC(=O)N[C@@H](CC(C)C)C(=O)N[C@@H](Cc1ccc(OC(=O)N2CCC(C(N)=O)CC2)cc1)C(=O)OC(C)OC(=O)C(C)C]

(S)-3-(44(4-Carbamoylpiperidine-1-carbonyl)oxy)phenyl)-2-((S)-4-methyl-2-(2-(o-tolyloxy)acetamido)pentanamido)propanoic acid (100 mg, 0.168 mmol) and DIPEA (100 μL 0.574 mmol) were dissolved in anhydrous DMF (4.0 mL). 1-Chloroethyl 2-methylpropanoate (40 μL, 0.251 mmol) was added and the mixture stirred at 50° C. for 18 h. The reaction mixture was partitioned between water (10 mL) and EtOAc (10 mL). The layers were separated, and the organic phase was washed successively with sat. NaHCO₃ solution (2×10 mL) and sat. brine solution (10 mL), dried (MgSO₄), filtered and the solvent evaporated in vacuo. The crude product was purified by preparative reversed-phase HPLC and the appropriate fractions were combined and the solution frozen (−78° C.). The solvent was evaporated in vacuo (lyophilisation) to yield 4-((2 S)-3-(1-(isobutyryloxy)ethoxy)-2-((S)-4-methyl-2-(2-(o-tolyloxy)acetamido)pentanamido)-3-oxopropyl)phenyl 4-carbamoylpiperidine-1-carboxylate (18 mg, 15%) as a white solid. LCMS (Method C): Rt=7.10 min; [M+H]⁺=711.5. ¹H NMR (400 MHz, DMSO-d₆): δ 8.48-8.60 (m, 1H), 7.85 (q, J=4.1 Hz, 1H), 7.09-7.30 (m, 5H), 6.97-7.01 (m, 2H), 6.64-6.88 (m, 4H), 4.39-4.55 (m, 4H), 4.03 (d, J=28.9 Hz, 2H), 2.86-3.04 (m, 4H), 2.53 (dd, J=7.6, 5.3 Hz, 1H), 2.32 (qd, J=7.6, 4.0 Hz, 1H), 2.18 (d, J=16.5 Hz, 3H), 1.75 (d, J=10.5 Hz, 2H), 1.29-1.56 (m, 8H), 1.05-1.08 (m, 6H), 0.82-0.90 (m, 6H).

The following compounds were synthesized via an analogous method:

| Compound | Analytical data |
|---|---|
| 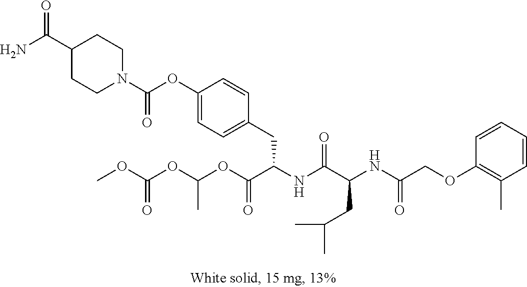 [SMILES: COC(=O)OC(C)OC(=O)[C@H](Cc1ccc(OC(=O)N2CCC(C(N)=O)CC2)cc1)NC(=O)[C@H](CC(C)C)NC(=O)COc1ccccc1C] | LCMS (Method C): Rt = 6.92 min; [M + H]⁺ = 699.5 ¹H NMR (400 MHz DMSO-d6): δ 8.48-8.54 (m, 1H), 7.82 (q, J = 4.4 Hz, 1H), 7.05-7.26 (m, 5H), 6.93-6.96 (m, 2H), 6.76-6.84 (m, 3H), 6.57 (dq, J = 19.2, 5.5 Hz, 1H), 4.34- 4.51 (m, 4H), 3.95 (t, J = 30.9 Hz, 2H), 3.69 (d, J = 1.4 Hz, 3H), 2.82- 3.05 (m, 4H), 2.29 (tt, J = 11.2, 3.6 Hz, 1H), 2.16 (s, 3H), 1.70-1.73 (m, 2H), 1.29-1.53 (m, 8H), 0.72-0.83 (m, 6H). |
| 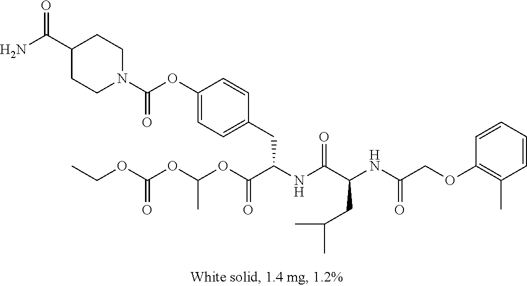 [SMILES: CCOC(=O)OC(C)OC(=O)[C@H](Cc1ccc(OC(=O)N2CCC(C(N)=O)CC2)cc1)NC(=O)[C@H](CC(C)C)NC(=O)COc1ccccc1C] | LCMS (Method C): Rt = 7.16 min; [M + H]⁺ = 713.5 |
| 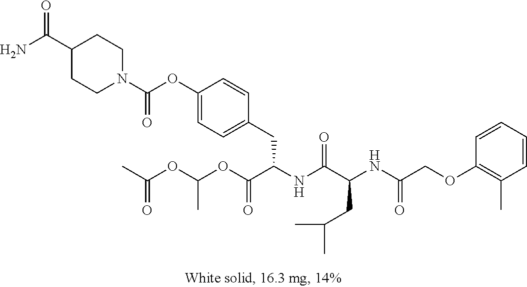 [SMILES: CC(=O)OC(C)OC(=O)[C@H](Cc1ccc(OC(=O)N2CCC(C(N)=O)CC2)cc1)NC(=O)[C@H](CC(C)C)NC(=O)COc1ccccc1C] | LCMS (Method C): Rt = 6.88 min; [M + H]⁺ = 683.5 ¹H NMR (400 MHz, DMSO-d₆): δ 8.54 (q, J = 7.6 Hz, 1H), 7.86 (dd, J = 8.5, 3.4 Hz, 1H), 7.09- 7.30 (m, 5H), 6.98-7.04 (m, 2H), 6.85 (td, J = 14.2, 7.3 Hz, 3H), 6.60-6.75 (m, 1H), 4.37-4.58 (m, 4H), 3.97-4.08 (m, 2H), 2.87-3.05 (m, 4H), 2.33 (qd, J = 7.6, 3.8 Hz, 1H), 2.18 (d, J = 18.3 Hz, 3H), 2.00 (d, J = 20.6 Hz, 3H), 1.74-1.77 (m, 2H), 1.38-1.58 (m, 6H), 1.31 (d, J = 5.5 Hz, 1H), 0.83-0.88 (m, 6H). |
| 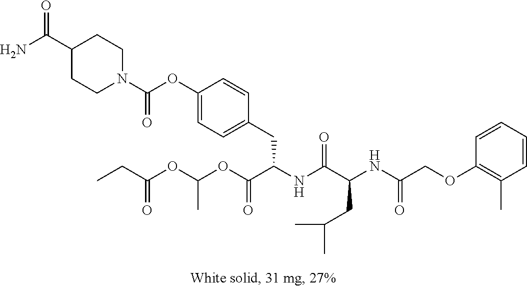 [SMILES: CCC(=O)OC(C)OC(=O)[C@H](Cc1ccc(OC(=O)N2CCC(C(N)=O)CC2)cc1)NC(=O)[C@H](CC(C)C)NC(=O)COc1ccccc1C] | LCMS (Method C): Rt = 7.14 min; [M + H]⁺ = 697.4 ¹H-NMR (400 MHz, ACETONE-D₆) δ 7.66 (dd, J = 20.6, 8.2 Hz, 1H), 7.33 (d, J = 7.8 Hz, 1H), 7.14-7.23 (m, 4H), 7.00-7.03 (m, 2H), 6.77-6.91 (m, 4H), 6.15 (s, 1H), 4.46- 4.77 (m, 4H), 4.17 (d, J = 34.3 Hz, 2H), 2.97-3.16 (m, 2H), 2.47 (tt, J = 11.3, 3.8 Hz, 1H), 2.31-2.38 (m, 2H), 2.16-2.26 (m, 4H), 1.84-1.90 (m, 3H), 1.50-1.65 (m, 5H), 1.39-1.46 (m, 3H), 1.07 (td, J = 7.6, 4.1 Hz, 3H), 0.86- 0.89 (m, 6H). |

Other compounds provided herein (e.g., in Table 1 and Table 3) are prepared according to a similar process as provided for the examples provided hereinabove (e.g., Chemical Synthesis Example 1, such as, for example, starting from 3-[4-(4-carbamoylpiperidine-1-carbonyl)oxyphenyl]-2-[[(2S)-4-methyl-2-[[2-(2-methylphenoxy)acetyl]amino]pentanoyl]amino]propanoic acid or Chemical Synthesis example 5, such as, for example, starting from lipoic acid).

### II. Biological Evaluation

### Example 1: Rabbit Cornea Homogenate Stability Assay

Determining Rabbit Cornea Homogenate stability of the test compounds was performed using HPLC-MS or UPLC-MS. The assay was performed at two concentrations of Rabbit Cornea Homogenate (0.15 mg/mL and 0.45 mg/mL total protein) so that any hydrolysis observed can be assigned as esterase dependent or not.

### Rabbit Cornea Homogenisation

Three to five rabbit corneas (e.g. New Zealand Whites (NZW) or Dutch Belted (DB)) of approx. 50 mg each were sliced and scraped with a scalpel and tweezers until reduced to small (1-3 mm), thin pieces. These were transferred into a glass vial containing approximately 2 mL of cold DPBS pH7.4 buffer.

Sample was cooled intermittently on ice and shear homogenized for 3 minutes, then centrifuged for 3 min at 13,000 g. The supernatant was pipetted off into a vial, and total protein concentration was determined at 280 nm. Sample was stored at −78° C.

### Rabbit Cornea Esterase Assay

### Method 1

### Preparation of Stock Solutions:

10 mM Compound stocks were diluted to 100 μM in a96 deep-well plate: 10 μL of 10 mM Compound stock was added to 990 μL 50 mM HEPES, pH7.5 buffer. Compounds were further diluted to 10 μMm 100 μL of 100 μM compound was added to 900 μL 50 mM HEPES, pH7.5 buffer. Esterase homogenate was diluted to 300 ng/μL and 900 ng/μL.

### Assay Conditions:

A heater shaker was set to 37° C. Into a suitable 96 well plate (Run Plate), 75 μL of 300 or 900 ng/μL esterase homogenate was pipetted into each of the required wells (2 min, 5 min, 10 min, 20 min and 45 min). The plate was sealed and then warmed at 37° C. for 5 min.

Another 96 well PCR plate was put on ice (Kill Plate). To this was added 100 μL of MeCN to each well, labelled 0 min 2 min, 5 min, 10 min, 20 min and 45 min. The plate was covered to minimize evaporation.

For the T=0 sample only, to the 100 μL cold MeCN stop solution was added 50 μL of 300 or 900 ng/μL esterase homogenate followed by 50 μL of 10 μM compound solution. For the remaining timepoints, 75 μL of 10 μM compound solution was added to the Run Plate starting from T=45 min row and ending with T=2 min row. At the appropriate time point, 100 μL of the assay mixture was added to the matching kill plate well containing 100 μL of cold MeCN. Samples were analysed as soon as practicable by LCMS (Waters Xevo TQ-S or Micromass Ultima).

### Method 2

### Preparation of Stock Solutions:

10 mM Compound DMSO stocks were diluted to 10 μM in a glass vial: 10 μL of 10 mM Compound stock was added to 9,990 μL DPBS, pH7.4 buffer. Esterase homogenate was diluted to 300 ng/μL and 900 ng/μL in DPBS.

### Assay Conditions:

A heater shaker was set to 37° C. Into a suitable 96 well plate (Run Plate), 70 μL of 300 or 900 ng/μL esterase homogenate was pipetted into two rows as compounds were analysed in duplicate (0 min, 2 min, 5 min, 10 min, 20 min and 45 min). The plate was sealed and then warmed at 37° C. for 5 min.

Two 96 deep-well plates were put on ice (Kill Plates). To these, 990 μL of 50:50 MeCN—H₂O were added to required rows, labelled 0 min 2 min, 5 min, 10 min, 20 min and 45 min. The plates were covered to minimise evaporation.

To both rows of the Run Plate, 70 μL of 10 μM compound solution was added. At the appropriate time point, 10 μL of the assay mixture was added to the matching kill plate well containing 990 μL of 50:50 cold MeCN—H₂O. Samples were analysed as soon as practicable by LCMS (Waters Xevo TQ-S).

Assay conditions for lipoic acid analysis:

A heater shaker was set to 37° C. Into a suitable 96 well plate (Run Plate), 80 μL of 300 or 900 ng/μL esterase homogenate was pipetted into two rows as compounds were analyzed in duplicate (0 min, 2 min, 5 min, 10 min, 20 min and 45 min). The plate was sealed and then warmed at 37° C. for 5 min.

Two 96 shallow-well plates were placed on ice (Kill Plates). To these, 180 μL of 60:40 MeCN—H₂O+0.1% acetic acid were added to required rows. The plates were sealed to minimise evaporation.

To both rows of the Run Plate, 80 μL of 10 μM compound solution was added. At the appropriate time point, 20 μL of the assay mixture was added to the matching kill plate well containing 180 μL of 60:40 cold MeCN—H₂O+0.1% acetic acid. For lipoic acid analysis, samples were analysed as soon as practicable by LCMS (Waters Xevo TQ-S). For parent conjugate and parent analysis the samples were diluted further 1 in 10: 20 μL supernatant was added to 180 of 50:50 MeCN—H₂O.

Parent conjugate, parent and keratolytic concentrations were determined against appropriate standard response curves and the half-life (T½) of the parent conjugate was calculated using the peak area or the measured concentration of the parent conjugate at each time point in the linear region of the log-linear plot.

### Hydrolysis Rates of Example Compounds

TABLE 5

| | Cornea | Esterase % | API | |
|---|---|---|---|---|
| | Homogenate | API formation | Formation | |
| Comp | Conc (mg/mL) | at 45 min | rate (%/min) | Method |
| 68 | 0.15 | B | b | 1 |
| | 0.45 | D | d | 1 |
| 52 | 0.15 | B | b | 1 |
| | 0.45 | C | c | 1 |
| 2 | 0.15 | C | c | 2 |
| | 0.45 | D | d | 2 |
| 3 | 0.15 | B | c | 2 |
| | 0.45 | D | d | 2 |
| 26 | 0.15 | A | a | 2 |
| | 0.45 | A | a | 2 |
| 46 | 0.15 | D | d | 2 |
| | 0.45 | D | d | 2 |
| 48 | 0.15 | D | d | 2 |
| | 0.45 | D | d | 2 |
| 27-30 | 0.15 | C | c | 2 |
| | 0.45 | D | d | 2 |
| 47 | 0.15 | D | d | 2 |
| | 0.45 | D | d | 2 |
| 23* | 0.15 | A | a | 2 |
| | 0.45 | A | a | 2 |

A: percent active pharmaceutical ingredient (API) formation <25%;
B: percent API formation 25% to 50%;
C: percent API formation 51% to 75%;
D: percent API formation >75%.
a: API formation rate <0.5%/min;
b: API formation rate 0.5-1.0%/min;
c: API formation rate 1.0-1.5%/min;
d: API formation rate >1.5%/min.
*mixture of diastereomers

### Example 2: Aqueous Hydrolysis Stability Assay

Determination of aqueous stability of the test compounds was performed using HPLC-MS or UPLC-MS. A test compound 10 mM stock solution was prepared in DMSO. Half-life (T½) of the parent conjugate was calculated using the peak area or measured concentration of the parent conjugate at each time point in the linear region of the log-linear plot.

### Method 1

μL of the DMSO stock solution was dissolved in 990 μL of 50 mM HEPES pH7.5 buffer or 1:1 (v/v) of Acetonitrile:Water to make a100 μmM solution. Final DMSO concentration was 1%. The solution was kept at room temperature and injected without delay into the LCMS (Waters Xevo TQ-S or Micromass Ultima). Additional injections were performed at appropriate time points.

### Method 2

10 μL of the DMSO stock solution was dissolved in 990 μL of DPBS pH7.4 buffer to prepare 100 ∞M solution. A further dilution was made by dissolving 75 μL of 100 μM stock solution into 225 μL of DPBS. Final DMSO concentration was 0.25%. The solution was kept at 37° C. and injected without delay into the LCMS (Waters Xevo XSQ-ToF). Additional injections were performed at appropriate time points.

Half-life (T½) of the parent conjugate was calculated using the peak area of the parent conjugate at each time point in the linear region of the log-linear plot.

TABLE 6

| | Hydrolytic % API | |
|---|---|---|
| Comp | formation at [time] | Method |
| 68 | B [1156 min] | 1 |
| 52 | A [1156 min] | 1 |
| 2 | C [45 min] | 2 |
| 3 | C [45 min] | 2 |
| 26 | A [45 min] | 2 |

A: percent active pharmaceutical ingredient (API) formation <1.5%;
B: percent API formation 1.5-4%;
C: percent API formation >4%.

### Example 3: Mouse Model of Experimental Dry Eye Disease

Female C57BL/6 mice (6-8 weeks old) or female HEL BCR Tg mice (6-8 weeks old) are commercially obtained. Experimental dry eye is induced as described by Niederkorn, et al. (J. Immunol. 2006, 176:3950-3957) and Dursun et al. (Invest. Ophthalmol. Vis. Sci. 2002, 43:632-638). In brief, mice are exposed to desiccating stress in perforated cages with constant airflow from fans positioned on both sides and room humidity maintained at 30% to 35%. Injection of scopolamine hydrobromide (0.5 mg/0.2 mL; Sigma-Aldrich, St. Louis, MO) is administered subcutaneously, three times a day (8:00 AM, 12:00 noon, and 5:00 PM), on alternating hind-flanks to augment disease. Mice are exposed to desiccating stress for 3 weeks. Untreated control mice are maintained in a nonstressed environment at 50% to 75% relative humidity without exposure to forced air. Test animals are exposed to test compound and subsequently tear samples are obtained to determine stability of test compounds, and tissue samples are taken to determine presence of pro-inflammatory biomarkers.

### Example 4: Thiol Assay

### Stratum Corneum Preparation

Epidermis pieces were transferred and incubated overnight from 25-37° C. in a container containing 100 mL of 0.0005% trypsin (diluted in PBS). The stratum corneum pieces were removed and washed twice with HPLC grade water in a petri dish (145 mm), removing intact cells. The dish and/or pieces were shaken, producing nearly transparent layers. The stratum corneum was then transferred to a petri dish (145 mm), washed with hexane, and shaken to remove fats. Each piece was gently mounted on an absorbent paper. Each piece was transferred to an Eppendorf tube, allowing residual solvents to evaporate for a few minutes.

### Thiol Assay

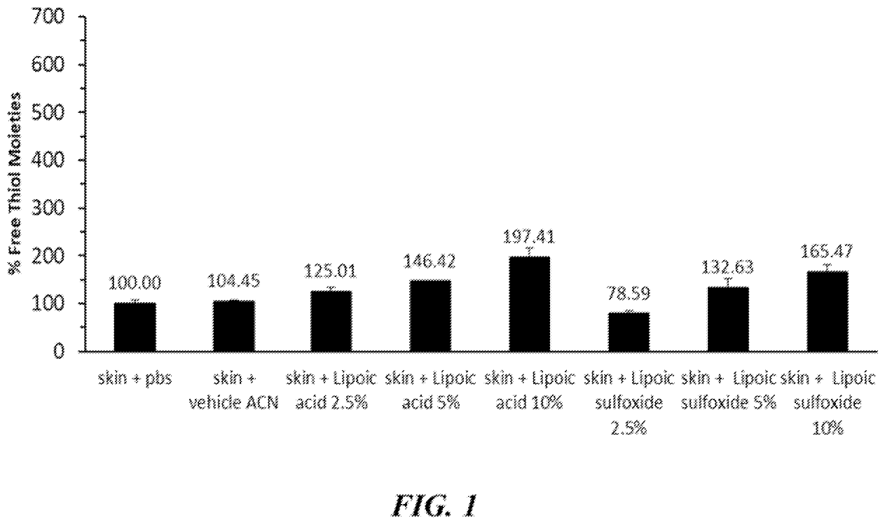

Compounds (e.g., 50 μL; 1 μM to 800 μM) were applied to the isolated stratum corneum at room temperature for a period of 1-24 hours. The pieces were gently mixed with the compounds by pipetting. Following incubation, about 200 μL of 10 M sodium hydroxide was added, incubating for 1 hour at room temperature with continuous blending (e.g., vortexing) until the stratum corneum disintegrates. About 200 μL of 10 M hydrochloric acid was added to normalize pH, vortexing the samples. The samples were centrifuged (e.g., for 20 min at 16,000×g) at room temperature. The supernatant (middle layer) was transferred to an Eppendorf tube. The free thiols were isolated by adding tricholoracetic acid (e.g., 400 μL) and vortexing. The tubes were centrifuged (e.g., for 10 min at 16,000×g) at room temperature. The supernatant was removed, and Ellman's reagent solution (e.g., 220 μL) was added to the remaining pellet. After mixing, 100 μL for each tube was transferred to a96 well plate in the dark. The plate was incubated for about 5 minutes at room temperature while shaking. The optical absorbance at 412 nm was detected and recorded (e.g., FIG. 1).

### III. Preparation of Pharmaceutical Dosage Forms

### Example 1: Solution for Topical Ophthalmic Use

The active ingredient is a compound of any one of Table 1, Table 2, Table 3, Table 4, or a pharmaceutically acceptable salt thereof, and is formulated as a solution with a concentration of from 0.1-1.5% w/v.

We claim:

1. A compound having the structure of Formula (Ia): 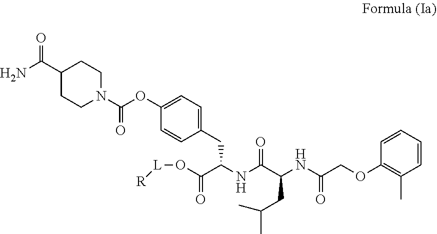
or a pharmaceutically acceptable salt or solvate (e.g., or a stereoisomer) thereof,
wherein:
L is bond, —O(C═O)(OCR⁸R⁹)_{z}—, or —(C═O)(OCR⁸R⁹)_{z}—;
each R⁸ and R⁹ is independently H, halogen, C₁-C₃-alkyl, C₁-C₃-haloalkyl, C₁-C₃-alkoxy, C₃-C₅-cycloalkyl, or R⁸ and R⁹ are taken together with the atoms to which they are attached to form a C₃-C₅-cycloalkyl;
z is 1-6; and
R is substituted or unsubstituted (e.g., straight or branched) alkyl, substituted or unsubstituted (e.g., straight or branched) heteroalkyl, or substituted or unsubstituted heterocycloalkyl (e.g., (N—) substituted with alkyl (e.g., further substituted with oxo and/or thiol)).

2. The compound of claim 1, wherein L is bond.

3. The compound of claim 1, wherein L is —(C═O)(OCR⁸R⁹)_{z}— or —O(C═O)(OCR⁸R⁹)_{z}—.

4. The compound of claim 3, wherein L is —(C═O)(OCR⁸R⁹)_{z}—.

5. The compound of claim 4, wherein L is —(C═O)OCH(CH₃)—.

6. The compound of claim 3, wherein L is —O(C═O)(OCR⁸R⁹)_{z}—.

7. The compound of claim 6, wherein L is —O(C═O)OCH(CH₃).

8. The compound of any one of claims 1-7, wherein R is unsubstituted alkyl (e.g., methyl, ethyl, isopropyl, or t-butyl).

9. The compound of claim 5, wherein: L is —(C═O)OCH(CH₃)— and R is unsubstituted alkyl (e.g., methyl, ethyl, isopropyl, or t-butyl).

10. The compound of claim 7, wherein: L is —O(C═O)OCH(CH₃)— and R is unsubstituted alkyl (e.g., methyl, ethyl, isopropyl, or t-butyl).

11. The compound of any one of claims 1-7, wherein R is substituted (e.g., straight or branched) alkyl, the (e.g., straight or branched) alkyl being substituted with one or more (alkyl) substituent, each (alkyl) substituent being independently selected from the group consisting of hydroxy, optionally substituted alkoxy (e.g., optionally substituted with oxo and hydroxy, or oxo and C₁-C₃ alkoxy), oxo, optionally substituted alkyl (e.g., optionally substituted with alkoxy further optionally substituted with oxo, C₁-C₄ alkyl, and/or hydroxy), optionally substituted heterocycloalkyl (e.g., optionally substituted dioxane (e.g., 1,3 dioxanyl optionally substituted with methyl), dithiolanyl, or dithiolanyl oxide)), hydroxyalkyl, thiol, acetamide, substituted unsaturated cycloalkyl (e.g., being substituted with one or more C₁-C₄ alkyl), and amino.

12. The compound of claim 11, wherein R is:

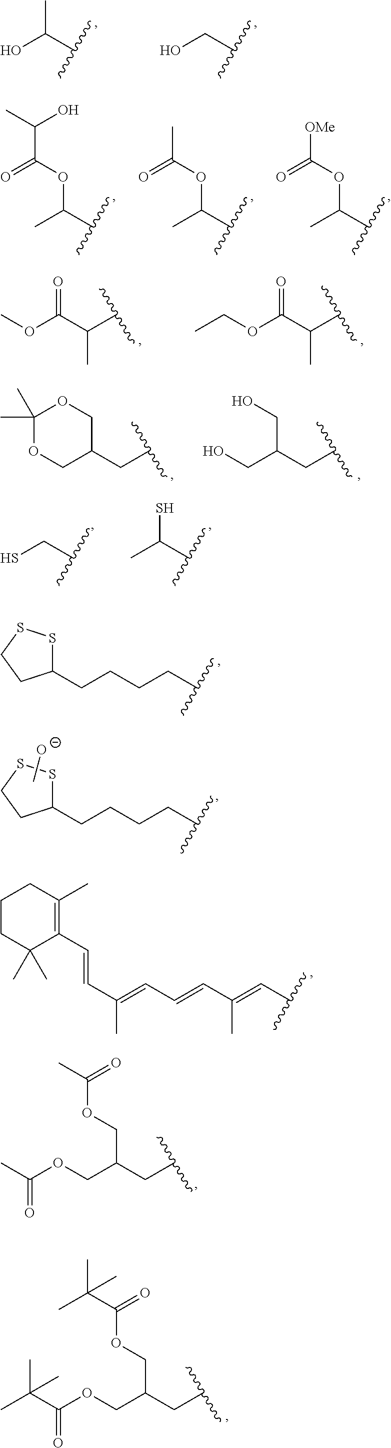

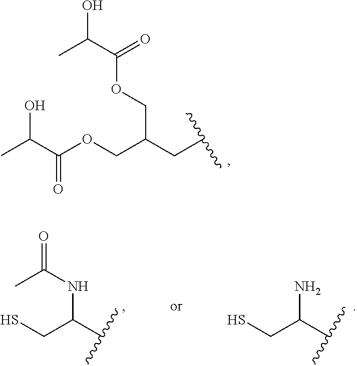

13. The compound of claim 11, wherein R is

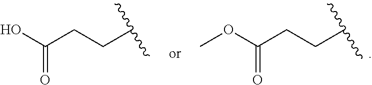

14. The compound of any one of claims 1-7, wherein R is substituted (e.g., linear or branched) heteroalkyl, the (e.g., linear or branched) heteroalkyl being substituted with one or more (heteroalkyl) substituent, each (heteroalkyl) substituent being independently selected from the group consisting of hydroxy, C₁-C₆ alkoxy, thioalkyl, amino, carboxylic acid, C₁-C₆ alkyl, acetamide, thiol, oxo, and optionally substituted (e.g., N-attached) heterocycloalkyl (e.g., optionally substituted with carboxylic acid).

15. The compound of claim 14, wherein R is:

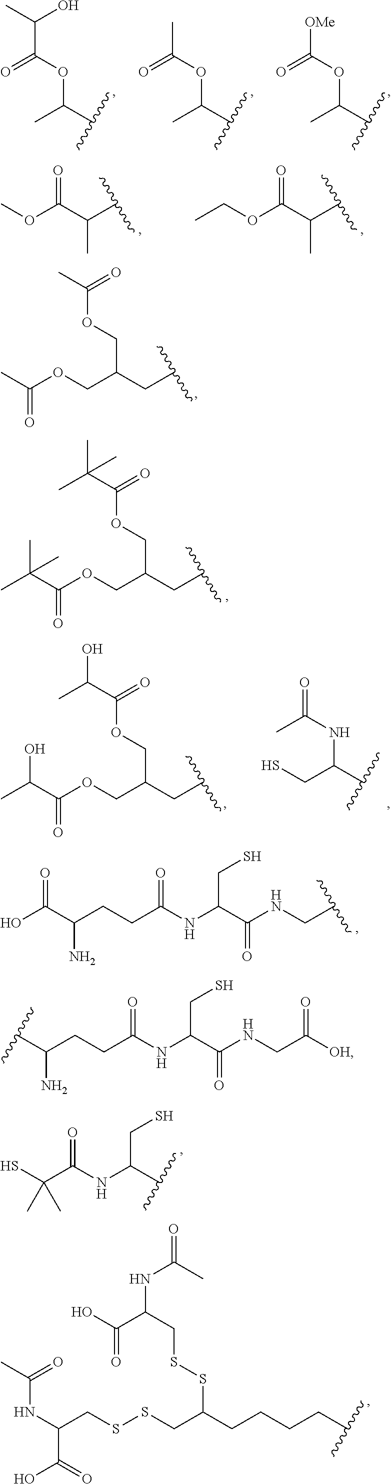

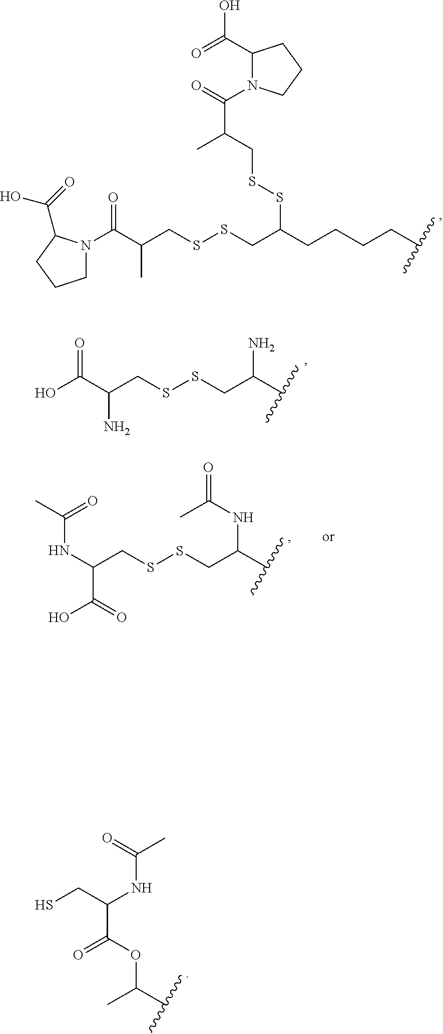 [SMILES: *CCCCC(CSSCC(C)C(=O)N1CCCC1C(=O)O)SSCC(C)C(=O)N1CCCC1C(=O)O ; *C(N)CSSCC(N)C(=O)O ; *C(CSSCC(NC(C)=O)C(=O)O)NC(C)=O ; *C(C)OC(=O)C(CS)NC(C)=O]

16. The compound of claim 14, wherein R is

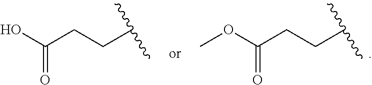

17. The compound of any one of claims 1-7, wherein R is substituted heterocycloalkyl (e.g., N-substituted with alkyl (e.g., further substituted with oxo and/or thiol)).

18. The compound of claim 17, wherein R is:

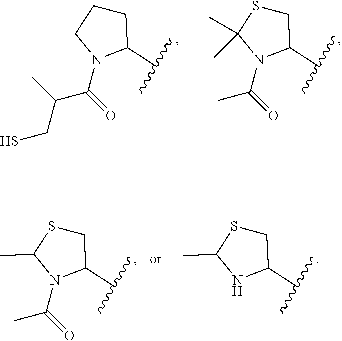

19. The compound of any one of claims 1-7, wherein R is:

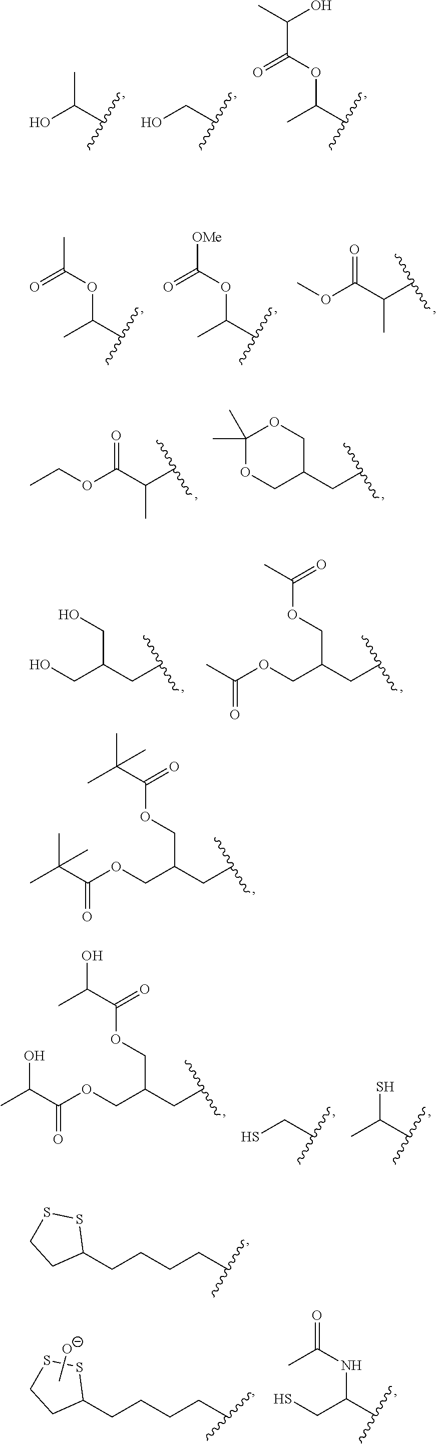

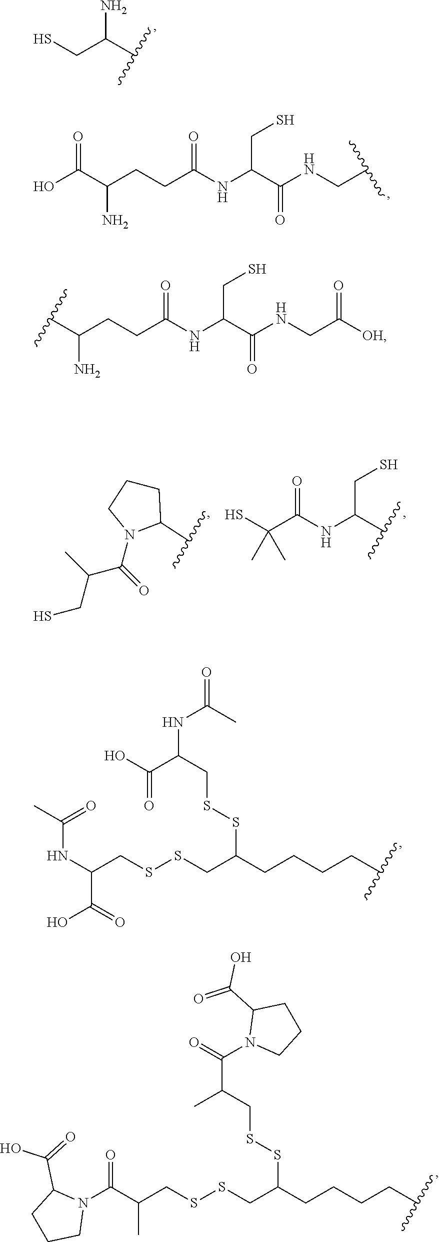

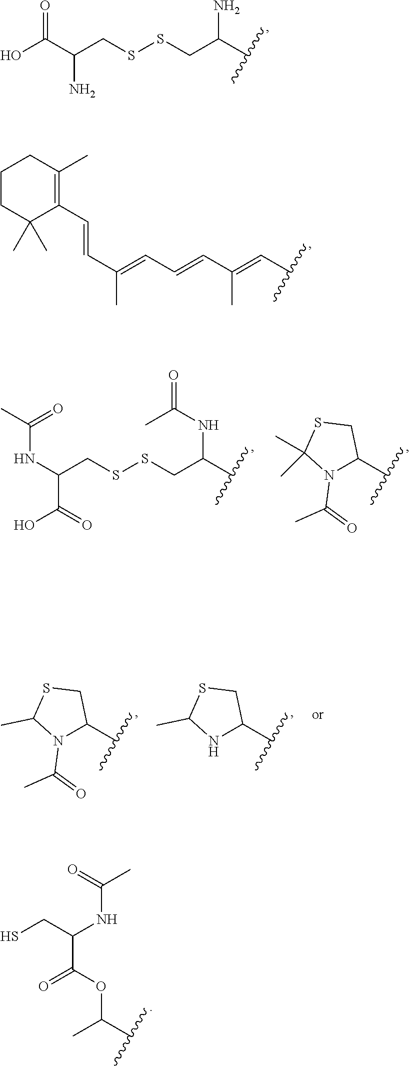

20. The compound of any one of claims 1-7, wherein R is

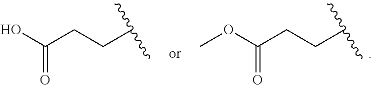

21. The compound of any one of the preceding claims, provided that the compound is other than a compound having the structure:

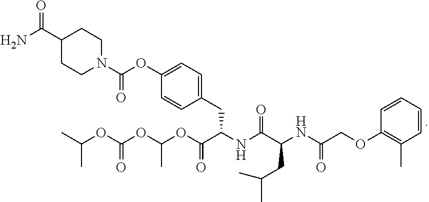 [SMILES: Cc1ccccc1OCC(=O)N[C@@H](CC(C)C)C(=O)N[C@@H](Cc1ccc(OC(=O)N2CCC(C(N)=O)CC2)cc1)C(=O)OC(C)OC(=O)OC(C)C]

22. A compound having the structure of Formula (Ib): 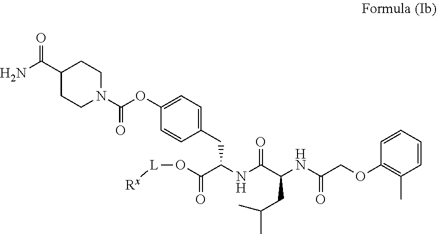
or a pharmaceutically acceptable salt or solvate (e.g., or a stereoisomer) thereof,
wherein:
L is bond, —O(C═O)(OCR⁸R⁹)_{z}—, or —(C═O)(OCR⁸R⁹)_{z}—;
each R⁸ and R⁹ is independently H, halogen, C₁-C₃-alkyl, C₁-C₃-haloalkyl, C₁-C₃-alkoxy, C₃-C₅-cycloalkyl, or R⁸ and R⁹ are taken together with the atoms to which they are attached to form a C₃-C₅-cycloalkyl;
z is 1-6; and
R^{x} is: 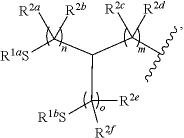
R^{1a} and R^{1b} are each independently —H or —SR^{1c};
each R^{1c} is independently substituted or unsubstituted (e.g., straight or branched) alkyl (e.g., substituted with one or more (alkyl) substituent, each (alkyl) substituent being independently selected from the group consisting of carboxylic acid, —SH, thioalkyl, acetamide, amino, oxo, and optionally substituted heterocycloalkyl (e.g., N-attached pyrrolidinyl substituted with —COOH)), or substituted or unsubstituted (e.g., straight or branched) heteroalkyl (e.g., substituted with one or more (heteroalkyl) substituent, each (heteroalkyl) substituent being independently selected from the group consisting of carboxylic acid, amino, thioalkyl, thiol, acetamide, and C₁-C₃ alkyl);
each R^{2a}, R^{2b}, R^{2c}, R^{2d}, R^{2e}, and R^{2f} is independently H, halogen, C₁-C₃-alkyl, C₁-C₃-haloalkyl, C₁-C₃-alkoxy, C₃-C₅-cycloalkyl, or any one of R^{2a} and R^{2b}, R^{2c} and R^{2d}, or R^{2e} and R^{2f} are taken together with the atoms to which they are attached to form a C₃-C₅-cycloalkyl;
m is an integer from 1-10; and
n and o are each independently an integer from 0-3.

23. The compound of claim 22, wherein each R^{2a}, R^{2b}, R^{2c}, R^{2d}, R^{2e}, and R^{2f} is independently H, halogen, C₁-C₃alkyl, or C₁-C₃haloalkyl.

24. The compound of any one of claims 22-23, wherein: each R^{2a}, R^{2b}, R^{2c}, R^{2d}, R^{2e}, and R^{2f} is H.

25. The compound of any one of claims 22-24, wherein: o is 0, and R^{x} is:

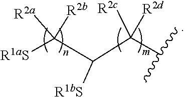

26. The compound of any one of claims 22-25, wherein: o is 0, n is 1, and R^{x} is:

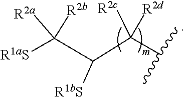

27. The compound of any one of claims 22-26, wherein m is an integer from 3-5.

28. The compound of any one of claims 22-27, wherein R^{x} is: 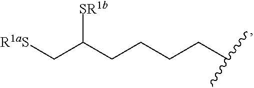
wherein:
R^{1a} and R^{1b} are each independently —H or —SR^{1c}, and
each R^{1c} is independently substituted or unsubstituted (e.g., straight or branched) alkyl (e.g., substituted with one or more (alkyl) substituent, each (alkyl) substituent being independently selected from the group consisting of carboxylic acid, —SH, thioalkyl, acetamide, amino, oxo, and optionally substituted heterocycloalkyl (e.g., N-attached pyrrolidinyl substituted with —COOH)), or substituted or unsubstituted (e.g., straight or branched) heteroalkyl (e.g., substituted with one or more (heteroalkyl) substituent, each (heteroalkyl) substituent being independently selected from the group consisting of carboxylic acid, amino, thioalkyl, thiol, acetamide, and C₁-C₃ alkyl).

29. The compound of any one of claims 22-28, wherein: R^{1a} is —H or SR^{1c} and R^{1b} is —SR^{1c}, or R^{1a} is —SR^{1c} and R^{1b} is —H or SR^{1c}.

30. The compound of any one of claims 22-29, wherein R^{1a} and R^{1b} are each —SR^{1c}.

31. The compound of claim 30, wherein R^{x} is: 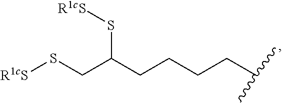
wherein:
each R^{1c} is independently substituted or unsubstituted (e.g., straight or branched) alkyl (e.g., substituted with one or more (alkyl) substituent, each (alkyl) substituent being independently selected from the group consisting of carboxylic acid, —SH, thioalkyl, acetamide, amino, oxo, and optionally substituted heterocycloalkyl (e.g., N-attached pyrrolidinyl substituted with —COOH)), or substituted or unsubstituted (e.g., straight or branched) heteroalkyl (e.g., substituted with one or more (heteroalkyl) substituent, each (heteroalkyl) substituent being independently selected from the group consisting of carboxylic acid, amino, thioalkyl, thiol, acetamide, and C₁-C₃ alkyl).

32. The compound of any one of claims 22-31, wherein R^{1a} and R^{2b} are each independently a radical of one or more keratolytic group (e.g., each radical of the one or more keratolytic group being independently selected from the group consisting of a radical of glycolic acid (GA), a radical of thioglycolic acid (TGA), a radical of lactic acid (Lac), a radical of thiolactic acid (TLac), a radical of lipoic acid (Lip), a radical of lipoic acid sulfoxide (Lipox), a radical of dihydrolipoic acid (diHLip), a radical of N-acetyl cysteine (NAC), a radical of cysteine (Cys), a radical of glutathione (GSH), a radical of captopril (Cap), and a radical of bucillamine (Buc)).

33. The compound of any one of claims 22-32, wherein R^{1a} and R^{2b} are each independently a (thiol) radical of one or more keratolytic group, each (thiol) radical of the one or more keratolytic group being independently selected from the group consisting of a (thiol) radical of thioglycolic acid (TGA), a (thiol) radical of thiolactic acid (TLac), a (thiol) radical of dihydrolipoic acid (diHLip), a (thiol) radical of N-acetyl cysteine (NAC), a (thiol) radical of cysteine (Cys), a (thiol) radical of glutathione (GSH), a (thiol) radical of captopril (Cap), and a (thiol) radical of bucillamine (Buc).

34. The compound of claim 32 or 33, wherein the radical comprises [Lac-Lac]·, [Lac-NAC]·, [Cys-Cys]·, [diHLip-NAC-NAC]·, [diHLip-NAC]·, [diHLip-Cap-Cap]·, [diHLip-Cap]·, [diHLip-Cys-Cys]·, [diHLip-Cys]·, [diHLip-Lipox-Lipox]·, [diHLip-Lipox]·, or any combination thereof.

35. The compound of any one of claims 22-34, R^{1b} and R^{2b} are each independently —H or:

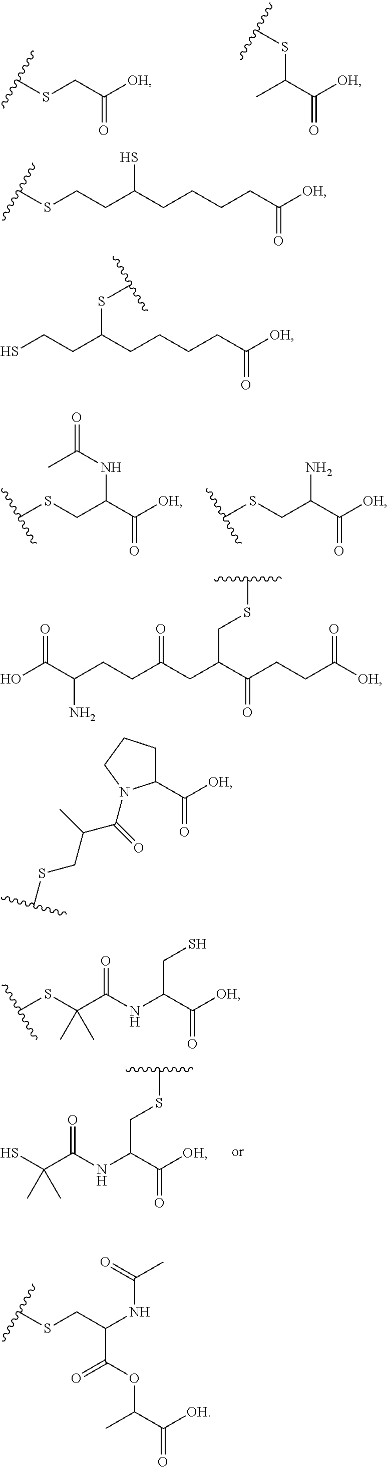

36. The compound of any one of claims 22-35, wherein R^{x} is:

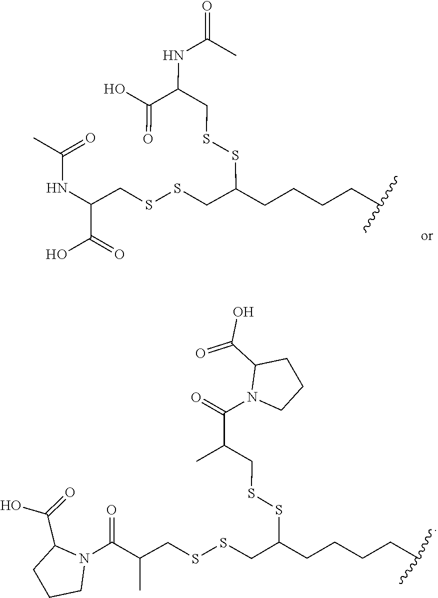 [SMILES: *CCCCC(CSSCC(NC(C)=O)C(=O)O)SSCC(NC(C)=O)C(=O)O ; *CCCCC(CSSCC(C)C(=O)N1CCCC1C(=O)O)SSCC(C)C(=O)N1CCCC1C(=O)O]

37. A compound having the structure of Formula (Ic): 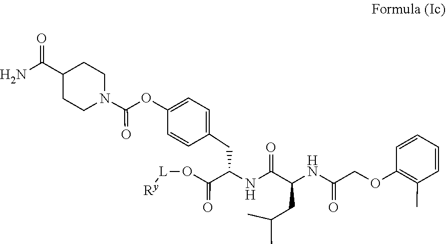
or a pharmaceutically acceptable salt or solvate (e.g., or a stereoisomer) thereof, wherein:
L is bond, —O(C═O)(OCR⁸R⁹)_{z}—, or —(C═O)(OCR⁸R⁹)_{z}—;
each R⁸ and R⁹ is independently H, halogen, C₁-C₃-alkyl, C₁-C₃-haloalkyl, C₁-C₃-alkoxy, C₃-C₅-cycloalkyl, or R⁸ and R⁹ are taken together with the atoms to which they are attached to form a C₃-C₅-cycloalkyl;
z is 1-6; and
R^{y} is: 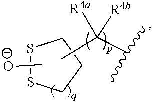
each R^{4a} and R^{4b} is independently H, halogen, or substituted or unsubstituted alkyl;
p is an integer from 1-10; and
q is an integer from 1-3.

38. The compound of claim 37, wherein q is 1.

39. The compound of any one of claims 37-38, wherein q is 1 and p is an integer from 3-5.

40. The compound of any one of claims 37-39, wherein each R^{4a} and R^{4b} is independently H, halogen, C₁-C₃alkyl, or C₁-C₃haloalkyl.

41. The compound of any one of claims 37-40, wherein each R^{4a} and R^{4b} is H.

42. The compound of any one of claims 37-41, wherein R^{y} is:

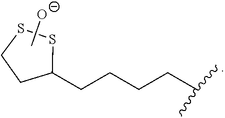

43. A compound having the structure of Formula (Id): 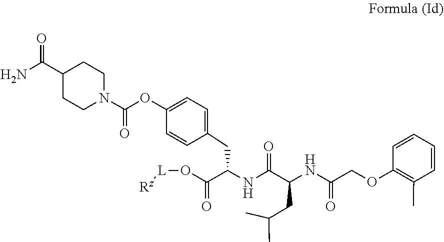
or a pharmaceutically acceptable salt or solvate (e.g., or a stereoisomer) thereof, wherein:
L is bond, —O(C═O)(OCR⁸R⁹)_{z}—, or —(C═O)(OCR⁸R⁹)_{z}—;
each R⁸ and R⁹ is independently H, halogen, C₁-C₃-alkyl, C₁-C₃-haloalkyl, C₁-C₃-alkoxy, C₃-C₅-cycloalkyl, or R⁸ and R⁹ are taken together with the atoms to which they are attached to form a C₃-C₅-cycloalkyl;
z is 1-6; and
R^{z} is: 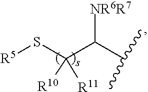
R⁵ is —SR^{1c};
R^{1c} is substituted or unsubstituted (e.g., straight or branched) alkyl (e.g., substituted with one or more (alkyl) substituent, each (alkyl) substituent being independently selected from the group consisting of carboxylic acid, —SH, thioalkyl, acetamide, amino, oxo, and optionally substituted heterocycloalkyl (e.g., N-attached pyrrolidinyl substituted with —COOH)), or substituted or unsubstituted (e.g., straight or branched) heteroalkyl (e.g., substituted with one or more (heteroalkyl) substituent, each (heteroalkyl) substituent being independently selected from the group consisting of carboxylic acid, amino, thioalkyl, thiol, acetamide, and C₁-C₃ alkyl);
R⁶ and R⁷ are each independently H, substituted or unsubstituted alkyl, or substituted or unsubstituted heteroalkyl;
each R¹⁰ and R¹¹ is independently H, halogen, C₁-C₃-alkyl, C₁-C₃-haloalkyl, C₁-C₃-alkoxy, C₃-C₅-cycloalkyl, or two of R¹⁰ and R¹¹ are taken together with the atoms to which they are attached to form a C₃-C₅-cycloalkyl; and
s is an integer from 1-10.

44. The compound of claim 43, wherein R⁶ and R⁷ are each independently H or substituted or unsubstituted alkyl (e.g., C₁-C₃ alkyl optionally substituted with oxo).

45. The compound of any one of claims 43-44, wherein R⁶ and R⁷ are H.

46. The compound of any one of claims 43-45, wherein each R¹⁰ and R¹¹ are independently H, halogen, C₁-C₃alkyl, or C₁-C₃haloalkyl.

47. The compound of any one of claims 43-46, wherein each R¹⁰ and R¹¹ are H.

48. The compound of any one of claims 43-47, wherein s is 1-3.

49. The compound of any one of claims 43-48, wherein R⁵ is:

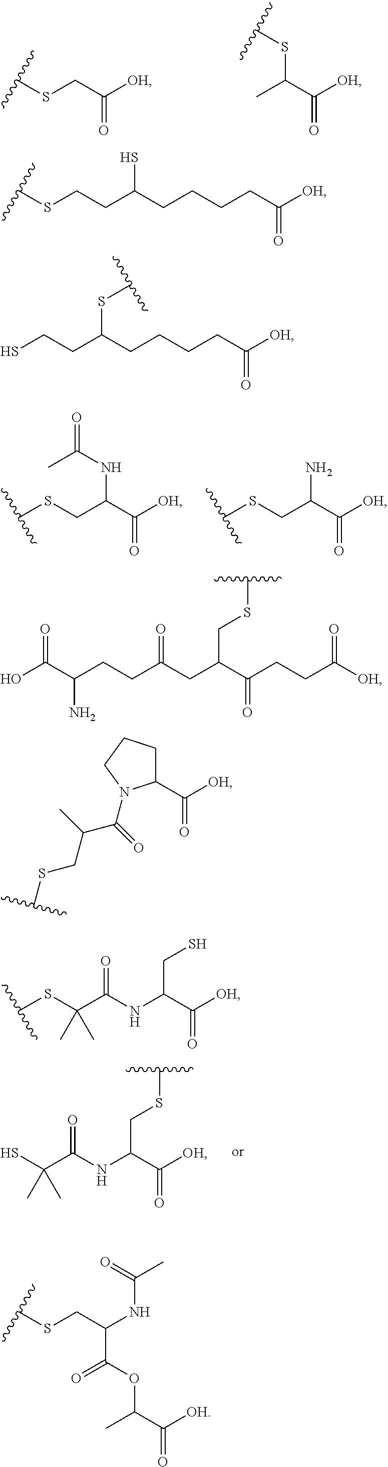

50. A compound having the structure of Formula (II): 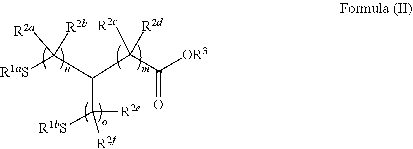
or a pharmaceutically acceptable salt or solvate (e.g., or a stereoisomer) thereof,
wherein:
R^{1a} and R^{1b} are each independently —H or —SR^{1c}, either or both of R^{1a} and R^{1b} being —SR^{1c};
each R^{1c} is independently substituted or unsubstituted (e.g., straight or branched) alkyl (e.g., substituted with one or more (alkyl) substituent, each (alkyl) substituent being independently selected from the group consisting of carboxylic acid, —SH, thioalkyl, acetamide, amino, oxo, and optionally substituted heterocycloalkyl (e.g., N-attached pyrrolidinyl substituted with —COOH)), or substituted or unsubstituted (e.g., straight or branched) heteroalkyl (e.g., substituted with one or more (heteroalkyl) substituent, each (heteroalkyl) substituent being independently selected from the group consisting of carboxylic acid, amino, thioalkyl, thiol, acetamide, and C₁-C₃ alkyl);
each R^{2a}, R^{2b}, R^{2c}, R^{2d}, R^{2e}, and R^{2f} is independently H, halogen, C₁-C₃-alkyl, C₁-C₃-haloalkyl, C₁-C₃-alkoxy, C₃-C₅-cycloalkyl, or two of R^{2a} and R^{2b}, R^{2c} and R^{2d}, or R^{2e} and R^{2f} are taken together with the atoms to which they are attached to form a C₃-C₅-cycloalkyl;
R³ is H or substituted or unsubstituted alkyl;
m is an integer from 1-10; and
n and o are each independently an integer from 0-3.

51. The compound of claim 50, wherein R^{1a} and R^{2b} are each independently H or:

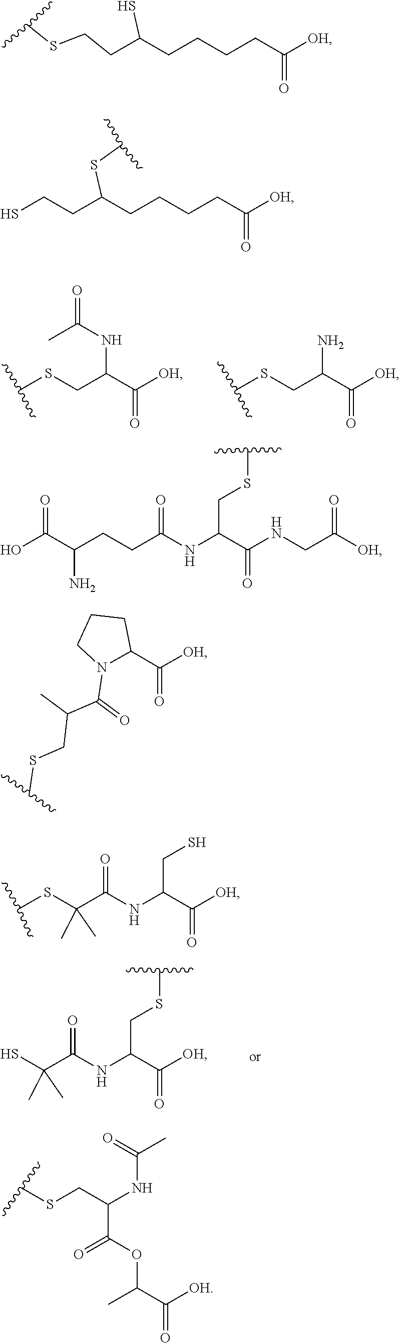

52. The compound of any one of claims 50-51, wherein the compound is: 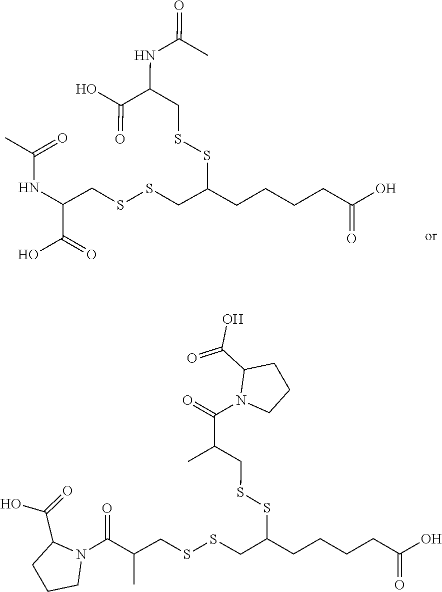 [SMILES: CC(=O)NC(CSSCC(CCCCC(=O)O)SSCC(NC(C)=O)C(=O)O)C(=O)O ; CC(CSSCC(CCCCC(=O)O)SSCC(C)C(=O)N1CCCC1C(=O)O)C(=O)N1CCCC1C(=O)O]
or any stereoisomer form thereof or a pharmaceutically acceptable salt, ester, or solvate of any of the foregoing.

53. A compound having the structure of Formula (III): 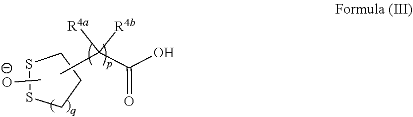
or a pharmaceutically acceptable salt or solvate (e.g., or a stereoisomer) thereof, wherein,
each R^{4a} and R^{4b} is independently H, halogen, or substituted or unsubstituted alkyl;
p is an integer from 1-10; and
q is an integer from 1-3.

54. The compound of claim 53, wherein the compound is: 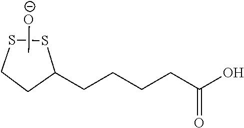
or any stereoisomer form thereof or a pharmaceutically acceptable salt or solvate of any of the foregoing.
